(19) United States
(12) Patent Application Publication
(10) Pub. No.: US 2025/0090651 A1
(43) Pub. Date: Mar. 20, 2025

(54) RESPIRATORY SYNCYTIAL VIRUS F PROTEINS AND NANOSTRUCTURES AND USES THEREOF

(71) Applicant: Icosavax, Inc., Seattle, WA (US)

(72) Inventors: Daniel ELLIS, Seattle, WA (US); Quinton DOWLING, Seattle, WA (US)

(21) Appl. No.: 18/885,333

(22) Filed: Sep. 13, 2024

Related U.S. Application Data
(60) Provisional application No. 63/583,097, filed on Sep. 15, 2023.

(51) Int. Cl. A61K 39/155; A61K 47/54; C07K 7/08; C12N 5/00; C12N 15/62

(52) U.S. Cl. CPC A61K 39/155; A61K 47/543; C07K 7/08; C12N 5/0068; C12N 15/62; C07K 2319/35

(57) ABSTRACT

Provided herein are recombinant polypeptides comprising an engineered ectodomain of a Respiratory Syncytial Virus (RSV) fusion (F) protein, wherein the ectodomain comprises an engineered C-terminal alpha-helical segment and/or amino acid substitutions that stabilize the F protein in a prefusion conformation. The disclosure also provides a two-component protein nanostructure comprising first trimeric component and second pentameric component. Provided herein are a composition for use in vaccinating, generating an immune response, or treating or preventing RSV disease.

### CROSS-REFERENCE TO RELATED APPLICATION

This application claims the benefit of U.S. Provisional Application No. 63/583,097, filed Sep. 15, 2023, the contents of which is incorporated by reference herein in its entirety.

### INCORPORATION BY REFERENCE OF SEQUENCE LISTING

The instant application contains a Sequence Listing which has been submitted electronically in XML format and is hereby incorporated by reference in its entirety. Said XML copy, created on Sep. 6, 2024, is named 061291-517001WO.xml and is 340 KB in size.

### BACKGROUND

When an enveloped virus encounters a target cell, its viral membrane fusion protein undergoes a conformational change that drives fusion of the viral envelope with the target cell's cell membrane. This fusion process delivers the viral genome into the target cell. For many enveloped viruses, the adaptive immune response to the viral membrane fusion protein is a key source of protective immunity, in part because neutralizing antibodies may inhibit this fusion process. Hence, vaccines for enveloped viruses often include a viral membrane fusion protein as an antigen.

Structural information about viral membrane fusion proteins has structure-based design of recombinant antigens for use in vaccines. See Graham et al. Annu Rev Med. 70:91-104 (2019). For example, WO 2014/160463 A1 describes stabilization of the fusion (F) protein of Respiratory Syncytial Virus (RSV) in its prefusion conformation by introducing into the F protein the amino acid substitutions S155C, S290C, S190F, and V207L (collectively termed “DS-Cav1”). Stabilization of the RSV F protein in a prefusion conformation improves the immune response to the protein. Because RSV F protein is trimeric in their prefusion conformation, another approach is to C-terminally fuse a trimerization domain to the engineered ectodomain of an RSV F protein. Another technology used in structure-based vaccine design is display of an engineered ectodomain of an RSV F protein on a protein nanostructure. WO 2018/187325 A1 describes a two-component computationally designed protein structure that self-assembles to display variant DS-Cav1 RSV F protein or other antigens.

There is an unmet need for viral membrane fusion proteins stabilized by designed amino acid substitutions. The present disclosure provides recombinant polypeptides and related compositions and methods that address this need for Respiratory Syncytial Virus (RSV).

### SUMMARY

The invention relates generally to recombinant polypeptides comprising an ectodomain of a viral membrane fusion (F) protein having (a) an engineered C-terminal alpha-helical segment; (b) amino acid substitutions that stabilize the F protein in a prefusion conformation; or combinations of (a) and (b). Advantageously, the disclosed modifications to the F protein ectodomain may improve thermal stability, conformational stability, antigenicity, and/or immunogenicity compared to reference proteins lacking these modifications. Further provided are trimeric protein complexes and self-assembling protein nanostructures that include such RSV F ectodomain polypeptides, as well as various other compositions, methods, and uses, including as vaccines. In another aspect, the disclosure provides a nanostructure comprising a trimeric component comprising a helix-forming segment as disclosed herein. In another aspect, the disclosure provides helix-forming segments as disclosed herein.

In one aspect, the disclosure provides a recombinant polypeptide, comprising an engineered ectodomain of a Respiratory Syncytial Virus (RSV) fusion (F) protein, wherein the ectodomain comprises (a) a C-terminal helix-forming segment, between about residue 500 and about residue 530 relative to SEQ ID NO: 1, comprising one or more amino acid substitutions selected such that the segment forms a stable alpha-helical homotrimer.

In another aspect, the disclosure provides a recombinant polypeptide, comprising an engineered ectodomain of a Respiratory Syncytial Virus (RSV) fusion (F) protein, wherein the ectodomain comprises (b) one, two, three or more amino acid substitutions at positions 140, 399, 400, 485, 486, 487, 488, 489, 494, or 498 relative to SEQ ID NO: 1.

In another aspect, the disclosure provides a recombinant polypeptide, comprising an engineered ectodomain of a Respiratory Syncytial Virus (RSV) fusion (F) protein, wherein the ectodomain comprises (c) one, two, three or more amino acid substitutions at positions 56, 58, 154, 187, 296, or 298 relative to SEQ ID NO: 1.

In another aspect, the disclosure provides a recombinant polypeptide, comprising an engineered ectodomain of a Respiratory Syncytial Virus (RSV) fusion (F) protein, wherein the ectodomain comprises (d) one, two, three or more amino acid substitutions at positions 75, 216, 218, or 219 relative to SEQ ID NO: 1.

In another aspect, the disclosure provides a recombinant polypeptide, comprising an engineered ectodomain of a Respiratory Syncytial Virus (RSV) fusion (F) protein, wherein the ectodomain comprises (e) one, two, three or more amino acid substitutions at positions 92, 232, 235, 238, 249, 250, or 254 relative to SEQ ID NO: 1. In another aspect, the disclosure provides a recombinant polypeptide, comprising an engineered ectodomain of a Respiratory Syncytial Virus (RSV) fusion (F) protein, wherein the ectodomain comprises (f) one, two, three or more amino acid substitutions at positions 67, 137, or 339 relative to SEQ ID NO: 1.

In another aspect, the disclosure provides a recombinant polypeptide, comprising an engineered ectodomain of a Respiratory Syncytial Virus (RSV) fusion (F) protein, wherein the ectodomain comprises (g) a substitution of a non-cleavable linker in place of a furin cleavage site at about residues 100 to about residue 140 relative to SEQ ID NO: 1.

In another aspect, the disclosure provides a recombinant polypeptide, comprising an engineered ectodomain of a Respiratory Syncytial Virus (RSV) fusion (F) protein, wherein the ectodomain comprises (h) any combination of (a)-(g).

In some embodiments, the ectodomain comprises (a) the C-terminal helix-forming segment, between about residue 500 and about residue 530 relative to SEQ ID NO: 1, comprising one or more amino acid substitutions selected such that the segment forms a stable alpha-helical homotrimer.

In some embodiments, the C-terminal helix-forming segment comprises between about 10 and about 30 residues.

In some embodiments, the segment comprises substitutions relative to the reference sequence SEQ ID NO: 1 at two or more, three or more, or four or more residues that generate hydrophobic contacts between the segments in the alpha-helical homotrimer.

In some embodiments, the segment comprises (a) an amino acid substitution at position F505 relative to SEQ ID NO: 1, wherein F is substituted with A, I, L, M, V, G, T; (b) an amino acid substitution at position I506 relative to SEQ ID NO: 1, wherein I is substituted with any amino acids except P, preferably D, E, K, N, Q, R, S, T, Y or A, I, L, V; (c) an amino acid substitution at position R507 relative to SEQ ID NO: 1, wherein R is substituted with any amino acids except P, preferably D, E, K, N, Q, R, S, T, Y or A, I, L, V; (d) an amino acid substitution at position K508 relative to SEQ ID NO: 1, wherein K is substituted with K, Q, R, preferably A, V, T, I; (e) an amino acid substitution at position S509 relative to SEQ ID NO: 1, wherein S is substituted with A, I, L, M, V, F, W, Y, G, T, preferably A, I, L, M, V; (f) an amino acid substitution at position D510 relative to SEQ ID NO: 1, wherein D is substituted with any amino acids, preferably D, E, K, N, Q, R, S, T, Y; (g) an amino acid substitution at position E511 relative to SEQ ID NO: 1, wherein E is substituted with any amino acids; (h) an amino acid substitution at position L512 relative to SEQ ID NO: 1, wherein L is substituted with D, E, K, N, Q, R, S, T, Y, preferably A, I, L, M, V, F, W, Y, G, T; (i) an amino acid substitution at position L513 relative to SEQ ID NO: 1, wherein L is substituted with any amino acids, preferably A, I, L, M, V, F, W, Y, G, more preferably D, E, K, N, Q, R, S, T, Y; (j) an amino acid substitution at position H514 relative to SEQ ID NO: 1, wherein H is substituted with any amino acids except P, preferably D, E, K, N, Q, R, S, T, Y; (k) an amino acid substitution at position N515 relative to SEQ ID NO: 1, wherein N is substituted with any amino acids except P, preferably A, I, L, M, V, F, W, Y, G; (l) an amino acid substitution at position V516 relative to SEQ ID NO: 1, wherein V is substituted with A, I, L, M, V, F, W, Y, G, or T, S, K; (m) an amino acid substitution at position N517 relative to SEQ ID NO: 1, wherein N is substituted with any amino acid except P, preferably D, E, K, N, Q, R, S, T, Y; (n) an amino acid substitution at position T518 relative to SEQ ID NO: 1, wherein T is substituted with Any except P, preferably D, E, K, N, Q, R, S, T, Y; (o) an amino acid substitution at position G519 relative to SEQ ID NO: 1, wherein G is substituted with any amino acid except P, preferably D, E, K, N, Q, R, S, T, Y; and/or (p) any combination of (a)-(o).

In some embodiments, the segment comprises (a) an amino acid substitution at position L503 relative to SEQ ID NO: 1, wherein L is substituted with Q, V, K, R, N, L; (b) an amino acid substitution at position A504 relative to SEQ ID NO: 1, wherein A is substituted with any amino acids except P, preferably S, T, L, A, Q, K, E, Y; (c) an amino acid substitution at position F505 relative to SEQ ID NO: 1, wherein F is substituted with I, V, N, T, L; (d) an amino acid substitution at position I506 relative to SEQ ID NO: 1, wherein I is substituted with any amino acids except P, preferably Q, N, K, R, V, S; (e) an amino acid substitution at position R507 relative to SEQ ID NO: 1, wherein R is substituted with any amino acids except P, preferably A, N, K, E, D, Q; (f) an amino acid substitution at position K508 relative to SEQ ID NO: 1, wherein K is substituted with T, M, V, R; (g) an amino acid substitution at position S509 relative to SEQ ID NO: 1, wherein S is substituted with T, I, K, Q, M, E, V, S; (h) an amino acid substitution at position D510 relative to SEQ ID NO: 1, wherein D is substituted with S, K, N, D, E; (i) an amino acid substitution at position E511 relative to SEQ ID NO: 1, wherein E is substituted with R, S, E, K, A, T, L; (j) an amino acid substitution at position L512 relative to SEQ ID NO: 1, wherein L is substituted with V, N, T, L; (k) an amino acid substitution at position L513 relative to SEQ ID NO: 1, wherein L is substituted with D, T, H, K, E, N, R; (l) an amino acid substitution at position H514 relative to SEQ ID NO: 1, wherein H is substituted with A, N, E, S, V, K, T, D; (m) an amino acid substitution at position N515 relative to SEQ ID NO: 1, wherein N is substituted with I, E, L, T, Q; (n) an amino acid substitution at position V516 relative to SEQ ID NO: 1, wherein V is substituted with E, I, K, N, R, Q; o) an amino acid substitution at position N517 relative to SEQ ID NO: 1, wherein N is substituted with A, S, K, E, R; (p) an amino acid substitution at position T518 relative to SEQ ID NO: 1, wherein T is substituted with K, S, Q, R, D, E; (q) an amino acid substitution at position G519 relative to SEQ ID NO: 1, wherein G is substituted with V, L, I; (r) an amino acid substitution at position 1520 relative to SEQ ID NO: 1, wherein I is substituted with K, Q, E, N, T; (s) an amino acid substitution at position P521 relative to SEQ ID NO: 1, wherein P is substituted with H, D, E, K, R, N, Q; (t) an amino acid substitution at position E522 relative to SEQ ID NO: 1, wherein E is substituted with L, R, I, V; (u) an amino acid substitution at position A523 relative to SEQ ID NO: 1, wherein A is substituted with E, V, L, K, R I; (v) an amino acid substitution at position P524 relative to SEQ ID NO: 1, wherein P is substituted with A, K, T, E, R; (w) an amino acid substitution at position R525 relative to SEQ ID NO: 1, wherein R is substituted with H, R, S, L, N, E, D; (x) an amino acid substitution at position D526 relative to SEQ ID NO: 1, wherein D is substituted with I, L, V, R; (y) an amino acid substitution at position G527 relative to SEQ ID NO: 1, wherein G is substituted with E, K, Q, D; (z) an amino acid substitution at position Q528 relative to SEQ ID NO: 1, wherein Q is substituted with D, K, S, R, A; (aa) an amino acid substitution at position A529 relative to SEQ ID NO: 1, wherein A is substituted with T, L; (ab) an amino acid substitution at position Y530 relative to SEQ ID NO: 1, wherein Y is substituted with L, E, T; (ac) an amino acid substitution at position V531 relative to SEQ ID NO: 1, wherein V is substituted with A, R, K; (ad) an amino acid substitution at position R532 relative to SEQ ID NO: 1, wherein R is substituted with V, A; and/or (ae) any combination of (a)-(ad).

In some embodiments, the segment comprises a polypeptide sequence listed in Table 2A or Table 2B, or a polypeptide sequence having between 1 and 5 amino acid substitutions thereto.

In some embodiments, the segment comprises the polypeptide sequence NQSREIIRAINIVRKIASEK (SEQ ID NO: 10), NQSALWLEAAKYVKQAREKS (SEQ ID NO: 11), NQSAKNAEAAKIAEETKRKD (SEQ ID NO: 12), or NQSRETAKAVSAVK (SEQ ID NO: 75), or a polypeptide sequence having between 1 and 5 amino acid substitutions thereto.

In some embodiments, the segment comprises the polypeptide sequence NQSREIIRAINIVRKIASEK (SEQ ID NO: 10), or a polypeptide sequence having between 1 and 5 amino acid substitutions thereto.

In some embodiments, the segment comprises the polypeptide sequence

(SEQ ID NO: 10)
NQSREIIRAINIVRKIASEK.

In some embodiments, the ectodomain comprises (b) one, two, three or more amino acid substitutions at positions 140, 399, 400, 485, 486, 487, 488, 489, 494, or 498 relative to SEQ ID NO: 1.

In some embodiments, the ectodomain comprises one or more of the following sets of amino acid substitutions relative to SEQ ID NO: 1: E487R+K498A; E487R+K498E; E487K+K498E; D486A+E487R+K498A; D486Q+E487R+K498A; D486E+E487A+D489A+T400D; D486A+E487M+K498A; E487Q; D486S; F488W+D489A+T400D+E487R+K498A; F140W+D489A+T400D+E487R+K498A; Q494I+S485I+K399A+487R+498A; Q494M+S485I+K399A; D486A+487M+498A; Q494L+S485A+K399V +D486A+487M+498A; Q494M+S485A+K399V+D486A+487M+498A; Q494A+S485F+K399V+D486A+487M+498Y; D489A+T400D+E487R+K498A; or D489A+T400D.

In some embodiments, the ectodomain comprises the amino acid substitutions F488W, D489A, T400D, E487R, K498A, and D486A.

In some embodiments, the ectodomain comprises the amino acid substitutions F488W, D489A, T400D, E487R, K498A, and T249P.

In some embodiments, the polypeptide comprises, C-terminal to the ectodomain, a heterologous multimerization domain.

In some embodiments, the multimerization domain is a trimerization domain.

In some embodiments, the multimerization domain comprises a polypeptide sequence at least 70%, at least 80%, at least 90%, at least 91%, at least 92%, at least 93%, at least 94%, at least 95%, at least 96%, at least 97%, at least 98%, at least 99%, or 100% identical to I53-50A (SEQ ID NO: 144) or I53-50A ΔCys (SEQ ID NO: 145).

In some embodiments, the ectodomain comprises the amino acid substitutions S155C, S290C, S190F, and V207L.

In some embodiments, the ectodomain comprises a polypeptide sequence at least 70%, at least 80%, at least 90%, at least 91%, at least 92%, at least 93%, at least 94%, at least 95%, at least 96%, at least 97%, at least 98%, at least 99%, or 100% identical to SEQ ID NO: 6, below, optionally lacking a p27 peptide shown in bold, and in which “X” refers to sites involving an added C-terminal helical segment and can be any amino acid:

(SEQ ID NO: 6)
QNITEEFYQSTCSAVSRGYLSALRTGWYTSVITIELSNIKETKCNGTDTKVKLIKQELDKYK
NAVTELQLLMQNTPAVNNRARREAPQYMNYTINTTKNLNVSISKKRKRRFLGFLLGVGSAIA
SGIAVCKVLHLEGEVNKIKNALQLTNKAVVSLSNGVSVLTFRVLDLKNYINNQLLPMLNRQS
CRISNIETVIEFQQKNSRLLEITREFSVNAGVTTPLSTYMLINSELLSLINDMPITNDQKKL
MSSNVQIVRQQSYSIMCIIKEEVLAYVVQLPIYGVIDTPCWKLHTSPLCTTNIKEGSNICLT
RTDRGWYCDNAGSVSFFPQADTCKVQSNRVFCDTMNSLTLPSEVSLCNTDIFNSKYDCKIMT
SKTDISSSVITSLGAIVSCYGKTKCTASNKNRGIIKTFSNGCDYVSNKGVDTVSVGNTLYYV
NKLEGKNLYVKGEPIINYYDPLVFPSDEFDASISQVNEKINQSXXXXXXXXXXXXXXXXX

In some embodiments, the ectodomain comprises a polypeptide sequence at least 70%, at least 80%, at least 90%, at least 91%, at least 92%, at least 93%, at least 94%, at least 95%, at least 96%, at least 97%, at least 98%, at least 99%, or 100% identical to SEQ ID NO: 7, below, optionally lacking a p27 peptide shown in bold, in which “X” refers to sites involving an added C-terminal helical segment and can be any amino acid:

(SEQ ID NO: 7)
QNITEEFYQSTCSAVSKGYLSALRTGWYTSVITIELSNIKENKCNGTDAKVKLIKQELDKYK
NAVTELQLLMQSTPAINNRARRELPREMNYTLNNAKKTNVTLSKKRKRRFLGFLLGVGSAIA
SGVAVCKVLHLEGEVNKIKSALLSTNKAVVSLSNGVSVLTFKVLDLKNYIDKQLLPILNKQS
CSISNIETVIEFQQKNNRLLEITREFSVNAGVTTPVSTYMLINSELLSLINDMPITNDQKKL
MSNNVQIVRQQSYSIMCIIKEEVLAYVVQLPLYGVIDTPCWKLHTSPLCTTNTKEGSNICLT
RTDRGWYCDNAGSVSFFPQAETCKVQSNRVFCDTMNSLTLPSEVNLCNVDIFNPKYDCKIMT
SKTDVSSSVITSLGAIVSCYGKTKCTASNKNRGIIKTFSNGCDYVSNKGVDTVSVGNTLYYV
NKQEGKSLYVKGEPIINFYDPLVFPSDEFDASISQVNEKINQSXXXXXXXXXXXXXXXXX

In some embodiments, the ectodomain comprises a polypeptide sequence at least 70%, at least 80%, at least 90%, at least 91%, at least 92%, at least 93%, at least 94%, at least 95%, at least 96%, at least 97%, at least 98%, at least 99%, or 100% identical to SEQ ID NO: 8, below, optionally lacking a p27 peptide shown in bold:

(SEQ ID NO: 8)
QNITEEFYQSTCSAVSRGYLSALRIGWYTSVITIELSNIKETKCNGTDTKVKLIKQELDKYK
NAVTELQLLMQNTPAVNNRARREAPQYMNYTINTTKNLNVSISKKRKRRFLGFLLGVGSAIA
SGIAVCKVLHLEGEVNKIKNALQLTNKAVVSLSNGVSVLTFRVLDLKNYINNQLLPMLNRQS
CRISNIETVIEFQQKNSRLLEITREFSVNAGVTTPLSTYMLINSELLSLINDMPIINDQKKL
MSSNVQIVRQQSYSIMCIIKEEVLAYVVQLPIYGVIDTPCWKLHTSPLCTTNIKEGSNICLT
RTDRGWYCDNAGSVSFFPQADTCKVQSNRVFCDTMNSLTLPSEVSLCNTDIFNSKYDCKIMT
SKTDISSSVITSLGAIVSCYGKTKCTASNKNRGIIKTFSNGCDYVSNKGVDTVSVGNTLYYV
NKLEGKNLYVKGEPIINYYDPLVFPSDEFDASISQVNEKINQSREIIRAINIVRKIASEK.

In some embodiments, the ectodomain comprises a polypeptide sequence at least 70%, at least 80%, at least 90%, at least 91%, at least 92%, at least 93%, at least 94%, at least 95%, at least 96%, at least 97%, at least 98%, at least 99%, or 100% identical to SEQ ID NO: 9, below, optionally lacking a p27 peptide shown in bold:

(SEQ ID NO: 9)
QNITEEFYQSTCSAVSKGYLSALRTGWYTSVITIELSNIKENKCNGTDAKVKLIKQELDKYK
NAVTELQLLMQSTPAINNRARRELPREMNYTLNNAKKTNVTLSKKRKRRFLGFLLGVGSAIA
SGVAVCKVLHLEGEVNKIKSALLSINKAVVSLSNGVSVLTFKVLDLKNYIDKQLLPILNKQS
CSISNIETVIEFQQKNNRLLEITREFSVNAGVTTPVSTYMLINSELLSLINDMPITNDQKKL
MSNNVQIVRQQSYSIMCIIKEEVLAYVVQLPLYGVIDTPCWKLHTSPLCTTNTKEGSNICLT
RTDRGWYCDNAGSVSFFPQAETCKVQSNRVFCDTMNSLTLPSEVNLCNVDIFNPKYDCKIMT
SKTDVSSSVITSLGAIVSCYGKTKCTASNKNRGIIKTFSNGCDYVSNKGVDTVSVGNTLYYV
NKQEGKSLYVKGEPIINFYDPLVFPSDEFDASISQVNEKINQSREIIRAINIVRKIASEK

In some embodiments, the polypeptide comprises a sequence at least 70%, at least 80%, at least 90%, at least 91%, at least 92%, at least 93%, at least 94%, at least 95%, at least 96%, at least 97%, at least 98%, at least 99%, or 100% identical to any one or more of SEQ ID NOs: 1-9, 76-296.

In another aspect, the disclosure provides a trimeric protein complex comprising a polypeptide of the disclosure.

In some embodiments, thermal stability, assayed by nanoDSF, is increased by at least 10° C., at least 15° C., at least 20° C., about 10° C. to about 30° C., about 10° C. to about 20° C., or about 20° C. to about 30° C. compared to a trimeric protein complex lacking modifications (a)-(h).

In some embodiments, stability, assayed by storage at about 40° C., is increased compared to a trimeric protein complex lacking modifications (a)-(h).

In some embodiments, the thermal stability is increased compared to a reference RSV F protein comprising amino acid substitutions consisting essentially of S155C, S290C, S190F, and V207L (DS-Cav1).

In another aspect, the disclosure provides a protein nanostructure comprising a trimeric component comprising a polypeptide described herein.

In some embodiments, the nanostructure is a two-component nanostructure comprising the first, trimeric component and a second, pentameric component.

In some embodiments, the first, trimeric component comprises an engineered ectodomain of a Respiratory Syncytial Virus (RSV) fusion (F) polypeptide and an I53-50A polypeptide.

In some embodiments, the first, trimeric component comprises a fusion protein comprising, in N- to C-terminal order, the RSV fusion (F) polypeptide, an amino acid linker, and the I53-50A polypeptide.

In some embodiments, the nanostructure is a two-component nanostructure comprising the first, trimeric component, wherein the first trimeric component comprises an engineered ectodomain of a RSV F polypeptide comprising an amino acid substitutions at position S155C, S290C, S190F, and V207L relative to SEQ ID NO: 1 and a C-terminal helix-forming segment comprising the polypeptide sequence NQSREIIRAINIVRKIASEK (SEQ ID NO: 10), and a multimerization domain comprising a polypeptide sequence at least 70%, at least 80%, at least 90%, at least 91%, at least 92%, at least 93%, at least 94%, at least 95%, at least 96%, at least 97%, at least 98%, at least 99%, or 100% identical to I53-50A (SEQ ID NO: 19) or I53-50A ΔCys (SEQ ID NO: 64), and/or the second pentameric component, wherein the pentameric component comprises a polypeptide sequence at least 70%, at least 80%, at least 90%, at least 91%, at least 92%, at least 93%, at least 94%, at least 95%, at least 96%, at least 97%, at least 98%, at least 99%, or 100% identical to SEQ ID NO: 20 or 71.

In some embodiments, the nanostructure is a two-component nanostructure comprising the first, trimeric component, wherein the first trimeric component comprises an engineered ectodomain of a RSV F polypeptide comprising an amino acid substitutions at position S155C, S290C, S190F, V207L, D489A, T400D, E487R, and K498A relative to SEQ ID NO: 1 and a C-terminal helix-forming segment comprising the polypeptide sequence NQSREIIRAINIVRKIASEK (SEQ ID NO: 10), and a multimerization domain comprising a polypeptide sequence at least 70%, at least 80%, at least 90%, at least 91%, at least 92%, at least 93%, at least 94%, at least 95%, at least 96%, at least 97%, at least 98%, at least 99%, or 100% identical to I53-50A (SEQ ID NO: 19) or I53-50A ΔCys (SEQ ID NO: 64) and/or the second pentameric component, wherein the pentameric component comprises a polypeptide sequence at least 70%, at least 80%, at least 90%, at least 91%, at least 92%, at least 93%, at least 94%, at least 95%, at least 96%, at least 97%, at least 98%, at least 99%, or 100% identical to SEQ ID NO: 20 or 71.

In some embodiments, the nanostructure is a two-component nanostructure comprising the first, trimeric component, wherein the first trimeric component comprises an engineered ectodomain of a RSV F polypeptide comprising an amino acid substitutions at position S155C, S290C, S190F, V207L, F488W, D489A, T400D, E487R, K498A, and T249P relative to SEQ ID NO: 1 and a C-terminal helix-forming segment comprising the polypeptide sequence NQSREIIRAINIVRKIASEK (SEQ ID NO: 10), and a multimerization domain comprising a polypeptide sequence at least 70%, at least 80%, at least 90%, at least 91%, at least 92%, at least 93%, at least 94%, at least 95%, at least 96%, at least 97%, at least 98%, at least 99%, or 100% identical to I53-50A (SEQ ID NO: 19) or I53-50A ΔCys (SEQ ID NO: 64), and/or the second pentameric component, wherein the pentameric component comprises a polypeptide sequence at least 70%, at least 80%, at least 90%, at least 91%, at least 92%, at least 93%, at least 94%, at least 95%, at least 96%, at least 97%, at least 98%, at least 99%, or 100% identical to SEQ ID NO: 20 or 71.

In some embodiments, the nanostructure is a two-component nanostructure comprising the first, trimeric component, wherein the first trimeric component comprises an engineered ectodomain of a RSV F polypeptide comprising an amino acid substitutions at position S155C, S290C, S190F, V207L, F488W, D489A, T400D, E487R, K498A, and D486A relative to SEQ ID NO: 1 and a C-terminal helix-forming segment comprising the polypeptide sequence NQSREIIRAINIVRKIASEK (SEQ ID NO: 10), and a multimerization domain comprising a polypeptide sequence at least 70%, at least 80%, at least 90%, at least 91%, at least 92%, at least 93%, at least 94%, at least 95%, at least 96%, at least 97%, at least 98%, at least 99%, or 100% identical to I53-50A (SEQ ID NO: 19) or I53-50A ΔCys (SEQ ID NO: 64), and/or the second pentameric component, wherein the pentameric component comprises a polypeptide sequence at least 70%, at least 80%, at least 90%, at least 91%, at least 92%, at least 93%, at least 94%, at least 95%, at least 96%, at least 97%, at least 98%, at least 99%, or 100% identical to SEQ ID NO: 20 or 71.

In some embodiments, the trimeric component comprises a polypeptide sequence at least 70%, at least 80%, at least 90%, at least 91%, at least 92%, at least 93%, at least 94%, at least 95%, at least 96%, at least 97%, at least 98%, at least 99%, or 100% identical to any one of the sequences listed in Table 14 or to any one of the sequences listed in Table 14 without the underlined and/or bold/italicized polypeptide sequences.

In some embodiments, the pentameric component comprises a polypeptide sequence at least 70%, at least 80%, at least 90%, at least 91%, at least 92%, at least 93%, at least 94%, at least 95%, at least 96%, at least 97%, at least 98%, at least 99%, or 100% identical to any one or more of SEQ ID NOs: 20, 44, 45, 52, 71, 73, 74.

In another aspect, the disclosure provides a pharmaceutical composition comprising a polypeptide, a protein complex, or a nanostructure of the disclosure.

In another aspect, the disclosure provides a vaccine comprising a polypeptide, a protein complex, or a nanostructure of the disclosure.

In another aspect, the disclosure provides a method of vaccinating a subject, comprising administering to the subject a composition described herein.

In another aspect, the disclosure provides a method of generating an immune response in a subject, comprising administering to the subject a composition described herein.

In another aspect, the disclosure provides a method of treating or preventing RSV disease in a subject, comprising administering to the subject a composition described herein.

In another aspect, the disclosure provides a composition of the disclosure for use in vaccinating, generating an immune response, or treating or preventing RSV disease.

In another aspect, the disclosure provides a composition, method, or use as described herein. In another aspect, the disclosure provides a method of making a compositions, comprising culturing host cells modified to express one or more polypeptides as described herein.

Any aspect or embodiment described herein can be combined with any other aspect or embodiment as disclosed herein. Further aspects, embodiments, and advantages of the invention will be apparent from the Detailed Description that follows.

### BRIEF DESCRIPTION OF THE DRAWINGS

These and other features, aspects, and advantages of the present invention will become better understood with regard to the following description, and accompanying drawings, where:

FIG. 1 shows a structural model of RSV F protein in the prefusion conformation (PDB 4MMU), with stabilizing elements separated into five different spaces. Spaces 1-4 were targeted by stabilizing mutations. Space 5 refers to the C terminus of the protein.

FIG. 2 shows a close-up view of the structure of C termini of RSV F protein determined by X-ray crystallography of prefusion RSV F (PDB 4MMU) before and after remodelling. Residues that are remodelled (residues 503-509) are outlined with a thicker black highlight (left) and additional structure added by remodeling is shown in black (right).

FIG. 3 shows ddG scoring with representative designs highlighted.

FIG. 4 shows hydrophobicity scoring of designs. Mean (solid line) and standard deviation (dashed lines), WT (dotted line).

FIG. 5 shows a representative electron micrograph of a protein nanostructure as described herein.

FIG. 6 shows neutralizing titers against RSV/B (B18537 strain) elicited by various nanostructure immunogens based on RSV/B antigens.

FIG. 7 shows neutralizing titers against RSV/A (Tracy strain) elicited by various nanostructure immunogens based on RSV/A antigens.

FIG. 8 shows a structural comparison of cryo-EM structures of the RSV F ectodomains of A) RSV/A.023, and B) DS-Cav1 fused to foldon (PDB 7LUE). The added C-terminal alpha-helical segment in RSV/A.023 is colored in dark gray and surrounded by a dashed box. Antibody structures were removed from the model of PDB 7LUE prior to generating images.

FIG. 9 shows a structural comparison of C-terminal regions for cryo-EM structures of the RSV F ectodomains of A) RSV/A.023, and B) DS-Cav1 fused to foldon (PDB 7LUE). The added C-terminal alpha-helical segment in RSV/A.023 is colored in dark gray and surrounded by a dashed box. Antibody structures were removed from the model of PDB 7LUE prior to generating images.

### DETAILED DESCRIPTION

Before the embodiments of the disclosure are described, it is to be understood that such embodiments are provided by way of example only, and that various alternatives to the embodiments of the disclosure described herein may be employed in practicing the invention. Numerous variations, changes, and substitutions will occur to those skilled in the art and may be practiced without departing from spirit of the invention.

Unless defined otherwise herein, all technical and scientific terms used herein have the same meaning as commonly understood by one of ordinary skill in the art to which this disclosure belongs. Various scientific dictionaries that include the terms included herein are well known and available to those in the art. Although any methods and materials similar or equivalent to those described herein find use in the practice or testing of the disclosure, some preferred methods and materials are described. Accordingly, the terms defined immediately below are more fully described by reference to the specification as a whole.

### I. Definitions

The singular forms “a,” “an,” and “the” are intended to include the plural forms as well, unless the context clearly indicates otherwise.

As used herein, the term “about” means a range of values including the specified value, which a person of ordinary skill in the art would consider reasonably similar to the specified value. For example, about means within a standard deviation using measurements generally acceptable in the art. For example, about means a range extending to +/−10%, +/−5%, +/−3%, or +/−1% of the specified value.

The term “at least” followed by a number is used herein to denote the start of a range beginning with that number (which may be a range having an upper limit or no upper limit, depending on the variable being defined). For example, “at least 1” means 1 or more than 1.

The term “at most” followed by a number is used herein to denote the end of a range ending with that number (which may be a range having 1 or 0 as its lower limit, or a range having no lower limit, depending upon the variable being defined). For example, “at most 4” means 4 or less than 4, and “at most 40%” means 40% or less than 40%. When, in this specification, a range is given as “(a first number) to (a second number)” or “(a first number)-(a second number)” this means a range whose lower limit is the first number and whose upper limit is the second number. For example, 25 to 100 mm means a range whose lower limit is 25 mm, and whose upper limit is 100 mm.

The term “identical” or percent “identity,” in the context of two or more nucleic acid or polypeptide sequences, refers to two or more sequences or subsequences that are the same or have a specified percentage of amino acid residues or nucleotides that are the same, when compared and aligned for maximum correspondence. Methods of alignment of sequences for comparison are well known in the art. Once aligned, the number of matches is determined by counting the number of positions where an identical nucleotide or amino acid residue is present in both sequences. The percent sequence identity is determined by dividing the number of matches in the alignment by the length of the reference sequence, followed by multiplying the resulting value by 100. For example, a peptide sequence that has 1166 matches when aligned with a reference sequence having 1554 amino acids is 75.0 percent identical to the test sequence (1166÷1554*100=75.0). As the terms are used herein, gaps in the alignment do not decrease the percent sequence identity. Unless otherwise specified, optimal alignment of sequences for comparison is conducted by the global alignment algorithm of Needleman and Wunsch, Mol. Biol. 48:443 (1970) as implemented by EMBOSS Needle (on the World Wide Web at ebi.ac.uk/Tools/psa/emboss_needle/) (Madeira et al. Nucleic Acids Res. 50(W1):W276-W279 (2022)). Other alignment methods may be used, including without limitation those described in Devereux, et al, Nucleic Acids Res. 12:387-95 (1984); Atschul et al. J. Mo. Biol. 215:403-10 (1990) (BLAST); Carrillo and Lipman Siam J. Appl. Math. 48(5) (1988); Computational Molecular Biology (Lesk, AM, ed., 1989); Biocomputing Informatics and Genome Projects, (Smith, DW, ed., 1993); Computer Analysis of Sequence Data, Part I, (Griffin and Griffin, eds., 1994); Sequence Analysis in Molecular Biology (von Heinje, 2012); Sequence Analysis Primer (Gribskov and Devereux, J., eds. 1993). Sequence identity is calculated using the implementation of the Needleman-Wunsch algorithm provided by the National Library of Medicine (on the World Wide Web at blast.ncbi.nlm.nih.gov/Blast.cgi?PAGE_TYPE=BlastSearch&BLAST_SPEC=GlobalAln).

For example, sequence identity can be determined by standard methods that are commonly used to compare the similarity of two polypeptide or two polynucleotide sequences. Using a computer program such as EMBOSS Needle or BLAST, two polypeptide or two polynucleotide sequences are aligned for optimal matching of their respective residues (either along the full length of one or both sequences, or along a pre-determined portion of one or both sequences). The programs provide a default opening penalty and a default gap penalty, and a scoring matrix such as PAM 250 (a standard scoring matrix; see Dayhoff et al., in Atlas of Protein Sequence and Structure, vol. 5, supp. 3 (1978)) that can be used in conjunction with the computer program.

As used herein, the term “helix-forming segment” refers to a portion of a protein or polypeptide that forms, or is predicted to form, an alpha-helix. An “alpha-helix” is an element of protein secondary structure stabilized by hydrogen bonds between carbonyl oxygen and the amino group of every third residue in the helical turn. The smallest segment of a protein that is generally considered to form an alpha-helix is about 6-7 amino acid results. Accordingly, in some embodiments, a helix-forming segment comprises between about 5 and about 30 amino acid residues, between about 7 and about 14 amino acid residues, between about 7 and about 21 amino acid residues, between about 7 and about 28 amino acid residues, between about 7 and about 35 amino acid residues, between about 7 and about 42 amino acid residues, or between about 7 and about 49 amino acid residues; or any values therebetween, such as without limitation 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, or more amino acids.

As used herein the term “alpha-helical homotrimer” refers to a three-helix bundle with helices in parallel orientation. The term excludes six-helical bundles such as those formed by assembly of three anti-parallel, two-helix bundles; i.e., the term “alpha-helical homotrimer” as used herein excludes heptad-repeat regions of gp41 or recombinant variants thereof.

As used herein, the term “stable” such as in “stable alpha-helical homotrimer” means that the protein structure (e.g., homotrimer) persists under suitable conditions. A stable protein structure may be detected by biophysical or biochemical methods known in the art-including but not limited to size exclusion chromotagraphy, dynamic light scattering, electron microscopy, analytical ultracentrifugation, X-ray crystallography, nuclear magnetic resonance spectroscopy, circular dichroism, thermal denaturation, or interaction measurements. A “stable” alpha-helical homotrimer may be distinguished from an unstable homotrimer in part by structural analysis (e.g., by X-ray crystallography, NMR, or EM), or by measuring the impact of the alpha-helical homotrimer, for example by binding studies (BLI, SPR) or biophysical studies (thermal denaturation). In some embodiments, the stable alpha-helical homotrimer may be stable at room temperature and/or at elevated temperatures (e.g., 40° C.). An alpha-helical homotrimer may either form a homotrimer in isolation, or as part of a larger trimeric protein complex (such as a trimeric antigen). In some embodiments, inclusion of the stable alpha-helical homotrimer stabilizes the trimeric protein complex by a AAG of at least −10, at least −20, at least −30, at least −40, at least −50, or at least −60, as predicted computationally or experimentally determined. In some embodiments, the stable alpha-helical homotrimer is an “obligate” homotrimer.

As used here, “conservative amino acid substitution” means that: hydrophobic amino acids (Ala, Gly, Met, Val, Ile, Leu, Thr) are substituted with other hydrophobic amino acids; hydrophobic amino acids with bulky side chains (Phe, Tyr, Trp) are substituted with other hydrophobic amino acids with bulky side chains; amino acids with positively charged side chains (Arg, His, Lys) are substituted with other amino acids with positively charged side chains; amino acids with negatively charged side chains (Asp, Glu) are substituted with other amino acids with negatively charged side chains; and polar amino acids (Cys, Ser, Thr, Asn, Gly, Tyr) are substituted with other polar amino acids.

| Amino Acid | Three letter symbol | One letter symbol |
|---|---|---|
| Alanine | Ala | A |
| Arginine | Arg | R |
| Asparagine | Asn | N |
| Aspartic acid | Asp | D |
| Cysteine | Cys | C |
| Glutamic acid | Glu | E |
| Glutamine | Gln | Q |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

Throughout this specification and the claims which follow, unless the context requires otherwise, the word “comprise”, and variations such as “comprises” and “comprising,” as well as “has” or “having” and “includes” or “including,” will be understood to imply the inclusion of a stated element or step or group of elements or steps but not the exclusion of any other element or step or group of elements or steps. “Consisting essentially of” or “consists essentially” indicates exclusion of elements or steps that materially affect the basic and novel characteristics of the claimed invention.

### II. Engineered Ectodomains

The disclosure provides recombinant polypeptides comprising an ectodomain of a viral membrane fusion (F) protein of Respiratory Syncytial Virus (RSV) having an engineered C-terminal alpha-helical segment. It further provides recombinant polypeptides comprising amino acid substitutions that stabilize the RSV F protein in a prefusion conformation. In further embodiments, the disclosure provides recombinant polypeptides comprising amino acid substitutions having an engineered C-terminal alpha-helical segment that stabilize the RSV F protein in a prefusion conformation.

### Respiratory Syncytial Virus (RSV) F Protein

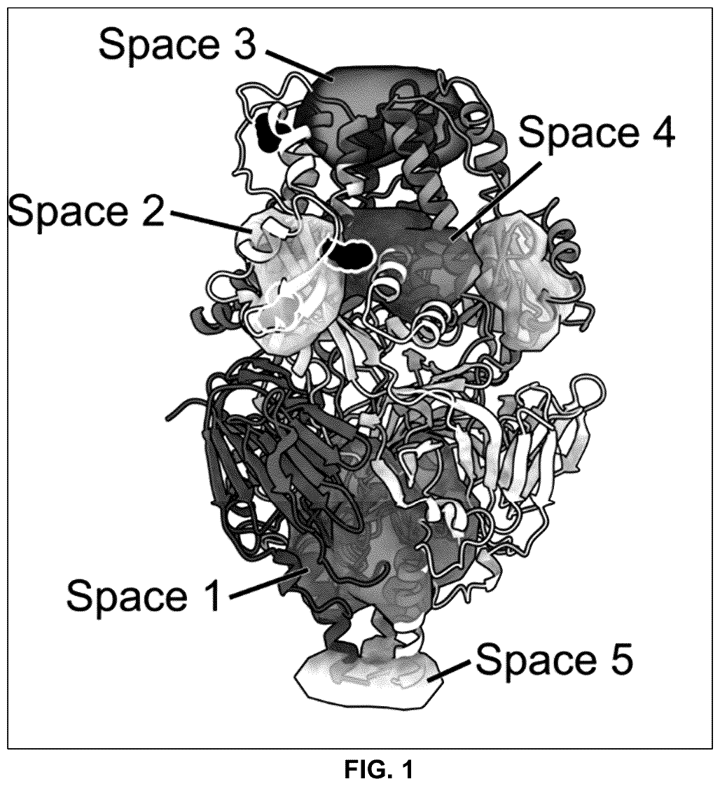

Respiratory Syncytial Virus (RSV) F protein is a major conserved surface antigen of RSV and antibodies against it are associated with protection against disease. RSV F protein is a validated target for protection against infection by RSV as demonstrated by the clinical efficacy of palivizumab, a monoclonal antibody that binds F-antigen and leads to neutralization of the virus (Johnson et al., J Infect Dis. 1997 November; 176(5):1215-24). RSV F protein is known to undergo a significant change in structure from prefusion to postfusion form which catalyzes viral and host membrane fusion to allow for viral entry into the cell (McLellan et al., Science. 2013; 342(6158):592-8). Prefusion F-protein has important epitopes that are lost during the transition to postfusion F-protein (Melero et al., Vaccine. 2017; 35(3):461-468). Antibody depletion studies with human sera absorbed with RSV F protein in either conformation demonstrate that the majority of the neutralizing response against RSV F protein targets the prefusion structure (Krarup et al., Nat Commun. 2015; 6:8143). These studies also demonstrate the potential for antibodies that bind postfusion F protein to interfere with neutralization (Ngwuta et al., Sci Transl Med. 2015; 7(309):309ra162). In general, high levels of antibodies against RSV F protein are associated with protection against severe disease. However, generating high-titers of neutralizing antibodies against RSV F protein remains challenging, due to the specific biochemical nature of the RSV F protein and the unpredictability of vaccine responses to RSV F. Structural model of RSV F protein in the prefusion conformation is shown in FIG. 1, with stabilizing elements separated into five different spaces. Spaces 1-4 were targeted by stabilizing mutations. Space 5 refers to the C terminus of the protein.

Illustrative sequences are shown in Table 1. A native RSV/B F protein sequence was used for design (GenBank: WDV37446.1). The (predicted) transmembrane region is residues 527-549 and is bold/underlined. The signal peptide is underlined with italic.

TABLE 1

| | | | SEQ |
|---|---|---|---|
| | | | ID |
| | Description | Sequence | NO: |
| RSV/B | GenBank: | MELLIHRSSAIFLTLAINALYLTSSQNIT | 1 |
| F protein | WDV37446.1 | EEFYQSTCSAVSRGYLSALRTGWYTSVIT | |
| | Reference | IELSNIKETKCNGTDTKVKLIKQELDKYK | |
| | sequence | NAVTELQLLMQNTPAVNNRARREAPQYMN | |
| | | YTINTTKNLNVSISKKRKRRFLGFLLGVG | |
| | | SAIASGIAVSKVLHLEGEVNKIKNALQLT | |
| | | NKAVVSLSNGVSVLTSRVLDLKNYINNQL | |
| | | LPMVNRQSCRISNIETVIEFQQKNSRLLE | |
| | | ITREFSVNAGVTTPLSTYMLINSELLSLI | |
| | | NDMPITNDQKKLMSSNVQIVRQQSYSIMS | |
| | | IIKEEVLAYVVQLPIYGVIDTPCWKLHTS | |
| | | PLCTTNIKEGSNICLTRTDRGWYCDNAGS | |
| | | VSFFPQADTCKVQSNRVFCDTMNSLTLPS | |
| | | EVSLCNTDIFNSKYDCKIMTSKTDISSSV | |
| | | ITSLGAIVSCYGKTKCTASNKNRGIIKTF | |
| | | SNGCDYVSNKGVDTVSVGNTLYYVNKLEG | |
| | | KNLYVKGEPIINYYDPLVFPSDEFDASIS | |
| | | QVNEKINQSLAFIRRSDELLHNVNTGKST | |
| | | TNIMITAITIVIIVVLLSLIAIGLLLYCK | |
| | | AKNTPVTLSKDQLSGINNIAFSK | |
| RSV/B | GenBank: | MELLIHRSSAIFLTLAINALYLTSSQNIT | 2 |
| F protein | WDV37446.1 | EEFYQSTCSAVSRGYLSALRTGWYTSVIT | |
| | DS-Cav 1 | IELSNIKETKCNGTDTKVKLIKQELDKYK | |
| | (S155C, S290C, | NAVTELQLLMQNTPAVNNRARREAPQYMN | |
| | S190F, V207L) | YTINTTKNLNVSISKKRKRRFLGFLLGVG | |
| | | SAIASGIAVCKVLHLEGEVNKIKNALQLT | |
| | | NKAVVSLSNGVSVLTCRVLDLKNYINNQL | |
| | | LPMLNRQSCRISNIETVIEFQQKNSRLLE | |
| | | ITREFSVNAGVTTPLSTYMLINSELLSLI | |
| | | NDMPITNDQKKLMSSNVQIVRQQSYSIMC | |
| | | IIKEEVLAYVVQLPIYGVIDTPCWKLHTS | |
| | | PLCTTNIKEGSNICLTRTDRGWYCDNAGS | |
| | | VSFFPQADTCKVQSNRVFCDTMNSLTLPS | |
| | | EVSLCNTDIFNSKYDCKIMISKTDISSSV | |
| | | ITSLGAIVSCYGKTKCTASNKNRGIIKTF | |
| | | SNGCDYVSNKGVDTVSVGNTLYYVNKLEG | |
| | | KNLYVKGEPIINYYDPLVFPSDEFDASIS | |
| | | QVNEKINQSLAFIRRSDELLHNVNTGKST | |
| | | TNIMITAITIVIIVVLLSLIAIGLLLYCK | |
| | | AKNTPVTLSKDQLSGINNIAFSK | |
| RSV/B | Without signal | QNITEEFYQSTCSAVARGYLSALRTGWYT | 3 |
| F protein | peptide | SVITIELSNIKETKCNGTDTKVKLIKQEL | |
| Ectodomain | | DKYKNAVTELQLLMQNTPAVNNRARREAP | |
| | | QYMNYTINTTKNLNVSISKKRKRRFLGFL | |
| | | LGVGSAIASGIAVSKVLHLEGEVNKIKNA | |
| | | LQLTNKAVVSLSNGVSVLTSRVLDLKNYI | |
| | | NNQLLPMVNRQSCRISNIETVIEFQQKNS | |
| | | RLLEITREFSVNAGVTTPLSTYMLINSEL | |
| | | LSLINDMPITNDQKKLMSSNVQIVRQQSY | |
| | | SIMSIIKEEVLAYVVQLPIYGVIDTPCWK | |
| | | LHTSPLCTTNIKEGSNICLTRTDRGWYCD | |
| | | NAGSVSFFPQADTCKVQSNRVFCDTMNSL | |
| | | TLPSEVSLCNTDIFNSKYDCKIMTSKTDI | |
| | | SSSVITSLGAIVSCYGKTKCTASNKNRGI | |
| | | IKTFSNGCDYVSNKGVDTVSVGNTLYYVN | |
| | | KLEGKNLYVKGEPIINYYDPLVFPSDEFD | |
| | | ASISQVNEKINQSLAFIRRSDELLHNVNT | |
| | | GKSTTNIMITAITIVIIVVLLSLIAIGLL | |
| | | LYCKAKNTPVTLSKDQLSGINNIAFSK | |
| RSV/B | Without signal | QNITEEFYQSTCSAVSRGYLSALRTGWYT | 4 |
| F protein | peptide | SVITIELSNIKETKCNGTDTKVKLIKQEL | |
| Ectodomain | DS-Cav 1 | DKYKNAVTELQLLMQNTPAVNNRARREAP | |
| | (S155C, S290C, | QYMNYTINTTKNLNVSISKKRKRRFLGFL | |
| | S190F, V207L) | LGVGSAIASGIAVCKVLHLEGEVNKIKNA | |
| | | LQLTNKAVVSLSNGVSVLTCRVLDLKNYI | |
| | | NNQLLPMLNRQSCRISNIETVIEFQQKNS | |
| | | RLLEITREFSVNAGVTTPLSTYMLTNSEL | |
| | | LSLINDMPITNDQKKLMSSNVQIVRQQSY | |
| | | SIMCIIKEEVLAYVVQLPIYGVIDTPCWK | |
| | | LHTSPLCTTNIKEGSNICLTRTDRGWYCD | |
| | | NAGSVSFFPQADTCKVQSNRVFCDTMNSL | |
| | | TLPSEVSLCNTDIFNSKYDCKIMTSKTDI | |
| | | SSSVITSLGAIVSCYGKTKCTASNKNRGI | |
| | | IKTFSNGCDYVSNKGVDTVSVGNTLYYVN | |
| | | KLEGKNLYVKGEPIINYYDPLVFPSDEFD | |
| | | ASISQVNEKINQSLAFIRRSDELLHNVNT | |
| | | GKSTTNIMITAITIVIIVVLLSLIAIGLL | |
| | | LYCKAKNTPVTLSKDQLSGINNIAFSK | |
| RSV/B | Without signal | QNITEEFYQSTCSAVSKGYLSALRTGWYT | 180 |
| F protein | peptide | SVITIELSNIKENKCNGTDAKVKLIKQEL | |
| Ectodomain | DS-Cav 1 | DKYKNAVTELQLLMQSTPAINNRARRELP | |
| | (S155C, S290C, | RFMNYTLNNAKKTNVTLSKKRKRRFLGFL | |
| | S190F, V207L) | LGVGSAIASGVAVCKVLHLEGEVNKIKSA | |
| | | LLSTNKAVVSLSNGVSVLTFKVLDLKNYI | |
| | | DKQLLPILNKQSCSISNIETVIEFQQKNN | |
| | | RLLEITREFSVNAGVTTPVSTYMLINSEL | |
| | | LSLINDMPITNDQKKLMSNNVQIVRQQSY | |
| | | SIMCIIKEEVLAYVVQLPLYGVIDTPCWK | |
| | | LHTSPLCTTNTKEGSNICLTRTDRGWYCD | |
| | | NAGSVSFFPQAETCKVQSNRVFCDTMNSL | |
| | | TLPSEVNLCNVDIFNPKYDCKIMTSKTDV | |
| | | SSSVITSLGAIVSCYGKTKCTASNKNRGI | |
| | | IKTFSNGCDYVSNKGVDTVSVGNTLYYVN | |
| | | KQEGKSLYVKGEPIINFYDPLVFPSDEFD | |
| | | ASISQVNEKINQSLAFIRKSDELL | |
| RSV/B | Without signal | QNITEEFYQSTCSAVSRGYFSALRIGWYT | 181 |
| F protein | peptide | SVITIELSNITETKCNGTDTKVKLIKQEL | |
| Ectodomain | | DKYKNAVTELQLLMQNTPAANNRARREAP | |
| | | QHMNYTINTTKNLNVSISKKRKRRFLGFL | |
| | | LGVGSAIASGIAVSKVLHLEGEVNKIKNA | |
| | | LLSTNKAVVSLSNGVSVLTSKVLDLKNYI | |
| | | NNQLLPIVNQQSCRIFNIETVIEFQQKNS | |
| | | RLLEITREFSVNAGVTTPLSTYMLINSEL | |
| | | LSLINDMPITNDQKKLMSSNVQIVRQQSY | |
| | | SIMSIIKEEVLAYVVQLPIYGVIDTPCWK | |
| | | LHTSPLCTTNIKEGSNICLTRTDRGWYCD | |
| | | NAGSVSFFPQADTCKVQSNRVFCDTMNSL | |
| | | TLPSEVSLCNTDIFNSKYDCKIMTSKTDI | |
| | | SSSVITSLGAIVSCYGKTKCTASNKNRGI | |
| | | IKTFSNGCDYVSNKGVDTVSVGNTLYYVN | |
| | | KLEGKNLYVKGEPIINYYDPLVFPSDEFD | |
| | | ASISQVNEKINQSLAFIRKSDELL | |
| RSV/B | Without signal | QNITEEFYQSTCSAVSRGYFSALRTGWYT | 182 |
| F protein | peptide | SVITIELSNITETKCNGTDTKVKLIKQEL | |
| Ectodomain | DS-Cav 1 | DKYKNAVTELQLLMQNTPAANNRARREAP | |
| | (S155C, S290C, | QHMNYTINTTKNLNVSISKKRKRRFLGFL | |
| | S190F, V207L) | LGVGSAIASGIAVCKVLHLEGEVNKIKNA | |
| | Stabilized | LLSTNKAVVSLSNGVSVLTFKVLDLKNYI | |
| | muation | NNQLLPILNQQSCRIFNIETVIEFQQKNS | |
| | | RLLEITREFSVNAGVTTPLSTYMLTNSEL | |
| | | LSLINDMPITNDQKKLMSSNVQIVRQQSY | |
| | | SIMCIIKEEVLAYVVQLPIYGVIDTPCWK | |
| | | LHTSPLCTTNIKEGSNICLTRTDRGWYCD | |
| | | NAGSVSFFPQADTCKVQSNRVFCDTMNSL | |
| | | TLPSEVSLCNTDIFNSKYDCKIMTSKTDI | |
| | | SSSVITSLGAIVSCYGKTKCTASNKNRGI | |
| | | IKTFSNGCDYVSNKGVDTVSVGNTLYYVN | |
| | | KLEGKNLYVKGEPIINYYDPLVFPSDEFD | |
| | | ASISQVNEKINQSLAFIRKSDELL | |
| RSV/A | Without signal | QNITEEFYQSTCSAVSKGYLSALRTGWYT | 5 |
| F protein | peptide | SVITIELSNIKENKCNGTDAKVKLIKQEL | |
| Ectodomain | DS-Cav 1 | DKYKNAVTELQLLMQSTPAINNRARRELP | |
| | (S155C, S290C, | RFMNYTLNNAKKTNVTLSKKRKRRFLGFL | |
| | S190F, V207L) | LGVGSAIASGVAVCKVLHLEGEVNKIKSA | |
| | | LLSTNKAVVSLSNGVSVLTFKVLDLKNYI | |
| | | DKQLLPILNKQSCSISNIETVIEFQQKNN | |
| | | RLLEITREFSVNAGVTTPVSTYMLINSEL | |
| | | LSLINDMPITNDQKKLMSNNVQIVRQQSY | |
| | | SIMCIIKEEVLAYVVQLPLYGVIDTPCWK | |
| | | LHTSPLCTTNTKEGSNICLTRTDRGWYCD | |
| | | NAGSVSFFPQAETCKVQSNRVFCDTMNSL | |
| | | TLPSEVNLCNVDIFNPKYDCKIMTSKTDV | |
| | | SSSVITSLGAIVSCYGKTKCTASNKNRGI | |
| | | IKTFSNGCDYVSNKGVDTVSVGNTLYYVN | |
| | | KQEGKSLYVKGEPIINFYDPLVFPSDEFD | |
| | | ASISQVNEKINQSLAFIRKSDELL | |
| RSV/A2 | GenBank GI: | MELLILKANAITTILTAVTFCFASGQNIT | 183 |
| F protein | 138251 | EEFYQSTCSAVSKGYLSALRTGWYTSVIT | |
| | Swiss Prot | IELSNIKENKCNGTDAKVKLIKQELDKYK | |
| | P03420 | NAVTELQLLMQSTPPTNNRARRELPREMN | |
| | | YTLNNAKKTNVTLSKKRKRRFLGFLLGVG | |
| | | SAIASGVAVSKVLHLEGEVNKIKSALLST | |
| | | NKAVVSLSNGVSVLTSKVLDLKNYIDKQL | |
| | | LPIVNKQSCSISNIETVIEFQQKNNRLLE | |
| | | ITREFSVNAGVTTPVSTYMLINSELLSLI | |
| | | NDMPITNDQKKLMSNNVQIVRQQSYSIMS | |
| | | IIKEEVLAYVVQLPLYGVIDTPCWKLHTS | |
| | | PLCTTNTKEGSNICLTRTDRGWYCDNAGS | |
| | | VSFFPQAETCKVQSNRVFCDTMNSLTLPS | |
| | | EINLCNVDIFNPKYDCKIMTSKTDVSSSV | |
| | | ITSLGAIVSCYGKIKCTASNKNRGIIKIF | |
| | | SNGCDYVSNKGMDTVSVGNTLYYVNKQEG | |
| | | KSLYVKGEPIINFYDPLVFPSDEFDASIS | |
| | | QVNEKINQSLAFIRKSDELLHNVNAGKST | |
| | | TNIMITTIIIVIIVILLSLIAVGLLLYCK | |
| | | ARSTPVTLSKDQLSGINNIAFSN | |
| RSV/B | 18537 strain | MELLIHRSSAIFLTLAVNALYLTSSQNIT | 184 |
| F protein | GenBank GI: | EEFYQSTCSAVSRGYFSALRIGWYTSVIT | |
| | 138250 | IELSNIKETKCNGTDTKVKLIKQELDKYK | |
| | Swiss Prot | NAVTELQLLMQNTPAANNRARREAPQYMN | |
| | P13843 | YTINTTKNLNVSISKKRKRRFLGFLLGVG | |
| | | SAIASGIAVSKVLHLEGEVNKIKNALLST | |
| | | NKAVVSLSNGVSVLTSKVLDLKNYINNRL | |
| | | LPIVNQQSCRISNIETVIEFQQMNSRLLE | |
| | | ITREFSVNAGVTTPLSTYMLTNSELLSLI | |
| | | NDMPITNDQKKLMSSNVQIVRQQSYSIMS | |
| | | IIKEEVLAYVVQLPIYGVIDTPCWKLHTS | |
| | | PLCTTNIKEGSNICLTRTDRGWYCDNAGS | |
| | | VSFFPQADTCKVQSNRVFCDTMNSLTLPS | |
| | | EVSLCNTDIFNSKYDCKIMTSKTDISSSV | |
| | | ITSLGAIVSCYGKTKCTASNKNRGIIKTF | |
| | | SNGCDYVSNKGVDTVSVGNTLYYVNKLEG | |
| | | KNLYVKGEPIINYYDPLVFPSDEFDASIS | |
| | | QVNEKINQSLAFIRRSDELLHNVNTGKST | |
| | | TNIMITTIIIVIIVVLLSLIAIGLLLYCK | |
| | | AKNTPVTLSKDQLSGINNIAFSK | |
| RSV F protein | | MELLILKANAITTILTAVTFCFASGQNIT | 185 |
| | | EEFYQSTCSAVSKGYLSALRTGWYTSVIT | |
| | | IELSNIKENKCNGTDAKVKLIKQELDKYK | |
| | | NAVTELQLLMQSTPATNNRARRELPREMN | |
| | | YTLNNAKKTNVTLSKKRKRRFLGFLLGVG | |
| | | SAIASGVAVCKVLHLEGEVNKIKSALLST | |
| | | NKAVVSLSNGVSVLTFKVLDLKNYIDKQL | |
| | | LPILNKQSCSISNIETVIEFQQKNNRLLE | |
| | | ITREFSVNAGVTTPVSTYMLINSELLSLI | |
| | | NDMPITNDQKKLMSNNVQIVRQQSYSIMC | |
| | | IIKEEVLAYVVQLPLYGVIDTPCWKLHTS | |
| | | PLCTTNTKEGSNICLTRTDRGWYCDNAGS | |
| | | VSFFPQAETCKVQSNRVFCDTMNSLTLPS | |
| | | EVNLCNVDIFNPKYDCKIMTSKTDVSSSV | |
| | | ITSLGAIVSCYGKTKCTASNKNRGIIKTF | |
| | | SNGCDYVSNKGVDTVSVGNTLYYVNKQEG | |
| | | KSLYVKGEPIINFYDPLVFPSDEFDASIS | |
| | | QVNEKINQSLAFIRKSDELLSAIGGYIPE | |
| | | APRDGQAYVRKDGEWVLLSTEL | |

In some embodiments, the ectodomain is at least 70%, at least 80%, at least 90%, at least 91%, at least 92%, at least 93%, at least 94%, at least 95%, at least 96%, at least 97%, at least 98%, at least 99%, or 100% identical to SEQ ID NO: 1. In some embodiments, the ectodomain is at least 70%, at least 80%, at least 90%, at least 91%, at least 92%, at least 93%, at least 94%, at least 95%, at least 96%, at least 97%, at least 98%, at least 99%, or 100% identical to SEQ ID NO: 2. In some embodiments, the ectodomain is at least 70%, at least 80%, at least 90%, at least 91%, at least 92%, at least 93%, at least 94%, at least 95%, at least 96%, at least 97%, at least 98%, at least 99%, or 100% identical to SEQ ID NO: 3. In some embodiments, the ectodomain is at least 70%, at least 80%, at least 90%, at least 91%, at least 92%, at least 93%, at least 94%, at least 95%, at least 96%, at least 97%, at least 98%, at least 99%, or 100% identical to SEQ ID NO: 4. In some embodiments, the ectodomain is at least 70%, at least 80%, at least 90%, at least 91%, at least 92%, at least 93%, at least 94%, at least 95%, at least 96%, at least 97%, at least 98%, at least 99%, or 100% identical to SEQ ID NO: 5. In some embodiments, the ectodomain is at least 70%, at least 80%, at least 90%, at least 91%, at least 92%, at least 93%, at least 94%, at least 95%, at least 96%, at least 97%, at least 98%, at least 99%, or 100% identical to SEQ ID NO: 180. In some embodiments, the ectodomain is at least 70%, at least 80%, at least 90%, at least 91%, at least 92%, at least 93%, at least 94%, at least 95%, at least 96%, at least 97%, at least 98%, at least 99%, or 100% identical to SEQ ID NO: 181. In some embodiments, the ectodomain is at least 70%, at least 80%, at least 90%, at least 91%, at least 92%, at least 93%, at least 94%, at least 95%, at least 96%, at least 97%, at least 98%, at least 99%, or 100% identical to SEQ ID NO: 182.

### C-Terminal Helix-Forming Segment

The C-terminal end of the ectodomain of many viral fusion proteins is, in at least some cases, known to be or predicted to be a helical bundle that interfaces with a helical transmembrane domain. The present inventors have observed that, in the RSV F protein, the C-terminal helical region of the ectodomain has suboptimal hydrophobic packing. Computational modeling (with RosettaRemodel) was used to generate artificial polypeptide sequences, each predicted to form a stable alpha helix. In illustrative, non-limiting Examples provided below, the helical backbone is first optimized with side-chains represented as centroids, and then the side-chains are designed in all-atom mode. Optimal linker length can be determined by a plot of ddG as a function of linker length (Rosetta remodel), or ddG normalized to linker length (RFdiffusion). Then 6-14 additional amino acids were modelled with helical constraints.

Illustrative sequences are shown in Table 2A. Residues 500-502 of the native RSV F protein are included as NQS (bold underline) and are, in these embodiments, conserved with the native sequence whereas many of the other amino acid residues are modified.

TABLE 2A
C-terminal Alpha-helical segments (Rosetta remodel)

| | | Remodeled | |
|---|---|---|---|
| Name | Sequence | Length | SEQ ID NO: |
| C-Term 1 | NQSREIIRAINIVRKIASEK | 17 | 10 |
| C-Term 2 | NQSALWLEAAKYVKQAREKS | 17 | 11 |
| C-Term 3 | NQSAKNAEAAKIAEETKRKD | 17 | 12 |
| C-Term 4 | NQSRETAKAVSAVK | 11 | 75 |
| C-Term 5 | NQSALLLEAAKYVKKAREKS | 17 | 119 |
| C-Term 6 | NQSRKLLEAAEEMEKMLKTS | 17 | 120 |
| C-Term 7 | NQSRKMLEAVEHAKKLKKES | 17 | 121 |
| C-Term 8 | NQSRKMLEAVEKAKKLDKES | 17 | 122 |
| C-Term 9 | NQSAKTEEAYQRTIKTQQKL | 17 | 123 |
| C-Term 10 | NQSRDLDTAAKQVKEMLKEKS | 18 | 124 |
| C-Term 11 | NQSRETEKTIRQVQEILKKWS | 18 | 125 |
| C-Term 12 | NQSREVKEAIKIIKKILKKQS | 18 | 126 |
| C-Term 13 | NQSREIKDAIKKAKEFIKTIK | 18 | 127 |
| C-Term 14 | NQSREIETAIKKAKEFIKTIK | 18 | 128 |
| C-Term 15 | NQSRKATETIKKFEESEKS | 16 | 129 |
| C-Term 16 | NQSRDTIKVAIIVKELYKKIS | 18 | 130 |
| C-Term 17 | NQSRKTLETIEWVKKVIKKQRS | 19 | 131 |
| C-Term 18 | NQSRKTLETIEWVEKVIKKQRS | 19 | 132 |
| C-Term 19 | NQSRKWNESSKKVQEQDS | 15 | 133 |
| C-Term 20 | NQSRKTEKAIRLVLKWLKES | 17 | 134 |
| C-Term 21 | NQSRDTLKAIEQTKRYLEELKKS | 20 | 135 |
| C-Term 22 | NQSRSWDIAAKFVKTVLSNQS | 18 | 136 |
| C-Term 23 | NQSRKTLEATEIAKKLAEDRS | 18 | 137 |
| C-Term 24 | NQSLEILKAAKEAKKLIEDLRRS | 20 | 138 |
| C-Term 25 | NQSKELLDAAKAVKKMLEKEKSS | 20 | 139 |
| C-Term 26 | NQSKKLLDAADAVKKMLEKEKSS | 20 | 140 |
| C-Term 27 | NQSKKVLETIRWIETVISRQRSS | 20 | 141 |
| C-Term 28 | NQSADLKKVAELVKKLMEEAKKKS | 21 | 142 |
| C-Term 29 | NQSTDTMKAARIMKEELKEKS | 18 | 143 |
| C-Term 30 | NQSRKTEEALRRADTIIKQLASKS | 21 | 144 |
| C-Term 31 | NQSKKLKSAADDVKKAKEKS | 17 | 145 |
| C-Term 32 | NQSKELKSAAEDVKKAKEKS | 17 | 146 |
| C-Term 33 | NQSRETKKATENVKTMLTKSKS | 19 | 147 |
| C-Term 34 | NQSLELKKAAKAANTDLTKKS | 18 | 148 |
| C-Term 35 | NQSLELKEAAKAANTDLTKKS | 18 | 149 |
| C-Term 36 | NQSRKLEEIARIVEQKKRTEEKRS | 21 | 150 |
| C-Term 37 | NQSAETKKAIERAREL | 13 | 151 |
| C-Term 38 | NQSRDLKKAAEIAKKS | 13 | 152 |
| C-Term 39 | NQSRTLLETAEIVTRS | 13 | 153 |
| C-Term 40 | NQSRTLLETAEIVKRS | 13 | 154 |
| C-Term 41 | NQSRKLDKAAEYVEKS | 13 | 155 |
| C-Term 42 | NQSKEAKKAIETAKKLS | 14 | 156 |
| C-Term 43 | NQSRKLETAAEKLKQTE | 14 | 157 |
| C-Term 44 | NQSRLMLEAVKIAQSQS | 14 | 158 |
| C-Term 45 | NQSRETKEAAESVKQMES | 15 | 159 |
| C-Term 46 | NQSRRTLKAIEITLKLLS | 15 | 160 |
| C-Term 47 | NQSRRTLTAITRVERKDS | 15 | 161 |
| C-Term 48 | NQSKKLADAADWVETVKSS | 16 | 162 |
| C-Term 49 | NQSKKTHSAIEWVERLVSS | 16 | 163 |
| C-Term 50 | NQSADTKKAAEIAKKLAKS | 16 | 164 |

In some embodiments, modeling suggests that the following substitutions will stabilize the portion of the F protein in a helical conformation.

In some embodiments, the ectodomain comprises (a) the C-terminal helix-forming segment, between about residue 500 and about residue 530 relative to SEQ ID NO: 1, comprising one or more amino acid substitutions selected such that the segment forms a stable alpha-helical homotrimer.

TABLE 2B
C-terminal Alpha-helical segments for RSV (RFdiffusion)

| | | Remodeled | SEQ ID |
|---|---|---|---|
| Name | Sequence | Length | NO: |
| C-Term 1 | NQSQSIQATTSRVDAIEAKVKHLEA | 23 | 165 |
| C-Term 2 | NQSVTINNMISSNTNEISSLQDRVKHIEDTLA | 31 | 166 |
| | L | | |
| C-Term 3 | NQSKLVKKVIKETHEIKKKLEDLLK | 23 | 167 |
| C-Term 4 | NQSRSNKKTKNKVKSIEKQVKEIEKRLEKLER | 31 | 168 |
| | A | | |
| C-Term 5 | NQSQAIRETQDEVKNLNKRINKIVTSI | 25 | 169 |
| C-Term 6 | NQSRAIKETQKRTTVLEEDLKRVKELLKS | 27 | 170 |
| C-Term 7 | NQSRQIVEVMKEVEELRKRVENIEKNL | 25 | 171 |
| C-Term 8 | NQSQKTRATEEALKKTQKEVTKLKKEIQKLT | 29 | 172 |
| C-Term 9 | NQSRSNKKTKNKVKSIEKQVKEIEKRLEKLEK | 31 | 173 |
| | A | | |
| C-Term 10 | NQSNTVRKTIETVNSLEKELKELRTEVDRLL | 29 | 174 |
| C-Term 11 | NQSKEIRNTVKKVRTIEKRLNKLETSL | 25 | 175 |
| C-Term 12 | NQSRTLKDTTELTKNLNKKLKKLEEEL | 25 | 176 |
| C-Term 13 | NQSKYISNRIKENTDQIKKLEERVTELEA | 27 | 177 |
| C-Term 14 | NQSLEIRQTSKRVESLERRVTQVERDR | 25 | 178 |

TABLE 2C
Possible substitutions at Positions 503-532 (RFdiffusion)

| Position | Preferred | Allowed residues | SEQ ID NO: |
|---|---|---|---|
| L503 | Polar | QVKRNL | 186 |
| A504 | Polar | STLAQKEY | 187 |
| F505 | Hydrophobic | IVNTL | 188 |
| I506 | Polar | QNKRVS | 189 |
| R507 | Polar | ANKEDQ | 190 |
| K508 | Hydrophobic | TMVR | 191 |
| S509 | Hydrophobic | TIKQMEVS | 192 |
| D510 | Polar | SKNDE | 193 |
| E511 | Polar | RSEKATL | 194 |
| L512 | Hydrophobic | VNTL | 195 |
| L513 | Polar | DTHKENR | 196 |
| H514 | Polar | ANESVKTD | 197 |
| N515 | Hydrophobic | IELTQ | 198 |
| V516 | Polar | EIKNRQ | 199 |
| N517 | Polar | ASKER | 200 |
| A518 | Polar | KSQRDE | 201 |
| G519 | Hydrophobic | VLI | 202 |
| I520 | Polar | KQENT | 203 |
| P521 | Polar | HDEKRNQ | 204 |
| E522 | Hydrophobic | LRIV | 205 |
| A523 | Polar | EVLKR | 206 |
| P524 | Polar | AKTER | 207 |
| R525 | Polar | HRSLNED | 208 |
| D526 | Hydrophobic | ILVR | 209 |
| G527 | Polar | EKQD | 210 |
| Q528 | Polar | DKSRA | 211 |
| A529 | Hydrophobic | TL | 212 |
| Y530 | Polar | LET | 213 |
| V531 | Polar | ARK | 214 |
| R532 | Hydrophobic | LA | 215 |

In some embodiments, the C-terminal helix-forming segment comprises between about 10 and about 30 residues. In some embodiments, the segment comprises substitutions relative to the reference sequence SEQ ID NO: 1 at two or more, three or more, or four or more residues that, without being bound by theory, may generate hydrophobic contacts between the segments in the alpha-helical homotrimer.

The computational design described herein has detailed yield information on desirable amino acid substitutions that, individually or in groups, may stabilize the RSV F protein ectodomain. Illustrative, non-limiting amino acid substitutions that may be used are described as follows. In some embodiments, the C-terminal helix-forming segment (“the segment”) comprises amino acid substitutions at one or more of positions 505-519 according to reference SEQ ID NO: 1. It will be readily understood by those skilled in the art that alignment to the reference sequence of this segment depends on preserving the helical structure of the segment, and therefore insertions and deletions in the alignment are not permitted in generating sequence alignment for this segment. The starting amino acid (e.g., F in F505) is included here for clarity only, it being understood that the modification provided herein may be used with other strains of RSV in which the starting amino acid is different from the amino acid in the RSV/B reference strain sequence SEQ ID NO: 1.

In some embodiments, the segment comprises (a) an amino acid substitution at position F505 relative to SEQ ID NO: 1, wherein F is substituted with A, I, L, M, V, G, T; (b) an amino acid substitution at position I506 relative to SEQ ID NO: 1, wherein I is substituted with any amino acids except P, preferably D, E, K, N, Q, R, S, T, Y or A, I, L, V; (c) an amino acid substitution at position R507 relative to SEQ ID NO: 1, wherein R is substituted with any amino acids except P, preferably D, E, K, N, Q, R, S, T, Y or A, I, L, V; (d) an amino acid substitution at position K508 relative to SEQ ID NO: 1, wherein K is substituted with K, Q, R, preferably A, V, T, I; (e) an amino acid substitution at position S509 relative to SEQ ID NO: 1, wherein S is substituted with A, I, L, M, V, F, W, Y, G, T, preferably A, I, L, M, V; (f) an amino acid substitution at position D510 relative to SEQ ID NO: 1, wherein D is substituted with any amino acids, preferably D, E, K, N, Q, R, S, T, Y; (g) an amino acid substitution at position E511 relative to SEQ ID NO: 1, wherein E is substituted with any amino acids; (h) an amino acid substitution at position L512 relative to SEQ ID NO: 1, wherein L is substituted with D, E, K, N, Q, R, S, T, Y, preferably A, I, L, M, V, F, W, Y, G, T; (i) an amino acid substitution at position L513 relative to SEQ ID NO: 1, wherein L is substituted with any amino acids, preferably A, I, L, M, V, F, W, Y, G, more preferably D, E, K, N, Q, R, S, T, Y; (j) an amino acid substitution at position H514 relative to SEQ ID NO: 1, wherein H is substituted with any amino acids except P, preferably D, E, K, N, Q, R, S, T, Y; (k) an amino acid substitution at position N515 relative to SEQ ID NO: 1, wherein N is substituted with any amino acids except P, preferably A, I, L, M, V, F, W, Y, G; (l) an amino acid substitution at position V516 relative to SEQ ID NO: 1, wherein V is substituted with A, I, L, M, V, F, W, Y, G, or T, S, K; (m) an amino acid substitution at position N517 relative to SEQ ID NO: 1, wherein N is substituted with any amino acid except P, preferably D, E, K, N, Q, R, S, T, Y; (n) an amino acid substitution at position T518 relative to SEQ ID NO: 1, wherein T is substituted with Any except P, preferably D, E, K, N, Q, R, S, T, Y; (o) an amino acid substitution at position G519 relative to SEQ ID NO: 1, wherein G is substituted with any amino acid except P, preferably D, E, K, N, Q, R, S, T, Y; and/or (p) any combination of (a)-(o).

In some embodiments, the segment comprises (a) an amino acid substitution at position L503 relative to SEQ ID NO: 1, wherein L is substituted with Q, V, K, R, N, L; (b) an amino acid substitution at position A504 relative to SEQ ID NO: 1, wherein A is substituted with any amino acids except P, preferably S, T, L, A, Q, K, E, Y; (c) an amino acid substitution at position F505 relative to SEQ ID NO: 1, wherein F is substituted with I, V, N, T, L; (d) an amino acid substitution at position I506 relative to SEQ ID NO: 1, wherein I is substituted with any amino acids except P, preferably Q, N, K, R, V, S; (e) an amino acid substitution at position R507 relative to SEQ ID NO: 1, wherein R is substituted with any amino acids except P, preferably A, N, K, E, D, Q; (f) an amino acid substitution at position K508 relative to SEQ ID NO: 1, wherein K is substituted with T, M, V, R; (g) an amino acid substitution at position S509 relative to SEQ ID NO: 1, wherein S is substituted with T, I, K, Q, M, E, V, S; (h) an amino acid substitution at position D510 relative to SEQ ID NO: 1, wherein D is substituted with S, K, N, D, E; (i) an amino acid substitution at position E511 relative to SEQ ID NO: 1, wherein E is substituted with R, S, E, K, A, T, L; (j) an amino acid substitution at position L512 relative to SEQ ID NO: 1, wherein L is substituted with V, N, T, L; (k) an amino acid substitution at position L513 relative to SEQ ID NO: 1, wherein L is substituted with D, T, H, K, E, N, R; (l) an amino acid substitution at position H514 relative to SEQ ID NO: 1, wherein H is substituted with A, N, E, S, V, K, T, D; (m) an amino acid substitution at position N515 relative to SEQ ID NO: 1, wherein N is substituted with I, E, L, T, Q; (n) an amino acid substitution at position V516 relative to SEQ ID NO: 1, wherein V is substituted with E, I, K, N, R, Q; o) an amino acid substitution at position N517 relative to SEQ ID NO: 1, wherein N is substituted with A, S, K, E, R; (p) an amino acid substitution at position T518 relative to SEQ ID NO: 1, wherein T is substituted with K, S, Q, R, D, E; (q) an amino acid substitution at position G519 relative to SEQ ID NO: 1, wherein G is substituted with V, L, I; (r) an amino acid substitution at position 1520 relative to SEQ ID NO: 1, wherein I is substituted with K, Q, E, N, T; (s) an amino acid substitution at position P521 relative to SEQ ID NO: 1, wherein P is substituted with H, D, E, K, R, N, Q; (t) an amino acid substitution at position E522 relative to SEQ ID NO: 1, wherein E is substituted with L, R, I, V; (u) an amino acid substitution at position A523 relative to SEQ ID NO: 1, wherein A is substituted with E, V, L, K, R I; (v) an amino acid substitution at position P524 relative to SEQ ID NO: 1, wherein P is substituted with A, K, T, E, R; (w) an amino acid substitution at position R525 relative to SEQ ID NO: 1, wherein R is substituted with H, R, S, L, N, E, D; (x) an amino acid substitution at position D526 relative to SEQ ID NO: 1, wherein D is substituted with I, L, V, R; (y) an amino acid substitution at position G527 relative to SEQ ID NO: 1, wherein G is substituted with E, K, Q, D; (z) an amino acid substitution at position Q528 relative to SEQ ID NO: 1, wherein Q is substituted with D, K, S, R, A; (aa) an amino acid substitution at position A529 relative to SEQ ID NO: 1, wherein A is substituted with T, L; (ab) an amino acid substitution at position Y530 relative to SEQ ID NO: 1, wherein Y is substituted with L, E, T; (ac) an amino acid substitution at position V531 relative to SEQ ID NO: 1, wherein V is substituted with A, R, K; (ad) an amino acid substitution at position R532 relative to SEQ ID NO: 1, wherein R is substituted with V, A; and/or (ae) any combination of (a)-(ad).

In some embodiments, the segment comprises a polypeptide sequence listed in Table 2A, or a polypeptide sequence having between 1 and 5 amino acid substitutions thereto. In some embodiments, a polypeptide sequence listed in Table 2A or having 1, 2, 3, 4, 5, or more amino acid substitutions thereto. In some embodiments, the segment comprises a polypeptide sequence listed in Table 2B, or a polypeptide sequence having between 1 and 5 amino acid substitutions thereto. In some embodiments, a polypeptide sequence listed in Table 2B or having 1, 2, 3, 4, 5, or more amino acid substitutions thereto.

In some embodiments, the segment comprises the polypeptide sequence NQSREIIRAINIVRKIASEK (SEQ ID NO: 10), NQSALWLEAAKYVKQAREKS (SEQ ID NO: 11), NQSAKNAEAAKIAEETKRKD (SEQ ID NO: 12), or NQSRETAKAVSAVK (SEQ ID NO: 75), or a polypeptide sequence having between 1 and 5 amino acid substitutions thereto. In some embodiments, the segment comprises the polypeptide sequence NQSREIIRAINIVRKIASEK (SEQ ID NO: 10) or a polypeptide sequence having between 1 and 5 amino acid substitutions thereto. In some embodiments, the segment comprises the polypeptide sequence NQSREIIRAINIVRKIASEK (SEQ ID NO: 10).

In some embodiments, the C-terminal helix-forming segment comprises between about 5 and about 30 residues. In some embodiments, the C-terminal helix-forming segment comprises between about 5 and about 25 residues. In some embodiments, the C-terminal helix-forming segment comprises between about 5 and about 20 residues. In some embodiments, the C-terminal helix-forming segment comprises between about 5 and about 15 residues. In some embodiments, the C-terminal helix-forming segment comprises between about 5 and about 10 residues. In some embodiments, the C-terminal helix-forming segment comprises between about 10 and about 30 residues. In some embodiments, the C-terminal helix-forming segment comprises between about 10 and about 25 residues. In some embodiments, the C-terminal helix-forming segment comprises between about 10 and about 20 residues. In some embodiments, the C-terminal helix-forming segment comprises between about 10 and about 15 residues. In some embodiments, the C-terminal helix-forming segment comprises between about 15 and about 30 residues. In some embodiments, the C-terminal helix-forming segment comprises between about 15 and about 25 residues. In some embodiments, the C-terminal helix-forming segment comprises between about 15 and about 20 residues.

In another aspect, the disclosure provides an alpha-helical segment, comprising a polypeptide sequence listed in Table 2A or Table 2B, or a polypeptide sequence having between 1 and 5 amino acid substitutions thereto. In some embodiments, the polypeptide comprises a trimeric pathogen protein, N-terminally or C-terminally linked to the alpha-helical segment. In some embodiments, the C-terminal helix-forming segment comprises at least 5 residues. In some embodiments, the C-terminal helix-forming segment comprises at least 10 residues. In some embodiments, the C-terminal helix-forming segment comprises at least 15 residues. In some embodiments, the C-terminal helix-forming segment comprises at least 20 residues. In some embodiments, the C-terminal helix-forming segment comprises at least 25 residues.

### Stabilizing Substitutions

Computational modeling was used to identify amino acid substitutions to stabilize RSV/B F protein in the prefusion conformation. Without being bound by theory, the following amino acid substitutions are described herein as “stabilizing substitutions” because they are predicted to stabilize the RSV F protein by increasing shape complementarity within the tertiary structure of RSV F protein in the prefusion conformation. The amino acid substitutions may have other effects on structure, such as generating hydrophobic or charge-charge interactions (e.g., salt bridges) within the structure. These mutations are listed in Table 3A.

TABLE 3A
Stabilizing substitutions

| Space | Substitutions |
|---|---|
| Space 1 | F140W, K399A, K399V, T400D, S485I, S485A, |
| | S485F, D486A, D486Q, D486E, D486S, E487R, |
| | E487K, E487A, E487M, E487Q, 487R, 487M, |
| | F488W, D489A, Q494I, Q494M, Q494L, Q494A, |
| | K498A, K498E, 498A, 498Y |
| Space 2 | V56L, V56A, T58A, T58S, T58M, V154I, V187L, |
| | V296A, A298M, A298L, A298I |
| Space 3 | K75Q, N216S, N216D, E218P, T219S |
| Space 4 | E92I, E92A, E232A, E232W, R235Y, R235W, |
| | S238A, S238L, T249P, Y250F, N254V, N254L |
| Other | T67V, F137D, F137S, R339E |

Embodiments of combinations of substitutions are shown in Table 3B.

TABLE 3B

E487R + K498A
E487R + K498E
E487K + K498E
D486A + E487R + K498A
D486Q + E487R + K498A
D486E + E487A + D489A + T400D
D486A + E487M + K498A
E487Q
D486S
F488W + D489A + T400D + E487R + K498A
F140W + D489A + T400D + E487R + K498A
Q494I + S485I + K399A + 487R + 498A
Q494M + S485I + K399A, D486A + 487M + 498A
Q494L + S485A + K399V + D486A + 487M + 498A
Q494M + S485A + K399V + D486A + 487M + 498A
Q494A + S485F + K399V + D486A + 487M + 498Y
D489A + T400D + E487R + K498A
D489A + T400D

In some embodiments, the ectodomain comprises the amino acid substitutions S155C, S290C, S190F, and V207L.

In some embodiments, the ectodomain comprises one, two, three or more amino acid substitutions at positions 140, 399, 400, 485, 486, 487, 488, 489, 494, or 498 relative to SEQ ID NO: 1.

In some embodiments, the ectodomain comprises one or more of the following sets of amino acid substitutions relative to SEQ ID NO: 1: E487R+K498A; E487R+K498E; E487K+K498E; D486A+E487R+K498A; D486Q+E487R+K498A; D486E+E487A+D489A+T400D; D486A+E487M+K498A; E487Q; D486S; F488W+D489A+T400D+E487R+K498A; F140W+D489A+T400D+E487R+K498A; Q494I+S485I+K399A+487R+498A; Q494M+S485I+K399A; D486A+487M+498A; Q494L+S485A+K399V+D486A+487M+498A; Q494M+S485A+K399V+D486A+487M+498A; Q494A+S485F+K399V+D486A+487M+498Y; D489A+T400D+E487R+K498A; or D489A+T400D. In some embodiments, the ectodomain comprises the amino acid substitutions F488W, D489A, T400D, E487R, K498A, and D486A. In some embodiments, the ectodomain comprises the amino acid substitutions F488W, D489A, T400D, E487R, K498A, and T249P.

### Additional Substitutions to Stabilize the F Protein in a Prefusion Conformation

Without being bound by theory, the following amino acid substitutions are predicted to stabilize the RSV F protein. The amino acid substitutions may have other effects on structure, such as generating hydrophobic or charge-charge interactions (e.g., salt bridges) within the structure. These mutations are listed in Table 4A.

TABLE 4A

Substitutions
T54H, S55C, T58M, K66E, N67I, T67I, T67V, N88C, E92C,
E92D, Q98C, Q101P, T103C, R106C, F140W, L142C,
V144C, I148C, A149C, V154I, S155C, L188C, S190I,
S215P, E232A, R235Y, S238C, T249P, N254C, Q279C,
V296A, V296I, A298L, Q361C, N371C, K399A, T400D,
N428C, Y458C, S485I, D486A, D486S, D486N, E487M,
E487Q, E487R, F488W, D489A, D489S, Q494M, V495Y,
K498A

In some embodiments, the ectodomain comprises one, two, three or more amino acid substitutions at positions 54, 55, 58, 66, 67, 88, 92, 98, 101, 103, 106, 140, 142, 144, 148, 149, 154, 155, 188, 190, 207, 215, 232, 235, 238, 249, 254, 279, 290, 296, 298, 361, 371, 399, 400, 428, 458, 485, 486, 487, 488, 489, 494, 495, or 498 relative to SEQ ID NO: 1. In some embodiments, the ectodomain comprises one, two, three or more amino acid substitutions at T54H, S55C, T58M, K66E, N67I, T67I, T67V, N88C, E92C, E92D, Q98C, Q101P, T103C, R106C, F140W, L142C, V144C, I148C, A149C, V154I, S155C, L188C, S190I, S215P, E232A, R235Y, S238C, T249P, N254C, Q279C, V296A, V296I, A298L, Q361C, N371C, K399A, T400D, N428C, Y458C, S485I, D486A, D486S, D486N, E487M, E487Q, E487R, F488W, D489A, D489S, Q494M, V495Y, or K498A relative to SEQ ID NO: 1.

Combinations of substitutions are shown in Table 4B.

TABLE 4B

S155C + S290C + S190F + V207L
S55C + L188C + L142C + N371C + T54H + V296I
S55C + L188C + D486S
S55C + L188C + T54H + S190I
T103C + I148C + S190I + D486S
T103C + I148C + T54H + S190I + V296I + D486S
S55C + L188C + T54H + D486S
S55C + L188C + S190I + D486S
S55C + L188C + T54H + S190I + D486S
S155C + S290C + S190I + D486S
S55C + L188C + L142C + N371C T54H + V296I +
D486S + E487Q + D498S
S155C + S290C + T54H + S190I + V296I

In some embodiments, the ectodomain comprises the amino acid substitutions at S155C, S290C, S190F, and V207L relative to SEQ ID NO: 1. In some embodiments, the ectodomain comprises the amino acid substitutions at S55C, L188C, L142C, N371C, T54H, and V296I relative to SEQ ID NO: 1. In some embodiments, the ectodomain comprises the amino acid substitutions at S55C, L188C, and D486S relative to SEQ ID NO: 1. In some embodiments, the ectodomain comprises the amino acid substitutions at S55C, L188C, T54H, and S190I relative to SEQ ID NO: 1. In some embodiments, the ectodomain comprises the amino acid substitutions at T103C, I148C, S190I, and D486S relative to SEQ ID NO: 1. In some embodiments, the ectodomain comprises the amino acid substitutions at T103C, I148C, T54H, S190I, V296I, and D486S relative to SEQ ID NO: 1. In some embodiments, the ectodomain comprises the amino acid substitutions at S55C, L188C, T54H, and D486S relative to SEQ ID NO: 1. In some embodiments, the ectodomain comprises the amino acid substitutions at S55C, L188C, S190I, and D486S relative to SEQ ID NO: 1. In some embodiments, the ectodomain comprises the amino acid substitutions at S55C, L188C, T54H, S190I, and D486S relative to SEQ ID NO: 1. In some embodiments, the ectodomain comprises the amino acid substitutions at S155C, S290C, S190I, and D486S relative to SEQ ID NO: 1. In some embodiments, the ectodomain comprises the amino acid substitutions at S55C, L188C, L142C, N371C T54H, V296I, D486S, E487Q, and D498S relative to SEQ ID NO: 1. In some embodiments, the ectodomain comprises the amino acid substitutions at S155C, S290C, T54H, S190I, and V296I relative to SEQ ID NO: 1.

In some embodiments, a RSV F protein mutant comprises a disulfide mutation selected from the group consisting of 55C and 188C; 155C and 290C; 103C and 148C; and 142C and 371C, such as S55C and L188C, S155C and S290C, T103C and I148C, or L142C and N371C. Examples of pairs of such mutations include: 508C and 509C; 515C and 516C; 522C and 523C, such as K508C and S509C, N515C and V516C, or T522C and T523C.

In some embodiments, a RSV F protein mutant comprises one or more cavity filling mutations selected from the groups shown in Table 4C.

TABLE 4C
Disulfide mutation

| | |
|---|---|
| Amino acid position | Substituted with |

| | | |
|---|---|---|
| S | 55, 62, 155, 190, 290 | I, Y, L, H, M |
| T | 54, 58, 189, 397 | I, Y, L, H, M |
| G | 151 | A, H |
| A | 147, 298 | I, L, H, M |
| V | 164, 187, 192, 207, 220, | I, Y, H |
| | 296, 300, 495 | |
| R | 106 | W |

In some embodiments, a RSV F protein mutant comprises at least one cavity filling mutation selected from the group consisting of: T54H, S190I, and V296L.

In some embodiments, a RSV F protein mutant comprises at least one electrostatic mutation selected from the groups shown in Table 4D.

TABLE 4D
Electrostatic mutations

| | |
|---|---|
| Amino acid position | Substituted with |

| | | |
|---|---|---|
| E | 82, 92, 487 | D, F, Q, T, S, L, H |
| K | 315, 394, 399 | F, M, R, S, L, I, Q, T |
| D | 392, 486, 489 | H, S, N, T, P |
| R | 106, 339 | F, Q, N, W |

In some embodiments, the RSV F protein mutant comprises mutation D486S.

Combinations of substitutions are shown in Table 4E.

TABLE 4E
T103C + I148C + S190I + D486S
T54H + S55C + L188C + D486S
T54H + T103C + I148C + S190I + V296I + D486S
T54H + S55C + L142C + L188C + V296I + N371C
S55C + L188C + D486S
T54H + S55C + L188C + S190I
S55C + L188C + S190I + D486S
T54H + S55C + L188C + S190I + D486S
S155C + S190I + S290C + D486S
T54H + S55C + L142C + L188C + V2961 + N371C + D486S + E487Q +
D489S
T54H + S155C + S190I + S290C + V296I
N67I + S215P
N671 + S215P + E487Q
V56C + V164C
I57C + S190C
T58C + V164C
N165C + V296C
K168C + V296C
M396C + F483C

In some embodiments, the ectodomain comprises the amino acid substitutions at T103C, I148C, S190I and D486S relative to SEQ ID NO: 1. In some embodiments, the ectodomain comprises the amino acid substitutions at T54H, S55C, Li 88C and D486S relative to SEQ ID NO: 1. In some embodiments, the ectodomain comprises the amino acid substitutions at T54H, T103C, I148C, S190I, V296I and D486S relative to SEQ ID NO: 1. In some embodiments, the ectodomain comprises the amino acid substitutions at T54H, S55C, L142C, L188C, V296I and N371C relative to SEQ ID NO: 1. In some embodiments, the ectodomain comprises the amino acid substitutions at S55C, L188C and D486S relative to SEQ ID NO: 1. In some embodiments, the ectodomain comprises the amino acid substitutions at T54H, S55C, L188C and S190I relative to SEQ ID NO: 1. In some embodiments, the ectodomain comprises the amino acid substitutions at S55C, L188C, S190I and D486S relative to SEQ ID NO: 1. In some embodiments, the ectodomain comprises the amino acid substitutions at T54H, S55C, L142C, L188C, V296I, N371C, D486S, E487Q and D489S relative to SEQ ID NO: 1. In some embodiments, the ectodomain comprises the amino acid substitutions at T54H, S155C, S190I, S290C and V296I relative to SEQ ID NO: 1. In some embodiments, the ectodomain comprises the amino acid substitutions at N67I and S215P relative to SEQ ID NO: 1. In some embodiments, the ectodomain comprises the amino acid substitutions at N67I, S215P and E487Q relative to SEQ ID NO: 1. In some embodiments, the ectodomain comprises the amino acid substitutions at V56C and V164C relative to SEQ ID NO: 1. In some embodiments, the ectodomain comprises the amino acid substitutions at I57C and S190C relative to SEQ ID NO: 1. In some embodiments, the ectodomain comprises the amino acid substitutions at T58C and V164C relative to SEQ ID NO: 1. In some embodiments, the ectodomain comprises the amino acid substitutions at N165C and V296C relative to SEQ ID NO: 1. In some embodiments, the ectodomain comprises the amino acid substitutions at K168C and V296C relative to SEQ ID NO: 1. In some embodiments, the ectodomain comprises the amino acid substitutions at M396C and F483C relative to SEQ ID NO: 1.

### Combination of C-Terminal Helix-Forming Segment and Stabilizing Substitutions

In some embodiments, the disclosure provides recombinant polypeptides comprising amino acid substitutions having an engineered C-terminal alpha-helical segment that stabilize the RSV F protein in a prefusion conformation.

The native sequence of RSV/B F protein (GenBank: WDV37446.1) is shown below with the (predicted) transmembrane region underlined and the C-terminal helix of the native sequence (residues 492-501) is also underlined. The signal peptide is underlined with bold/italic.

| (SEQ ID NO: 53) | |
|---|---|
| 1 | MELLIHRSSA IFLTLAINAL YLISSQNITE EFYQSTCSAV SRGYLSALRT |
| 51 | GWYTSVITIE LSNIKETKCN GTDTKVKLIK QELDKYKNAV TELQLLMQNT |
| 101 | PAVNNRARRE APQYMNYTIN TTKNLNVSIS KKRKRRFLGF LLGVGSAIAS |
| 151 | GIAVSKVLHL EGEVNKIKNA LQLINKAVVS LSNGVSVLTS RVLDLKNYIN |
| 201 | NQLLPMVNRQ SCRISNIETV IEFQQKNSRL LEITREFSVN AGVTTPLSTY |
| 251 | MLTNSELLSL INDMPITNDQ KKLMSSNVQI VRQQSYSIMS IIKEEVLAYV |
| 301 | VQLPIYGVID TPCWKLHTSP LCTTNIKEGS NICLTRTDRG WYCDNAGSVS |
| 351 | FFPQADTCKV QSNRVFCDTM NSLTLPSEVS LCNTDIFNSK YDCKIMTSKT |
| 401 | DISSSVITSL GAIVSCYGKT KCTASNKNRG IIKTFSNGCD YVSNKGVDTV |
| 451 | SVGNTLYYVN KLEGKNLYVK GEPIINYYDP LVFPSDEFDA SISQVNEKIN |
| 501 | QSLAFIRRSD ELLHNVNTGK STTNIMITAI TIVIIVVLLS LIAIGLLLYC |
| 551 | KAKNTPVTLS KDQLSGINNI AFSK |

In some embodiments, the ectodomain comprises a polypeptide sequence at least 70%, at least 80%, at least 90%, at least 91%, at least 92%, at least 93%, at least 94%, at least 95%, at least 96%, at least 97%, at least 98%, at least 99%, or 100% identical to SEQ ID NO: 6, below, optionally lacking a p27 peptide shown in bold, and in which “X” refers to sites involving an added C-terminal helical segment and can be any amino acid:

(SEQ ID NO: 6)
QNITEEFYQSTCSAVSRGYLSALRTGWYTSVITIELSNIKETKCNGTDT
KVKLIKQELDKYKNAVTELQLLMONTPAVNNRARREAPQYMNYTINTTK
NLNVSISKKRKRRFLGFLLGVGSAIASGIAVCKVLHLEGEVNKIKNALQ
LINKAVVSLSNGVSVLTFRVLDLKNYINNQLLPMLNRQSCRISNIETVI
EFQQKNSRLLEITREFSVNAGVTTPLSTYMLINSELLSLINDMPITNDQ
KKLMSSNVQIVRQQSYSIMCIIKEEVLAYVVOLPIYGVIDTPCWKLHTS
PLCTTNIKEGSNICLTRTDRGWYCDNAGSVSFFPQADTCKVOSNRVFCD
TMNSLTLPSEVSLCNTDIENSKYDCKIMISKTDISSSVITSLGAIVSCY
GKIKCTASNKNRGIIKTFSNGCDYVSNKGVDTVSVGNTLYYVNKLEGKN
LYVKGEPIINYYDPLVFPSDEFDASISQVNEKINQSXXXXXXXXXXXXX
XXXX.

In some embodiments, the ectodomain comprises a polypeptide sequence at least 70%, at least 80%, at least 90%, at least 91%, at least 92%, at least 93%, at least 94%, at least 95%, at least 96%, at least 97%, at least 98%, at least 99%, or 100% identical to SEQ ID NO: 7, below, optionally lacking a p27 peptide shown in bold, in which “X” refers to sites involving an added C-terminal helical segment and can be any amino acid:

(SEQ ID NO: 7)
QNITEEFYQSTCSAVSKGYLSALRIGWYTSVITIELSNIKENKCNGTDA
KVKLIKQELDKYKNAVTELQLLMQSTPAINNRARRELPREMNYTLNNAK
KTNVTLSKKRKRRFLGFLLGVGSAIASGVAVCKVLHLEGEVNKIKSALL
STNKAVVSLSNGVSVLTFKVLDLKNYIDKQLLPILNKQSCSISNIETVI
EFQQKNNRLLEITREFSVNAGVTTPVSTYMLINSELLSLINDMPITNDQ
KKLMSNNVQIVRQQSYSIMCIIKEEVLAYVVQLPLYGVIDTPCWKLHTS
PLCTTNTKEGSNICLTRTDRGWYCDNAGSVSFFPQAETCKVQSNRVFCD
TMNSLTLPSEVNLCNVDIFNPKYDCKIMTSKTDVSSSVITSLGAIVSCY
GKTKCTASNKNRGIIKTESNGCDYVSNKGVDTVSVGNTLYYVNKQEGKS
LYVKGEPIINFYDPLVFPSDEFDASISQVNEKINQSXXXXXXXXXXXXX
XXXX.

In some embodiments, the ectodomain comprises a polypeptide sequence at least 70%, at least 80%, at least 90%, at least 91%, at least 92%, at least 93%, at least 94%, at least 95%, at least 96%, at least 97%, at least 98%, at least 99%, or 100% identical to SEQ ID NO: 8, below, optionally lacking a p27 peptide shown in bold:

(SEQ ID NO: 8)
QNITEEFYQSTCSAVSRGYLSALRTGWYTSVITIELSNIKETKCNGTDT
KVKLIKQELDKYKNAVTELQLLMQNTPAVNNRARREAPQYMNYTINTTK
NLNVSISKKRKRRFLGFLLGVGSAIASGIAVCKVLHLEGEVNKIKNALQ
LINKAVVSLSNGVSVLTFRVLDLKNYINNQLLPMLNRQSCRISNIETVI
EFQQKNSRLLEITREFSVNAGVTTPLSTYMLINSELLSLINDMPITNDQ
KKLMSSNVQIVRQQSYSIMCIIKEEVLAYVVQLPIYGVIDTPCWKLHTS
PLCTTNIKEGSNICLTRTDRGWYCDNAGSVSFFPQADTCKVQSNRVFCD
TMNSLTLPSEVSLCNTDIFNSKYDCKIMTSKTDISSSVITSLGAIVSCY
GKTKCTASNKNRGIIKTFSNGCDYVSNKGVDTVSVGNTLYYVNKLEGKN
LYVKGEPIINYYDPLVFPSDEFDASISQVNEKINQSREIIRAINIVRKI
ASEK.

In some embodiments, the ectodomain comprises a polypeptide sequence at least 70%, at least 80%, at least 90%, at least 91%, at least 92%, at least 93%, at least 94%, at least 95%, at least 96%, at least 97%, at least 98%, at least 99%, or 100% identical to SEQ ID NO: 9, below, optionally lacking a p27 peptide shown in bold:

(SEQ ID NO: 9)
QNITEEFYQSTCSAVSKGYLSALRTGWYTSVITIELSNIKENKCNGTDA
KVKLIKQELDKYKNAVTELQLLMQSTPAINNRARRELPREMNYTLNNAK
KTNVTLSKKRKRRFLGFLLGVGSAIASGVAVCKVLHLEGEVNKIKSALL
STNKAVVSLSNGVSVLTFKVLDLKNYIDKQLLPILNKQSCSISNIETVI
EFQQKNNRLLEITREFSVNAGVTTPVSTYMLINSELLSLINDMPITNDQ
KKLMSNNVQIVRQQSYSIMCIIKEEVLAYVVQLPLYGVIDTPCWKLHTS
PLCTINTKEGSNICLTRTDRGWYCDNAGSVSFFPQAETCKVQSNRVFCD
TMNSLTLPSEVNLCNVDIFNPKYDCKIMTSKTDVSSSVITSLGAIVSCY
GKTKCTASNKNRGIIKTFSNGCDYVSNKGVDTVSVGNTLYYVNKQEGKS
LYVKGEPIINFYDPLVFPSDEFDASISQVNEKINQSREIIRAINIVRKI
ASEK.

In some embodiments, the polypeptide comprises a sequence at least 70%, at least 80%, at least 90%, at least 91%, at least 92%, at least 93%, at least 94%, at least 95%, at least 96%, at least 97%, at least 98%, at least 99%, or 100% identical to any one or more of SEQ ID NOs: 1-9.

Illustrative sequences comprising various RSV F protein ectodomains and a C-terminal alpha-helical segment are shown in Table 4F. The signal peptide is underlined.

In some embodiments, the ectodomain comprises a polypeptide sequence at least 70%, at least 80%, at least 90%, at least 91%, at least 92%, at least 93%, at least 94%, at least 95%, at least 96%, at least 97%, at least 98%, at least 99%, or 100% identical to sequences shown in Table 4F.

TABLE 4F

| | | SEQ ID |
|---|---|---|
| Sequence | Mutations | NO: |
| MELLILKANAITTILTAVTFCFASGQNITEEFYQST | Introduced mutations: | 216 |
| CSAVSKGYLSALRTGWYTSVITIELSNIKENKCN | T103C, I148C, S190I, | |
| GTDAKVKLIKQELDKYKNAVTELQLLMQSTPAC | D486S | |
| NNRARRELPRFMNYTLNNAKKTNVTLSKKRK | Naturally occurring | |
| RRFLGFLLGVGSACASGVAVSKVLHLEGEVNKI | substitutions: | |
| KSALLSTNKAVVSLSNGVSVLTIKVLDLKNYIDK | P102A, 1379V, | |
| QLLPIVNKQSCSISNIETVIEFQQKNNRLLEITREF | M447V | |
| SVNAGVTTPVSTYMLTNSELLSLINDMPITNDQK | | |
| KLMSNNVQIVRQQSYSIMSIIKEEVLAYVVQLPL | | |
| YGVIDTPCWKLHTSPLCTTNTKEGSNICLTRTDR | | |
| GWYCDNAGSVSFFPQAETCKVQSNRVFCDTMN | | |
| SLTLPSEVNLCNVDIFNPKYDCKIMTSKTDVSSSV | | |
| ITSLGAIVSCYGKTKCTASNKNRGIIKTFSNGCDY | | |
| VSNKGVDTVSVGNTLYYVNKQEGKSLYVKGEPI | | |
| INFYDPLVFPSSEFDASISQVNEKINQSREIIRAINI | | |
| VRKIASEK | | |
| MELLILKANAITTILTAVTFCFASGQNITEEFYQST | Introduced mutations: | 217 |
| CSAVSKGYLSALRTGWYHSVITIELSNIKENKCN | T54H, T103C, I148C, | |
| GTDAKVKLIKQELDKYKNAVTELQLLMQSTPAC | S190I, V296I, D486S | |
| NNRARRELPRFMNYTLNNAKKTNVTLSKKRK | Naturally occuring | |
| RRFLGFLLGVGSACASGVAVSKVLHLEGEVNKI | substitutions: | |
| KSALLSTNKAWSLSNGVSVLTIKVLDLKNYIDKQ | P102A, 1379V , | |
| LLPIVNKQSCSISNIETVIEFQQKNNRLLEITREFS | M447V | |
| VNAGVTTPVSTYMLTNSELLSLINDMPITNDQKK | | |
| LMSNNVQIVRQQSYSIMSIIKEEILAYVVQLPLYG | | |
| VIDTPCWKLHTSPLCTTNTKEGSNICLTRTDRGW | | |
| YCDNAGSVSFFPQAETCKVQSNRVFCDTMNSLT | | |
| LPSEVNLCNVDIFNPKYDCKIMTSKTDVSSSVITS | | |
| LGAIVSCYGKTKCTASNKNRGIIKTFSNGCDYVS | | |
| NKGVDTVSVGNTLYYVNKQEGKSLYVKGEPIIN | | |
| FYDPLVFPSSEFDASISQVNEKINQSREIIRAINIVR | | |
| KIASEK | | |
| MELLILKANAITTILTAVTFCFASGQNITEEFYQST | Introduced mutations: | 218 |
| CSAVSKGYLSALRTGWYHCVITIELSNIKENKCN | T54H, S55C, L188C, | |
| GTDAKVKLIKQELDKYKNAVTELQLLMQSTPAT | D486S | |
| NNRARRELPRFMNYTLNNAKKTNVTLSKKRK | Naturally occuring | |
| RRFLGFLLGVGSAIASGVAVSKVLHLEGEVNKIK | substitutions: | |
| SALLSTNKAVVSLSNGVSVCTSKVLDLKNYIDK | P102A, 1379V, | |
| QLLPIVNKQSCSISNIETVIEFQQKNNRLLEITREF | M447V | |
| SVNAGVTTPVSTYMLTNSELLSLINDMPITNDQK | | |
| KLMSNNVQIVRQQSYSIMSIIKEEVLAYVVQLPL | | |
| YGVIDTPCWKLHTSPLCTTNTKEGSNICLTRTDR | | |
| GWYCDNAGSVSFFPQAETCKVQSNRVFCDTMN | | |
| SLTLPSEVNLCNVDIFNPKYDCKIMTSKTDVSSSV | | |
| ITSLGAIVSCYGKTKCTASNKNRGIIKTFSNGCDY | | |
| VSNKGVDTVSVGNTLYYVNKQEGKSLYVKGEPI | | |
| INFYDPLVFPSSEFDASISQVNEKINQSREIIRAINI | | |
| VRKIASEK | | |
| MELLILKANAITTILTAVTFCFASGQNITEEFYQST | Introduced mutations: | 219 |
| CSAVSKGYLSALRTGWYHCVITIELSNIKENKCN | T54H, S55C, L142C, | |
| GTDAKVKLIKQELDKYKNAVTELQLLMQSTPAT | L188C, V296I, N371C | |
| NNRARRELPRFMNYTLNNAKKTNVTLSKKRK | Naturally occuring | |
| RRFLGFLCGVGSAIASGVAVSKVLHLEGEVNKIK | substitutions: | |
| SALLSTNKAVVSLSNGVSVCTSKVLDLKNYIDK | P102A, I379V, | |
| QLLPIVNKQSCSISNIETVIEFQQKNNRLLEITREF | M447V | |
| SVNAGVTTPVSTYMLTNSELLSLINDMPITNDQK | | |
| KLMSNNVQIVRQQSYSIMSIIKEEILAYVVQLPLY | | |
| GVIDTPCWKLHTSPLCTTNTKEGSNICLTRTDRG | | |
| WYCDNAGSVSFFPQAETCKVQSNRVFCDTMCSL | | |
| TLPSEVNLCNVDIFNPKYDCKIMTSKTDVSSSVIT | | |
| SLGAIVSCYGKTKCTASNKNRGIIKTFSNGCDYV | | |
| SNKGVDTVSVGNTLYYVNKQEGKSLYVKGEPII | | |
| NFYDPLVFPSDEFDASISQVNEKINQSREIIRAINI | | |
| VRKIASEK | | |
| MELLILKANAITTILTAVTFCFASGQNITEEFYQST | Introduced mutations: | 220 |
| CSAVSKGYLSALRTGWYTCVITIELSNIKENKCN | S55C, L188C, D486S | |
| GTDAKVKLIKQELDKYKNAVTELQLLMQSTPAT | Naturally occuring | |
| NNRARRELPRFMNYTLNNAKKTNVTLSKKRK | substitutions: | |
| RRFLGFLLGVGSAIASGVAVSKVLHLEGEVNKIK | P102A, I379V, | |
| SALLSTNKAVVSLSNGVSVCTSKVLDLKNYIDK | M447V | |
| QLLPIVNKQSCSISNIETVIEFQQKNNRLLEITREF | | |
| SVNAGVTTPVSTYMLTNSELLSLINDMPITNDQK | | |
| KLMSNNVQIVRQQSYSIMSIIKEEVLAYWQLPLY | | |
| GVIDTPCWKLHTSPLCTTNTKEGSNICLTRTDRG | | |
| WYCDNAGSVSFFPQAETCKVQSNRVFCDTMNSL | | |
| TLPSEVNLCNVDIFNPKYDCKIMTSKTDVSSSVIT | | |
| SLGAIVSCYGKTKCTASNKNRGIIKTFSNGCDYV | | |
| SNKGVDTVSVGNTLYYVNKQEGKSLYVKGEPII | | |
| NFYDPLVFPSSEFDASISQVNEKINQSREIIRAINIV | | |
| RKIASEK | | |
| MELLILKANAITTILTAVTFCFASGQNITEEFYQST | Introduced mutations: | 221 |
| CSAVSKGYLSALRTGWYHCVITIELSNIKENKCN | T54H, S55C, L188C, | |
| GTDAKVKLIKQELDKYKNAVTELQLLMQSTPAT | S190I | |
| NNRARRELPRFMNYTLNNAKKTNVTLSKKRK | Naturally occuring | |
| RRFLGFLLGVGSAIASGVAVSKVLHLEGEVNKIK | substitutions: | |
| SALLSTNKAVVSLSNGVSVCTIKVLDLKNYIDKQ | P102A, I379V, | |
| LLPIVNKQSCSISNIETVIEFQQKNNRLLEITREFS | M447V | |
| VNAGVTTPVSTYMLTNSELLSLINDMPITNDQKK | | |
| LMSNNVQIVRQQSYSIMSIIKEEVLAYVVQLPLY | | |
| GVIDTPCWKLHTSPLCTTNTKEGSNICLTRTDRG | | |
| WYCDNAGSVSFFPQAETCKVQSNRVFCDTMNSL | | |
| TLPSEVNLCNVDIFNPKYDCKIMTSKTDVSSSVIT | | |
| SLGAIVSCYGKTKCTASNKNRGIIKTFSNGCDYV | | |
| SNKGVDTVSVGNTLYYVNKQEGKSLYVKGEPII | | |
| NFYDPLVFPSDEFDASISQVNEKINQSREIIRAINI | | |
| VRKIASEK | | |
| MELLILKANAITTILTAVTFCFASGQNITEEFYQST | Introduced mutations: | 222 |
| CSAVSKGYLSALRTGWYTCVITIELSNIKENKCN | S55C, L188C, S190I, | |
| GTDAKVKLIKQELDKYKNAVTELQLLMQSTPAT | D486S | |
| NNRARRELPRFMNYTLNNAKKTNVTLSKKRK | Naturally occuring | |
| RRFLGFLLGVGSAIASGVAVSKVLHLEGEVNKIK | substitutions: | |
| SALLSTNKAVVSLSNGVSVCTIKVLDLKNYIDKQ | P102A, I379V, | |
| LLPIVNKQSCSISNIETVIEFQQKNNRLLEITREFS | M447V | |
| VNAGVTTPVSTYMLTNSELLSLINDMPITNDQKK | | |
| LMSNNVQIVRQQSYSIMSIIKEEVLAYVVQLPLY | | |
| GVIDTPCWKLHTSPLCTTNTKEGSNICLTRTDRG | | |
| WYCDNAGSVSFFPQAETCKVQSNRVFCDTMNSL | | |
| TLPSEVNLCNVDIFNPKYDCKIMTSKTDVSSSVIT | | |
| SLGAIVSCYGKTKCTASNKNRGIIKTFSNGCDYV | | |
| SNKGVDTVSVGNTLYYVNKQEGKSLYVKGEPII | | |
| NFYDPLVFPSSEFDASISQVNEKINQSREIIRAINIV | | |
| RKIASEK | | |
| MELLILKANAITTILTAVTFCFASGQNITEEFYQST | Introduced mutations: | 223 |
| CSAVSKGYLSALRTGWYHCVITIELSNIKENKCN | T54H, S55C, L188C, | |
| GTDAKVKLIKQELDKYKNAVTELQLLMQSTPAT | S190I, D486S | |
| NNRARRELPRFMNYTLNNAKKTNVTLSKKRK | Naturally occuring | |
| RRFLGFLLGVGSAIASGVAVSKVLHLEGEVNKIK | substitutions: | |
| SALLSTNKAVVSLSNGVSVCTIKVLDLKNYIDKQ | P102A, I379V, | |
| LLPIVNKQSCSISNIETVIEFQQKNNRLLEITREFS | M447V | |
| VNAGVTTPVSTYMLTNSELLSLINDMPITNDQKK | | |
| LMSNNVQIVRQQSYSIMSIIKEEVLAYVVQLPLY | | |
| GVIDTPCWKLHTSPLCTTNTKEGSNICLTRTDRG | | |
| WYCDNAGSVSFFPQAETCKVQSNRVFCDTMNSL | | |
| TLPSEVNLCNVDIFNPKYDCKIMTSKTDVSSSVIT | | |
| SLGAIVSCYGKTKCTASNKNRGIIKTFSNGCDYV | | |
| SNKGVDTVSVGNTLYYVNKQEGKSLYVKGEPII | | |
| NFYDPLVFPSSEFDASISQVNEKINQSREIIRAINIV | | |
| RKIASEK | | |
| MELLILKANAITTILTAVTFCFASGQNITEEFYQST | Introduced mutations: | 224 |
| CSAVSKGYLSALRTGWYTSVITIELSNIKENKCN | S155C, S1901, S290C, | |
| GTDAKVKLIKQELDKYKNAVTELQLLMQSTPAT | D486S | |
| NNRARRELPRFMNYTLNNAKKTNVTLSKKRK | Naturally occuring | |
| RRFLGFLLGVGSAIASGVAVCKVLHLEGEVNKIK | substitutions: | |
| SALLSTNKAVVSLSNGVSVLTIKVLDLKNYIDKQ | P102A, I379V, | |
| LLPIVNKQSCSISNIETVIEFQQKNNRLLEITREFS | M447V | |
| VNAGVTTPVSTYMLTNSELLSLINDMPITNDQKK | | |
| LMSNNVQIVRQQSYSIMCIIKEEVLAYVVQLPLY | | |
| GVIDTPCWKLHTSPLCTTNTKEGSNICLTRTDRG | | |
| WYCDNAGSVSFFPQAETCKVQSNRVFCDTMNSL | | |
| TLPSEVNLCNVDIFNPKYDCKIMTSKTDVSSSVIT | | |
| SLGAIVSCYGKTKCTASNKNRGIIKTFSNGCDYV | | |
| SNKGVDTVSVGNTLYYVNKQEGKSLYVKGEPII | | |
| NFYDPLVFPSSEFDASISQVNEKINQSREIIRAINIV | | |
| RKIASEK | | |
| MELLILKANAITTILTAVTFCFASGQNITEEFYQST | Introduced mutations: | 225 |
| CSAVSKGYLSALRTGWYHCVITIELSNIKENKCN | T54H, S55C, L142C, | |
| GTDAKVKLIKQELDKYKNAVTELQLLMQSTPAT | L188C, V296I, | |
| NNRARRELPRFMNYTLNNAKKTNVTLSKKRK | N371C, D486S, | |
| RRFLGFLCGVGSAIASGVAVSKVLHLEGEVNKIK | E487Q, D489S | |
| SALLSTNKAVVSLSNGVSVCTSKVLDLKNYIDK | Naturally occuring | |
| QLLPIVNKQSCSISNIETVIEFQQKNNRLLEITREF | substitutions: | |
| SVNAGVTTPVSTYMLTNSELLSLINDMPITNDQK | P102A, I379V, | |
| KLMSNNVQIVRQQSYSIMSIIKEEILAYVVQLPLY | M447V | |
| GVIDTPCWKLHTSPLCTTNTKEGSNICLTRTDRG | | |
| WYCDNAGSVSFFPQAETCKVQSNRVFCDTMCSL | | |
| TLPSEVNLCNVDIFNPKYDCKIMTSKTDVSSSVIT | | |
| SLGAIVSCYGKTKCTASNKNRGIIKTFSNGCDYV | | |
| SNKGVDTVSVGNTLYYVNKQEGKSLYVKGEPII | | |
| NFYDPLVFPSSQFSASISQVNEKINQSREIIRAINIV | | |
| RKIASEK | | |
| MELLILKANAITTILTAVTFCFASGQNITEEFYQST | Introduced mutations: | 226 |
| CSAVSKGYLSALRTGWYHSVITIELSNIKENKCN | T54H, S155C, S190I, | |
| GTDAKVKLIKQELDKYKNAVTELQLLMQSTPAT | S290C, V296I | |
| NNRARRELPRFMNYTLNNAKKTNVTLSKKRK | Naturally occuring | |
| RRFLGFLLGVGSAIASGVAVCKVLHLEGEVNKIK | substitutions: | |
| SALLSTNKAVVSLSNGVSVLTIKVLDLKNYIDKQ | P102A, I379V, | |
| LLPIVNKQSCSISNIETVIEFQQKNNRLLEITREFS | M447V | |
| VNAGVTTPVSTYMLTNSELLSLINDMPITNDQKK | | |
| LMSNNVQIVRQQSYSIMCIIKEEILAYWQLPLYG | | |
| VIDTPCWKLHTSPLCTTNTKEGSNICLTRTDRGW | | |
| YCDNAGSVSFFPQAETCKVQSNRVFCDTMNSLT | | |
| LPSEVNLCNVDIFNPKYDCKIMTSKTDVSSSVITS | | |
| LGAIVSCYGKTKCTASNKNRGIIKTFSNGCDYVS | | |
| NKGVDTVSVGNTLYYVNKQEGKSLYVKGEPIIN | | |
| FYDPLVFPSDEFDASISQVNEKINQSREIIRAINIVR | | |
| KIASEK | | |
| MELLILKANAITTILTAVTFCFASGQNITEEFYQST | | 227 |
| CSAVSKGYLSALRTGWYHCVITIELSNIKENKCN | | |
| GTDAKVKLIKQELDKYKNAVTELQLLMQSTPAT | | |
| NNRARRELPRFMNYTLNNAKKTNVTLSKKRK | | |
| RRFLGFLLGVGSAIASGVAVSKVLHLEGEVNKIK | | |
| SALLSTNKAVVSLSNGVSVCTSKVLDLKNYIDK | | |
| QLLPIVNKQSCSISNIETVIEFQQKNNRLLEITREF | | |
| SVNAGVTTPVSTYMLTNSELLSLINDMPITNDQK | | |
| KLMSNNVQIVRQQSYSIMSIIKEEVLAYWQLPLY | | |
| GVIDTPCWKLHTSPLCTTNTKEGSNICLTRTDRG | | |
| WYCDNAGSVSFFPQAETCKVQSNRVFCDTMNSL | | |
| TLPSEVNLCNVDIFNPKYDCKIMTSKTDVSSSVIT | | |
| SLGAIVSCYGKTKCTASNKNRGIIKTFSNGCDYV | | |
| SNKGVDTVSVGNTLYYVNKQEGKSLYVKGEPII | | |
| NFYDPLVFPSSEFDASISQVNEKINQSREIIRAINIV | | |
| RKIASEK | | |
| MELLILKAAITTILTAVTFCFASGQNITEEFYQSTC | V56C + V164C | 228 |
| SAVSKGYLSALRTGWYTSCITIELSNIKENKCNG | | |
| TDAVKLIKQELDKYKNAVTELQLLMQSTPATNN | | |
| RARRELPRFMYTLNNAKKTVTLSKKRKRRFL | | |
| GFLLGVGSAIASGVAVSKVLHLEGECNKIKSALL | | |
| STNKAVVSLSNGVSVLTSKVLDLKNYIDKQLLPI | | |
| VKQSCSISNIETVIEFQQKNNRLLEITREFSVAGVT | | |
| TPVSTYMLTNSELLSLINDMPITNDQKKLMSNNV | | |
| QIVRQQSYSIMSIIKEEVLAYWQLPLYGVIDTPC | | |
| WKLHTSPLCTTNTKEGSNICLTRTDRGWYCDNA | | |
| GSVSFFPQAETCKVQSNRVFCDTMSLTLPSEVNL | | |
| CNVDIFNPKYDCKIMTSKTDVSSSVITSLGAIVSC | | |
| YGKTKCTASNKNRGIIKTFSNGCDYVSNKGVDT | | |
| VSVGNTLYYVKQEGKSLYVKGEPIINFYDPLVFP | | |
| SDEFDASISQVEKINQSREIIRAINIVRKIASEK | | |
| MELLILKAAITTILTAVTFCFASGQNITEEFYQSTC | I57C + S190C | 229 |
| SAVSKGYLSALRTGWYTSVCTIELSNIKENKCNG | | |
| TDAVKLIKQELDKYKNAVTELQLLMQSTPATNN | | |
| RARRELPRFMYTLNNAKKTVTLSKKRKRRFL | | |
| GFLLGVGSAIASGVAVSBVLHLEGEVKIKSALLS | | |
| TNKAWSLSNGVSVLTCBVLDLKNYIDKQLLPIV | | |
| KQSCSISNIETVIEFQQKNNRLLEITREFSVAGVTT | | |
| PVSTYMLTNSELLSLINDMPITNDQKKLMSNNVQ | | |
| IVRQQSYSIMSIIKEEVLAYWQLPLYGVIDTPCW | | |
| KLHTSPLCTTNTKEGSNICLTRTDRGWYCDNAG | | |
| SVSFFPQAETCKVQSNRVFCDTMSLTLPSEVNLC | | |
| NVDIFNPKYDCKIMTSKTDVSSSVITSLGAIVSCY | | |
| GKTKCTASNKNRGIIKTFSNGCDYVSNKGVDTV | | |
| SVGNTLYYVKQEGKSLYVKGEPIINFYDPLVFPS | | |
| DEFDASISQVEKINQSREIIRAINIVRKIASEK | | |
| MELLILKAAITTILTAVTFCFASGQNITEEFYQSTC | T58C + V164C | 230 |
| SAVSKGYLSALRTGWYTSVICIELSNIKENKCNG | | |
| TDAVKLIKQELDKYKNAVTELQLLMQSTPATNN | | |
| RARRELPRFMYTLNNAKKTVTLSKKRKRRFL | | |
| GFLLGVGSAIASGVAVSKVLHLEGECNKIKSALL | | |
| STNKAVVSLSNGVSVLTSKVLDLKNYIDKQLLPI | | |
| VKQSCSISNIETVIEFQQKNNRLLEITREFSVAGVT | | |
| TPVSTYMLTNSELLSLINDMPITNDQKKLMSNNV | | |
| QIVRQQSYSIMSIIKEEVLAYWQLPLYGVIDTPC | | |
| WKLHTSPLCTTNTKEGSNICLTRTDRGWYCDNA | | |
| GSVSFFPQAETCKVQSNRVFCDTMSLTLPSEVNL | | |
| CNVDIFNPKYDCKIMTSKTDVSSSVITSLGAIVSC | | |
| YGKTKCTASNKNRGIIKTFSNGCDYVSNKGVDT | | |
| VSVGNTLYYVKQEGKSLYVKGEPIINFYDPLVFP | | |
| SDEFDASISQVEKINQSREIIRAINIVRKIASEK | | |
| MELLILKAAITTILTAVTFCFASGQNITEEFYQSTC | N165C + V296C | 231 |
| SAVSKGYLSALRTGWYTSVITIELSNIKENKCNG | | |
| TDAVKLIKQELDKYKNAVTELQLLMQSTPATNN | | |
| RARRELPRFMYTLNNAKKTVTLSKKRKRRFL | | |
| GFLLGVGSAIASGVAVSBVLHLEGEVCKIKSALL | | |
| STNKAWSLSNGVSVLTSBVLDLKNYIDKQLLPIV | | |
| KQSCSISNIETVIEFQQKNNRLLEITREFSVAGVTT | | |
| PVSTYMLTNSELLSLINDMPITNDQKKLMSNNVQ | | |
| IVRQQSYSIMSIIKEECLAYWQLPLYGVIDTPCWK | | |
| LHTSPLCTTNTKEGSNICLTRTDRGWYCDNAGS | | |
| VSFFPQAETCKVQSNRVFCDTMSLTLPSEVNLCN | | |
| VDIFNPKYDCKIMTSKTDVSSSVITSLGAIVSCYG | | |
| KTKCTASNKNRGIIKTFSNGCDYVSNKGVDTVS | | |
| VGNTLYYVKQEGKSLYVKGEPIINFYDPLVFPSD | | |
| EFDASISQVEKINQSREIIRAINIVRKIASEK | | |
| MELLILKAAITTILTAVTFCFASGQNITEEFYQSTC | K168C + V296C | 232 |
| SAVSKGYLSALRTGWYTSVITIELSNIKENKCNG | | |
| TDAKVKLIKQELDKYKNAVTELQLLMQSTPATI | | |
| WRARRELPRFMYTLAKKTVTLSKKRKRRFLG | | |
| FLLGVGSAIASGVAVSBVLHLEGEVKICSALLST | | |
| NKAWSLSNGVSVLTSBVLDLKNYIDKQLLPIVK | | |
| QSCSISNIETVIEFQQKNNRLLEITREFSVAGVTTP | | |
| VSTYMLTNSELLSLINDMPITNDQKKLMSNNVQI | | |
| VRQQSYSIMSIIKEECLAYWQLPLYGVIDTPCWK | | |
| LHTSPLCTTNTKEGSNICLTRTDRGWYCDNAGS | | |
| VSFFPQAETCKVQSNRVFCDTMSLTLPSEVNLCN | | |
| VDIFNPKYDCKIMTSKTDVSSSVITSLGAIVSCYG | | |
| KTKCTASNKNRGIIKTFSNGCDYVSNKGVDTVS | | |
| VGNTLYYVKQEGKSLYVKGEPIINFYDPLVFPSD | | |
| EFDASISQVEKINQSREIIRAINIVRKIASEK | | |
| MELLILKAAITTILTAVTFCFASGQNITEEFYQSTC | M396C + F483C | 233 |
| SAVSKGYLSALRTGWYTSVITIELSNIKENKCNG | | |
| TDAVKLIKQELDKYKNAVTELQLLMQSTPATNN | | |
| RARRELPRFMYTLNNAKKTVTLSKKRKRRFL | | |
| GFLLGVGSAIASGVAVSKVLHLEGEVKIKSALLS | | |
| TNKAVVSLSNGVSVLTSKVLDLKNYIDKQLLPIV | | |
| KQSCSISNIETVIEFQQKNNRLLEITREFSVAGVTT | | |
| PVSTYMLTNSELLSLINDMPITNDQKKLMSNNVQ | | |
| IVRQQSYSIMSIIKEEVLAYWQLPLYGVIDTPCW | | |
| KLHTSPLCTTNTKEGSNICLTRTDRGWYCDNAG | | |
| SVSFFPQAETCKVQSNRVFCDTMSLTLPSEVNLC | | |
| NVDIFNPKYDCKICTSKTDVSSSVITSLGAIVSCY | | |
| GKTKCTASNKNRGIIKTFSNGCDYVSNKGVDTV | | |
| SVGNTLYYVKQEGKSLYVKGEPIINFYDPLVCPS | | |
| DEFDASISQVEKINQSREIIRAINIVRKIASEK | | |
| MELLILKANAITTILTAVTFCFASGQNITEEFYQST | | 234 |
| CSAVSKGYLSALRTGWYTSVITIELSNIKKNKCN | | |
| GTDAKVKLIKQELDKYKNAVTELQLLMQSTQAT | | |
| NNRARRELPRFMNYTLNNAKKTNVTLSKKRK | | |
| RRFLGFLLGVGSAIASGVAVSKVLHLEGEVNKIK | | |
| SALLSTNKAVVSLSNGVSVLTSKVLDLKNYIDKQ | | |
| LLPIVNKQSCSISNIETVIEFQQKNNRLLEITREFS | | |
| VNAGVTTPVSTYMLTNSELLSLINDMPITNDQKK | | |
| LMSNNVQIVRQQSYSIMSIIKEEVLAYVVQLPLY | | |
| GVIDTPCWKLHTSPLCTTNTKEGSNICLTRTDRG | | |
| WYCDNAGSVSFFPQAETCKVQSNRVFCDTMNSL | | |
| TLPSEVNLCNVDIFNPKYDCKIMTSKTDVSSSVIT | | |
| SLGAIVSCYGKTKCTASNKNRGIIKTFSNGCDYV | | |
| SNKGVDTVSVGNTLYYVNKQEGKSLYVKGEPII | | |
| NFYDPLVFPSDEFDASISQVNEKINQSREIIRAINI | | |
| VRKIASEK | | |
| MELLILKANAITTILTAVTFCFASGQNITEEFYQST | | 235 |
| CSAVSKGYLSALRTGWYTSVITIELSNIKENKCN | | |
| GTDAKVKLIKQELDKYKNAVTELQLLMOSTPAT | | |
| NNRARRELPRFMNYTLNNAKKTNVTLSKKRK | | |
| RRFLGFLLGVGSAIASGVAVCKVLHLEGEVNKIK | | |
| SALLSTNKAVVSLSNGVSVLTFKVLDLKNYIDKQ | | |
| LLPILNKQSCSISNIETVIEFQQKNNRLLEITREFSV | | |
| NAGVTTPVSTYMLTNSELLSLINDMPITNDQKKL | | |
| MSNNVQIVRQQSYSIMCIIKEEVLAYVVQLPLYG | | |
| VIDTPCWKLHTSPLCTTNTKEGSNICLTRTDRGW | | |
| YCDNAGSVSFFPQAETCKVQSNRVFCDTMNSLT | | |
| LPSEVNLCNVDIFNPKYDCKIMTSKTDVSSSVITS | | |
| LGAIVSCYGKTKCTASNKNRGIIKTFSNGCDYVS | | |
| NKGVDTVSVGNTLYYVNKQEGKSLYVKGEPIIN | | |
| FYDPLVFPSDEFDASISQVNEKINQSREIIRAINIVR | | |
| KIASEK | | |
| MELLILKANAITTILTAVTFCFASGQNITEEFYQST | | 236 |
| CSAVSKGYLSALRTGWYTSVITIELSNIKKNKCN | | |
| GTDAKVKLIKQELDKYKNAVTELQLLMQSTQAT | | |
| NNRARRELPRFMNYTLNNAKKTNVTLSKKRK | | |
| RRFLGFLLGVGSAIASGVAVSKVLHLEGEVNKIK | | |
| SALLSTNKAVVSLSNGVSVLTSKVLDLKNYIDKQ | | |
| LLPIVNKQSCSISNIETVIEFQQKNNRLLEITREFS | | |
| VNAGVTTPVSTYMLTNSELLSLINDMPITNDQKK | | |
| LMSNNVQIVRQQSYSIMSIIKEEVLAYVVQLPLY | | |
| GVIDTPCWKLHTSPLCTTNTKEGSNICLTRTDRG | | |
| WYCDNAGSVSFFPQAETCKVQSNRVFCDTMNSL | | |
| TLPSEVNLCNVDIFNPKYDCKIMTSKTDVSSSVIT | | |
| SLGAIVSCYGKTKCTASNKNRGIIKTFSNGCDYV | | |
| SNKGVDTVSVGNTLYYVNKQEGKSLYVKGEPII | | |
| NFYDPLVFPSDEFDASISQVNEKINQSREIIRAINI | | |
| VRKIASEK | | |
| MELLILKANAITTILTAVTFCFASGQNITEEFYQST | DS-Cav1 | 237 |
| CSAVSKGYLSALRTGWYTSVITIELSNIKENKCN | | |
| GTDAKVKLIKQELDKYKNAVTELQLLMQSTPAT | | |
| NNRARRELPRFMNYTLNNAKKTNVTLSKKRK | | |
| RRFLGFLLGVGSAIASGVAVCKVLHLEGEVNKIK | | |
| SALLSTNKAVVSLSNGVSVLTFKVLDLKNYIDKQ | | |
| LLPILNKQSCSISNIETVIEFQQKNNRLLEITREFSV | | |
| NAGVTTPVSTYMLTNSELLSLINDMPITNDQKKL | | |
| MSNNVQIVRQQSYSIMCIIKEEVLAYVVQLPLYG | | |
| VIDTPCWKLHTSPLCTTNTKEGSNICLTRTDRGW | | |
| YCDNAGSVSFFPQAETCKVQSNRVFCDTMNSLT | | |
| LPSEVNLCNVDIFNPKYDCKIMTSKTDVSSSVITS | | |
| LGAIVSCYGKTKCTASNKNRGIIKTFSNGCDYVS | | |
| NKGVDTVSVGNTLYYVNKQEGKSLYVKGEPIIN | | |
| FYDPLVFPSDEFDASISQVNEKINQSREIIRAINIVR | | |
| KIASEK | | |
| MELLILKANAITTILTAVTFCFASGQNITEEFYQST | | 238 |
| CSAVSKGYLSALRTGWYTSVITIELSNIKKNKCN | | |
| GTDAKVKLIKQELDKYKNAVTELQLLMQSTQAT | | |
| NNRARQQQQRFLGFLLGVGSAIASGVAVSKVLH | | |
| LEGEVNKIKSALLSTNKAVVSLSNGVSVLTSKVL | | |
| DLKNYIDKQLLPIVNKQSCSISNIETVIEFQQKNN | | |
| RLLEITREFSVNAGVTTPVSTYMLTNSELLSLIND | | |
| MPITNDQKKLMSNNVQIVRQQSYSIMSIIKEEVL | | |
| AYVVQLPLYGVIDTPCWKLHTSPLCTTNTKEGS | | |
| NICLTRTDRGWYCDNAGSVSFFPQAETCKVQSN | | |
| RVFCDTMNSLTLPSEVNLCNVDIFNPKYDCKIMT | | |
| SKTDVSSSVITSLGAIVSCYGKTKCTASNKNRGII | | |
| KTFSNGCDYVSNKGVDTVSVGNTLYYVNKQEG | | |
| KSLYVKGEPIINFYDPLVFPSDEFDASISQVNEKIN | | |
| QSREIIRAINIVRKIASEK | | |
| MELLILKTNAITAILAAVTLCFASSQNITEEFYQST | Deletion of p27 | 239 |
| CSAVSKGYLSALRTGWYTSVITIELSNIKENKCN | sequence | |
| GTDAKVKLIKQELDKYKSAVTELQLLMQSTPAT | | |
| NNKFLGFLLGVGSAIASGIAVSKVLHLEGEVNKI | | |
| KSALLSTNKAVVSLSNGVSVLTSKVLDLKNYIDK | | |
| QLLPIVNKQSCSISNIETVIEFQQKNNRLLEITREF | | |
| SVNAGVTTPVSTYMLTNSELLSLINDMPITNDQK | | |
| KLMSNNVQIVRQQSYSIMSIIKEEVLAYVVQLPL | | |
| YGVIDTPCWKLHTSPLCTTNTKEGSNICLTRTDR | | |
| GWYCDNAGSVSFFPLAETCKVQSNRVFCDTMNS | | |
| LTLPSEVNLCNIDIFNPKYDCKIMTSKTDVSSSVIT | | |
| SLGAIVSCYGKTKCTASNKNRGIIKTFSNGCDYV | | |
| SNKGVDTVSVGNTLYYVNKQEGKSLYVKGEPII | | |
| NFYDPLVFPSDEFDASISQVNEKINQSREIIRAINI | | |
| VRKIASEK | | |
| MELLILKANAITTILTAVTFCFASGQNITEEFYQST | P27 mutation | 240 |
| CSAVSKGYLSALRTGWYTSVITIELSNIKENKCN | | |
| GTDAKVKLIKQELDKYKNAVTELQLLMQSTPAT | | |
| NNRARRELPRFMNYTLNNAKKTNVTLSKKQKQ | | |
| QAIASGVAVSKVLHLEGEVNKIKSALLSTNKAV | | |
| VSLSNGVSVLTSKVLDLKNYIDKQLLPIVNKQSC | | |
| SISNIETVIEFQQKNNRLLEITREFSVNAGVTTPVS | | |
| TYMLTNSELLSLINDMPITNDQKKLMSNNVQIVR | | |
| QQSYSIMSIIKEEVLAYVVQLPLYGVIDTPCWKL | | |
| HTSPLCTTNTKEGSNICLTRTDRGWYCDNAGSVS | | |
| FFPQAETCKVQSNRVFCDTMNSLTLPSEVNLCNV | | |
| DIFNPKYDCKIMTSKTDVSSSVITSLGAIVSCYGK | | |
| TKCTASNKNRGIIKTFSNGCDYVSNKGVDTVSV | | |
| GNTLYYVNKQEGKSLYVKGEPIINFYDPLVFPSD | | |
| EFDASISQVNEKINQSREIIRAINIVRKIASEK | | |
| METPAQLLFLLLLWLPDTTGFASGQNITEEFYQS | Deletion of p27 | 241 |
| TCSAVSKGYLSALRTGWYTSVITIELSNIKKNKC | sequence | |
| NGTDAKVKLIKQELDKYKNAVTELQLLMQSTQ | | |
| ATNNRARQQQQRFLGFLLGVGSAIASGVAVSKV | | |
| LHLEGEVNKIKSALLSTNKAVVSLSNGVSVLTSK | | |
| VLDLKNYIDKQLLPIVNKQSCSISNIETVIEFQQK | | |
| NNRLLEITREFSVNAGVTTPVSTYMLTNSELLSLI | | |
| NDMPITNDQKKLMSNNVQIVRQQSYSIMSIIKEE | | |
| VLAYVVQLPLYGVIDTPCWKLHTSPLCTTNTKE | | |
| GSNICLTRTDRGWYCDNAGSVSFFPQAETCKVQ | | |
| SNRVFCDTMNSLTLPSEVNLCNVDIFNPKYDCKI | | |
| MTSKTDVSSSVITSLGAIVSCYGKTKCTASNKNR | | |
| GIIKTFSNGCDYVSNKGVDTVSVGNTLYYVNKQ | | |
| EGKSLYVKGEPIINFYDPLVFPSDEFDASISQVNE | | |
| KINQSREIIRAINIVRKIASEK | | |
| METPAQLLFLLLLWLPDTTGFASGQNITEEFYQS | Deletion of p27 | 242 |
| TCSAVSKGYLSALRTGWYTSVITIELSNIKKNKC | sequence | |
| NGTDAKVKLIKQELDKYKNAVTELQLLMQSTQ | | |
| ATNNRARQQQQRFLGFLLGVGSAIASGVAVSKV | | |
| LHLEGEVNKIKSALLSTNKAVVSLSNGVSVLTSK | | |
| VLDLKNYIDKQLLPIVNKQSCSISNIETVIEFQQK | | |
| NNRLLEITREFSVNAGVTTPVSTYMLTNSELLSLI | | |
| NDMPITNDQKKLMSNNVQIVRQQSYSIMSIIKEE | | |
| VLAYVVQLPLYGVIDTPCWKLHTSPLCTTNTKE | | |
| GSNICLTRTDRGWYCDNAGSVSFFPQAETCKVQ | | |
| SNRVFCDTMNSLTLPSEVNLCNVDIFNPKYDCKI | | |
| MTSKTDVSSSVITSLGAIVSCYGKTKCTASNKNR | | |
| GIIKTFSNGCDYVSNKGVDTVSVGNTLYYVNKQ | | |
| EGKSLYVKGEPIINFYDPLVFPSDEFDASISQVNE | | |
| KINQSREIIRAINIVRKIASEK | | |
| MELLILKANAITTILTAVTFCFASGQNITEEFYQST | DS-Cav1 | 243 |
| CSAVSKGYLSALRTGWYTSVITIELSNIKENKCN | | |
| GTDAKVKLIKQELDKYKNAVTELQLLMQSTPAT | | |
| NNRARRELPRFMNYTLNNAKKTNVTLSKKRK | | |
| RRFLGFLLGVGSAIASGVAVCKVLHLEGEVNKIK | | |
| SALLSTNKAVVSLSNGVSVLTFKVLDLKNYIDKQ | | |
| LLPILNKQSCSISNIETVIEFQQKNNRLLEITREFSV | | |
| NAGVTTPVSTYMLTNSELLSLINDMPITNDQKKL | | |
| MSNNVQIVRQQSYSIMCIIKEEVLAYVVQLPLYG | | |
| VIDTPCWKLHTSPLCTTNTKEGSNICLTRTDRGW | | |
| YCDNAGSVSFFPQAETCKVQSNRVFCDTMNSLT | | |
| LPSEVNLCNVDIFNPKYDCKIMTSKTDVSSSVITS | | |
| LGAIVSCYGKTKCTASNKNRGIIKTFSNGCDYVS | | |
| NKGVDTVSVGNTLYYVNKQEGKSLYVKGEPIIN | | |
| FYDPLVFPSDEFDASISQVNEKINQSREIIRAINIVR | | |
| KIASEK | | |
| MELLILKANAITTILTAVTFCFASQNITEEFYQSTC | | 244 |
| SAVSKGYLSALRTGWYTSVITIELSNIKENKCNG | | |
| TDAKVKLIKQELDKYKNAVTELQLLMQSTPATN | | |
| NRARRELPRFMNYTLNNAKKINVILSKKRKRRFL | | |
| GFLLGVGSAIASGVAVCKVLHLEGEVNKIKSALL | | |
| SINKAVVSLSNGVSVLIFKVLDLKNYIDKQLLPIL | | |
| NKQSCSISNIETVIEFQQKNNRLLEITREFSVNAG | | |
| VITPVSTYMLINSELLSLINDMPITNDQKKLMSNN | | |
| VQIVRQQSYSIMCIIKEEVLAYVVQLPLYGVIDTP | | |
| CWKLHISPLCTINTKEGSNICLTRIDRGWYCDNA | | |
| GSVSFFPQAETCKVQSNRVFCDTMNSLTLPSEVN | | |
| LCNVDIFNPKYDCKIMISKTDVSSSVITSLGAIVSC | | |
| YGKTKCIASNKNRGIIKTFSNGCDYVSNKGVDTV | | |
| SVGNTLYYVNKQEGKSLYVKGEPIINFYDPLVFP | | |
| SDEFDASISQVNEKINQSREIIRAINIVRKIASEK | | |

In some embodiments, the ectodomain comprises any of the stabilizing mutations of RSV F protein disclosed in U.S. Pat. Nos. 9,950,058, 8,563,002, 11,261,239, 11,629,181, and 11,655,284, each of which is hereby incorporated by reference in its entirety.

### Furin Cleavage Site

RSV F proteins are cleaved during expression by the protease furin. Constructs that replace the cleavage site for furin with a glycine-serine linker are provided herein. Sequences are provided in Table 5A. In some embodiments, RSV F protein ectodomain comprises an uncleaved furin cleavage site.

TABLE 5A
Furin cleavage linkers

| Sequence | Length | SEQ ID NO: |
|---|---|---|
| NNQARGSGSGRSLGF | 15 | 245 |
| NNQARGGSGGRSLGF | 15 | 246 |
| NNGARGGSGGRSLGF | 15 | 247 |
| NNQARGGSGGDSLGF | 15 | 248 |
| NNQARGGSGSGGDSLGF | 17 | 249 |
| NNQARGGSGGGDLG | 14 | 250 |
| NNQARGGSGSGGDLGF | 16 | 251 |

### Linkers

In some embodiments, the recombinant polypeptide and a protein nanostructure may be genetically fused such that they are both present in a single polypeptide, termed a “fusion protein.” The linkage between the polypeptide and the protein nanostructure allows the recombinant polypeptide to be displayed on the exterior of the self-assembling protein nanostructure.

A wide variety of polypeptide sequences can be used to link the proteins, or antigenic fragments thereof and the protein nanostructure. In some cases the linker comprises a polypeptide sequence that can be included in the encoding polynucleotide sequence. Any suitable linker polypeptide can be used. In some embodiments, the linker imposes a rigid relative orientation of the antigenic protein (e.g., ectodomain from the RSV Fusion protein) or antigenic fragment thereof to the protein nanostructure. In some embodiments, the linker flexibly links the antigenic protein (e.g., ectodomain from the RSV Fusion protein) or antigenic fragment thereof to the protein nanostructure. In some embodiments, the encoded polypeptides can include a linker between regions. In some embodiments, the polypeptide is a fusion protein which includes the recombinant RSV polypeptide, a linker, and the protein nanostructure component polypeptide. In some embodiments, the polypeptide is a fusion protein, which includes, in N- to C-terminal order, the recombinant RSV polypeptide, a linker, and the protein nanostructure component polypeptide. The linker can be a polypeptide. A wide variety of polypeptide sequences can be used and are well known in the art. In some embodiments, the linker may comprise a Gly-Ser linker (i.e., a linker consisting of glycine and serine residues) of any suitable length. In some embodiments, the Gly-Ser linker may be 3, 4, 5, 6, 7, 8, 9, 10, 11, 12, 13, 14, 15, 16, 17, 18, 19, 20 or more amino acid residues in length. Non-limiting examples of Glys-Ser linkers are presented in Table 5B.

TABLE 5B

| | | SEQ |
|---|---|---|
| Sequence | Length | ID NO: |
| GSS | 3 | |
| GSGS | 4 | 252 |
| GGSGEKP | 7 | 253 |
| GGSGQKP | 7 | 254 |
| GGSGGSGS | 8 | 255 |
| GGSGGSGEKP | 10 | 256 |
| GGSGGSGQKP | 10 | 257 |
| GGSGGSGGSGGS | 12 | 258 |
| GSGGSGSGSGGS | 12 | 259 |
| GGGGGSGGGSGGGGS | 15 | 260 |
| GGGGSGGGGSGGGGS | 15 | 261 |
| GGSGGSGSGGSGGSGS | 16 | 262 |
| GGGGSGGGGSGGGGSGG | 17 | 263 |
| SGGGSGGSGSGGSGGSGS | 18 | 264 |
| EPEGGSGGSGSGGSGGSGS | 19 | 265 |
| YGGSGGSGGSGSGGSGGSGS | 20 | 266 |
| GGSGGSGSGGSGGSGSGGSGSGGS | 24 | 267 |
| GSGGSGGSGGSGGSGSGGSGGSGS | 24 | 268 |
| KSDELLGSGGSGSGSGGSEKAAKAEEAARK | 30 | 269 |

In some embodiments, the linker comprises between 3 and 30 amino acid residues. In some embodiments, the linker comprises between 4 and 24 amino acid residues. In some embodiments, the linker comprises between 8 and 24 amino acid residues. In some embodiments, the linker comprises between 10 and 24 amino acid residues. In some embodiments, the linker comprises between 12 and 24 amino acid residues. In some embodiments, the linker comprises between 16 and 24 amino acid residues. In some embodiments, the linker comprises between 18 and 24 amino acid residues. In some embodiments, the linker comprises between 20 and 24 amino acid residues. In some embodiments, the linker comprises between 4 and 20 amino acid residues. In some embodiments, the linker comprises between 8 and 20 amino acid residues. In some embodiments, the linker comprises between 10 and 20 amino acid residues. In some embodiments, the linker comprises between 12 and 20 amino acid residues. In some embodiments, the linker comprises between 16 and 20 amino acid residues. In some embodiments, the linker comprises between 8 and 18 amino acid residues. In some embodiments, the linker comprises between 12 and 16 amino acid residues. In some embodiments, the linker comprises 3 amino acid residues. In some embodiments, the linker comprises 4 amino acid residues. In some embodiments, the linker comprises 5 amino acid residues. In some embodiments, the linker comprises 6 amino acid residues. In some embodiments, the linker comprises 7 amino acid residues. In some embodiments, the linker comprises 8 amino acid residues. In some embodiments, the linker comprises 8 amino acid residues. In some embodiments, the linker comprises 10 amino acid residues. In some embodiments, the linker comprises 11 amino acid residues. In some embodiments, the linker comprises 12 amino acid residues. In some embodiments, the linker comprises 13 amino acid residues. In some embodiments, the linker comprises 14 amino acid residues. In some embodiments, the linker comprises 15 amino acid residues. In some embodiments, the linker comprises 16 amino acid residues. In some embodiments, the linker comprises 17 amino acid residues. In some embodiments, the linker comprises 18 amino acid residues. In some embodiments, the linker comprises 19 amino acid residues. In some embodiments, the linker comprises 20 amino acid residues. In some embodiments, the linker comprises 21 amino acid residues. In some embodiments, the linker comprises 22 amino acid residues. In some embodiments, the linker comprises 23 amino acid residues. In some embodiments, the linker comprises 24 amino acid residues. In some embodiments, the linker comprises 25 amino acid residues. In some embodiments, the linker comprises 26 amino acid residues. In some embodiments, the linker comprises 27 amino acid residues. In some embodiments, the linker comprises 28 amino acid residues. In some embodiments, the linker comprises 29 amino acid residues. In some embodiments, the linker comprises 30 amino acid residues.

In some embodiments, the encoded polypeptides can include a linker between regions. In some embodiments, the polypeptide is a fusion protein which includes the recombinant RSV polypeptide, a linker, a N-terminal extension linker, and the protein nanostructure component polypeptide. In some embodiments, the polypeptide is a fusion protein, which includes, in N- to C-terminal order, the recombinant RSV polypeptide, a linker, a N-terminal extension linker, and the protein nanostructure component polypeptide. In some embodiments, the N-terminal extension linker is I53-50A helical extension. In some embodiments, polypeptide sequence of N-terminal extension linker is EKAAKAEEAARK (SEQ ID NO: 320).

### Trimerization Domains

In some embodiments, the polypeptide may comprise a trimerization domain, such as FoldOn or a GCN4 trimerization. In some embodiments, the linker sequence comprises a FoldOn, wherein the FoldOn sequence is GYIPEAPRDGQAYVRKDGEWVLLSTFL (SEQ ID NO: 179).

In some embodiments, the polypeptide may comprise a trimerization domain, wherein the trimerization domain sequence is DKIEEILSKIYHIENEIARIKKLIGE (GEN) (SEQ ID NO: 270). In some embodiments, the polypeptide may comprise a trimerization domain, wherein the trimerization domain sequence is EKFHQIEKEFSEVEGRIQDLEK (HA) (SEQ ID NO: 271).

In some embodiments, the polypeptide may comprise a trimerization domain, wherein the trimerization domain sequence is EDKIEEILSKIYHIENEIARIKKLIGEA (coiled-coil isoleucine zipper) (SEQ ID NO: 272).

In some embodiments, the polypeptide may comprise a trimerization domain, wherein the trimerization domain sequence is GSGYIPEAPRDGQAYVRKDGEWVLLSTFL (bacteriophage T4 fibritin) (SEQ ID NO: 273).

In some embodiments, a trimerization sequence is RMKQIEDKIEEILSKIYHIENEIARIKKLIGEA (GCN4) (SEQ ID NO: 274). In some embodiments, a trimerization domain is a GCN4 variant. In some embodiments, the GCN4 variant sequence is RMKQIEDKIEEILSKIYHIENEIARIKKLIGERGGR (SEQ ID NO: 275), RMKQIEDKIEEILSKIYHIENEIARIKKLIGNRTGGR (SEQ ID NO: 276), RMKQIEDKIENITSKIYHIENEIARIKKLIGNRTGGR (SEQ ID NO: 277), RMKQIEDKIEEILSKIYNITNEIARIKKLIGNRTGGR (SEQ ID NO: 278), or RMKQIEDKIENITSKIYNITNEIARIKKLIGNRTGGR (SEQ ID NO: 279).

Illustrative sequences comprising various RSV F protein ectodomains, a C-terminal alpha-helical segment, and FoldOn are shown in Table 5C. The signal peptide is underlined with italic. The underlined FoldOn sequence may be substituted with any one of the trimerization domains described herein or any one of the multimerization domains described in Table 6 to generate embodiments that comprise such other trimerization domains.

In some embodiments, the trimeric protein complex comprises a polypeptide sequence at least 70%, at least 80%, at least 90%, at least 91%, at least 92%, at least 93%, at least 94%, at least 95%, at least 96%, at least 97%, at least 98%, at least 99%, or 100% identical to sequences shown in Table 5C. In some embodiments, the trimeric protein complex can be used as a trimeric component of a protein nanostructure. The approximate region surrounding the p27 peptide is bold. In some embodiments, the p27 peptide may be removed from the RSV F protein ectodomain through furin-based cleavage during production of antigens in cell culture.

TABLE 5C

| | | SEQ ID |
|---|---|---|
| Sequence | Mutations | NO: |
| MELLILKANAITTILTAVTFCFASGQNITEEFYQSTCSA | Introduced mutations: | 280 |
| VSKGYLSALRTGWYTSVITIELSNIKENKCNGTDA | T103C, I148C, S190I, | |
| KVKLIKQELDKYKNAVTELQLLMQSTPACNNRAR | D486S | |
| RELPRFMNYTLNNAKKTNVTLSKKRKRRFLGFL | Naturally occurring | |
| LGVGSACASGVAVSKVLHLEGEVNKIKSALLSTNK | substitutions: | |
| AVVSLSNGVSVLTIKVLDLKNYIDKQLLPIVNKQS | P102A, I379V, M447V | |
| CSISNIETVIEFQQKNNRLLEITREFSVNAGVTTPVS | | |
| TYMLTNSELLSLINDMPITNDQKKLMSNNVQIVRQ | | |
| QSYSIMSIIKEEVLAYVVQLPLYGVIDTPCWKLHTS | | |
| PLCTTNTKEGSNICLTRTDRGWYCDNAGSVSFFPQ | | |
| AETCKVQSNRVFCDTMNSLTLPSEVNLCNVDIFNP | | |
| KYDCKIMTSKTDVSSSVITSLGAIVSCYGKTKCTAS | | |
| NKNRGIIKTFSNGCDYVSNKGVDTVSVGNTLYYV | | |
| NKQEGKSLYVKGEPIINFYDPLVFPSSEFDASISQV | | |
| NEKINQSREIIRAINIVRKIASEKSAIGGYIPEAPRD | | |
| GQAYVRKDGEWVLLSTFL | | |
| MELLILKANAITTILTAVTFCFASGQNITEEFYQSTCSA | Introduced mutations: | 281 |
| VSKGYLSALRTGWYHSVITIELSNIKENKCNGTDA | T54H, T103C, I148C, | |
| KVKLIKQELDKYKNAVTELQLLMQSTPACNNRAR | S190I, V296I, D486S | |
| RELPRFMNYTLNNAKKTNVTLSKKRKRRFLGFL | Naturally occurring | |
| LGVGSACASGVAVSKVLHLEGEVNKIKSALLSTNK | substitutions: | |
| AWSLSNGVSVLTIKVLDLKNYIDKQLLPIVNKQSC | P102A, I379V, M447V | |
| SISNIETVIEFQQKNNRLLEITREFSVNAGVTTPVST | | |
| YMLTNSELLSLINDMPITNDQKKLMSNNVQIVRQQ | | |
| SYSIMSIIKEEILAYVVQLPLYGVIDTPCWKLHTSPL | | |
| CTTNTKEGSNICLTRTDRGWYCDNAGSVSFFPQAE | | |
| TCKVQSNRVFCDTMNSLTLPSEVNLCNVDIFNPKY | | |
| DCKIMTSKTDVSSSVITSLGAIVSCYGKTKCTASNK | | |
| NRGIIKTFSNGCDYVSNKGVDTVSVGNTLYYVNK | | |
| QEGKSLYVKGEPIINFYDPLVFPSSEFDASISQVNEK | | |
| INQSREIIRAINIVRKIASEKSAIGGYIPEAPRDGQA | | |
| YVRKDGEWVLLSTFL | | |
| MELLILKANAITTILTAVTFCFASGQNITEEFYQSTCSA | Introduced mutations: | 282 |
| VSKGYLSALRTGWYHCVITIELSNIKENKCNGTDA | T54H, S55C, L188C, | |
| KVKLIKQELDKYKNAVTELQLLMQSTPATNNRAR | D486S | |
| RELPRFMNYTLNNAKKTNVTLSKKRKRRFLGFL | Naturally occurring | |
| LGVGSAIASGVAVSKVLHLEGEVNKIKSALLSTNK | substitutions: | |
| AVVSLSNGVSVCTSKVLDLKNYIDKQLLPIVNKQS | P102A, I379V, M447V | |
| CSISNIETVIEFQQKNNRLLEITREFSVNAGVTTPVS | | |
| TYMLTNSELLSLINDMPITNDQKKLMSNNVQIVRQ | | |
| QSYSIMSIIKEEVLAYVVQLPLYGVIDTPCWKLHTS | | |
| PLCTTNTKEGSNICLTRTDRGWYCDNAGSVSFFPQ | | |
| AETCKVQSNRVFCDTMNSLTLPSEVNLCNVDIFNP | | |
| KYDCKIMTSKTDVSSSVITSLGAIVSCYGKTKCTAS | | |
| NKNRGIIKTFSNGCDYVSNKGVDTVSVGNTLYYV | | |
| NKQEGKSLYVKGEPIINFYDPLVFPSSEFDASISQV | | |
| NEKINQSREIIRAINIVRKIASEKSAIGGYIPEAPRD | | |
| GQAYVRKDGEWVLLSTFL | | |
| MELLILKANAITTILTAVTFCFASGQNITEEFYQSTCSA | Introduced mutations: | 283 |
| VSKGYLSALRTGWYHCVITIELSNIKENKCNGTDA | T54H, S55C, L142C, | |
| KVKLIKQELDKYKNAVTELQLLMQSTPATNNRAR | L188C, V296I, N371C | |
| RELPRFMNYTLNNAKKTNVTLSKKRKRRFLGFL | Naturally occurring | |
| CGVGSAIASGVAVSKVLHLEGEVNKIKSALLSTNK | substitutions: | |
| AVVSLSNGVSVCTSKVLDLKNYIDKQLLPIVNKQS | P102A, I379V, M447V | |
| CSISNIETVIEFQQKNNRLLEITREFSVNAGVTTPVS | | |
| TYMLTNSELLSLINDMPITNDQKKLMSNNVQIVRQ | | |
| QSYSIMSIIKEEILAYVVQLPLYGVIDTPCWKLHTSP | | |
| LCTTNTKEGSNICLTRTDRGWYCDNAGSVSFFPQA | | |
| ETCKVQSNRVFCDTMCSLTLPSEVNLCNVDIFNPK | | |
| YDCKIMTSKTDVSSSVITSLGAIVSCYGKTKCTASN | | |
| KNRGIIKTFSNGCDYVSNKGVDTVSVGNTLYYVN | | |
| KQEGKSLYVKGEPIINFYDPLVFPSDEFDASISQVN | | |
| EKINQSREIIRAINIVRKIASEKSAIGGYIPEAPRDG | | |
| QAYVRKDGEWVLLSTFL | | |
| MELLILKANAITTILTAVTFCFASGQNITEEFYQSTCSA | Introduced mutations: | 284 |
| VSKGYLSALRTGWYTCVITIELSNIKENKCNGTDA | S55C, L188C, D486S | |
| KVKLIKQELDKYKNAVTELQLLMQSTPATNNRAR | Naturally occurring | |
| RELPRFMNYTLNNAKKTNVTLSKKRKRRFLGFL | substitutions: | |
| LGVGSAIASGVAVSKVLHLEGEVNKIKSALLSTNK | P102A, I379V, M447V | |
| AVVSLSNGVSVCTSKVLDLKNYIDKQLLPIVNKQS | | |
| CSISNIETVIEFQQKNNRLLEITREFSVNAGVTTPVS | | |
| TYMLTNSELLSLINDMPITNDQKKLMSNNVQIVRQ | | |
| QSYSIMSIIKEEVLAYWQLPLYGVIDTPCWKLHTSP | | |
| LCTTNTKEGSNICLTRTDRGWYCDNAGSVSFFPQA | | |
| ETCKVQSNRVFCDTMNSLTLPSEVNLCNVDIFNPK | | |
| YDCKIMTSKTDVSSSVITSLGAIVSCYGKTKCTASN | | |
| KNRGIIKTFSNGCDYVSNKGVDTVSVGNTLYYVN | | |
| KQEGKSLYVKGEPIINFYDPLVFPSSEFDASISQVNE | | |
| KINQSREIIRAINIVRKIASEKSAIGGYIPEAPRDGQ | | |
| AYVRKDGEWVLLSTFL | | |
| MELLILKANAITTILTAVTFCFASGQNITEEFYQSTCSA | Introduced mutations: | 285 |
| VSKGYLSALRTGWYHCVITIELSNIKENKCNGTDA | T54H, S55C, L188C, | |
| KVKLIKQELDKYKNAVTELQLLMQSTPATNNRAR | S190I | |
| RELPRFMNYTLNNAKKTNVTLSKKRKRRFLGFL | Naturally occurring | |
| LGVGSAIASGVAVSKVLHLEGEVNKIKSALLSTNK | substitutions: | |
| AVVSLSNGVSVCTIKVLDLKNYIDKQLLPIVNKQS | P102A, I379V, M447V | |
| CSISNIETVIEFQQKNNRLLEITREFSVNAGVTTPVS | | |
| TYMLTNSELLSLINDMPITNDQKKLMSNNVQIVRQ | | |
| QSYSIMSIIKEEVLAYVVQLPLYGVIDTPCWKLHTS | | |
| PLCTTNTKEGSNICLTRTDRGWYCDNAGSVSFFPQ | | |
| AETCKVQSNRVFCDTMNSLTLPSEVNLCNVDIFNP | | |
| KYDCKIMTSKTDVSSSVITSLGAIVSCYGKTKCTAS | | |
| NKNRGIIKTFSNGCDYVSNKGVDTVSVGNTLYYV | | |
| NKQEGKSLYVKGEPIINFYDPLVFPSDEFDASISQV | | |
| NEKINQSREIIRAINIVRKIASEKSAIGGYIPEAPRD | | |
| GQAYVRKDGEWVLLSTFL | | |
| MELLILKANAITTILTAVTFCFASGQNITEEFYQSTCSA | Introduced mutations: | 286 |
| VSKGYLSALRTGWYTCVITIELSNIKENKCNGTDA | S55C, L188C, S190I, | |
| KVKLIKQELDKYKNAVTELQLLMQSTPATNNRAR | D486S | |
| RELPRFMNYTLNNAKKTNVTLSKKRKRRFLGFL | Naturally occurring | |
| LGVGSAIASGVAVSKVLHLEGEVNKIKSALLSTNK | substitutions: | |
| AVVSLSNGVSVCTIKVLDLKNYIDKQLLPIVNKQS | P102A, I379V, M447V | |
| CSISNIETVIEFQQKNNRLLEITREFSVNAGVTTPVS | | |
| TYMLTNSELLSLINDMPITNDQKKLMSNNVQIVRQ | | |
| QSYSIMSIIKEEVLAYVVQLPLYGVIDTPCWKLHTS | | |
| PLCTTNTKEGSNICLTRTDRGWYCDNAGSVSFFPQ | | |
| AETCKVQSNRVFCDTMNSLTLPSEVNLCNVDIFNP | | |
| KYDCKIMTSKTDVSSSVITSLGAIVSCYGKTKCTAS | | |
| NKNRGIIKTFSNGCDYVSNKGVDTVSVGNTLYYV | | |
| NKQEGKSLYVKGEPIINFYDPLVFPSSEFDASISQV | | |
| NEKINQSREIIRAINIVRKIASEKSAIGGYIPEAPRD | | |
| GQAYVRKDGEWVLLSTFL | | |
| MELLILKANAITTILTAVTFCFASGQNITEEFYQSTCSA | Introduced mutations: | 287 |
| VSKGYLSALRTGWYHCVITIELSNIKENKCNGTDA | T54H, S55C, L188C, | |
| KVKLIKQELDKYKNAVTELQLLMQSTPATNNRAR | S190I, D486S | |
| RELPRFMNYTLNNAKKTNVTLSKKRKRRFLGFL | Naturally occurring | |
| LGVGSAIASGVAVSKVLHLEGEVNKIKSALLSTNK | substitutions: | |
| AVVSLSNGVSVCTIKVLDLKNYIDKQLLPIVNKQS | P102A, I379V, M447V | |
| CSISNIETVIEFQQKNNRLLEITREFSVNAGVTTPVS | | |
| TYMLTNSELLSLINDMPITNDQKKLMSNNVQIVRQ | | |
| QSYSIMSIIKEEVLAYVVQLPLYGVIDTPCWKLHTS | | |
| PLCTTNTKEGSNICLTRTDRGWYCDNAGSVSFFPQ | | |
| AETCKVQSNRVFCDTMNSLTLPSEVNLCNVDIFNP | | |
| KYDCKIMTSKTDVSSSVITSLGAIVSCYGKTKCTAS | | |
| NKNRGIIKTFSNGCDYVSNKGVDTVSVGNTLYYV | | |
| NKQEGKSLYVKGEPIINFYDPLVFPSSEFDASISQV | | |
| NEKINQSREIIRAINIVRKIASEKSAIGGYIPEAPRD | | |
| GQAYVRKDGEWVLLSTFL | | |
| MELLILKANAITTILTAVTFCFASGQNITEEFYQSTCSA | Introduced mutations: | 288 |
| VSKGYLSALRTGWYTSVITIELSNIKENKCNGTDA | S155C, S190I, S290C, | |
| KVKLIKQELDKYKNAVTELQLLMQSTPATNNRAR | D486S | |
| RELPRFMNYTLNNAKKTNVTLSKKRKRRFLGFL | Naturally occurring | |
| LGVGSAIASGVAVCKVLHLEGEVNKIKSALLSTNK | substitutions: | |
| AVVSLSNGVSVLTIKVLDLKNYIDKQLLPIVNKQS | P102A, I379V, M447V | |
| CSISNIETVIEFQQKNNRLLEITREFSVNAGVTTPVS | | |
| TYMLTNSELLSLINDMPITNDQKKLMSNNVQIVRQ | | |
| QSYSIMCIIKEEVLAYVVQLPLYGVIDTPCWKLHTS | | |
| PLCTTNTKEGSNICLTRTDRGWYCDNAGSVSFFPQ | | |
| AETCKVQSNRVFCDTMNSLTLPSEVNLCNVDIFNP | | |
| KYDCKIMTSKTDVSSSVITSLGAIVSCYGKTKCTAS | | |
| NKNRGIIKTFSNGCDYVSNKGVDTVSVGNTLYYV | | |
| NKQEGKSLYVKGEPIINFYDPLVFPSSEFDASISQV | | |
| NEKINQSREIIRAINIVRKIASEKSAIGGYIPEAPRD | | |
| GQAYVRKDGEWVLLSTFL | | |
| MELLILKANAITTILTAVTFCFASGQNITEEFYQSTCSA | Introduced mutations: | 289 |
| VSKGYLSALRTGWYHCVITIELSNIKENKCNGTDA | T54H, S55C, L142C, | |
| KVKLIKQELDKYKNAVTELQLLMQSTPATNNRAR | L188C, V296I, N371C, | |
| RELPRFMNYTLNNAKKTNVTLSKKRKRRFLGFL | D486S, E487Q, D489S | |
| CGVGSAIASGVAVSKVLHLEGEVNKIKSALLSTNK | Naturally occurring | |
| AVVSLSNGVSVCTSKVLDLKNYIDKQLLPIVNKQS | substitutions: | |
| CSISNIETVIEFQQKNNRLLEITREFSVNAGVTTPVS | P102A, I379V, M447V | |
| TYMLTNSELLSLINDMPITNDQKKLMSNNVQIVRQ | | |
| QSYSIMSIIKEEILAYVVQLPLYGVIDTPCWKLHTSP | | |
| LCTTNTKEGSNICLTRTDRGWYCDNAGSVSFFPQA | | |
| ETCKVQSNRVFCDTMCSLTLPSEVNLCNVDIFNPK | | |
| YDCKIMTSKTDVSSSVITSLGAIVSCYGKTKCTASN | | |
| KNRGIIKTFSNGCDYVSNKGVDTVSVGNTLYYVN | | |
| KQEGKSLYVKGEPIINFYDPLVFPSSQFSASISQVNE | | |
| KINQSREIIRAINIVRKIASEKSAIGGYIPEAPRDGQ | | |
| AYVRKDGEWVLLSTFL | | |
| MELLILKANAITTILTAVTFCFASGQNITEEFYQSTCSA | Introduced mutations: | 290 |
| VSKGYLSALRTGWYHSVITIELSNIKENKCNGTDA | T54H, S155C, S190I, | |
| KVKLIKQELDKYKNAVTELQLLMQSTPATNNRAR | S290C, V296I | |
| RELPRFMNYTLNNAKKTNVTLSKKRKRRFLGFL | Naturally occurring | |
| LGVGSAIASGVAVCKVLHLEGEVNKIKSALLSTNK | substitutions: | |
| AVVSLSNGVSVLTIKVLDLKNYIDKQLLPIVNKQS | P102A, I379V, M447V | |
| CSISNIETVIEFQQKNNRLLEITREFSVNAGVTTPVS | | |
| TYMLTNSELLSLINDMPITNDQKKLMSNNVQIVRQ | | |
| QSYSIMCIIKEEILAYWQLPLYGVIDTPCWKLHTSP | | |
| LCTTNTKEGSNICLTRTDRGWYCDNAGSVSFFPQA | | |
| ETCKVQSNRVFCDTMNSLTLPSEVNLCNVDIFNPK | | |
| YDCKIMTSKTDVSSSVITSLGAIVSCYGKTKCTASN | | |
| KNRGIIKTFSNGCDYVSNKGVDTVSVGNTLYYVN | | |
| KQEGKSLYVKGEPIINFYDPLVFPSDEFDASISQVN | | |
| EKINQSREIIRAINIVRKIASEKSAIGGYIPEAPRDG | | |
| QAYVRKDGEWVLLSTFL | | |
| MELLILKANAITTILTAVTFCFASGQNITEEFYQSTCSA | | 291 |
| VSKGYLSALRTGWYHCVITIELSNIKENKCNGTDA | | |
| KVKLIKQELDKYKNAVTELQLLMQSTPATNNRAR | | |
| RELPRFMNYTLNNAKKTNVTLSKKRKRRFLGFL | | |
| LGVGSAIASGVAVSKVLHLEGEVNKIKSALLSTNK | | |
| AVVSLSNGVSVCTSKVLDLKNYIDKQLLPIVNKQS | | |
| CSISNIETVIEFQQKNNRLLEITREFSVNAGVTTPVS | | |
| TYMLTNSELLSLINDMPITNDQKKLMSNNVQIVRQ | | |
| QSYSIMSIIKEEVLAYWQLPLYGVIDTPCWKLHTSP | | |
| LCTTNTKEGSNICLTRTDRGWYCDNAGSVSFFPQA | | |
| ETCKVQSNRVFCDTMNSLTLPSEVNLCNVDIFNPK | | |
| YDCKIMTSKTDVSSSVITSLGAIVSCYGKTKCTASN | | |
| KNRGIIKTFSNGCDYVSNKGVDTVSVGNTLYYVN | | |
| KQEGKSLYVKGEPIINFYDPLVFPSSEFDASISQVNE | | |
| KINQSREIIRAINIVRKIASEKSAIGGYIPEAPRDGQ | | |
| AYVRKDGEWVLLSTEL | | |
| MELLILKAAITTILTAVTFCFASGQNITEEFYQSTCSA | V56C + V164C | 292 |
| VSKGYLSALRTGWYTSCITIELSNIKENKCNGTDA | | |
| VKLIKQELDKYKNAVTELQLLMQSTPATNNRARR | | |
| ELPRFMYTLNNAKKTVTLSKKRKRRFLGFLLGV | | |
| GSAIASGVAVSKVLHLEGECNKIKSALLSTNKAVV | | |
| SLSNGVSVLTSKVLDLKNYIDKQLLPIVKQSCSISNI | | |
| ETVIEFQQKNNRLLEITREFSVAGVTTPVSTYMLTN | | |
| SELLSLINDMPITNDQKKLMSNNVQIVRQQSYSIMS | | |
| IIKEEVLAYWQLPLYGVIDTPCWKLHTSPLCTTNT | | |
| KEGSNICLTRTDRGWYCDNAGSVSFFPQAETCKV | | |
| QSNRVFCDTMSLTLPSEVNLCNVDIFNPKYDCKIM | | |
| TSKTDVSSSVITSLGAIVSCYGKTKCTASNKNRGII | | |
| KTFSNGCDYVSNKGVDTVSVGNTLYYVKQEGKSL | | |
| YVKGEPIINFYDPLVFPSDEFDASISQVEKINQSREII | | |
| RAINIVRKIASEKSAIGGYIPEAPRDGQAYVRKDG | | |
| EWVLLSTFL | | |
| MELLILKAAITTILTAVTFCFASGQNITEEFYQSTCSA | I57C + S190C | 293 |
| VSKGYLSALRTGWYTSVCTIELSNIKENKCNGTDA | | |
| VKLIKQELDKYKNAVTELQLLMQSTPATNNRARR | | |
| ELPRFMYTLNNAKKTVTLSKKRKRRFLGFLLGV | | |
| GSAIASGVAVSBVLHLEGEVKIKSALLSTNKAWSL | | |
| SNGVSVLTCBVLDLKNYIDKQLLPIVKQSCSISNIET | | |
| VIEFQQKNNRLLEITREFSVAGVTTPVSTYMLTNSE | | |
| LLSLINDMPITNDQKKLMSNNVQIVRQQSYSIMSII | | |
| KEEVLAYWQLPLYGVIDTPCWKLHTSPLCTTNTKE | | |
| GSNICLTRTDRGWYCDNAGSVSFFPQAETCKVQSN | | |
| RVFCDTMSLTLPSEVNLCNVDIFNPKYDCKIMTSK | | |
| TDVSSSVITSLGAIVSCYGKTKCTASNKNRGIIKTFS | | |
| NGCDYVSNKGVDTVSVGNTLYYVKQEGKSLYVK | | |
| GEPIINFYDPLVFPSDEFDASISQVEKINQSREIIRAIN | | |
| IVRKIASEKSAIGGYIPEAPRDGQAYVRKDGEWV | | |
| LLSTFL | | |
| MELLILKAAITTILTAVTFCFASGQNITEEFYQSTCSA | T58C + V164C | 294 |
| VSKGYLSALRTGWYTSVICIELSNIKENKCNGTDA | | |
| VKLIKQELDKYKNAVTELQLLMQSTPATNNRARR | | |
| ELPRFMYTLNNAKKTVTLSKKRKRRFLGFLLGV | | |
| GSAIASGVAVSKVLHLEGECNKIKSALLSTNKAVV | | |
| SLSNGVSVLTSKVLDLKNYIDKQLLPIVKQSCSISNI | | |
| ETVIEFQQKNNRLLEITREFSVAGVTTPVSTYMLTN | | |
| SELLSLINDMPITNDQKKLMSNNVQIVRQQSYSIMS | | |
| IIKEEVLAYWQLPLYGVIDTPCWKLHTSPLCTTNT | | |
| KEGSNICLTRTDRGWYCDNAGSVSFFPQAETCKV | | |
| QSNRVFCDTMSLTLPSEVNLCNVDIFNPKYDCKIM | | |
| TSKTDVSSSVITSLGAIVSCYGKTKCTASNKNRGII | | |
| KTFSNGCDYVSNKGVDTVSVGNTLYYVKQEGKSL | | |
| YVKGEPIINFYDPLVFPSDEFDASISQVEKINQSREII | | |
| RAINIVRKIASEKSAIGGYIPEAPRDGQAYVRKDG | | |
| EWVLLSTFL | | |
| MELLILKAAITTILTAVTFCFASGQNITEEFYQSTCSA | N165C + V296C | 295 |
| VSKGYLSALRTGWYTSVITIELSNIKENKCNGTDA | | |
| VKLIKQELDKYKNAVTELQLLMQSTPATNNRARR | | |
| ELPRFMYTLNNAKKTVTLSKKRKRRFLGFLLGV | | |
| GSAIASGVAVSBVLHLEGEVCKIKSALLSTNKAWS | | |
| LSNGVSVLTSBVLDLKNYIDKQLLPIVKQSCSISNIE | | |
| TVIEFQQKNNRLLEITREFSVAGVTTPVSTYMLTNS | | |
| ELLSLINDMPITNDQKKLMSNNVQIVRQQSYSIMSI | | |
| IKEECLAYWQLPLYGVIDTPCWKLHTSPLCTTNTK | | |
| EGSNICLTRTDRGWYCDNAGSVSFFPQAETCKVQS | | |
| NRVFCDTMSLTLPSEVNLCNVDIFNPKYDCKIMTS | | |
| KTDVSSSVITSLGAIVSCYGKTKCTASNKNRGIIKT | | |
| FSNGCDYVSNKGVDTVSVGNTLYYVKQEGKSLYV | | |
| KGEPIINFYDPLVFPSDEFDASISQVEKINQSREIIRAI | | |
| NIVRKIASEKSAIGGYIPEAPRDGQAYVRKDGEW | | |
| VLLSTFL | | |
| MELLILKAAITTILTAVTFCFASGQNITEEFYQSTCSA | K168C + V296C | 296 |
| VSKGYLSALRTGWYTSVITIELSNIKENKCNGTDA | | |
| KVKLIKQELDKYKNAVTELQLLMQSTPATIWRAR | | |
| RELPRFMYTLAKKTVTLSKKRKRRFLGFLLGVG | | |
| SAIASGVAVSBVLHLEGEVKICSALLSTNKAWSLS | | |
| NGVSVLTSBVLDLKNYIDKQLLPIVKQSCSISNIET | | |
| VIEFQQKNNRLLEITREFSVAGVTTPVSTYMLTNSE | | |
| LLSLINDMPITNDQKKLMSNNVQIVRQQSYSIMSII | | |
| KEECLAYWQLPLYGVIDTPCWKLHTSPLCTTNTKE | | |
| GSNICLTRTDRGWYCDNAGSVSFFPQAETCKVQSN | | |
| RVFCDTMSLTLPSEVNLCNVDIFNPKYDCKIMTSK | | |
| TDVSSSVITSLGAIVSCYGKTKCTASNKNRGIIKTFS | | |
| NGCDYVSNKGVDTVSVGNTLYYVKQEGKSLYVK | | |
| GEPIINFYDPLVFPSDEFDASISQVEKINQSREIIRAIN | | |
| IVRKIASEKSAIGGYIPEAPRDGQAYVRKDGEWV | | |
| LLSTFL | | |
| MELLILKAAITTILTAVTFCFASGQNITEEFYQSTCSA | M396C + F483C | 297 |
| VSKGYLSALRTGWYTSVITIELSNIKENKCNGTDA | | |
| VKLIKQELDKYKNAVTELQLLMQSTPATNNRARR | | |
| ELPRFMYTLNNAKKTVTLSKKRKRRFLGFLLGV | | |
| GSAIASGVAVSKVLHLEGEVKIKSALLSTNKAVVS | | |
| LSNGVSVLTSKVLDLKNYIDKQLLPIVKQSCSISNIE | | |
| TVIEFQQKNNRLLEITREFSVAGVTTPVSTYMLTNS | | |
| ELLSLINDMPITNDQKKLMSNNVQIVRQQSYSIMSI | | |
| IKEEVLAYWQLPLYGVIDTPCWKLHTSPLCTTNTK | | |
| EGSNICLTRTDRGWYCDNAGSVSFFPQAETCKVQS | | |
| NRVFCDTMSLTLPSEVNLCNVDIFNPKYDCKICTS | | |
| KTDVSSSVITSLGAIVSCYGKTKCTASNKNRGIIKT | | |
| FSNGCDYVSNKGVDTVSVGNTLYYVKQEGKSLYV | | |
| KGEPIINFYDPLVCPSDEFDASISQVEKINQSREIIRA | | |
| INIVRKIASEKSAIGGYIPEAPRDGQAYVRKDGE | | |
| WVLLSTFL | | |
| METPAQLLFLLLLWLPDTTGFASGQNITEEFYQSTC | Ectodomain + Igk signal | 298 |
| SAVSKGYLSALRTGWYTSVITIELSNIKKNKCNGT | + foldon | |
| DAKVKLIKQELDKYKNAVTELQLLMQSTQATNNR | | |
| ARRELPRFMNYTLNNAKKTNVTLSKKRKRRFL | | |
| GFLLGVGSAIASGVAVSKVLHLEGEVNKIKSALLS | | |
| TNKAVVSLSNGVSVLTSKVLDLKNYIDKQLLPIVN | | |
| KQSCSISNIETVIEFQQKNNRLLEITREFSVNAGVTT | | |
| PVSTYMLTNSELLSLINDMPITNDQKKLMSNNVQI | | |
| VRQQSYSIMSIIKEEVLAYVVQLPLYGVIDTPCWKL | | |
| HTSPLCTTNTKEGSNICLTRTDRGWYCDNAGSVSF | | |
| FPQAETCKVQSNRVFCDTMNSLTLPSEVNLCNVDI | | |
| FNPKYDCKIMTSKTDVSSSVITSLGAIVSCYGKTKC | | |
| TASNKNRGIIKTFSNGCDYVSNKGVDTVSVGNTLY | | |
| YVNKQEGKSLYVKGEPIINFYDPLVFPSDEFDASIS | | |
| QVNEKINQSREIIRAINIVRKIASEKSAIGGYIPEAP | | |
| RDGQAYVRKDGEWVLLSTFL | | |
| METPAQLLFLLLLWLPDTTGFASGQNITEEFYQSTC | Ectodomain + Igk signal | 299 |
| SAVSKGYLSALRTGWYTSVITIELSNIKENKCNGTD | + foldon | |
| AKVKLIKQELDKYKNAVTELQLLMQSTPATNNRA | | |
| RRELPRFMNYTLNNAKKTNVTLSKKRKRRFLGF | | |
| LLGVGSAIASGVAVCKVLHLEGEVNKIKSALLSTN | | |
| KAVVSLSNGVSVLTFKVLDLKNYIDKQLLPILNKQ | | |
| SCSISNIETVIEFQQKNNRLLEITREFSVNAGVTTPV | | |
| STYMLTNSELLSLINDMPITNDQKKLMSNNVQIVR | | |
| QQSYSIMCIIKEEVLAYVVQLPLYGVIDTPCWKLH | | |
| TSPLCTTNTKEGSNICLTRTDRGWYCDNAGSVSFF | | |
| PQAETCKVQSNRVFCDTMNSLTLPSEVNLCNVDIF | | |
| NPKYDCKIMTSKTDVSSSVITSLGAIVSCYGKTKCT | | |
| ASNKNRGIIKTFSNGCDYVSNKGVDTVSVGNTLYY | | |
| VNKQEGKSLYVKGEPIINFYDPLVFPSDEFDASISQ | | |
| VNEKINQSREIIRAINIVRKIASEKSAIGGYIPEAPR | | |
| DGQAYVRKDGEWVLLSTFL | | |
| MELLILKANAITTILTAVTFCFASGQNITEEFYQSTCS | | 300 |
| AVSKGYLSALRTGWYTSVITIELSNIKKNKCNGTD | | |
| AKVKLIKQELDKYKNAVTELQLLMQSTQATNNRA | | |
| RRELPRFMNYTLNNAKKTNVTLSKKRKRRFLGF | | |
| LLGVGSAIASGVAVSKVLHLEGEVNKIKSALLSTN | | |
| KAVVSLSNGVSVLTSKVLDLKNYIDKQLLPIVNKQ | | |
| SCSISNIETVIEFQQKNNRLLEITREFSVNAGVTTPV | | |
| STYMLTNSELLSLINDMPITNDQKKLMSNNVQIVR | | |
| QQSYSIMSIIKEEVLAYVVQLPLYGVIDTPCWKLHT | | |
| SPLCTTNTKEGSNICLTRTDRGWYCDNAGSVSFFP | | |
| QAETCKVQSNRVFCDTMNSLTLPSEVNLCNVDIFN | | |
| PKYDCKIMTSKTDVSSSVITSLGAIVSCYGKTKCTA | | |
| SNKNRGIIKTFSNGCDYVSNKGVDTVSVGNTLYYV | | |
| NKQEGKSLYVKGEPIINFYDPLVFPSDEFDASISQV | | |
| NEKINQSREIIRAINIVRKIASEKSAIGGYIPEAPRD | | |
| GQAYVRKDGEWVLLSTFL | | |
| MELLILKANAITTILTAVTFCFASGQNITEEFYQSTCS | S155C, S290C, S190F, | 301 |
| AVSKGYLSALRTGWYTSVITIELSNIKENKCNGTD | V207L | |
| AKVKLIKQELDKYKNAVTELQLLMQSTPATNNRA | | |
| RRELPRFMNYTLNNAKKTNVTLSKKRKRRFLGF | | |
| LLGVGSAIASGVAVCKVLHLEGEVNKIKSALLSTN | | |
| KAVVSLSNGVSVLTFKVLDLKNYIDKQLLPILNKQ | | |
| SCSISNIETVIEFQQKNNRLLEITREFSVNAGVTTPV | | |
| STYMLTNSELLSLINDMPITNDQKKLMSNNVQIVR | | |
| QQSYSIMCIIKEEVLAYVVQLPLYGVIDTPCWKLH | | |
| TSPLCTTNTKEGSNICLTRTDRGWYCDNAGSVSFF | | |
| PQAETCKVQSNRVFCDTMNSLTLPSEVNLCNVDIF | | |
| NPKYDCKIMTSKTDVSSSVITSLGAIVSCYGKTKCT | | |
| ASNKNRGIIKTFSNGCDYVSNKGVDTVSVGNTLYY | | |
| VNKQEGKSLYVKGEPIINFYDPLVFPSDEFDASISQ | | |
| VNEKINQSREIIRAINIVRKIASEKSAIGGYIPEAPR | | |
| DGQAYVRKDGEWVLLSTFL | | |
| MELLILKANAITTILTAVTFCFASGQNITEEFYQSTC | Deletion of p27 | 302 |
| SAVSKGYLSALRTGWYTSVITIELSNIKKNKCNGT | sequence | |
| DAKVKLIKQELDKYKNAVTELQLLMQSTQATNNR | | |
| ARQQQQRFLGFLLGVGSAIASGVAVSKVLHLEGE | | |
| VNKIKSALLSTNKAVVSLSNGVSVLTSKVLDLKNY | | |
| IDKQLLPIVNKQSCSISNIETVIEFQQKNNRLLEITRE | | |
| FSVNAGVTTPVSTYMLTNSELLSLINDMPITNDQK | | |
| KLMSNNVQIVRQQSYSIMSIIKEEVLAYVVQLPLY | | |
| GVIDTPCWKLHTSPLCTTNTKEGSNICLTRTDRGW | | |
| YCDNAGSVSFFPQAETCKVQSNRVFCDTMNSLTLP | | |
| SEVNLCNVDIFNPKYDCKIMTSKTDVSSSVITSLGA | | |
| IVSCYGKTKCTASNKNRGIIKTFSNGCDYVSNKGV | | |
| DTVSVGNTLYYVNKQEGKSLYVKGEPIINFYDPLV | | |
| FPSDEFDASISQVNEKINQSREIIRAINIVRKIASEKS | | |
| AIGGYIPEAPRDGQAYVRKDGEWVLLSTFL | | |
| MELLILKTNAITAILAAVTLCFASSQNITEEFYQSTC | Deletion of p27 | 303 |
| SAVSKGYLSALRTGWYTSVITIELSNIKENKCNGTD | sequence | |
| AKVKLIKQELDKYKSAVTELQLLMQSTPATNNKF | | |
| LGFLLGVGSAIASGIAVSKVLHLEGEVNKIKSALLS | | |
| TNKAVVSLSNGVSVLTSKVLDLKNYIDKQLLPIVN | | |
| KQSCSISNIETVIEFQQKNNRLLEITREFSVNAGVTT | | |
| PVSTYMLTNSELLSLINDMPITNDQKKLMSNNVQI | | |
| VRQQSYSIMSIIKEEVLAYVVQLPLYGVIDTPCWKL | | |
| HTSPLCTTNTKEGSNICLTRTDRGWYCDNAGSVSF | | |
| FPLAETCKVQSNRVFCDTMNSLTLPSEVNLCNIDIF | | |
| NPKYDCKIMTSKTDVSSSVITSLGAIVSCYGKTKCT | | |
| ASNKNRGIIKTFSNGCDYVSNKGVDTVSVGNTLYY | | |
| VNKQEGKSLYVKGEPIINFYDPLVFPSDEFDASISQ | | |
| VNEKINQSREIIRAINIVRKIASEKSAIGGYIPEAPR | | |
| DGQAYVRKDGEWVLLSTFL | | |
| MELLILKANAITTILTAVTFCFASGQNITEEFYQSTC | | 304 |
| SAVSKGYLSALRTGWYTSVITIELSNIKENKCNGTD | | |
| AKVKLIKQELDKYKNAVTELQLLMQSTPATNNRA | | |
| RRELPRFMNYTLNNAKKTNVTLSKKRKRRFLGF | | |
| LLGVGSAIASGVAVCKVLHLEGEVNKIKSALLSTN | | |
| KAVVSLSNGVSVLTFKVLDLKNYIDKQLLPILNKQ | | |
| SCSISNIETVIEFQQKNNRLLEITREFSVNAGVTTPV | | |
| STYMLTNSELLSLINDMPITNDQKKLMSNNVQIVR | | |
| QQSYSIMCIIKEEVLAYVVQLPLYGVIDTPCWKLH | | |
| TSPLCTTNTKEGSNICLTRTDRGWYCDNAGSVSFF | | |
| PQAETCKVQSNRVFCDTMNSLTLPSEVNLCNVDIF | | |
| NPKYDCKIMTSKTDVSSSVITSLGAIVSCYGKTKCT | | |
| ASNKNRGIIKTFSNGCDYVSNKGVDTVSVGNTLYY | | |
| VNKQEGKSLYVKGEPIINFYDPLVFPSDEFDASISQ | | |
| VNEKINQSREIIRAINIVRKIASEKSAIGGYIPEAPR | | |
| DGQAYVRKDGEWVLLSTFL | | |
| MELLILKANAITTILTAVTFCFASQNITEEFYQSTCS | | 305 |
| AVSKGYLSALRTGWYTSVITIELSNIKENKCNGTD | | |
| AKVKLIKQELDKYKNAVTELQLLMQSTPATNNRA | | |
| RRELPRFMNYTLNNAKKINVILSKKRKRRFLGFLL | | |
| GVGSAIASGVAVCKVLHLEGEVNKIKSALLSINKA | | |
| VVSLSNGVSVLIFKVLDLKNYIDKQLLPILNKQSCS | | |
| ISNIETVIEFQQKNNRLLEITREFSVNAGVITPVSTY | | |
| MLINSELLSLINDMPITNDQKKLMSNNVQIVRQQS | | |
| YSIMCIIKEEVLAYVVQLPLYGVIDTPCWKLHISPL | | |
| CTINTKEGSNICLTRIDRGWYCDNAGSVSFFPQAET | | |
| CKVQSNRVFCDTMNSLTLPSEVNLCNVDIFNPKYD | | |
| CKIMISKTDVSSSVITSLGAIVSCYGKTKCIASNKNR | | |
| GIIKTFSNGCDYVSNKGVDTVSVGNTLYYVNKQE | | |
| GKSLYVKGEPIINFYDPLVFPSDEFDASISQVNEKIN | | |
| QSREIIRAINIVRKIASEKSAIGGYIPEAPRDGQAY | | |
| VRKDGEWVLLSTEL | | |

In another aspect, the disclosure provides a recombinant polypeptide, comprising an alpha-helical segment and a multimerization domain, wherein the segment comprises a polypeptide sequence listed in Table 2A or Table 2B, or a polypeptide sequence having between 1 and 5 amino acid substitutions thereto. In some embodiments, the polypeptide comprises a trimeric pathogen protein, N-terminally or C-terminally linked to the alpha-helical segment. In some embodiments, the segment comprises the polypeptide sequence NQSREIIRAINIVRKIASEK (SEQ ID NO: 10), NQSALWLEAAKYVKQAREKS (SEQ ID NO: 11), NQSAKNAEAAKIAEETKRKD (SEQ ID NO: 12), or NQSRETAKAVSAVK (SEQ ID NO: 75), or a polypeptide sequence having between 1 and 5 amino acid substitutions thereto. In some embodiments, polypeptide comprises, N-terminal to the segment, an antigen.

### III. Protein Nanostructures

The disclosure further provides protein nanostructures comprising any of the engineered ectodomains described herein. For example, the disclosure provides protein nanostructures comprising a trimeric component comprising a recombinant polypeptides comprising an ectodomain of a viral membrane fusion (F) protein of Respiratory Syncytial Virus (RSV) having an engineered C-terminal alpha-helical segment that stabilizes the F protein in a prefusion conformation and pentameric component.

The protein nanostructures of the present invention may comprise multimeric protein assemblies adapted for display of molecules such as antigens (e.g., engineered ectodomains). The protein nanostructures, in some embodiments described herein, comprise at least a first component displaying an engineered ectodomain and, optionally, a second component. The engineered ectodomain may include one or more amino acid substitutions, a C-terminal helix-forming segment, or a combination thereof. The first component may comprising or consist of three copies of a fusion protein. In some embodiments, the fusion protein comprises an assembly domain having a protein sequence designed by computational methods to assemble to form a nanostructure. In some embodiments, the first component is a trimeric component in which the assembly domains form trimers related by 3-fold rotational symmetry, and/or the second component is a pentameric component, in which the assembly domains form pentamers related by 5-fold rotational symmetry. In some embodiments, the combination of the two components form an “icosahedral particle” having 153 symmetry. Together these components may be arranged such that the members of each component are related to one another by symmetry operators. A general computational method for designing self-assembling protein materials, involving symmetrical docking of protein building blocks in a target symmetric architecture, is disclosed in Patent Pub. No. US 2015/0356240 A1.

The “core” of the protein nanostructure is used herein to describe the central portion of the protein nanostructure. For clarity, the term “core” as used herein excludes molecules displayed by the nanostructure. The core may serve to assemble multiple copies of the displayed molecule, such as an antigen (e.g., an engineered ectodomain). Without being bound by theory, this may increase the immunogenicity of an antigen. The disclosure envisions nanostructures in which the core is either non-covalentaly associated with the displayed antigen; covalently linked to the display antigen (such as by chemical conjugation); or, in preferred embodiments, linked to the displayed antigen through a polypeptide linker in a fusion protein. In some embodiments, the fusion protein comprises a first polypeptide comprising an antigen (e.g., an ectodomain), and a first assembly domain. In some embodiments, an antigen (e.g., an ectodomain) is non-covalently or covalently linked to the assembly domain. For example, an antigen (e.g., an ectodomain) may be fused to the first component and configured to bind a portion of the first component, or a chemical tag on the first component. For example, a streptavidin-biotin (or neuravidin-biotin) linker can be employed. Alternatively, various bioconjugate linkers may be used. In some embodiments of the present disclosure, the antigen comprises further polypeptide sequences in addition to RSV F protein.

In some embodiments, three copies of an antigen (e.g., an ectodomain) polypeptide are displayed on a 3-fold axis. Thus, the protein nanostructure is capable of displaying 60 monomeric antigen (e.g., an ectodomain) polypeptide. In some embodiments, the protein nanostructure is adapted for display of up to 12, 24, or 60 monomers. In some embodiments, a component may comprise a polypeptide linked to diverse engineered ectodomains, such that the protein nanostructure displays different ectodomains on the same nanostructure. In some embodiments, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more different ectodomains are displayed. Non-limiting illustrative protein nanostructure are provided in Bale et al. Science 353:389-94 (2016); Heinze et al. J. Phys. Chem B. 120:5945-5952 (2016); King et al. Nature 510:103-108 (2014); and King et al. Science 336:1171-71 (2012).

### Attachment Modalities

The protein nanostructures of the present disclosure display antigenic proteins in various ways including as gene fusion or by other means disclosed herein. As used herein, “linked to” or “attached to” denotes any means known in the art for causing two polypeptides to associate. The association may be direct or indirect, reversible or irreversible, weak or strong, covalent or non-covalent, and selective or nonselective.

In some embodiments, attachment is achieved by genetic engineering to create an N- or C-terminal fusion of potentially antigenic polypeptides of the protein nanostructure.

In some embodiments, attachment is achieved by post-translational covalent attachment of one or more pluralities of antigenic protein. In some embodiments, chemical cross-linking is used to non-specifically attach the antigen to a protein nanostructure. In some embodiments, chemical cross-linking is used to specifically attach the antigenic protein to a protein nanostructure (e.g., to the first polypeptide or the second polypeptide). Various specific and non-specific cross-linking chemistries are known in the art, such as Click chemistry and other methods. In general, any cross-linking chemistry/bioconjugate used to link two proteins may be adapted for use in the presently disclosed protein nanostructures. In particular, chemistries used in creation of immunoconjugates or antibody drug conjugates may be used. In some embodiments, a protein nanostructure is created using a cleavable or non-cleavable linker. Processes and methods for conjugation of antigens to carriers are provided by, e.g., Patent Pub. No. US 2008/0145373 A1.

The protein nanostructures may employ a variety of coupling techniques to attach an antigen to the core, including but not limited to the SpyCatcher system described in, e.g., Escolano et al. Nature 570:468-473 (2019), He et al. Sci Adv. 7(12):eabf1591 (2021), and Tan et al. Nat. Commun. 12(1):542 (2021).

In some embodiments, attachment is achieved by non-covalent attachment between a component and the ectodomain. In some embodiments the ectodomain is engineered to be negatively charged on at least one surface and the core polypeptide is engineered to be positively charged on at least one surface, or positively and negatively charged, respectively. This can promote intermolecular association between the ectodomain and the component core polypeptide by electrostatic force. In some embodiments, shape complementarity is employed to cause linkage of ectodomain to component core. Shape complementarity can be pre-existing or rationally designed. In some embodiments, computational design of protein-protein interfaces is used to achieve attachment.

### Polypeptide Sequences

Patent Pub No. US 2015/0356240 A1 describes various methods for designing protein assemblies. As described in US Patent Pub No. US 2016/0122392 A1 and in International Patent Pub. No. WO 2014/124301 A1, the isolated polypeptides of SEQ ID NOs:13-63 were designed for their ability to self-assemble in pairs to form protein nanostructures, such as icosahedral particles. The design involved design of suitable interface residues for each member of the polypeptide pair that can be assembled to form the protein nanostructures. The protein nanostructures so formed include symmetrically repeated, non-natural, non-covalent polypeptide-polypeptide interfaces that orient a first assembly domain and a second assembly domain into protein nanostructures, such as one with an icosahedral symmetry. Thus, in one embodiment a first assembly domain and second assembly domain of the component are selected from the group consisting of SEQ ID NOs:13-63. In each case, an N-terminal methionine residue present in the full length protein is included, but may be removed to make a fusion that is not included in the sequence. The identified residues in Table 6 are numbered beginning with an N-terminal methionine (not shown). In various embodiments, one or more additional residues are deleted from the N-terminus and/or additional residues are added to the N-terminus (e.g., to form a helical extension).

TABLE 6

| | | | Identified |
|---|---|---|---|
| | Component | | interface |
| Name | Multimer | Amino Acid Sequence | residues |
| I53-34A | trimer | EGMDPLAVLAESRLLPLLTVRGGEDLAGLATVLELMG | I53-34A: |
| SEQ ID | | VGALEITLRTEKGLEALKALRKSGLLLGAGTVRSPKE | 28, 32, 36, |
| NO: 13 | | AEAALEAGAAFLVSPGLLEEVAALAQARGVPYLPGVL | 37, 186, |
| | | TPTEVERALALGLSALKFFPAEPFQGVRVLRAYAEVF | 188, 191, |
| | | PEVRFLPTGGIKEEHLPHYAALPNLLAVGGSWLLQGD | 192, 195 |
| | | LAAVMKKVKAAKALLSPQAPG | |
| I53-34B | pentamer | TKKVGIVDITFARVDMAEAAIRTLKALSPNIKIIRKT | I53-34B: |
| SEQ ID | | VPGIKDLPVACKKLLEEEGCDIVMALGMPGKAEKDKV | 19, 20, 23, |
| NO: 14 | | CAHEASLGLMLAQLMTNKHIIEVFVHEDEAKDDDELD | 24, 27, 109, |
| | | ILALVRAIEHAANVYYLLFKPEYLTRMAGKGLRQGRE | 113, 116, |
| | | DAGPARE | 117, 120, |
| | | | 124, 148 |
| I53-40A | pentamer | TKKVGIVDTTFARVDMASAAILTLKMESPNIKIIRKT | I53-40A: |
| SEQ ID | | VPGIKDLPVACKKLLEEEGCDIVMALGMPGKAEKDKV | 20, 23, 24, |
| NO: 15 | | CAHEASLGLMLAQLMTNKHIIEVFVHEDEAKDDAELK | 27, 28, 109, |
| | | ILAARRAIEHALNVYYLLFKPEYLTRMAGKGLRQGFE | 112, 113, |
| | | DAGPARE | 116, 120, |
| | | | 124 |
| I53-40B | trimer | STINNQLKALKVIPVIAIDNAEDIIPLGKVLAENGLP | I53-40B: |
| SEQ ID | | AAEITFRSSAAVKAIMLLRSAQPEMLIGAGTILNGVQ | 47,51, 54, |
| NO: 16 | | ALAAKEAGATFVVSPGFNPNTVRACQIIGIDIVPGVN | 58, 74, 102 |
| | | NPSTVEAALEMGLTTLKFFPAEASGGISMVKSLVGPY | |
| | | GDIRLMPTGGITPSNIDNYLAIPQVLACGGTWMVDKK | |
| | | LVINGEWDEIARLTRIVEQVNP | |
| I53-47A | trimer | PIFTLNTNIKATDVPSDFLSLTSRLVGLILSKPGSYV | I53-47A: |
| SEQ ID | | AVHINTDQQLSFGGSINPAAFGTLMSIGGIEPSKNRD | 22, 25, 29, |
| NO: 17 | | HSAVLFDHLNAMLGIPKNRMYIHFVNLNGDDVGWNGT | 72, 79, 86, |
| | | TF | 87 |
| I53-47B | pentamer | NQHSHKDYETVRIAVVRARWHADIVDACVEAFEIAMA | I53-47B: |
| SEQ ID | | AIGGDRFAVDVFDVPGAYEIPLHARTLAETGRYGAVL | 28, 31, 35, |
| NO: 18 | | GTAFVVNGGIYRHEFVASAVIDGMMNVQLSTGVPVLS | 36, 39, 131, |
| | | AVLTPHRYRDSAEHHRFFAAHFAVKGVEAARACIEIL | 132, 135, |
| | | AAREKIAA | 139, 146 |
| I53-50A | trimer | EELFKKHKIVAVLRANSVEEAIEKAVAVFAGGVHLIE | I53-50A: |
| SEQ ID | | ITFTVPDADTVIKALSVLKEKGAIIGAGTVTSVEQCR | 25, 29, 33, |
| NO: 19 | | KAVESGAEFIVSPHLDEEISQFCKEKGVFYMPGVMTP | 54,57 |
| | | TELVKAMKLGHTILKLFPGEVVGPQFVKAMKGPFPNV | |
| | | KFVPTGGVNLDNVCEWFKAGVLAVGVGSALVKGTPDE | |
| | | VREKAKAFVEKIRGCTE | |
| I53-50B | pentamer | NQHSHKDYETVRIAVVRARWHAEIVDACVSAFEAAMA | I53-50B: |
| SEQ ID | | DIGGDRFAVDVFDVPGAYEIPLHARTLAETGRYGAVL | 24, 28, 36, |
| NO: 20 | | GTAFVVNGGIYRHEFVASAVIDGMMNVQLSTGVPVLS | 124, 125, |
| | | AVLTPHRYRDSDAHILLFLALFAVKGMEAARACVEIL | 127, 128, |
| | | AAREKIAA | 129, 131, |
| | | | 132, 133, |
| | | | 135, 139 |
| I53-51A | trimer | FTKSGDDGNTNVINKRVGKDSPLVNFLGDLDELNSFI | I53-51A: |
| SEQ ID | | GFAISKIPWEDMKKDLERVQVELFEIGEDLSTQSSKK | 80, 83, 86, |
| NO: 21 | | KIDESYVLWLLAATAIYRIESGPVKLFVIPGGSEEAS | 87, 88, 90, |
| | | VLHVTRSVARRVERNAVKYTKELPEINRMIIVYLNRL | 91, 94, 166, |
| | | SSLLFAMALVANKRRNQSEKIYEIGKSW | 172, 176 |
| I53-51B | pentamer | NQHSHKDYETVRIAVVRARWHADIVDQCVRAFEEAMA | I53-51B: |
| SEQ ID | | DAGGDRFAVDVFDVPGAYEIPLHARTLAETGRYGAVL | 31, 35, 36, |
| NO: 22 | | GTAFVVNGGIYRHEFVASAVIDGMMNVQLSTGVPVLS | 40, 122, |
| | | AVLTPHRYRSSREHHEFFREHFMVKGVEAAAACITIL | 124, 128, |
| | | AAREKIAA | 131, 135, |
| | | | 139, 143, |
| | | | 146, 147 |
| I52-03A | pentamer | GHTKGPTPQQHDGSALRIGIVHARWNKTIIMPLLIGT | I52-03A: |
| SEQ ID | | IAKLLECGVKASNIVVQSVPGSWELPIAVQRLYSASQ | 28, 32, 36, |
| NO: 23 | | LQTPSSGPSLSAGDLLGSSTTDLTALPTTTASSTGPF | 39, 44, 49 |
| | | DALIAIGVLIKGETMHFEYIADSVSHGLMRVQLDTGV | |
| | | PVIFGVLTVLTDDQAKARAGVIEGSHNHGEDWGLAAV | |
| | | EMGVRRRDWAAGKTE | |
| I52-03B | dimer | YEVDHADVYDLFYLGRGKDYAAEASDIADLVRSRTPE | I52-03B: |
| SEQ ID | | ASSLLDVACGTGTHLEHFTKEFGDTAGLELSEDMLTH | 94, 115, |
| NO: 24 | | ARKRLPDATLHQGDMRDFQLGRKFSAVVSMFSSVGYL | 116, 206, |
| | | KTVAELGAAVASFAEHLEPGGVVVVEPWWFPETFADG | 213 |
| | | WVSADVVRRDGRTVARVSHSVREGNATRMEVHFTVAD | |
| | | PGKGVRHFSDVHLITLFHQREYEAAFMAAGLRVEYLE | |
| | | GGPSGRGLFVGVPA | |
| I52-32A | dimer | GMKEKFVLIITHGDFGKGLLSGAEVIIGKQENVHTVG | I52-32A: |
| SEQ ID | | LNLGDNIEKVAKEVMRIIIAKLAEDKEIIIVVDLFGG | 47, 49, 53, |
| NO: 25 | | SPFNIALEMMKTFDVKVITGINMPMLVELLTSINVYD | 54, 57, 58, |
| | | TTELLENISKIGKDGIKVIEKSSLKM | 61,83, 87, |
| | | | 88 |
| I52-32B | pentamer | KYDGSKLRIGILHARWNLEIIAALVAGAIKRLQEFGV | I52-32B: |
| SEQ ID | | KAENIIIETVPGSFELPYGSKLFVEKQKRLGKPLDAI | 19, 20, 23, |
| NO: 26 | | IPIGVLIKGSTMHFEYICDSTTHQLMKLNFELGIPVI | 30, 40 |
| | | FGVLTCLTDEQAEARAGLIEGKMHNHGEDWGAAAVEM | |
| | | ATKFN | |
| I52-33A | pentamer | AVKGLGEVDQKYDGSKLRIGILHARWNRKIILALVAG | I52-33A: |
| SEQ ID | | AVLRLLEFGVKAENIIIETVPGSFELPYGSKLFVEKQ | 33, 41, 44, |
| NO: 27 | | KRLGKPLDAIIPIGVLIKGSTMHFEYICDSTTHQLMK | 50 |
| | | LNFELGIPVIFGVLTCLTDEQAEARAGLIEGKMHNHG | |
| | | EDWGAAAVEMATKFN | |
| I52-33B | dimer | GANWYLDNESSRLSFTSTKNADIAEVHRFLVLHGKVD | I52-33B: |
| SEQ ID | | PKGLAEVEVETESISTGIPLRDMLLRVLVFQVSKFPV | 61, 63, 66, |
| NO: 28 | | AQINAQLDMRPINNLAPGAQLELRLPLTVSLRGKSHS | 67, 72, 147, |
| | | YNAELLATRLDERRFQVVTLEPLVIHAQDFDMVRAFN | 148, 154, |
| | | ALRLVAGLSAVSLSVPVGAVLIFTAR | 155 |
| I32-06A | dimer | TDYIRDGSAIKALSFAIILAEADLRHIPQDLQRLAVR | I32-06A: 9, |
| SEQ ID | | VIHACGMVDVANDLAFSEGAGKAGRNALLAGAPILCD | 12, 13, 14, |
| NO: 29 | | ARMVAEGITRSRLPADNRVIYTLSDPSVPELAKKIGN | 20, 30, 33, |
| | | TRSAAALDLWLPHIEGSIVAIGNAPTALFRLFELLDA | 34 |
| | | GAPKPALIIGMPVGFVGAAESKDELAANSRGVPYVIV | |
| | | RGRRGGSAMTAAAVNALASERE | |
| I32-06B | trimer | ITVFGLKSKLAPRREKLAEVIYSSLHLGLDIPKGKHA | I32-06B: |
| SEQ ID | | IRFLCLEKEDFYYPFDRSDDYTVIEINLMAGRSEETK | 24, 71, 73, |
| NO: 30 | | MLLIFLLFIALERKLGIRAHDVEITIKEQPAHCWGFR | 76, 77, 80, |
| | | GRTGDSARDLDYDIYV | 81, 84, 85, |
| | | | 88, 114, 118 |
| I32-19A | trimer | GSDLQKLQRFSTCDISDGLLNVYNIPTGGYFPNLTAI | I32-19A: |
| SEQ ID | | SPPQNSSIVGTAYTVLFAPIDDPRPAVNYIDSVPPNS | 208, 213, |
| NO: 31 | | ILVLALEPHLQSQFHPFIKITQAMYGGLMSTRAQYLK | 218, 222, |
| | | SNGTVVFGRIRDVDEHRTLNHPVFAYGVGSCAPKAVV | 225, 226, |
| | | KAVGTNVQLKILTSDGVTQTICPGDYIAGDNNGIVRI | 229, 233 |
| | | PVQETDISKLVTYIEKSIEVDRLVSEAIKNGLPAKAA | |
| | | QTARRMVLKDYI | |
| I32-19B | dimer | SGMRVYLGADHAGYELKQAIIAFLKMTGHEPIDCGAL | I32-19B: |
| SEQ ID | | RYDADDDYPAFCIAAATRIVADPGSLGIVLGGSGNGE | 20, 23, 24, |
| NO: 32 | | QIAANKVPGARCALAWSVQTAALAREHNNAQLIGIGG | 27, 117, |
| | | RMHTLEEALRIVKAFVTTPWSKAQRHQRRIDILAEYE | 118, 122, |
| | | RTHEAPPVPGAPA | 125 |
| I32-28A | trimer | GDDARIAAIGDVDELNSQIGVLLAEPLPDDVRAALSA | I32-28A: |
| SEQ ID | | IQHDLFDLGGELCIPGHAAITEDHLLRLALWLVHYNG | 60, 61, 64, |
| NO: 33 | | QLPPLEEFILPGGARGAALAHVCRTVCRRAERSIKAL | 67, 68, 71, |
| | | GASEPLNIAPAAYVNLLSDLLFVLARVLNRAAGGADV | 110, 120, |
| | | LWDRTRAH | 123, 124, |
| | | | 128 |
| I32-28B | dimer | ILSAEQSFTLRHPHGQAAALAFVREPAAALAGVQRLR | I32-28B: |
| SEQ ID | | GLDSDGEQVWGELLVRVPLLGEVDLPFRSEIVRTPQG | 35, 36, 54, |
| NO: 34 | | AELRPLTLTGERAWVAVSGQATAAEGGEMAFAFQFQA | 122, 129, |
| | | HLATPEAEGEGGAAFEVMVQAAAGVTLLLVAMALPQG | 137, 140, |
| | | LAAGLPPA | 141, 144, |
| | | | 148 |
| I53-40A.1 | pentamer | TKKVGIVDTTFARVDMASAAILTLKMESPNIKIIRKT | I53-40A: |
| SEQ ID | | VPGIKDLPVACKKLLEEEGCDIVMALGMPGKKEKDKV | 20, 23, 24, |
| NO: 35 | | CAHEASLGLMLAQLMTNKHIIEVFVHEDEAKDDAELK | 27, 28, 109, |
| | | ILAARRAIEHALNVYYLLFKPEYLTRMAGKGLRQGFE | 112, 113, |
| | | DAGPARE | 116, 120, |
| | | | 124 |
| I53-40B.1 | trimer | DDINNQLKRLKVIPVIAIDNAEDIIPLGKVLAENGLP | I53-40B: |
| SEQ ID | | AAEITERSSAAVKAIMLLRSAQPEMLIGAGTILNGVQ | 47, 51,54, |
| NO: 36 | | ALAAKEAGADFVVSPGFNPNTVRACQIIGIDIVPGVN | 58, 74, 102 |
| | | NPSTVEQALEMGLTTLKFFPAEASGGISMVKSLVGPY | |
| | | GDIRLMPTGGITPDNIDNYLAIPQVLACGGTWMVDKK | |
| | | LVRNGEWDEIARLTREIVEQVNP | |
| I53-47A.1 | trimer | PIFTLNTNIKADDVPSDFLSLTSRLVGLILSKPGSYV | I53-47A: |
| SEQ ID | | AVHINTDQQLSFGGSTNPAAFGTLMSIGGIEPDKNRD | 22, 25, 29, |
| NO: 37 | | HSAVLFDHLNAMLGIPKNRMYIHFVNLNGDDVGWNGT | 72, 79, 86, |
| | | TF | 87 |
| I53- | trimer | PIFTLNTNIKADDVPSDFLSLTSRLVGLILSEPGSYV | I53-47A: |
| 47A.1NegT2 | | AVHINTDQQLSFGGSTNPAAFGTLMSIGGIEPDKNED | 22, 25, 29, |
| SEQ ID | | HSAVLFDHLNAMLGIPKNRMYIHFVDLDGDDVGWNGT | 72, 79, 86, |
| NO: 38 | | TF | 87 |
| I53-47B.1 | pentamer | NQHSHKDHETVRIAVVRARWHADIVDACVEAFEIAMA | I53-47B: |
| SEQ ID | | AIGGDRFAVDVEDVPGAYEIPLHARTLAETGRYGAVL | 28, 31, 35, |
| NO: 39 | | GTAFVVNGGIYRHEFVASAVIDGMMNVQLDTGVPVLS | 36, 39, 131, |
| | | AVLTPHRYRDSDEHHRFFAAHFAVKGVEAARACIEIL | 132, 135, |
| | | NAREKIAA | 139, 146 |
| I53- | pentamer | NQHSHKDHETVRIAVVRARWHADIVDACVEAFEIAMA | I53-47B: |
| 47B.1NegT2 | | AIGGDRFAVDVFDVPGAYEIPLHARTLAETGRYGAVL | 28, 31, 35, |
| SEQ ID | | GTAFVVDGGIYDHEFVASAVIDGMMNVQLDTGVPVLS | 36, 39, 131, |
| NO: 40 | | AVLTPHEYEDSDEDHEFFAAHFAVKGVEAARACIEIL | 132, 135, |
| | | NAREKIAA | 139, 146 |
| I53-50A.1 | trimer | EELFKKHKIVAVLRANSVEEAIEKAVAVFAGGVHLIE | I53-50A: |
| SEQ ID | | ITFTVPDADTVIKALSVLKEKGAIIGAGTVTSVEQCR | 25, 29, 33, |
| NO: 41 | | KAVESGAEFIVSPHLDEEISQFCKEKGVFYMPGVMTP | 54, 57 |
| | | TELVKAMKLGHDILKLFPGEVVGPQFVKAMKGPFPNV | |
| | | KFVPTGGVNLDNVCEWFKAGVLAVGVGDALVKGDPDE | |
| | | VREKAKKFVEKIRGCTE | |
| I53- | trimer | EELFKKHKIVAVLRANSVEEAIEKAVAVFAGGVHLIE | I53-50A: |
| 50A.1NegT2 | | ITFTVPDADTVIKALSVLKEKGAIIGAGTVTSVEQCR | 25, 29, 33, |
| SEQ ID | | KAVESGAEFIVSPHLDEEISQFCKEKGVFYMPGVMTP | 54,57 |
| NO: 42 | | TELVKAMKLGHDILKLFPGEVVGPEFVEAMKGPFPNV | |
| | | KFVPTGGVDLDDVCEWFDAGVLAVGVGDALVEGDPDE | |
| | | VREDAKEFVEEIRGCTE | |
| I53- | trimer | EELFKKHKIVAVLRANSVEEAIEKAVAVFAGGVHLIE | I53-50A: |
| 50A.1PosT1 | | ITFTVPDADTVIKALSVLKEKGAIIGAGTVTSVEQCR | 25, 29, 33, |
| SEQ ID | | KAVESGAEFIVSPHLDEEISQFCKEKGVFYMPGVMTP | 54,57 |
| NO: 43 | | TELVKAMKLGHDILKLFPGEVVGPQFVKAMKGPFPNV | |
| | | KFVPTGGVNLDNVCKWFKAGVLAVGVGKALVKGKPDE | |
| | | VREKAKKFVKKIRGCTE | |
| I53- | pentamer | NQHSHKDHETVRIAVVRARWHAEIVDACVSAFEAAMR | I53-50B: |
| 50B.1 | | DIGGDRFAVDVFDVPGAYEIPLHARTLAETGRYGAVL | 24, 28, 36, |
| SEQ ID | | GTAFVVNGGIYRHEFVASAVIDGMMNVQLDTGVPVLS | 124, 125, |
| NO:44 | | AVLTPHRYRDSDAHTLLFLALFAVKGMEAARACVEIL | 127, 128, |
| | | AAREKIAA | 129, 131, |
| | | | 132, 133, |
| | | | 135, 139 |
| I53- | pentamer | NQHSHKDHETVRIAVVRARWHAEIVDACVSAFEAAMR | I53-50B: |
| 50B.1NegT2 | | DIGGDRFAVDVFDVPGAYEIPLHARTLAETGRYGAVL | 24, 28, 36, |
| SEQ ID | | GTAFVVDGGIYDHEFVASAVIDGMMNVQLDTGVPVLS | 124, 125, |
| NO: 45 | | AVLTPHEYEDSDADTLLFLALFAVKGMEAARACVEIL | 127, 128, |
| | | AAREKIAA | 129, 131, |
| | | | 132, 133, |
| | | | 135, 139 |
| I53- | trimer | NQHSHKDHETVRIAVVRARWHAEIVDACVSAFEAAMR | I53-50B: |
| 50B.4PosT1 | | DIGGDRFAVDVFDVPGAYEIPLHARTLAETGRYGAVL | 24, 28, 36, |
| SEQ ID | | GTAFVVNGGIYRHEFVASAVINGMMNVQLNTGVPVLS | 124, 125, |
| NO: 46 | | AVLTPHNYDKSKAHTLLFLALFAVKGMEAARACVEIL | 127, 128, |
| | | AAREKIAA | 129, 131, |
| | | | 132, 133, |
| | | | 135, 139 |
| I53- | pentamer | TKKVGIVDITFARVDMASAAILTLKMESPNIKIIRKT | |
| 40Agenus | | VPGIKDLPVACKKLLEEEGCDIVMALGMPGK(A/K)E | |
| SEQ ID | | KDKVCAHEASLGLMLAQLMTNKHIIEVFVHEDEAKDD | |
| NO: 47 | | AELKILAARRAIEHALNVYYLLFKPEYLTRMAGKGLR | |
| | | QGFEDAGPARE | |
| I53- | trimer | (S/D)(T/D)INNQLK(A/R)LKVIPVIAIDNAEDII | |
| 40Bgenus | | PLGKVLAENGLPAAEITERSSAAVKAIMLLRSAQPEM | |
| SEQ ID | | LIGAGTILNGVQALAAKEAGA(T/D)FVVSPGENPNT | |
| NO: 48 | | VRACQIIGIDIVPGVNNPSTVE(A/Q)ALEMGLTTLK | |
| | | FFPAEASGGISMVKSLVGPYGDIRLMPTGGITP | |
| | | (S/D)NIDNYLAIPQVLACGGTWMVDKKLV(T/R)NG | |
| | | EWDEIARLTREIVEQVNP | |
| I53- | trimer | PIFTLNTNIKA(T/D)DVPSDFLSLTSRLVGLILS | |
| 47Agenus | | (K/E)PGSYVAVHINTDQQLSFGGSTNPAAFGTLMSI | |
| SEQ ID | | GGIEP(S/D)KN(R/E)DHSAVLFDHLNAMLGIPKNR | |
| NO: 49 | | MYIHFV(N/D)L(N/D)GDDVGWNGTTF | |
| I53- | pentamer | NQHSHKD(Y/H)ETVRIAVVRARWHADIVDACVEAFE | |
| 47Bgenus | | IAMAAIGGDRFAVDVFDVPGAYEIPLHARTLAETGRY | |
| SEQ ID | | GAVLGTAFVV(N/D)GGIY(R/D)HEFVASAVIDGMM | |
| NO: 50 | | NVQL(S/D)TGVPVLSAVLTPH(R/E)Y(R/E)DS | |
| | | (A/D)E(H/D)H(R/E)FFAAHFAVKGVEAARACIEI | |
| | | L(A/N)AREKIAA | |
| I53- | trimer | EELFKKHKIVAVLRANSVEEAIEKAVAVFAGGVHLIE | |
| 50Agenus | | ITFTVPDADTVIKALSVLKEKGAIIGAGTVTSVEQCR | |
| SEQ ID | | KAVESGAEFIVSPHLDEEISQFCKEKGVFYMPGVMTP | |
| NO: 51 | | TELVKAMKLGH(T/D)ILKLFPGEVVGP(Q/E)FV | |
| | | (K/E)AMKGPFPNVKFVPTGGV(N/D)LD(N/D)VC | |
| | | (E/K)WF(K/D)AGVLAVGVG(S/K/D)ALV(K/E)G | |
| | | (T/D/K)PDEVRE(K/D)AK(A/E/K)FV(E/K) | |
| | | (K/E)IRGCTE | |
| I53- | pentamer | NQHSHKD(Y/H)ETVRIAVVRARWHAEIVDACVSAFE | |
| 50Bgenus | | AAM(A/R)DIGGDRFAVDVFDVPGAYEIPLHARTLAE | |
| SEQ ID | | TGRYGAVLGTAFVV(N/D)GGIY(R/D)HEFVASAVI | |
| NO: 52 | | (D/N)GMMNVQL(S/D/N)TGVPVLSAVLTPH | |
| | | (R/E/N)Y(R/D/E)(D/K)S(D/K)A(H/D)TLLFL | |
| | | ALFAVKGMEAARACVEILAAREKIAA | |
| T32-28A | dimer | GEVPIGDPKELNGMEIAAVYLQPIEMEPRGIDLAASL | |
| SEQ ID | | ADIHLEADIHALKNNPNGFPEGFWMPYLTIAYALANA | |
| NO: 53 | | DTGAIKTGTLMPMVADDGPHYGANIAMEKDKKGGFGV | |
| | | GTYALTFLISNPEKQGFGRHVDEETGVGKWFEPFVVT | |
| | | YFFKYTGTPK | |
| T32-28B | trimer | SQAIGILELTSIAKGMELGDAMLKSANVDLLVSKTIS | |
| SEQ ID | | PGKFLLMLGGDIGAIQQAIETGTSQAGEMLVDSLVLA | |
| NO: 54 | | NIHPSVLPAISGLNSVDKRQAVGIVETWSVAACISAA | |
| | | DLAVKGSNVTLVRVHMAFGIGGKCYMVVAGDVLDVAA | |
| | | AVATASLAAGAKGLLVYASIIPRPHEAMWRQMVEG | |
| T33-09A | trimer | EEVVLITVPSALVAVKIAHALVEERLAACVNIVPGLT | |
| SEQ ID | | SIYRWQGSVVSDHELLLLVKTTTHAFPKLKERVKALH | |
| NO: 55 | | PYTVPEIVALPIAEGNREYLDWLRENTG | |
| T33-09B | trimer | VRGIRGAITVEEDTPAAILAATIELLLKMLEANGIQS | |
| SEQ ID | | YEELAAVIFTVTEDLTSAFPAEAARLIGMHRVPLLSA | |
| NO: 56 | | REVPVPGSLPRVIRVLALWNTDTPQDRVRHVYLNEAV | |
| | | RLRPDLESAQ | |
| T33-15A | trimer | SKAKIGIVTVSDRASAGITADISGKAIILALNLYLTS | |
| SEQ ID | | EWEPIYQVIPDEQDVIETTLIKMADEQDCCLIVTTGG | |
| NO: 57 | | TGPAKRDVTPEATEAVCDRMMPGFGELMRAESLKEVP | |
| | | TAILSRQTAGLRGDSLIVNLPGDPASISDCLLAVEPA | |
| | | IPYCIDLMEGPYLECNEAMIKPFRPKAK | |
| T33-15B | trimer | VRGIRGAITVNSDTPTSIIIATILLLEKMLEANGIQS | |
| SEQ ID | | YEELAAVIFTVTEDLISAFPAEAARQIGMHRVPLLSA | |
| NO: 58 | | REVPVPGSLPRVIRVLALWNTDTPQDRVRHVYLSEAV | |
| | | RLRPDLESAQ | |
| T33-21A | trimer | RITTKVGDKGSTRLFGGEEVWKDSPIIEANGTLDELT | |
| SEQ ID | | SFIGEAKHYVDEEMKGILEEIQNDIYKIMGEIGSKGK | |
| NO: 59 | | IEGISEERIAWLLKLILRYMEMVNLKSFVLPGGTLES | |
| | | AKLDVCRTIARRALRKVLIVTREFGIGAEAAAYLLAL | |
| | | SDLLFLLARVIEIEKNKLKEVRS | |
| T33-21B | trimer | PHLVIEATANLRLETSPGELLEQANKALFASGQFGEA | |
| SEQ ID | | DIKSRFVTLEAYRQGTAAVERAYLHACLSILDGRDIA | |
| NO: 60 | | TRTLLGASLCAVLAEAVAGGGEEGVQVSVEVREMERL | |
| | | SYAKRVVARQR | |
| T33-28A | trimer | ESVNTSFLSPSLVTIRDFDNGQFAVLRIGRTGFPADK | |
| SEQ ID | | GDIDLCLDKMIGVRAAQIFLGDDTEDGEKGPHIRIRC | |
| NO: 61 | | VDIDDKHTYNAMVYVDLIVGTGASEVERETAEEEAKL | |
| | | ALRVALQVDIADEHSCVTQFEMKLREELLSSDSFHPD | |
| | | KDEYYKDFL | |
| T33-28B | trimer | PVIQTFVSTPLDHHKRLLLAIIYRIVTRVVLGKPEDL | |
| SEQ ID | | VMMTFHDSTPMHFFGSTDPVACVRVEALGGYGPSEPE | |
| NO: 62 | | KVTSIVTAAITAVCGIVADRIFVLYFSPLHCGWNGTN | |
| | | F | |
| T33-31A | trimer | EEVVLITVPSALVAVKIAHALVEERLAACVNIVPGLT | |
| SEQ ID | | SIYREEGSVVSDHELLLLVKTTTDAFPKLKERVKELH | |
| NO: 63 | | PYEVPEIVALPIAEGNREYLDWLRENTG | |
| I53- | trimer | EELFKKHKIVAVLRANSVEEAIEKAVAVFAGGVHLIE | |
| 50AACys | | ITFTVPDADTVIKALSVLKEKGAIIGAGTVTSVEQAR | |
| SEQ ID | | KAVESGAEFIVSPHLDEEISQFAKEKGVFYMPGVMTP | |
| NO: 64 | | TELVKAMKLGHTILKLFPGEVVGPQFVKAMKGPFPNV | |
| | | KFVPTGGVNLDNVAEWFKAGVLAVGVGSALVKGTPDE | |
| | | VREKAKAFVEKIRGATE | |
| T33_dn2A | | NLAEKMYKAGNAMYRKGQYTIAIIAYTLALLKDPNNA | |
| SEQ | | EAWYNLGNAAYKKGEYDEAIEAYQKALELDPNNAEAW | |
| ID NO: | | YNLGNAYYKQGDYDEAIEYYKKALRLDPRNVDAIENL | |
| 65 | | IEAEEKQG | |
| T33_dn2B | | EEAELAYLLGELAYKLGEYRIAIRAYRIALKRDPNNA | |
| SEQ | | EAWYNLGNAYYKQGDYREAIRYYLRALKLDPENAEAW | |
| ID NO: | | YNLGNALYKQGKYDLAIIAYQAALEEDPNNAEAKQNL | |
| 66 | | GNAKQKQG | |
| T33_dn5A | | NSAEAMYKMGNAAYKQGDYILAIIAYLLALEKDPNNA | |
| SEQ | | EAWYNLGNAAYKQGDYDEAIEYYQKALELDPNNAEAW | |
| ID NO: | | YNLGNAYYKQGDYDEAIEYYEKALELDPNNAEALKNL | |
| 67 | | LEAIAEQD | |
| T33_dn5A | | TDPLAVILYIAILKAEKSIARAKAAEALGKIGDERAV | |
| SEQ | | EPLIKALKDEDALVRAAAADALGQIGDERAVEPLIKA | |
| ID NO: | | LKDEEGLVRASAAIALGQIGDERAVQPLIKALTDERD | |
| 68 | | LVRVAAAVALGRIGDEKAVRPLIIVLKDEEGEVREAA | |
| | | AIALGSIGGERVRAAMEKLAERGTGFARKVAVNYLET | |
| | | HK | |
| T33_dn10A | | EEAELAYLLGELAYKLGEYRIAIRAYRIALKRDPNNA | |
| SEQ | | EAWYNLGNAYYKQGDYDEAIEYYQKALELDPNNAEAW | |
| ID NO: | | YNLGNAYYKQGDYDEAIEYYEKALELDPENLEALQNL | |
| 69 | | LNAMDKQG | |
| T33_dn10B | | IEEVVAEMIDILAESSKKSIEELARAADNKTTEKAVA | |
| SEQ | | EAIEEIARLATAAIQLIEALAKNLASEEFMARAISAI | |
| ID NO: | | AELAKKAIEAIYRLADNHTTDTFMARAIAAIANLAVT | |
| 70 | | AILAIAALASNHTTEEFMARAISAIAELAKKAIEAIY | |
| | | RLADNHTTDKFMAAAIEAIALLATLAILAIALLASNH | |
| | | TTEKFMARAIMAIAILAAKAIEAIYRLADNHTSPTYI | |
| | | EKAIEAIEKIARKAIKAIEMLAKNITTEEYKEKAKKI | |
| | | IDIIRKLAKMAIKKLEDNRT | |
| I53_dn5A | pentamer | KYDGSKLRIGILHARWNAEIILALVLGALKRLQEFGV | |
| SEQ | | KRENIIIETVPGSFELPYGSKLFVEKQKRLGKPLDAI | |
| ID NO: | | IPIGVLIKGSTMHFEYICDSTTHQLMKLNFELGIPVI | |
| 71 | | FGVLTCLTDEQAEARAGLIEGKMHNHGEDWGAAAVEM | |
| | | ATKFN | |
| I53_dn5B | trimer | EEAELAYLLGELAYKLGEYRIAIRAYRIALKRDPNNA | |
| SEQ | | EAWYNLGNAYYKQGRYREAIEYYQKALELDPNNAEAW | |
| ID NO: | | YNLGNAYYERGEYEEAIEYYRKALRLDPNNADAMQNL | |
| 72 | | LNAKMREE | |
| I53_dn5A.1 | pentamer | KYDGSKLRIGILHARGNAEIILALVLGALKRLQEFGV | |
| SEQ | | KRENIIIETVPGSFELPYGSKLFVEKQKRLGKPLDAI | |
| ID NO: | | IPIGVLIRGSTPHFDYIADSTTHQLMKLNFELGIPVI | |
| 73 | | FGVITADTDEQAEARAGLIEGKMHNHGEDWGAAAVEM | |
| | | ATKFN | |
| I53_dn5A.2 | pentamer | KYDGSKLRIGILHARGNAEIILELVLGALKRLQEFGV | |
| SEQ | | KRENIIIETVPGSFELPYGSKLFVEKQKRLGKPLDAI | |
| ID NO: | | IPIGVLIRGSTAHFDYIADSTTHQLMKLNFELGIPVI | |
| 74 | | FGVLTTESDEQAEERAGTKAGNHGEDWGAAAVEMATK | |
| | | FN | |
| I3-01 | | MKMEELFKKHKIVAVLRANSVEEAKKKALAVFLGGVH | |
| SEQ ID | | LIEITFTVPDADTVIKELSFLKEMGAIIGAGTVTSVE | |
| NO: 105 | | QCRKAVESGAEFIVSPHLDEEISQFCKEKGVFYMPGV | |
| | | MTPTELVKAMKLGHTILKLFPGEVVGPQFVKAMKGPF | |
| | | PNVKFVPTGGVNLDNVCEWFKAGVLAVGVGSALVKGT | |
| | | PVEVAEKAKAFVEKIRGCTE | |
| I3-01 | | MKIEELFKKHKIVAVLRANSVEEAKKKALAVELGGVH | |
| (M31) | | LIEITFTVPDADTVIKELSFLKEMGAIIGAGTVTSVE | |
| SEQ ID | | QCRKAVESGAEFIVSPHLDEEISQFCKEKGVEYMPGV | |
| NO: 106 | | MTPTELVKAMKLGHTILKLFPGEVVGPQFVKAMKGPF | |
| | | PNVKFVPTGGVNLDNVCEWFKAGVLAVGVGSALVKGT | |
| | | PVEVAEKAKAFVEKIRGCTE | |
| 1WA3-ref | | MKMEELFKKHKIVAVLRANSVEEAKEKALAVFEGGVH | |
| SEQ ID | | LIEITFTVPDADTVIKELSFLKEKGAIIGAGTVTSVE | |
| NO: 107 | | QCRKAVESGAEFIVSPHLDEEISQFCKEKGVFYMPGV | |
| | | MTPTELVKAMKLGHTILKLFPGEVVGPQFVKAMKGPF | |
| | | PNVKFVPTGGVNLDNVCEWFKAGVLAVGVGSALVKGT | |
| | | PDEVREKAKAFVEKIRGCTE | |
| 1WA3-1 | | (MK)MEELFKKHKIVAVLRANSVEEAKKKALAVFLGG | |
| SEQ ID | | VHLIEITFTVPDADTVIKELSFLKEMGAIIGAGTVTS | |
| NO: 108 | | VEQCRKAVESGAEFIVSPHLDEEISQFCKEKGVFYMP | |
| | | GVMTPTELVKAMKLGHTILKLFPGEVVGPQFVKAMKG | |
| | | PFPNVKFVPTGGVNLDNVCEWFKAGVLAVGVGSALVK | |
| | | GTPVEVAEKAKAFVEKIRGCTE | |
| 1WA3-2 | | (MK)IEELFKKHKIVAVLRANSVEEAKKKALAVELGG | |
| SEQ ID | | VHLIEITFTVPDADTVIKELSFLKEMGAIIGAGTVTS | |
| NO: 109 | | VEQCRKAVESGAEFIVSPHLDEEISQFCKEKGVFYMP | |
| | | GVMTPTELVKAMKLGHTILKLFPGEVVGPQFVKAMKG | |
| | | PFPNVKFVPTGGVNLDNVCEWFKAGVLAVGVGSALVK | |
| | | GTPVEVAEKAKAFVEKIRGCTE | |
| 1WA3-3 | | (MK)MEELFKKHKIVAVLRANSVEEAKKKALAVFLGG | |
| SEQ ID | | VHLIEITFTVPDADTVIKELSFLKEMGAIIGAGTVTS | |
| NO: 110 | | VEQARKAVESGAEFIVSPHLDEEISQFAKEKGVFYMP | |
| | | GVMTPTELVKAMKLGHTILKLFPGEVVGPQFVKAMKG | |
| | | PFPNVKFVPTGGVNLDNVCEWFKAGVLAVGVGSALVK | |
| | | GTPVEVAEKAKAFVEKIRGCTE | |
| 1WA3-4 | | (MK)MEELFKKHKIVAVLRANSVEEAKMKALAVFVGG | |
| SEQ ID | | VHLIEITFTVPDADTVIKELSFLKELGAIIGAGTVTS | |
| NO: 111 | | VEQCRKAVESGAEFIVSPHLDEEISQFCKEKGVFYMP | |
| | | GVMTPTELVKAMKLGHTILKLFPGEVVGPQFVKAMKG | |
| | | PFPNVKFVPTGGVNLDNVCEWFKAGVLAVGVGSALVK | |
| | | GTIAEVAAKAAAFVEKIRGCTE | |
| 1WA3-5 | | (MK)MEELFKKHKIVAVLRANSVEEAKKKALAVELGG | |
| SEQ ID | | VDLIEITFTVPDADTVIKELSFLKEMGAIIGAGTVTS | |
| NO: 112 | | VEQCRKAVESGAEFIVSPHLDEEISQFCKEKGVFYMP | |
| | | GVMTPTELVKAMKLGHTILKLFPGEVVGPQFVKAMKG | |
| | | PFPNVKFVPTGGVNLDNVCEWFKAGVQAVGVGEALNK | |
| | | GTPVEVAEKAKAFVEKIRGCTE | |
| 1WA3-6 | | (MK)MEELFKKHKIVAVLRANSVEEAKKKALAVEMGG | |
| SEQ ID | | VDLIEITFTVPDADTVIKELSFLKELGAIIGAGTVTS | |
| NO: 113 | | VEQCRKAVESGAEFIVSPHLDEEISQFCKEKGVFYMP | |
| | | GVMTPTELVKAMKLGHTILKLFPGEVVGPQFVKAMKG | |
| | | PFPNVKFVPTGGVNLDNVCEWFKAGVQAVGVGEALNK | |
| | | GTPAEVAEKAKAFVEKIRGCTE | |
| I3-01 | | (METDTLLLWVLLLWVPGSTGDYKDEK)MEELFKKHK | |
| H35D | | IVAVLRANSVEEAKKKALAVFLGGVDLIEITFTVPDA | |
| SEQ ID | | DTVIKELSFLKEMGAIIGAGTVTSVEQCRKAVESGAE | |
| NO: 306 | | FIVSPHLDEEISQFCKEKGVFYMPGVMTPTELVKAMK | |
| | | LGHTILKLFPGEVVGPQFVKAMKGPFPNVKFVPTGGV | |
| | | NLDNVCEWFKAGVLAVGVGSALVKGTPVEVAEKAKAF | |
| | | VEKIRGCTE(QKLISEEDLHHHHHH) | |
| I3-01 | | (METDTLLLWVLLLWVPGSTGDYKDEK)MEELFKKHK | |
| K25D | | IVAVLRANSVEEAKKDALAVFLGGVHLIEITFTVPDA | |
| SEQ ID | | DTVIKELSFLKEMGAIIGAGTVTSVEQCRKAVESGAE | |
| NO: 307 | | FIVSPHLDEEISQFCKEKGVFYMPGVMTPTELVKAMK | |
| | | LGHTILKLFPGEVVGPQFVKAMKGPFPNVKFVPTGGV | |
| | | NLDNVCEWFKAGVLAVGVGSALVKGTPVEVAEKAKAF | |
| | | VEKIRGCTE(QKLISEEDLHHHHHH) | |
| I3-01 | | (METDTLLLWVLLLWVPGSTGDYKDEK)MEELFKKHK | |
| K25N | | IVAVLRANSVEEAKKNALAVFLGGVHLIEITFTVPDA | |
| SEQ ID | | DTVIKELSFLKEMGAIIGAGTVTSVEQCRKAVESGAE | |
| NO: 308 | | FIVSPHLDEEISQFCKEKGVFYMPGVMTPTELVKAMK | |
| | | LGHTILKLFPGEVVGPQFVKAMKGPFPNVKFVPTGGV | |
| | | NLDNVCEWFKAGVLAVGVGSALVKGTPVEVAEKAKAF | |
| | | VEKIRGCTE(QKLISEEDLHHHHHH) | |
| I3-01 | | (METDTLLLWVLLLWVPGSTGDYKDEK)MEELFKKHK | |
| L171Q | | IVAVLRANSVEEAKKKALAVFLGGVHLIEITFTVPDA | |
| SEQ ID | | DTVIKELSFLKEMGAIIGAGTVTSVEQCRKAVESGAE | |
| NO: 309 | | FIVSPHLDEEISQFCKEKGVFYMPGVMTPTELVKAMK | |
| | | LGHTILKLFPGEVVGPQFVKAMKGPFPNVKFVPTGGV | |
| | | NLDNVCEWFKAGVQAVGVGSALVKGTPVEVAEKAKAF | |
| | | VEKIRGCTE(QKLISEEDLHHHHHH) | |
| I3-01 | | (METDTLLLWVLLLWVPGSTGDYKDEK)MEELFKKHK | |
| L171Q/S177E/ | | IVAVLRANSVEEAKKKALAVFLGGVHLIEITFTVPDA | |
| V180N | | DTVIKELSFLKEMGAIIGAGTVTSVEQCRKAVESGAE | |
| SEQ ID | | FIVSPHLDEEISQFCKEKGVFYMPGVMTPTELVKAMK | |
| NO: 310 | | LGHTILKLFPGEVVGPQFVKAMKGPFPNVKFVPTGGV | |
| | | NLDNVCEWFKAGVQAVGVGEALNKGTPVEVAEKAKAF | |
| | | VEKIRGCTE(QKLISEEDLHHHHHH) | |
| I3-01 | | (METDTLLLWVLLLWVPGSTGDYKDEK)MEELFKKHK | |
| ‘secretion | | IVAVLRANSVEEAKKKALAVFLGGVDLIEITFTVPDA | |
| mutations’ | | DTVIKELSFLKEMGAIIGAGTVTSVEQCRKAVESGAE | |
| (H35D/L171Q/ | | FIVSPHLDEEISQFCKEKGVFYMPGVMTPTELVKAMK | |
| S177E/V180N) | | LGHTILKLFPGEVVGPQFVKAMKGPFPNVKFVPTGGV | |
| SEQ ID | | NLDNVCEWFKAGVQAVGVGEALNKGTPVEVAEKAKAF | |
| NO: 311 | | VEKIRGCTE(QKLISEEDLHHHHHH) | |
| I3-01 | | (METDTLLLWVLLLWVPGSTGD)MEELFKEHKIVAVL | |
| ‘negative | | RANSVEEAKKKALAVFLGGVHLIEITFTVPDADTVIK | |
| interior’ | | ELSFLKEMGAIIGAGTVTSVEQAREAVESGAEFIVSP | |
| SEQ ID | | HLDEEISQFAKEEGVFYMPGVMTPTELVKAMKLGHTI | |
| NO: 312 | | LKLFPGEVVGPQFVEAMKGPFPNVKFVPTGGVNLCNV | |
| | | AEWFEAGVLAVGVGSALVEGTPVEVAEKAKAFVEKIE | |
| | | GATE(QKLISEEDLHHHHHH) | |
| I3-01 | | (METDTLLLWVLLLWVPGSTGD)MEELFKEHKIVAVL | |
| ‘negative | | RANSVEEAKKKALAVFLGGVDLIEITFTVPDADTVIK | |
| interior | | ELSFLKEMGAIIGAGTVTSVEQAREAVESGAEFIVSP | |
| with | | HLDEEISQFAKEEGVFYMPGVMTPTELVKAMKLGHTI | |
| secretion | | LKLFPGEVVGPQFVEAMKGPFPNVKFVPTGGVNLDNV | |
| mutations’ | | AEWFEAGVQAVGVGEALNEGTPVEVAEKAKAFVEKIE | |
| SEQ ID | | GATE(QKLISEEDLHHHHHH) | |
| NO: 313 | | | |

Table 6 provides the amino acid sequence of a first assembly domain and second assembly domain of embodiments of the present disclosure. In each case, the pairs of sequences together form an 153 multimer with icosahedral symmetry. The right hand column in Table 6 identifies the residue numbers in each illustrative polypeptide that were identified as present at the interface of resulting assembled protein nanostructures (i.e., “identified interface residues”). As can be seen, the number of interface residues for the illustrative polypeptides of SEQ ID NO: 13-46 range from 4-13. In various embodiments, a first assembly domain and second assembly domain comprise an amino acid sequence that is at least 70%, 75%, 80%, 85%, 90%, 91%, 92%, 93%, 94%, 95%, 96%, 97%, 98%, 99%, or 100% identical over its length, and identical at least at 1, 2, 3, 4, 5, 6, 7, 8, 9, 10, 11, 12, or 13 identified interface positions (depending on the number of interface residues for a given polypeptide), to the amino acid sequence of a polypeptide selected from the group consisting of SEQ ID NOs: 13-46. SEQ ID NOs: 47-63 represent other amino acid sequences of a first assembly domain and second assembly domain from embodiments of the present disclosure. In other embodiments, a first assembly domain and/or second assembly domain comprise an amino acid sequence that is at least 70%, 75%, 80%, 85%, 90%, 91%, 92%, 93%, 94%, 95%, 96%, 97%, 98%, 99%, or 100% identical over its length, and identical at least at 20%, 25%, 33%, 40%, 50%, 60%, 70%, 75%, 80%, 90%, or 100% of the identified interface positions, to the amino acid sequence of a polypeptide selected from the group consisting of SEQ ID NOs:13-63.

As is the case with proteins in general, the polypeptides are expected to tolerate some variation in the designed sequences without disrupting subsequent assembly into protein nanostructures: particularly when such variation comprises conservative amino acid substitutions. As used herein, “conservative amino acid substitution” means that: hydrophobic amino acids (Ala, Gly, Met, Val, Ile, Leu, Phe, Thr, Trp) are substituted with other hydrophobic amino acids; hydrophobic amino acids with bulky side chains (Phe, Tyr, Trp) are substituted with other hydrophobic amino acids with bulky side chains; polar amino acids (Asp, Glu, Lys, Arg, Ser, Thr, Asn, Gly, Tyr, Gln) are substituted with other polar amino acids; amino acids with positively charged side chains (Arg, His, Lys) are substituted with other amino acids with positively charged side chains; and amino acids with negatively charged side chains (Asp, Glu) are substituted with other amino acids with negatively charged side chains.

In various embodiments of the protein nanostructures of the invention, a first assembly domain and second assembly domain, or the vice versa, comprise polypeptides with the amino acid sequence selected from the following pairs, or modified versions thereof (i.e., permissible modifications as disclosed for the polypeptides of the invention: isolated polypeptides comprising an amino acid sequence that is at least 75%, at least 80%, at least 85%, at least 90%, at least 91%, at least 92%, at least 93%, at least 94%, at least 95%, at least 96%, at least 97%, at least 98%, at least 99%, at least 100% identical over its length, and/or identical at least at one identified interface position, to the amino acid sequence indicated by the SEQ ID NO:
SEQ ID NO:13 and SEQ ID NO:14 (I53-34A and I53-34B);
SEQ ID NO:15 and SEQ ID NO:16 (I53-40A and I53-40B);
SEQ ID NO:15 and SEQ ID NO:36 (I53-40A and I53-40B.1);
SEQ ID NO:35 and SEQ ID NO:16 (I53-40A.1 and I53-40B);
SEQ ID NO:47 and SEQ ID NO:48 (I53-40A genus and I53-40B genus);
SEQ ID NO:17 and SEQ ID NO:18 (I53-47A and I53-47B);
SEQ ID NO:17 and SEQ ID NO:39 (I53-47A and I53-47B.1);
SEQ ID NO:17 and SEQ ID NO:40 (I53-47A and I53-47B.1NegT2);
SEQ ID NO:37 and SEQ ID NO:18 (I53-47A.1 and I53-47B);
SEQ ID NO:37 and SEQ ID NO:39 (I53-47A.1 and I53-47B.1);
SEQ ID NO:37 and SEQ ID NO:40 (I53-47A.1 and I53-47B.1NegT2);
SEQ ID NO:38 and SEQ ID NO:18 (I53-47A.1NegT2 and I53-47B);
SEQ ID NO:38 and SEQ ID NO:39 (I53-47A.1NegT2 and I53-47B.1);
SEQ ID NO:38 and SEQ ID NO:40 (I53-47A.1NegT2 and I53-47B.1NegT2);
SEQ ID NO:49 and SEQ ID NO:50 (I53-47A genus and I53-47B genus);
SEQ ID NO:19 and SEQ ID NO:20 (I53-50A and I53-50B);
SEQ ID NO:19 and SEQ ID NO:44 (I53-50A and I53-50B.1);
SEQ ID NO:19 and SEQ ID NO:45 (I53-50A and I53-50B.1NegT2);
SEQ ID NO:19 and SEQ ID NO:46 (I53-50A and I53-50B.4PosT1);
SEQ ID NO:41 and SEQ ID NO:20 (I53-50A.1 and I53-50B);
SEQ ID NO:41 and SEQ ID NO:44 (I53-50A.1 and I53-50B.1);
SEQ ID NO:41 and SEQ ID NO:45 (I53-50A.1 and I53-50B.1NegT2);
SEQ ID NO:41 and SEQ ID NO:46 (I53-50A.1 and I53-50B.4PosT1);
SEQ ID NO:42 and SEQ ID NO:20 (I53-50A.1NegT2 and I53-50B);
SEQ ID NO:42 and SEQ ID NO:44 (I53-50A.1NegT2 and I53-50B.1);
SEQ ID NO:42 and SEQ ID NO:45 (I53-50A.1NegT2 and I53-50B.1NegT2);
SEQ ID NO:42 and SEQ ID NO:46 (I53-50A.1NegT2 and I53-50B.4PosT1);
SEQ ID NO:43 and SEQ ID NO:20 (I53-50A.1PosT1 and I53-50B);
SEQ ID NO:43 and SEQ ID NO:44 (I53-50A.1PosT1 and I53-50B.1);
SEQ ID NO:43 and SEQ ID NO:45 (I53-50A.1PosT1 and I53-50B.1NegT2);
SEQ ID NO:43 and SEQ ID NO:46 (I53-50A.1PosT1 and I53-50B.4PosT1);
SEQ ID NO:51 and SEQ ID NO:52 (I53-50A genus and I53-50B genus);
SEQ ID NO:21 and SEQ ID NO:22 (I53-51A and I53-51B);
SEQ ID NO:23 and SEQ ID NO:24 (I52-03A and I52-03B);
SEQ ID NO:25 and SEQ ID NO:26 (I52-32A and I52-32B);
SEQ ID NO:27 and SEQ ID NO:28 (I52-33A and I52-33B)
SEQ ID NO:29 and SEQ ID NO:30 (I32-06A and I32-06B);
SEQ ID NO:31 and SEQ ID NO:32 (I32-19A and I32-19B);
SEQ ID NO:33 and SEQ ID NO:34 (I32-28A and I32-28B);
SEQ ID NO:35 and SEQ ID NO:36 (I53-40A.1 and I53-40B.1);
SEQ ID NO:53 and SEQ ID NO:54 (T32-28A and T32-28B);
SEQ ID NO:55 and SEQ ID NO:56 (T33-09A and T33-09B);
SEQ ID NO:57 and SEQ ID NO:58 (T33-15A and T33-15B);
SEQ ID NO:59 and SEQ ID NO:60 (T33-21A and T33-21B);
SEQ ID NO:61 and SEQ ID NO:62 (T33-28A and T32-28B); and
SEQ ID NO:63 and SEQ ID NO:56 (T33-31A and T33-09B (also referred to as T33-31B)).

In some embodiments, the assembly domains are I53_dn5B (trimer, optionally linked to the antigen) and I53_dn5A or I53_dn5A.1 or I53_dn5A.2 (pentamer). I53_dn5 nanostructures are described in US 2022/0072120 A1, the contents of which are incorporated by reference. I53_dn5 variants may include one or more amino acid substitutions, such as C94A, C119A, W18G, K84R, M88P, E91D, L117I, or L120D (together “I53_dn5A.1”; Ueda et al. eLife 9:e57659 (2020)) or A25E, M88A, C119T, L120E, A127E, L131T, I132K, E133A, or a deletion of positions 135-137 (“I53_dn5A.2”; Wang et al. bioRxiv 2022.08.04.502842).

In some embodiments, the ectodomains are expressed as a fusion protein with a first assembly domain. In some embodiments, the first assembly domain and the ectodomain are joined by a linker sequence.

Non-limiting examples of designed protein complexes useful in protein nanostructures of the present disclosure include those disclosed in U.S. Pat. No. 9,630,994; Int'l Pat. Pub No. WO2018187325A1; U.S. Pat. Pub. No. 2018/0137234 A1; U.S. Pat. Pub. No. 2019/0155988 A2, each of which is incorporated herein in its entirety.

In various embodiments of the protein nanostructures of the disclosure, the assembly domains are polypeptides with the amino acid sequence selected from the following pairs, or modified versions thereof (i.e., permissible modifications as disclosed for the polypeptides of the invention: isolated polypeptides comprising an amino acid sequence that is at least 70%, 75%, 80%, 85%, 90%, 91%, 92%, 93%, 94%, 95%, 96%, 97%, 98%, 99%, or 100% identical over its length, and/or identical at least at one identified interface position, to the amino acid sequence indicated by the SEQ ID NO):
SEQ ID NO: 65 and SEQ ID NO: 66 (T33_dn2A and T33_dn2B);
SEQ ID NO: 67 and SEQ ID NO: 68 (T33_dn5A and T33_dn5B);
SEQ ID NO: 69 and SEQ ID NO: 70 (T33_dn10A and T33_dn10B); or
SEQ ID NO: 71 and SEQ ID NO: 72 (I53_dn5A and I53_dn5B).

Various protein nanostructures are known in the art and described, for example in U.S. Pat. Pub. Nos. US 2015/0356240 A1; US 2016/0122392 A1, US 2018/0030429 A1, US 2019/0341124 A1, and US 2022/0072120 A1, the contents of which are incorporated by reference herein. In some embodiments, the protein nanostructure comprises, as an assembly domain, a variant of KDPG aldolase (Protein Data Bank code 1WA3) engineered to self-assemble into a protein nanostructure. In its native form, 1WA3 non-covalently assembles to form a trimer via a first interface (the trimer interface). When 20 copies of the trimer (60 monomers) are computationally docked to form a one-component icosahedral protein nanostructure, sets of five monomers of 1WA3 contact one another via a second interface (the pentamer interface). By introducing amino acid substitutions, the pentamer interface may be stabilized such that the protein nanostructure will spontaneously self-assemble, e.g., within the expressing cell or when isolated trimers (or monomers) are mixed under suitable conditions.

In some embodiments, the pentamer interface comprises 1, 2, 3, 4 or more interface residues, such as residues in positions 33, 61, 187, and 190 numbered according to SEQ ID NO: 107. In some embodiments, the assembly domain comprises a polypeptide sequence at least 70%, at least 80%, at least 90%, at least 91%, at least 92%, at least 93%, at least 94%, at least 95%, at least 96%, at least 97%, at least 98%, at least 99%, or 100% identical to SEQ ID NO: 107. In embodiments, the assembly domain comprises amino acid substitutions at 1, 2, 3, 4 of positions 33, 61, 187, and 190 compared to SEQ ID NO: 107. In some embodiments, the assembly domain comprises amino acid substitutions at 1, 2, 3, 4 of positions 33, 61, 187, and 190 compared to SEQ ID NO: 107. In some embodiments, a plurality of the amino acid substitutions are substitutions of a polar residue for a non-poplar residue (e.g., A, L, I, M, V, F, or W). In some embodiments, some or all of the amino acid substitutions are substitutions of a polar residue for a small, non-polar residue (e.g., A, L, I, M, or V). In some embodiments, the protein nanostructure comprises amino acid substitutions E33L or E33V; K61L or K61M; D187A or D187V; and/or R190A. In some embodiments, the protein nanostructure comprises amino acid substitutions E33L, K61M, D187V, and R190A. In some embodiments, the protein nanostructure comprises amino acid substitutions E33V, K61L, D187A, and R190A. In some embodiments, the assembly domain comprises an amino acid substitution to negate the enzymatic activity of the assembly domain (e.g., K129A). In embodiments, the assembly domain may comprise further amino acid substitutions (e.g., MI3; E56M or E56K; P186I; E191A; and/or K194A). In some embodiments, the assembly domain comprises amino acid substitutions that remove cysteine residues. In some embodiments, the assembly domain comprises C76A and/or C100A substitutions.

In one aspect, the disclosure provides a protein nanostructure comprising recombinant polypeptide, comprising an engineered ectodomain of a Respiratory Syncytial Virus (RSV) fusion (F) protein, wherein the ectodomain comprises (a) a C-terminal helix-forming segment, between about residue 500 and about residue 530 relative to SEQ ID NO: 1, comprising one or more amino acid substitutions selected such that the segment forms a stable alpha-helical homotrimer; (b) one, two, three or more amino acid substitutions at positions 140, 399, 400, 485, 486, 487, 488, 489, 494, or 498 relative to SEQ ID NO: 1; (c) one, two, three or more amino acid substitutions at positions 56, 58, 154, 187, 296, or 298 relative to SEQ ID NO: 1; (d) one, two, three or more amino acid substitutions at positions 75, 216, 218, or 219 relative to SEQ ID NO: 1; (e) one, two, three or more amino acid substitutions at positions 92, 232, 235, 238, 249, 250, or 254 relative to SEQ ID NO: 1; (f) one, two, three or more amino acid substitutions at positions 67, 137, or 339 relative to SEQ ID NO: 1; (g) a substitution of a non-cleavable linker in place of a furin cleavage site at about residues 100 to about residue 140 relative to SEQ ID NO: 1; or (h) any combination of (a)-(g).

In some embodiments, the polypeptide comprises, C-terminal to the ectodomain, a heterologous multimerization domain. In some embodiments, the multimerization domain is a trimerization domain. In some embodiments, the multimerization domain comprises a polypeptide sequence at least 70%, at least 80%, at least 90%, at least 91%, at least 92%, at least 93%, at least 94%, at least 95%, at least 96%, at least 97%, at least 98%, at least 99%, or 100% identical to I53-50A (SEQ ID NO: 19) or I53-50A ΔCys (SEQ ID NO: 64).

In another aspect, the disclosure provides a trimeric protein complex comprising a polypeptide of the disclosure. In some embodiments, thermal stability, assayed by nanoDSF, is increased by at least 10° C., at least 15° C., at least 20° C., about 10° C. to about 30° C., about 10° C. to about 20° C., or about 20° C. to about 30° C. compared to a trimeric protein complex lacking modifications (a)-(h). In some embodiments, stability, assayed by storage at about 40° C., is increased compared to a trimeric protein complex lacking modifications (a)-(h). In some embodiments, the thermal stability is increased compared to a reference RSV F protein comprising amino acid substitutions consisting essentially of S155C, S290C, S190F, and V207L (DS-Cav1).

In some embodiments, the first, trimeric component comprises an engineered ectodomain of a Respiratory Syncytial Virus (RSV) fusion (F) polypeptide and an I53-50A polypeptide.

In some embodiments, the first, trimeric component comprises a fusion protein comprising, in N- to C-terminal order, the RSV fusion (F) polypeptide, an amino acid linker, and the I53-50A polypeptide.

In some embodiments, the nanostructure is a two-component nanostructure comprising the first, trimeric component, wherein the first trimeric component comprises an engineered ectodomain of a RSV F polypeptide comprising an amino acid substitutions at position S155C, S290C, S190F, and V207L relative to SEQ ID NO: 1 and a C-terminal helix-forming segment comprising the polypeptide sequence NQSREIIRAINIVRKIASEK (SEQ ID NO: 10), and a multimerization domain comprising a polypeptide sequence at least 70%, at least 80%, at least 90%, at least 91%, at least 92%, at least 93%, at least 94%, at least 95%, at least 96%, at least 97%, at least 98%, at least 99%, or 100% identical to I53-50A (SEQ ID NO: 19) or I53-50A ΔCys (SEQ ID NO: 64), and/or the second pentameric component, wherein the pentameric component comprises a polypeptide sequence at least 70%, at least 80%, at least 90%, at least 91%, at least 92%, at least 93%, at least 94%, at least 95%, at least 96%, at least 97%, at least 98%, at least 99%, or 100% identical to SEQ ID NO: 20 or 71.

In some embodiments, the nanostructure is a two-component nanostructure comprising the first, trimeric component, wherein the first trimeric component comprises an engineered ectodomain of a RSV F polypeptide comprising an amino acid relative to SEQ ID NO: 1 and a C-terminal helix-forming segment comprising the polypeptide sequence NQSREIIRAINIVRKIASEK (SEQ ID NO: 10), and a multimerization domain comprising a polypeptide sequence at least 70%, at least 80%, at least 90%, at least 91%, at least 92%, at least 93%, at least 94%, at least 95%, at least 96%, at least 97%, at least 98%, at least 99%, or 100% identical to I53-50A (SEQ ID NO: 19) or I53-50A ΔCys (SEQ ID NO: 64), and/or the second pentameric component, wherein the pentameric component comprises a polypeptide sequence at least 70%, at least 80%, at least 90%, at least 91%, at least 92%, at least 93%, at least 94%, at least 95%, at least 96%, at least 97%, at least 98%, at least 99%, or 100% identical to SEQ ID NO: 20 or 71

In some embodiments, the nanostructure is a two-component nanostructure comprising the first, trimeric component, wherein the first trimeric component comprises an engineered ectodomain of a RSV F polypeptide comprising an amino acid substitutions at position S155C, S290C, S190F, V207L, D489A, T400D, E487R, and K498A relative to SEQ ID NO: 1 and a C-terminal helix-forming segment comprising the polypeptide sequence NQSREIIRAINIVRKIASEK (SEQ ID NO: 10), and a multimerization domain comprising a polypeptide sequence at least 70%, at least 80%, at least 90%, at least 91%, at least 92%, at least 93%, at least 94%, at least 95%, at least 96%, at least 97%, at least 98%, at least 99%, or 100% identical to I53-50A (SEQ ID NO: 19) or I53-50A ΔCys (SEQ ID NO: 64) and/or the second pentameric component, wherein the pentameric component comprises a polypeptide sequence at least 70%, at least 80%, at least 90%, at least 91%, at least 92%, at least 93%, at least 94%, at least 95%, at least 96%, at least 97%, at least 98%, at least 99%, or 100% identical to SEQ ID NO: 20 or 71.

In some embodiments, the nanostructure is a two-component nanostructure comprising the first, trimeric component, wherein the first trimeric component comprises an engineered ectodomain of a RSV F polypeptide comprising an amino acid substitutions at position D489A, T400D, E487R, and K498A relative to SEQ ID NO: 1 and a C-terminal helix-forming segment comprising the polypeptide sequence NQSREIIRAINIVRKIASEK (SEQ ID NO: 10), and a multimerization domain comprising a polypeptide sequence at least 70%, at least 80%, at least 90%, at least 91%, at least 92%, at least 93%, at least 94%, at least 95%, at least 96%, at least 97%, at least 98%, at least 99%, or 100% identical to I53-50A (SEQ ID NO: 19) or I53-50A ΔCys (SEQ ID NO: 64) and/or the second pentameric component, wherein the pentameric component comprises a polypeptide sequence at least 70%, at least 80%, at least 90%, at least 91%, at least 92%, at least 93%, at least 94%, at least 95%, at least 96%, at least 97%, at least 98%, at least 99%, or 100% identical to SEQ ID NO: 20 or 71.

In some embodiments, the nanostructure is a two-component nanostructure comprising the first, trimeric component, wherein the first trimeric component comprises an engineered ectodomain of a RSV F polypeptide comprising an amino acid substitutions at position S155C, S290C, S190F, V207L, F488W, D489A, T400D, E487R, K498A, and T249P relative to SEQ ID NO: 1 and a C-terminal helix-forming segment comprising the polypeptide sequence NQSREIIRAINIVRKIASEK (SEQ ID NO: 10), and a multimerization domain comprising a polypeptide sequence at least 70%, at least 80%, at least 90%, at least 91%, at least 92%, at least 93%, at least 94%, at least 95%, at least 96%, at least 97%, at least 98%, at least 99%, or 100% identical to I53-50A (SEQ ID NO: 19) or I53-50A ΔCys (SEQ ID NO: 64), and/or the second pentameric component, wherein the pentameric component comprises a polypeptide sequence at least 70%, at least 80%, at least 90%, at least 91%, at least 92%, at least 93%, at least 94%, at least 95%, at least 96%, at least 97%, at least 98%, at least 99%, or 100% identical to SEQ ID NO: 20 or 71.

In some embodiments, the nanostructure is a two-component nanostructure comprising the first, trimeric component, wherein the first trimeric component comprises an engineered ectodomain of a RSV F polypeptide comprising an amino acid substitutions at position F488W, D489A, T400D, E487R, K498A, and T249P relative to SEQ ID NO: 1 and a C-terminal helix-forming segment comprising the polypeptide sequence NQSREIIRAINIVRKIASEK (SEQ ID NO: 10), and a multimerization domain comprising a polypeptide sequence at least 70%, at least 80%, at least 90%, at least 91%, at least 92%, at least 93%, at least 94%, at least 95%, at least 96%, at least 97%, at least 98%, at least 99%, or 100% identical to I53-50A (SEQ ID NO: 19) or I53-50A ΔCys (SEQ ID NO: 64), and/or the second pentameric component, wherein the pentameric component comprises a polypeptide sequence at least 70%, at least 80%, at least 90%, at least 91%, at least 92%, at least 93%, at least 94%, at least 95%, at least 96%, at least 97%, at least 98%, at least 99%, or 100% identical to SEQ ID NO: 20 or 71.

In some embodiments, the nanostructure is a two-component nanostructure comprising the first, trimeric component, wherein the first trimeric component comprises an engineered ectodomain of a RSV F polypeptide comprising an amino acid substitutions at position S155C, S290C, S190F, V207L, F488W, D489A, T400D, E487R, K498A, and D486A relative to SEQ ID NO: 1 and a C-terminal helix-forming segment comprising the polypeptide sequence NQSREIIRAINIVRKIASEK (SEQ ID NO: 10), and a multimerization domain comprising a polypeptide sequence at least 70%, at least 80%, at least 90%, at least 91%, at least 92%, at least 93%, at least 94%, at least 95%, at least 96%, at least 97%, at least 98%, at least 99%, or 100% identical to I53-50A (SEQ ID NO: 19) or I53-50A ΔCys (SEQ ID NO: 64), and/or the second pentameric component, wherein the pentameric component comprises a polypeptide sequence at least 70%, at least 80%, at least 90%, at least 91%, at least 92%, at least 93%, at least 94%, at least 95%, at least 96%, at least 97%, at least 98%, at least 99%, or 100% identical to SEQ ID NO: 20 or 71.

In some embodiments, the nanostructure is a two-component nanostructure comprising the first, trimeric component, wherein the first trimeric component comprises an engineered ectodomain of a RSV F polypeptide comprising an amino acid substitutions at position F488W, D489A, T400D, E487R, K498A, and D486A relative to SEQ ID NO: 1 and a C-terminal helix-forming segment comprising the polypeptide sequence NQSREIIRAINIVRKIASEK (SEQ ID NO: 10), and a multimerization domain comprising a polypeptide sequence at least 70%, at least 80%, at least 90%, at least 91%, at least 92%, at least 93%, at least 94%, at least 95%, at least 96%, at least 97%, at least 98%, at least 99%, or 100% identical to I53-50A (SEQ ID NO: 19) or I53-50A ΔCys (SEQ ID NO: 64), and/or the second pentameric component, wherein the pentameric component comprises a polypeptide sequence at least 70%, at least 80%, at least 90%, at least 91%, at least 92%, at least 93%, at least 94%, at least 95%, at least 96%, at least 97%, at least 98%, at least 99%, or 100% identical to SEQ ID NO: 20 or 71.

In some embodiments, the nanostructure is a two-component nanostructure comprising the first, trimeric component, wherein the first trimeric component comprises an engineered ectodomain of a RSV F polypeptide comprising a polypeptide sequence at least 70%, at least 80%, at least 90%, at least 91%, at least 92%, at least 93%, at least 94%, at least 95%, at least 96%, at least 97%, at least 98%, at least 99%, or 100% identical to any one of the sequences listed in Table 14 and a C-terminal helix-forming segment comprising the polypeptide sequence NQSREIIRAINIVRKIASEK (SEQ ID NO: 10), and a multimerization domain comprising a polypeptide sequence at least 70%, at least 80%, at least 90%, at least 91%, at least 92%, at least 93%, at least 94%, at least 95%, at least 96%, at least 97%, at least 98%, at least 99%, or 100% identical to I53-50A (SEQ ID NO: 19) or I53-50A ΔCys (SEQ ID NO: 64), and/or the second pentameric component, wherein the pentameric component comprises a polypeptide sequence at least 70%, at least 80%, at least 90%, at least 91%, at least 92%, at least 93%, at least 94%, at least 95%, at least 96%, at least 97%, at least 98%, at least 99%, or 100% identical to SEQ ID NO: 20 or 71.

In some embodiments, the trimeric component comprises a polypeptide sequence at least 70%, at least 80%, at least 90%, at least 91%, at least 92%, at least 93%, at least 94%, at least 95%, at least 96%, at least 97%, at least 98%, at least 99%, or 100% identical to any one of the sequences listed in Table 14 or to any one of the sequences listed in Table 14 without the underlined and/or bold/italicized polypeptide sequences.

In another aspect, the disclosure provides a protein nanostructure comprising a trimeric component comprising a polypeptide described herein. In some embodiments, the nanostructure is a two-component nanostructure comprising the first, trimeric component and a second, pentameric component. In some embodiments, the pentameric component comprises a polypeptide sequence at least 70%, at least 80%, at least 90%, at least 91%, at least 92%, at least 93%, at least 94%, at least 95%, at least 96%, at least 97%, at least 98%, at least 99%, or 100% identical to any one or more of SEQ ID NOs: 20, 44, 45, 52, 71, 73, 74.

### Ferritin-Based Nanostructures

In some embodiments, the assembly domain is a ferritin polypeptide. In some embodiments, the assembly domain of a ferritin protein nanostructure comprises a polypeptide sequence at least 70%, at least 80%, at least 90%, at least 91%, at least 92%, at least 93%, at least 94%, at least 95%, at least 96%, at least 97%, at least 98%, at least 99%, or 100% identical to any one of the following sequences:

(SEQ ID NO: 114)
MLSKDIIKLLNEQVNKEMNSSNLYMSMSSWCYTHSLDGAGLFLFDHAAE
EYEHAKKLIIFLNENNVPVQLTSISAPEHKFEGLTQIFQKAYEHEQHIS
ESINNIVDHAIKSKDHATFNFLQWYVAEQHEEEVLFKDILDKVELIGNE
NHGLYLADQYVKGIAKSRKS.
(SEQ ID NO: 115)
MLKPEMIEKLNEQMNLELYSSLLYQQMSAWCSYHTFEGAAAFLRRHAQE
EMTHMQRLFDYLTDTGNLPRINTVESPFAEYSSLDELFQETYKHEQLIT
QKINELAHAAMTNQDYPTFNFLQWYVSEQHEEEKLFKSIIDKLSLAGKS
GEGLYFIDKELSTLDAQN.
(SEQ ID NO: 116)
NFHQDCEAGLNRTVNLKFHSSYVYLSMASYFNRDDVALSNFAKFFRERS
EEEKEHAEKLIEYQNQRGGRVFLQSVEKPERDDWANGLEALQTALKLQK
SVNQALLDLHAVAADKSDPHMTDFLESPYLSESVETIKKLGDHITSLKK
LWSSHPGMAEYLFNKHTLG.
(SEQ ID NO: 117)
QFSKDIEKLLNEQVNKEMQSSNLYMSMSSWCYTHSLDGAGLFLFDHAAE
EYEHAKKLIIFLNENNVPVQLTSISAPEHKFEGLTQIFQKAYEHEQHIS
ESINNIVDHAIKSKDHATFNFLQWYVAEQHEEEVLFKDILDKIELIGNE
NHGLYLADQYVKGIAKSRKSGS.
(SEQ ID NO: 118)
SGESQVRQNFKPEMEEKLNEQMNLELYSSLLYQQMSAWCSYHTFEGAAA
FLRRHAQEEMTHMQRLFDYLTDTGNLPRINTVESPFAEYSSLDELFQET
YKHEQLITQKINELAHAAMTNQDYPTFNFLQWYVSEQHEEEKLFKSIID
KLSLAGKSGEGLYFIDKELSTLDGS.

### Other Nanostructures or Nanoparticles

In some embodiments, the ectodomains described herein are displayed on any nanostructure or nanoparticle known in the art. Illustrative nanostructures and nanoparticles include, but are not limited to Human papillomavirus (HPV) virus-like particles (VLPs), Chikungunya VLPs, AP205 capsid protein VLPs, phage VLPs (e.g., bacteriophage). Display on these and other platforms may be performed by creating a fusion protein of the ectodomain to a relevant protein of the system, by bioconjugate chemistry (e.g., SpyCatcher), or other means known in the art. The protein nanostructure may be a lumazine synthase nanoparticle as described, e.g., in Geng et al. PLoS Pathog. 17(9):e1009897 (2021). The protein nanostructure may be a ferritin nanoparticle as described, e.g., in Joyce et al. bioRxiv 2021.05.09.443331 and in U.S. Pat. Pub. No. US 2019/0330279 A1.

### IV. Polynucleotides

In another aspect, the present disclosure provides polynucleotides encoding an antigen, a first component, and/or a second component, of the present disclosure. The polynucleotides sequences may comprise RNA or DNA. As used herein, “polynucleotides” are those that have been removed from their normal surrounding polynucleotides sequences in the genome or in cDNA sequences. Such polynucleotides sequences may comprise additional sequences useful for promoting expression and/or purification of the encoded protein, including but not limited to polyA sequences, modified Kozak sequences, and sequences encoding epitope tags, export signals, and secretory signals, nuclear localization signals, and plasma membrane localization signals. It will be apparent to those of skill in the art, based on the teachings herein, what nucleic acid sequences will encode the proteins of the disclosure.

### V. Delivery Vehicles

In some embodiments, polynucleotides (e.g., mRNA) encoding protein nanostructures including a component comprising a viral protein monomer of a trimeric viral antigen are formulated in a delivery vehicle. In some embodiments, the delivery vehicle is a non-viral vector. In some embodiments, the delivery vehicle is a lipid nanoparticle (LNP). In some embodiments, the delivery vehicle is a liposome. In some embodiments, the delivery vehicle is a polymeric-non-viral vector, such as spermine, Polyethylenimine, chitosan, or polyurethane. In some embodiments, the delivery vehicle is a polymer delivery system, such as poly-amido-amine (PAA), poly-beta aminoesters (PBAEs) or polyethylenimine (PEI). In some embodiments, the delivery vehicle is a ferritin nanoparticle. In some embodiments, the delivery vehicle is an encapsulin.

In some embodiments, polynucleotides (e.g., mRNA) encoding protein nanostructures including a component comprising a viral protein monomer of a trimeric viral antigen are formulated in a nanoparticle. In some embodiments, the nanoparticle is a lipid nanoparticle (LNP). In some embodiments, the polynucleotides are formulated in a lipid-polycation complex, referred to as a cationic LNP. As a non-limiting example, the polycation may include a cationic peptide or a polypeptide such as, but not limited to, polylysine, polyornithine and/or polyarginine. In some embodiments, the polynucleotides are formulated in a LNP that includes a non-cationic lipid such as, but not limited to, cholesterol or dioleoyl phosphatidylethanolamine (DOPE).

In various embodiments, the lipid nanoparticles have a mean diameter from about 30 nm to about 150 nm, from about 40 nm to about 150 nm, from about 50 nm to about 150 nm, from about 60 nm to about 130 nm, from about 70 nm to about 110 nm, from about 70 nm to about 100 nm, from about 80 nm to about 100 nm, from about 90 nm to about 100 nm, from about 70 to about 90 nm, from about 80 nm to about 90 nm, from about 70 nm to about 80 nm, or about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm. In some embodiments, the LNPs are substantially non-toxic. In certain embodiments, polynucleotides, when present in the LNPs, are resistant in aqueous solution to degradation with a nuclease. Lipids and LNPs comprising polynucleotides and their method of preparation are described in, e.g., U.S. Pat. Nos. 8,569,256, 5,965,542 and U.S. Patent Publication Nos. 2021/0323914, 2016/0199485, 2016/0009637, 2015/0273068, 2015/0265708, 2015/0203446, 2015/0005363, 2014/0308304, 2014/0200257, 2013/086373, 2013/0338210, 2013/0323269, 2013/0245107, 2013/0195920, 2013/0123338, 2013/0022649, 2013/0017223, 2012/0295832, 2012/0183581, 2012/0172411, 2012/0027803, 2012/0058188, 2011/0311583, 2011/0311582, 2011/0262527, 2011/0216622, 2011/0117125, 2011/0091525, 2011/0076335, 2011/0060032, 2010/0130588, 2007/0042031, 2006/0240093, 2006/0083780, 2006/0008910, 2005/0175682, 2005/017054, 2005/0118253, 2005/0064595, 2004/0142025, 2007/0042031, 1999/009076 and PCT Pub. Nos. WO 99/39741, WO 2017/004143, WO 2017/075531, WO 2015/199952, WO 2014/008334, WO 2013/086373, WO 2013/086322, WO 2013/016058, WO 2013/086373, WO2011/141705, WO 2017/049245, WO 2010/144740, WO/2017/075531, and WO 2001/07548, the contents of which are incorporated by reference herein.

Further exemplary lipids and LNPs and their manufacture are known in the art—for example in U.S. Pat. Pub. No. U.S. 2012/0276209, Semple et al., 2010, Nat Biotechnol., 28(2):172-176; Akinc et al., 2010, Mol Ther., 18(7): 1357-1364; Basha et al., 2011, Mol Ther, 19(12): 2186-2200; Leung et al., 2012, J Phys Chem C Nanomater Interfaces, 116(34): 18440-18450; Lee et al., 2012, Int J Cancer., 131(5): E781-90; Belliveau et al., 2012, Mol Ther nucleic Acids, 1: e37; Jayaraman et al., 2012, Angew Chem Int Ed Engl., 51(34): 8529-8533; Mui et al., 2013, Mol Ther Nucleic Acids. 2, e139; Maier et al., 2013, Mol Ther., 21(8): 1570-1578; and Tam et al., 2013, Nanomedicine, 9(5): 665-74, each of which are incorporated by reference herein. Lipids and their manufacture can be found, for example, in U.S. Pat. Pub. Nos. 2015/0376115 and 2016/0376224, the contents of which are incorporated by reference herein.

### VI. Pharmaceutical Compositions

The disclosure also provides pharmaceutical compositions. Such pharmaceutical compositions can be used for generating an immune response against an infectious disease in a subject. The pharmaceutical compositions of the disclosure may include a pharmaceutically acceptable carrier. A thorough discussion of such carriers is available in Chapter 30 of Remington: The Science and Practice of Pharmacy (23^{rd} ed., 2021).

In some embodiments, the pharmaceutical composition can also include excipients and/or additives. Examples of these are surfactants, stabilizers, complexing agents, antioxidants, or preservatives which prolong the duration of use of the finished pharmaceutical formulation, flavorings, vitamins, or other additives known in the art. Complexing agents include, but are not limited to, ethylenediaminetetraacetic acid (EDTA) or a salt thereof, such as the disodium salt, citric acid, nitrilotriacetic acid and the salts thereof. In some embodiments, preservatives include, but are not limited to, those that protect the solution from contamination with pathogenic particles, including benzalkonium chloride or benzoic acid, or benzoates such as sodium benzoate. Antioxidants include, but are not limited to, vitamins, provitamins, ascorbic acid, vitamin E, salts or esters thereof.

Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethyl cellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the disclosure. One of skill in the art will recognize that other pharmaceutical excipients are useful in the present disclosure.

In some embodiments, one or more tonicity agents may be added to provide the desired ionic strength. Tonicity agents for use herein include those which display no or only negligible pharmacological activity after administration. Both inorganic and organic tonicity adjusting agents may be used.

In another aspect, the disclosure provides a pharmaceutical composition comprising a polypeptide, a protein complex, or a nanostructure of the disclosure.

### VII. Vaccines

In another aspect, the disclosure provides a vaccine comprising a polypeptide, a protein complex, or a nanostructure as disclosed herein.

In some embodiments, the vaccine comprises an adjuvant.

In some embodiments, the pharmaceutical composition provided herein is administered as a RSV vaccine, for example, an RSV/A vaccine, and RSV/B vaccine, or a bivalent RSV A/B vaccine.

### Adjuvants

Adjuvants or immune potentiators may also be administered with or in combination with lipid nanoparticle composition. Advantages of adjuvants include, but are not limited to, the enhancement of the immunogenicity of antigens, modification of the nature of the immune response, the reduction of the antigen amount needed for a successful immunization, the reduction of the frequency of booster immunizations needed and an improved immune response in elderly and immunocompromised vaccinees. These may be co-administered by any route, e.g., intramuscular, subcutaneous, intravenous, or intradermal injections.

Adjuvants may include, but are not limited to, a natural or a synthetic adjuvant. Adjuvants may be organic or inorganic.

Adjuvants may be selected from any of the classes (1) mineral salts, e.g., aluminum hydroxide and aluminum or calcium phosphate gels; (2) emulsions including: oil emulsions and surfactant based formulations, e.g., microfluidised detergent stabilized oil-in-water emulsion, purified saponin, oil-in-water emulsion, stabilized water-in-oil emulsion; (3) particulate adjuvants, e.g., virosomes (unilamellar liposomal vehicles incorporating influenza hemagglutinin), structured complex of saponins and lipids, polylactide co-glycolide (PLG); (4) microbial derivatives; (5) endogenous human immunomodulators; (6) inert vehicles, such as gold particles; (7) microorganism derived adjuvants; (8) tensoactive compounds; (9) carbohydrates; or combinations thereof.

Adjuvants for nucleic acid vaccines (DNA) have been disclosed in, for example, Kobiyama, et al., Vaccines, 2013, 1(3), 278-292, the contents of which are incorporated herein by reference in their entirety. Any of the adjuvants disclosed by Kobiyama et al., may be used in the vaccines as described herein.

Other adjuvants which may be utilized include any of those listed on the web-based vaccine adjuvant database, on the World Wide Web at violinet.org/vaxjo/and described in for example Sayers, et al., J. Biomedicine and Biotechnology, volume 2012 (2012), Article ID 831486, 13 pages, the contents of which are incorporated herein by reference in their entirety.

Specific adjuvants may include cationic liposome-DNA complex JVRS-100, aluminum hydroxide vaccine adjuvant, aluminum phosphate vaccine adjuvant, aluminum potassium sulfate adjuvant, alhydrogel, ISCOM(s)™, Freund's Complete Adjuvant, Freund's Incomplete Adjuvant, CpG DNA Vaccine Adjuvant, Cholera toxin, Cholera toxin B subunit, Liposomes, Saponin Vaccine Adjuvant, DDA Adjuvant, Squalene-based Adjuvants, Etx B subunit Adjuvant, IL-12 Vaccine Adjuvant, LTK63 Vaccine Mutant Adjuvant, TiterMax Gold Adjuvant, Ribi Vaccine Adjuvant, Montanide ISA 720 Adjuvant, Corynebacterium-derived P40 Vaccine Adjuvant, MPL™ Adjuvant, AS04, AS02, ASO1_{E}, Lipopolysaccharide Vaccine Adjuvant, Muramyl Dipeptide Adjuvant, CRL1005, Killed Corynebacterium parvum Vaccine Adjuvant, Montanide ISA 51, Bordetella pertussis component Vaccine Adjuvant, Cationic Liposomal Vaccine Adjuvant, Adamantylamide Dipeptide Vaccine Adjuvant, Arlacel A, VSA-3 Adjuvant, Aluminum vaccine adjuvant, Polygen Vaccine Adjuvant, ADJUMER™, Algal Glucan, Bay R1005, Theramide®, Stearyl Tyrosine, Specol, Algammulin, AVRIDINE®, Calcium Phosphate Gel, CTA1-DD gene fusion protein, DOC/Alum Complex, Gamma Inulin, Gerbu Adjuvant, GM-CSF, GMDP, Recombinant hIFN-gamma/Interferon-g, Interleukin-1β, Interleukin-2, Interleukin-7, Sclavo peptide, Rehydragel LV, Rehydragel HPA, Loxoribine, MF59, MTP-PE Liposomes, Murametide, Murapalmitine, D-Murapalmitine, NAGO, Non-Ionic Surfactant Vesicles, PMMA, Protein Cochleates, QS-21, SPT (Antigen Formulation), nanoemulsion vaccine adjuvant, AS03, Quil-A vaccine adjuvant, RC529 vaccine adjuvant, LTR192G Vaccine Adjuvant, E. coli heat-labile toxin, LT, amorphous aluminum hydroxyphosphate sulfate adjuvant, Calcium phosphate vaccine adjuvant, Montanide Incomplete Seppic Adjuvant, Imiquimod, Resiquimod, AF03, Flagellin, Poly(I:C), ISCOMATRIX®, Abisco-100 vaccine adjuvant, Albumin-heparin microparticles vaccine adjuvant, AS-2 vaccine adjuvant, B7-2 vaccine adjuvant, DHEA vaccine adjuvant, Immunoliposomes Containing Antibodies to Costimulatory Molecules, SAF-1, Sendai Proteoliposomes, Sendai-containing Lipid Matrices, Threonyl muramyl dipeptide (TMDP), Ty Particles vaccine adjuvant, Bupivacaine vaccine adjuvant, DL-PGL (Polyester poly (DL-lactide-co-glycolide)) vaccine adjuvant, IL-15 vaccine adjuvant, LTK72 vaccine adjuvant, MPL-SE vaccine adjuvant, non-toxic mutant E112K of Cholera Toxin mCT-E112K, and/or Matrix-S.

In some embodiments, the adjuvant comprises squalene. In some embodiments, the adjuvant comprises aluminum hydroxide. In some embodiments, the adjuvant comprises AS01E.

### VIII. Methods of Use

In another aspect, the disclosure provides methods of administration for the composition, the pharmaceutical composition, or the vaccine described herein.

In another aspect, the disclosure provides a method of vaccinating a subject, comprising administering to the subject a composition described herein. In another aspect, the disclosure provides a method of generating an immune response in a subject, comprising administering to the subject a composition described herein. In another aspect, the disclosure provides a method of treating or preventing RSV disease in a subject, comprising administering to the subject a composition described herein. In another aspect, the disclosure provides a composition of the disclosure for use in vaccinating, generating an immune response, or treating or preventing RSV disease. In another aspect, the disclosure provides a composition, method, or use as described herein. In another aspect, the disclosure provides a method of making a compositions, comprising culturing host cells modified to express one or more polypeptides as described herein.

In some embodiments, the method comprising administering the vaccine described herein. In some embodiments, the subject is simultaneously immunized against infection by respiratory syncytial virus (RSV). In some embodiments, the vaccine is administered by subcutaneous injection. In some embodiments, the vaccine is administered by intramuscular injection. In some embodiments, the vaccine is administered by intradermal injection. In some embodiments, the vaccine is administered intranasally. In one aspect, the disclosure provides a pre-filled syringe comprising the vaccine described herein. In one aspect, the disclosure provides a kit comprising the vaccine described herein or the pre-filled syringe described herein.

In some embodiments, the unit dose of the pharmaceutical composition comprises about 0.5 μg to about 1 μg, about 20 μg to about 25 μg, about 70 μg to about 75 μg, about 100 μg to about 125 μg, about 100 μg to about 150 μg, about 125 μg to about 175 μg, about 200 μg to about 250 μg, about 225 μg to about 300 μg, or about 250 μg to about 350 μg of the protein nanostructures.

In some embodiments, the unit dose of the pharmaceutical composition comprises about 0.5 μg to about 1 μg, about 20 μg to about 25 μg, about 25 μg to about 50 μg, about 50 μg to about 70 μg, about 70 μg to about 75 μg, about 75 μg to about 100 μg, about 100 μg to about 125 μg, about 125 μg to about 150 μg, about 150 μg to about 175 μg, about 175 μg to about 200 μg, about 200 μg to about 250 μg, or about 250 μg to about 300 μg of the protein nanostructures.

In some embodiments, the subject is at risk of RSV disease. In some embodiments, the subject is an adult of over 60 years of age. In some embodiments, the subject is a healthy adult of 18-45 years of age. In some embodiments, the subject is a pregnant women between week 32 and week 36 of pregnancy. In some embodiments, the subject is a pregnant women between week 30 and week 38 of pregnancy. In some embodiments, the subject is a pregnant women between week 28 and week 38 of pregnancy.

Any aspect or embodiment described herein can be combined with any other aspect or embodiment as disclosed herein.

### EXAMPLES

The following Examples are merely illustrative and are not intended to limit the scope or content of the invention in any way.

### Example 1. Remodeling the C-Terminus of RSV F Protein

This Example describes remodeling the C terminus of the RSV F protein to create a stable helix-forming segment.

RSV F protein, like other class I viral membrane fusion protein, forms a trimer with two primary conformations (prefusion and postfusion). The C terminus of the ectodomain, adjacent to the transmembrane domain, is believed to form a helical bundle in the context of the native protein. Structures of the prefusion F protein generally model the C terminus as alpha-helical, with structured density ending at about residue 510 or 512 (e.g., PDB 5C6B and 5UDD, respectively). The native sequence after residue 513 is often replaced with a four-residue linker (SAIG) and the trimeric FoldOn domain. The predicted transmembrane domain begins at residue 527. The sequence of a native RSV/B F protein sequence (GenBank: WDV37446.1) is shown here with the transmembrane domain bold/underlined:

(SEQ ID NO: 1)

| | |
|---|---|
| 1 | MELLIHRSSA IFLTLAINAL YLISSQNITE EFYQSTCSAV |
| 41 | SRGYLSALRT GWYTSVITIE LSNIKETKCN GTDTKVKLIK |
| 81 | QELDKYKNAV TELQLLMQNT PAVNNRARRE APQYMNYTIN |
| 121 | TTKNLNVSIS KKRKRRFLGF LLGVGSAIAS GIAVSKVLHL |
| 161 | EGEVNKIKNA LQLINKAVVS LSNGVSVLTS RVLDLKNYIN |
| 201 | NQLLPMVNRQ SCRISNIETV IEFQQKNSRL LEITREFSVN |
| 241 | AGVTTPLSTY MLINSELLSL INDMPITNDQ KKLMSSNVQI |
| 281 | VRQQSYSIMS IIKEEVLAYV VQLPIYGVID TPCWKLHTSP |
| 321 | LCTTNIKEGS NICLTRTDRG WYCDNAGSVS FFPQADTCKV |
| 361 | QSNRVFCDTM NSLILPSEVS LCNTDIFNSK YDCKIMTSKT |
| 401 | DISSSVITSL GAIVSCYGKT KCTASNKNRG IIKTFSNGCD |
| 441 | YVSNKGVDTV SVGNTLYYVN KLEGKNLYVK GEPIINYYDP |
| 481 | LVFPSDEFDA SISQVNEKIN QSLAFIRRSD ELLHNVNTGK |
| 521 | STTNIMITAI TIVIIVVLLS LIAIGLLLYC KAKNTPVTLS |
| 561 | KDQLSGINNI AFSK |

We hypothesized that the poor structural resolution of the C terminus of the ectodomain reflects imperfect hydrophobic packing of the helical bundle in the native protein when it is expressed recombinantly. We developed a pipeline to remodel the C terminus of the ectodomain to generate improved antigens for use in vaccines. Our method structurally remodels the segment (corresponding to about residue 500 and about residue 530 relative to native sequence) into a more structurally stable helical bundle by substituting residues (e.g., to generate new non-covalent interactions, prevent clashing of residues, or adjust the polypeptide backbone), as well as preserve or enhance polar exposed surfaces, and thereby decrease the free energy of self-association of the protomers (as predicted ddG and measuring thermal denaturation temperature). The remodeling pipeline included manual selection of sequences predicted to form structures capable of serving as adaptors to connect the C terminus of the ectodomain to a trimerization domain, such as an I53-50A multimerization domain. Manual selection was performed based on a combination of polypeptide sequence diversity and computational metrics, which included geometry design space, hydrophobic core packages, termini availability, and lack of obvious errors in conformation (i.e., solvent exposed trytophans).

Structural models from the Protein Data Bank (PDB) were prepared for design by symmetrization, removal of hetero-atoms, renumbering, relaxing, and marking of glycosylation sites. Rosetta blueprint files were written to generate a gradient between the native structure and the remodeled domain. Generally, the blueprint included a two-residue native sequence that is remodeled using helical constraints; followed by one or more existing helical residues that contribute to hydrophobic contacts at the C-terminus, which are allowed to design; followed by an alpha-helical segment ranging in length from approximately 1-10 amino acids in length, determined empirically by the designer. For example, to remodel this sequence:

(SEQ ID NO: 314)
481 LVFPSDEFDA SISQVNEKIN QSLAFIRRSD ELLHNVNTG

a blueprint may be generated were the amino acid residue is set to match the native sequence (A), to start with native sequence but allow substitutions (A), to newly modelled as any amino acid (X) (top line), while the three-dimensional structure of the polypeptide is set to either match the native structure (.) or to be constrained to be helical (H):

481 LVFPSDEFDA SISQVNEKIN QSLAFIRRSX XXXXXXXXX (SEQ ID NO: 315)
.......... .......... HHHHHHHHHH HHHHHHHHH (SEQ ID NO: 316)

Using this or similar blueprints, designs were generated with Rosetta Remodel. Remodel results were filtered by ddG calculations from the design model directly, and manually reverted to remove any introduced glycosylation sites, exposed hydrophobic residues, or buried polar residues. The resulting models were relaxed and then ddG's were again calculated.

Alternatively, remodeling was performed using RFdiffusion. Relaxed structures used as input for remodel were also used as input for RFdiffusion, except that only the C-terminal helices were used as input for diffusion. This protocol significantly reduces computational time. Positions with WT sequence identities from Rosetta Remodel were likewise preserved for diffusion. Unlike with Rosetta Remodel all structural data of subsequent residues was ignored. This is a limitation of RFdiffusion, not a scientific constraint placed on the design problem. Intra-protomer and inter-protomer weights were both set to 1, with a quadratic guide decay and a guide scale of 2. C-terminal lengths varied between 12 and 31 residues depending on the source virus, and 15 remodeled helices were generated for each length. Sequences were generated using Protein MPNN, with a sampling temperature of 0.4 and a negative bias against C (−10), and F, G, P, W, and Y (−1). 100 sequences were generated for each remodeled domain. The top 2% based on MPNN sample score were selected for computational characterization.

Designs were analyzed based on the following criteria: 1) ColabFold validates the design performed with Rossetta by predicting ordered terminal helix consistent with design model (assuming Colabfold method can provide reliable results for a particular fusion protein); 2) decrease in ddG by 5-15 Rosetta Energy Unit (REU) and 3); design has a well-packed hydrophobic core without extraneous elements (i.e., helical segments with no interprotomer hydrophobic packing). To calculate ddG, two models are generated, one in which all protomers are correctly in contact as trimers and one in which the protomers are moved distant from each other. Sidechains in both models are repacked and minimized, and then both models are scored. The ddG is the difference in the scores, as in (Distant state)−(Trimeric state).

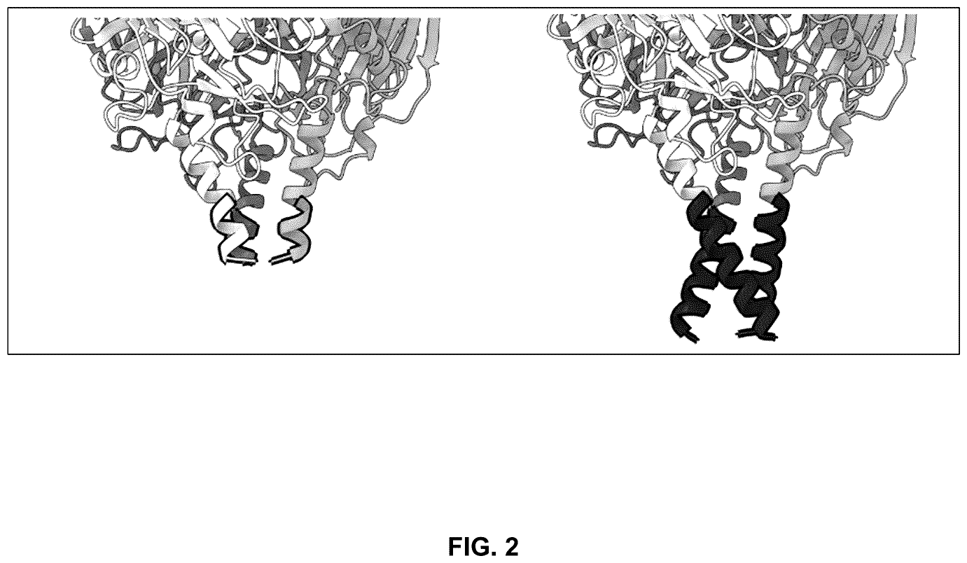

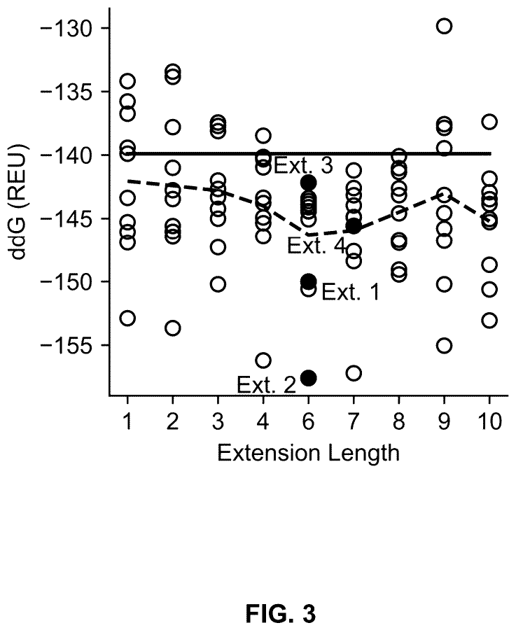

FIG. 2 shows a structural model of a representative experimental model of the RSV F protein (left) compared to the predicted structure of a representative design (right), provided from PDB 4MMU. The optimal length for the remodeled C terminus was determined by plotting average ddG against the length of the C-terminal helix, as shown in FIG. 3. When using Rosetta Remodel, the average ddG will decrease until an optimum length is achieved, at which point the ddG will tend to stay the same or increase again. This may be because Remodel can struggle when building larger segments due to increasing degrees of freedom. Ideal linker lengths are those near the minimum ddG. In this case, it was determined that an optimal C-terminal helix would terminate at about position 519. It was observed empirically that a ddG was minimized when the helical segment extended about 6 residues past the native position 513 (i.e., to position 519).

Computational modeling (with Rosetta Remodel) of the RSV/B protein was used to generate artificial polypeptide sequences, each predicted to form a stable alpha helix, shown in Table 7. Residues 500-502 of the native RSV F protein are included as NQS. Residues Q501 and S502 were remodeled with helical constraints while preserving the native sequence identities. This optimizes the helical backbone of these residues with side chains represented as centroids and then repacks the side chains in all-atom mode. Residues 503-509 were remodeled with helical constraints and without sequence constraints. The helical backbone is first optimized with side chains represented as centroids, and the side chains are designed in all-atom mode. As a result there is some bias towards the native sequence. Six to 14 additional amino acids were added with helical constraints. Side chains are represented as valine centroids during backbone sampling, then the sequence is sampled in all-atom mode. All backbone sampling of these elements in centroid mode is performed simultaneously and sequence design in all-atom mode is likewise performed simultaneously. Designs were manually refined to remove exposed hydrophobic residues or buried polar residues with identities preferentially selected from the nearest residue in the WT sequence or rationally where the WT residue was suboptimal.

The I53-50A molecule is well-suited for genetic fusion to many trimeric antigens, and features symmetric N-termini that are approximately 5 nm apart. Due to the remodelled C-terminus of the C-Term 1 design being more distanced laterally from the symmetric axis of the antigen (FIG. 2), it appeared possible that this modification could minimize strain in genetic fusions to I53-50A relative to commonly-studied antigen fragments that end at residue 513. Four sequences were selected for experimental testing (Table 7) as genetic fusions to a version of I53-50A (I53-50ΔΔcys), with antigens also containing DS-Cav1 mutations. Table 7. Illustrative C-terminal helix-forming segments

TABLE 7
Illustrative C-terminal helix-forming segments

| | | Re- | SEQ |
|---|---|---|---|
| | | modeled | ID |
| Name | Sequence | Length | NO: |
| C-Term 1 | NOSREIIRAINIVRKIASEK | 17 | 10 |
| C-Term 2 | NOSALWLEAAKYVKQAREKS | 17 | 11 |
| C-Term 3 | NOSAKNAEAAKIAEETKRKD | 17 | 12 |
| C-Term 4 | NOSRETAKAVSAVK | 11 | 75 |
| C-Term 5 | NOSALLLEAAKYVKKAREKS | 17 | 119 |
| C-Term 6 | NOSRKLLEAAEEMEKMLKTS | 17 | 120 |
| C-Term 7 | NOSRKMLEAVEHAKKLKKES | 17 | 121 |
| C-Term 8 | NQSRKMLEAVEKAKKLDKES | 17 | 122 |
| C-Term 9 | NOSAKTEEAYQRTIKTQQKL | 17 | 123 |
| C-Term 10 | NOSRDLDTAAKQVKEMLKEKS | 18 | 124 |
| C-Term 11 | NOSRETEKTIRQVQEILKKWS | 18 | 125 |
| C-Term 12 | NOSREVKEAIKIIKKILKKOS | 18 | 126 |
| C-Term 13 | NOSREIKDAIKKAKEFIKTIK | 18 | 127 |
| C-Term 14 | NOSREIETAIKKAKEFIKTIK | 18 | 128 |
| C-Term 15 | NOSRKATETIKKFEESEKS | 16 | 129 |
| C-Term 16 | NOSRDTIKVAIIVKELYKKIS | 18 | 130 |
| C-Term 17 | NOSRKTLETIEWVKKVIKKORS | 19 | 131 |
| C-Term 18 | NQSRKTLETIEWVEKVIKKORS | 19 | 132 |
| C-Term 19 | NOSRKWNESSKKVQEQDS | 15 | 133 |
| C-Term 20 | NOSRKTEKAIRLVLKWLKES | 17 | 134 |
| C-Term 21 | NQSRDTLKAIEQTKRYLEELKKS | 20 | 135 |
| C-Term 22 | NOSRSWDIAAKFVKTVLSNOS | 18 | 136 |
| C-Term 23 | NOSRKTLEATEIAKKLAEDRS | 18 | 137 |
| C-Term 24 | NOSLEILKAAKEAKKLIEDLRRS | 20 | 138 |
| C-Term 25 | NOSKELLDAAKAVKKMLEKEKSS | 20 | 139 |
| C-Term 26 | NOSKKLLDAADAVKKMLEKEKSS | 20 | 140 |
| C-Term 27 | NOSKKVLETIRWIETVISRORSS | 20 | 141 |
| C-Term 28 | NQSADLKKVAELVKKLMEEAKKKS | 21 | 142 |
| C-Term 29 | NOSTDTMKAARIMKEELKEKS | 18 | 143 |
| C-Term 30 | NOSRKTEEALRRADTIIKQLASKS | 21 | 144 |
| C-Term 31 | NQSKKLKSAADDVKKAKEKS | 17 | 145 |
| C-Term 32 | NOSKELKSAAEDVKKAKEKS | 17 | 146 |
| C-Term 33 | NOSRETKKATENVKTMLTKSKS | 19 | 147 |
| C-Term 34 | NOSLELKKAAKAANTDLTKKS | 18 | 148 |
| C-Term 35 | NOSLELKEAAKAANTDLTKKS | 18 | 149 |
| C-Term 36 | NQSRKLEEIARIVEQKKRTEEKRS | 21 | 150 |
| C-Term 37 | NOSAETKKAIERAREL | 13 | 151 |
| C-Term 38 | NQSRDLKKAAEIAKKS | 13 | 152 |
| C-Term 39 | NOSRTLLETAEIVTRS | 13 | 153 |
| C-Term 40 | NOSRTLLETAEIVKRS | 13 | 154 |
| C-Term 41 | NOSRKLDKAAEYVEKS | 13 | 155 |
| C-Term 42 | NOSKEAKKAIETAKKLS | 14 | 156 |
| C-Term 43 | NOSRKLETAAEKLKQTE | 14 | 157 |
| C-Term 44 | NOSRLMLEAVKIAQSOS | 14 | 158 |
| C-Term 45 | NOSRETKEAAESVKOMES | 15 | 159 |
| C-Term 46 | NQSRRTLKAIEITLKLLS | 15 | 160 |
| C-Term 47 | NOSRRTLTAITRVERKDS | 15 | 161 |
| C-Term 48 | NOSKKLADAADWVETVKSS | 16 | 162 |
| C-Term 49 | NOSKKTHSAIEWVERLVSS | 16 | 163 |
| C-Term 50 | NOSADTKKAAEIAKKLAKS | 16 | 164 |

The native sequence includes the C-terminal alpha-helical segment

(SEQ ID NO: 317)
ISQVNEKINQSLAFIRRSDE

In context the C-terminal alpha-helix of the modified construct is ISQVNEKINQSREIIRAINIVRKIASEK (SEQ ID NO: 319) and is only nine residues longer than the portion of the native structure known to be helical, and two residues lower than the predicted helical segment. Contact residues are bold and underlined.

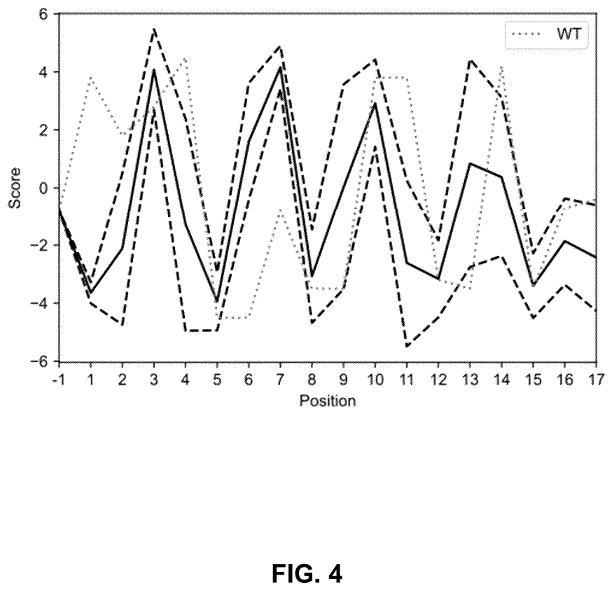

Native
(SEQ Id NO: 318)
ISQVNEKINQSLAFIRRSDELLHNVN
Remodel
(SEQ ID NO: 319)
ISQVNEKINQSREIIRAINIVRKIASEK

Whereas the WT sequence has a three-residue hydrophobic segment leading into the designed helix, and a five-residue polar segment in the middle, which contributes to suboptimal packing, the remodeled sequences are characterized by a pattern of alternating hydrophobic and polar segments with no hydrophobic segment longer than two consecutive residues and no polar segment longer than three consecutive residues (FIG. 4). The remodeled helix has at minimum two hydrophobic segments at positions 508 and/or 509 and 511 and/or 512 and optimally four hydrophobic segments at positions 505 and/or 506, 508 and/or 509, 511 and/or 512, and 515 and/or 516.

Published structure of the RSV protein generally does not include the residues C-terminal to about residue 500. Either the residues are not included in the recombinant protein studied, or they are not visible in the electronic density observed. Nonetheless, modelling suggests that the following substitutions will stabilize the this portion of the F protein in a helical conformation.

TABLE 8
Possible substitutions at Position 505-516

| Position | Preferences suggested by modeling | Illustrative Substitutions |
|---|---|---|
| F505 | Hydrophobic or threonine, not WFY | A, I, L, M, V, G, T; not F, Y, W |
| I506 | Any amino acid except P, preferably | Any amino acids except P; |
| | polar or AILV | preferably D, E K, N, Q, R, S, T, Y |
| | | or A, I, L, V |
| R507 | Any amino acid except P, preferably | Any amino acids except P; |
| | polar or AILV | preferably D, E, K, N, Q, R, S, T, Y |
| | | or A, I, L, V |
| K508 | AVTI preferred, K, Q, R possible | A, V, T, I; possibly K, Q, R |
| S509 | Hydrophobic or Thr. Preferred | A, I, L, M, V, F, W, Y, G, T; |
| | AILVM | preferablyA, I, L, M, V |
| D510 | Any amino acid, preferably polar | Any amino acids; preferably D, E, |
| | | K, N, Q, R, S, T, Y |
| E511 | Any amino acid depending on the rest | Any amino acids depending on the |
| | of the design | rest of the design |
| L512 | Preferred hydrophobic, can be T and | Preferably A, I, L, M, V, F, W, Y, G, |
| | in some cases other polar | T; in some cases D, E, K, N, Q, R, S, |
| | | T, Y |
| L513 | Any amino acid, preferred polar but | Any amino acids; preferably D, E, |
| | occasionally hydrophobic | K, N, Q, R, S, T, Y; occasionally A, |
| | | I, L, M, V, F, W, Y, G |
| H514 | Any amino acid except P, preferably | Any amino acids except P; |
| | polar | preferably D, E, K, N, Q, R, S, T, Y |
| N515 | Any amino acid except P, preferably | Any amino acids except P; |
| | hydrophobic | preferably A, I, L, M, V, F, W, Y, G |
| V516 | Hydrophobic or TSK | A, I, L, M, V, F, W, Y, G, or T, S, K |
| N517 | Any amino acid except P, preferably | Any amino acids except P; |
| | polar | preferably D, E, K, N, Q, R, S, T, Y |
| A518 | Any amino acid except P, preferably | Any amino acids except P; |
| | polar | preferably D, E, K, N, Q, R, S, T, Y |
| G519 | Any amino acid except P, preferably | Any amino acids except P; |
| | polar | preferably D, E, K, N, Q, R, S, T, Y |

In some embodiments, polar amino acids refer to D, E, K, N, Q, R, S, T, and Y. In some embodiments, polar amino acids includes charged amino acid residues. In some embodiments, charged amino acids refer to E, D, R, K, and H. In some embodiments, hydrophobic amino acids refer to A, I, L, M, V, F, Y, and W.

A small-scale screen showed that three of the four selected designs expressed. Table 9 shows binding of antibodies D25, AM14, and 4D7 to RSV/B F proteins fused to 153-50A to form trimeric protein complexes (but not assembled with IS3-50B). Both D25 and AM 14 are specific to the prefusion state, however D25 can bind both prefusion monomers and trimers while AM14 can only bind closed trimeric prefusion trimers. 4D7 is specific to the postfusion state. C-Term 1 was well expressed and showed the highest binding to AM14.

TABLE 9
Summary of antibody binding screening
data for designed RSV/B F proteins

| Name | Expression | D25 | AM14 | 4D7 |
|---|---|---|---|---|
| C-Term1 | ++ | +++ | +++ | + |
| C-Term 2 | − | NA | NA | NA |
| C-Term 3 | ++ | +++ | ++ | ++ |
| C-Term 4 | +++ | +++ | ++ | + |
| DS-Cav1 | +++ | +++ | ++ | ++ |
| RSV/B.002 | | | | |

### Example 2. Design of Stabilizing Substitutions for RSV F Proteins

This Example describes sets of stabilizing mutations for stabilization of the prefusion state of RSV F protein. Based on a structure of RSV F in the prefusion conformation (FIG. 1) compared to its postfusion conformation (not shown), stabilizing mutations at the interfaces between protomers were designed to either lower the energy of the prefusion state or raise the energy of the postfusion state.

Computational modeling was used to identify amino acid substitutions to stabilize RSV/B F protein in the prefusion conformation. These mutations are listed in Table 10.

TABLE 10
Stabilizing substitutions

| Space | Substitutions |
|---|---|
| Space 1 | F140W, K399A, K399V, |
| | T400D, S485I, S485A, S485F, |
| | D486A, D486Q, D486E, D486S, |
| | E487R, E487K, E487A, E487M, |
| | E487Q, 487R, 487M, F488W, |
| | D489A, Q494I, Q494M, Q494L, |
| | Q494A, K498A, K498E, 498A, |
| | 498Y |
| Space 2 | V56L, V56A, T58A, T58S, |
| | T58M, V154I, V187L, V296A, |
| | A298M, A298L, A298I |
| Space 3 | K75Q, N216S, N216D, E218P, |
| | T219S |
| Space 4 | E92I, E92A, E232A, E232W, |
| | R235Y, R235W, S238A, S238L, |
| | T249P, Y250F, N254V, N254L |
| Other | T67V, F137D, F137S, R339E |

Based on molecular modelling, combinations of substitutions expected to synergize include:

E487R + K498A
E487R + K498E
E487K + K498E
D486A + E487R + K498A
D486Q + E487R + K498A
D486E + E487A + D489A + T400D
D486A + E487M + K498A
E487Q
D486S
F488W + D489A + T400D + E487R + K498A
F140W + D489A + T400D + E487R + K498A
Q494I + S485I + K399A + 487R + 498A
Q494M + S485I + K399A, D486A + 487M + 498A
Q494L + S485A + K399V + D486A + 487M + 498A
Q494M + S485A + K399V + D486A + 487M + 498A
Q494A + S485F + K399V + D486A + 487M + 498Y
D489A + T400D + E487R + K498A
D489A + T400D

RSV F proteins are cleaved during expression by the protease furin. Constructs that replace the cleavage site for furin (residues 104-140) with a native linker were also tested. Linker sequences are provided in Table 11, which were tested in between residues 103 and 141.

TABLE 11
Furin cleavage linkers

| Sequence | Length | SEQ ID NO: |
|---|---|---|
| NNQARGSGSGRSLGF | 15 | 245 |
| NNQARGGSGGRSLGF | 15 | 246 |
| NNGARGGSGGRSLGF | 15 | 247 |
| NNQARGGSGGDSLGF | 15 | 248 |
| NNQARGGSGSGGDSLGF | 17 | 249 |
| NNQARGGSGGGDLG | 14 | 250 |
| NNQARGGSGSGGDLGF | 16 | 251 |

### Example 3. Experimental Evaluation of RSV F Proteins

This Example shows that the C-terminal helix-forming segments described in Example 1 increase thermal stability of the recombinant polypeptides by as much as about 20-25° C. or more and increase storage stability under accelerated degradation conditions (storage at 40° C.). Further improvement is observed when the C-terminal helix-forming segment is combined with stabilizing mutations described in Example 2. The recombinant polypeptides retain the ability to self-assemble to form a two-component I53-50-type nanostructure.

Recombinant polypeptides that include RSV/B F protein ectodomains (B18537 strain with DS-Cav1 mutations) fused to I53-50ΔΔcys were tested using small-scale HEK293 expression. Supernatants were screened for relative expression by bio-layer interferometry (BLI) with a monoclonal antibody (16A8) that binds specifically to 153-50A. BLI was used to measure binding to known RSV F protein antibodies D25 (specific to prefusion state), AM14 (specific to closed trimeric prefusion state), and 4D7 (specific to postfusion state). Measurements were normalized to binding by palivizumab (conformation independent). Increased AM14 was observed to several designs featuring mutations in Space 1, C-terminal remodelling, or both.

Scaled-up protein preparation for select designs were incubated for six days at either 4° C. or 40° C. Designs were identified which showed less loss in D25 or AM14 binding at 40° C. compared to DS-Cav1 mutations alone, as well as smaller increases in binding to 4D7 at 40° C. The C-term1 design (Example 1) that includes a remodelled C terminus showed nearly no decrease in AM14 binding and no increase in 4D7 binding.

Sets of mutations were selected for analysis in combination with each other. In these experiments, an ectodomain sequence from a contemporary RSV/B strain was used (hRSV/B/Australia/VIC-RCH056/2019). Antibody binding was normalized to 16A8 mAb, which is specific to the I53-50A fusion partner. Multiple designs were characterized that increased ratios of binding to AM14 (prefusion) or decreased the binding to 4D7 (postfusion) (FIG. 1). Six-day thermal stress tests were performed for select scaled-up proteins.

Fourteen designs were selected for further analysis after scale-up and purification. Antigenic measurements confirmed increases in AM14 binding for all tested designs relative to DS-Cav1 mutations alone. Constructs incorporating C-terminal remodelling generally showed greater thermal stability under storage (i.e., reduced rate of decrease in 4D7 binding).

Constructs selected for thermal denaturation and storage testing are shown in Table 12. All tested RSV/B constructs were based on the sequence of strain hRSV/B/Australia/VIC-RCH056/2019, including the DS-Cav1 mutations, fused to I53-50ΔΔcys. All proteins were tested as soluble, trimeric fusions (prior to assembly with I53-50B to form a nanostructure). RSV/A.03 (based on the A2 strain) and RSV/B.002 were controls containing the DS-Cav1 substitutions. The data in Table 12 show that the C-terminal alpha-helical segment by itself can increase thermal stability by up to about 25° C. (compare construct RSV/B.002 to RSV/B.195, construct RSV/B.093 to RSV/B.189). Furthermore, all constructs having the C-terminal alpha-helical segment maintain the prefusion conformation when stored at 40° C. for seven days. One construct without the C-terminal alpha-helical segment, RSV/B.093, was also stable prefusion at 40° C., but its melting temperature was lower than constructs containing C-terminal remodelling.

TABLE 12

| | | | Alpha- | NanoDSF | | |
|---|---|---|---|---|---|---|
| | | | helical | Tonset | | Storage |
| Construct | Serotype | Substitutions⁴ | segment | (° C.) | Tm (° C.) | Stable at 40° C. |
| RSV/A.03 | A³ | | | 44.4 | 51.5 | − |
| RSV/B.002 | B¹ | | | 43.4 | 50.1 | − |
| RSV/B.081 | B¹ | D489A | | 51.2 | 56.5 | + |
| | | T400D | | | | |
| | | E487R | | | | |
| | | K498A | | | | |
| | | D486A | | | | |
| RSV/B.093 | B¹ | F488W | | 51.2 | 56.5 | ++ |
| | | D489A | | | | |
| | | T400D | | | | |
| | | E487R | | | | |
| | | K498A | | | | |
| | | D486A | | | | |
| RSV/B.099 | B¹ | E487R | | 43.4 | 50.1 | − |
| | | K498A | | | | |
| | | T67V | | | | |
| RSV/B/100 | B¹ | E487R | | 46.3 | 51.5 | − |
| | | K498A | | | | |
| | | T249P | | | | |
| | | T67V | | | | |
| RSV/B.123 | B¹ | D489A | | 49.9 | 54.9 | + |
| | | T400D | | | | |
| | | E487R | | | | |
| | | K498A | | | | |
| | | T67V | | | | |
| RSV/B.147 | B¹ | E487R | Yes² | 59.0 | 69.7 | ++ |
| | | K498A | | | | |
| RSV/B.148 | B¹ | E487R | Yes² | 64.4 | 77.3 | ++ |
| | | K498A | | | | |
| | | T249P | | | | |
| RSV/B.160 | B¹ | F488W | Yes² | 66.6 | 77.2 | ++ |
| | | D489A | | | | |
| | | T400D | | | | |
| | | E487R | | | | |
| | | K498A | | | | |
| | | T249P | | | | |
| RSV/B.171 | B¹ | D489A | Yes² | 69.0 | 80.9 | ++ |
| | | T400D | | | | |
| | | E487R | | | | |
| | | K498A | | | | |
| RSV/B.172 | B¹ | D489A | Yes² | 65.7 | 77.3 | ++ |
| | | T400D | | | | |
| | | E487R | | | | |
| | | K498A | | | | |
| | | T249P | | | | |
| RSV/B.178 | B¹ | D489A | Yes² | 69.7 | 80.3 | ++ |
| | | T400D | | | | |
| | | E487R | | | | |
| | | K498A | | | | |
| | | D486A | | | | |
| | | T249P | | | | |
| RSV/B.189 | B¹ | F488W | Yes² | 70.8 | 81.1 | ++ |
| | | D489A | | | | |
| | | T400D | | | | |
| | | E487R | | | | |
| | | K498A | | | | |
| | | D486A | | | | |
| RSV/B.195 | B¹ | | Yes² | 56.2 | 68.2 | ++ |
| RSV/A.013 | A³ | | Yes² | 51.6 | 56.0 | ++ |
| RSV/A.023 | A³ | D489A | Yes² | 63.9 | 70.5 | ++ |
| | | T400D | | | | |
| | | E487R | | | | |
| | | K498A | | | | |

¹Based on RSV/B/Australia/VIC-RCH056/2019 strain
²NQSREIIRAINIVRKIASEK (SEQ ID NO: 10)
³Based on A2 strain
⁴In addition to DS-Cav1 (S155C, S290C, S190F, and V207L

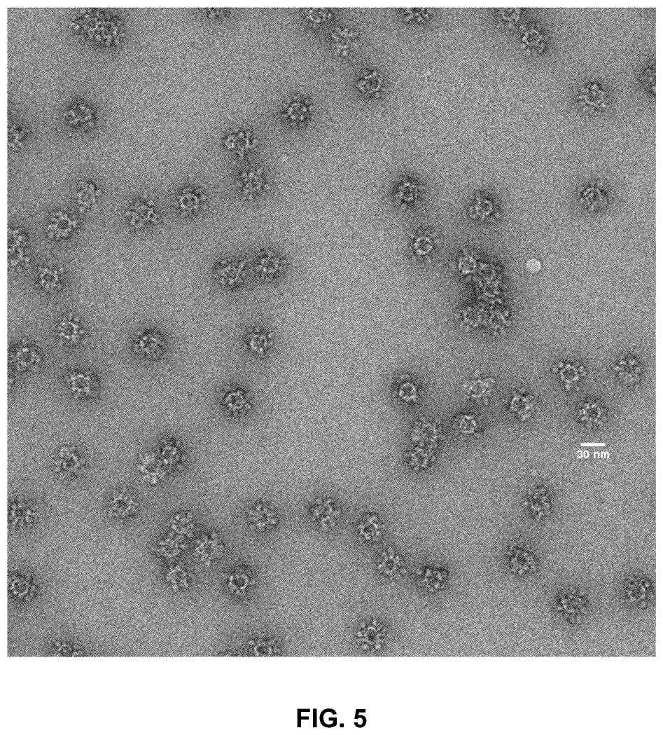

Selected constructs were incubated with a second component, 153-50B, to form nanostructures. Dynamic Light Scattering (DLS) and negative-stain electron microscopy (nsEM) confirm assembly as nanostructure. Results are shown in Table 13. A representative electron micrograph is shown in FIG. 5 (RSV/B.195, having the DS-Cav).

TABLE 13

| | | | | Nanostructure | | |
|---|---|---|---|---|---|---|
| | | | Alpha- | Self- | Compact | |
| | | | helical | assembly | trimer | Tested in |
| Construct | Serotype | Substitutions² | segment³ | (DLS) | (nsEM) | vivo |
| RSV/A.03 | A⁴ | | | Yes | + | Yes |
| RSV/B.002 | B¹ | | | Yes | + | Yes |
| RSV/B.081 | B¹ | D489A | | Yes | Not tested | No |
| | | T400D | | | | |
| | | E487R | | | | |
| | | K498A | | | | |
| | | D486A | | | | |
| RSV/B.093 | B¹ | F488W | | Yes | ++ | Yes |
| | | D489A | | | | |
| | | T400D | | | | |
| | | E487R | | | | |
| | | K498A | | | | |
| | | D486A | | | | |
| RSV/B.099 | B¹ | E487R | | Yes | Not tested | No |
| | | K498A | | | | |
| | | T67V | | | | |
| RSV/B/100 | B¹ | E487R | | Yes | Not tested | No |
| | | K498A | | | | |
| | | T249P | | | | |
| | | T67V | | | | |
| RSV/B.123 | B¹ | D489A | | Yes | Not tested | No |
| | | T400D | | | | |
| | | E487R | | | | |
| | | K498A | | | | |
| | | T67V | | | | |
| RSV/B.147 | B¹ | E487R | Yes | Yes | Not tested | No |
| | | K498A | | | | |
| RSV/B.148 | B¹ | E487R | Yes | Yes | Not tested | No |
| | | K498A | | | | |
| | | T249P | | | | |
| RSV/B.160 | B¹ | F488W | Yes | Yes | ++ | Yes |
| | | D489A | | | | |
| | | T400D | | | | |
| | | E487R | | | | |
| | | K498A | | | | |
| | | T249P | | | | |
| RSV/B.171 | B¹ | D489A | Yes | Yes | ++ | Yes |
| | | T400D | | | | |
| | | E487R | | | | |
| | | K498A | | | | |
| RSV/B.172 | B¹ | D489A | Yes | Yes | Not tested | No |
| | | T400D | | | | |
| | | E487R | | | | |
| | | K498A | | | | |
| | | T249P | | | | |
| RSV/B.178 | B¹ | D489A | Yes | Yes | Not tested | No |
| | | T400D | | | | |
| | | E487R | | | | |
| | | K498A | | | | |
| | | D486A | | | | |
| | | T249P | | | | |
| RSV/B.189 | B¹ | F488W | Yes | Yes | Not tested | No |
| | | D489A | | | | |
| | | T400D | | | | |
| | | E487R | | | | |
| | | K498A | | | | |
| | | D486A | | | | |
| RSV/B.195 | B¹ | | Yes | Yes | ++ | Yes |
| RSV/A.013 | A⁴ | | Yes | Yes | Not tested | Yes |
| RSV/A.023 | A⁴ | D489A | Yes | Yes | Not tested | Yes |
| | | T400D | | | | |
| | | E487R | | | | |
| | | K498A | | | | |

¹Based on hRSV/B/Australia/VIC-RCH056/2019 strain
²In addition to DS-Cav1 (S155C, S290C, S190F, and V207L)
³NQSREIIRAINIVRKIASEK (SEQ ID NO: 10)
⁴Based on A2 strain

Sequences for designed constructs used in Table 13 are shown in Table 14. SEQ ID NO: 1 is used for the reference sequence. In each case, the signal peptide at the N terminus or the tag at the C terminus, shown in underlined may be inserted with known alternatives or deleted. RSV F protein is known to be cleaved at two furin cleavage sites leading to loss of a peptide sequence known as “p27.” (Rezende et al. Front. Microbiol., Vol. 14 (2023).) As used herein, the term “polypeptide” includes polypeptides lacking the p27 peptide due to this cleavage reaction. The approximate region surrounding the p27 peptide is bold and italicized, and may be removed through furin-based cleavage during production of antigens in cell culture.

TABLE 14

| | | SEQ |
|---|---|---|
| | | ID |
| Construct | Sequence | NO: |
| RSV/A.03 | MELLILKANAITTILTAVTFCFASGQNITEEFYQSTCSAVSK | 76 |
| | GYLSALRTGWYTSVITIELSNIKENKCNGTDAKVKLIKQEL | |
| | DKYKNAVTELQLLMQSTPATNNRARRELPRFMNYTLNNA | |
| | KKTNVTLSKKRKRRFLGFLLGVGSAIASGVAVCKVLHLEGE | |
| | VNKIKSALLSTNKAVVSLSNGVSVLTFKVLDLKNYIDKQLL | |
| | PILNKQSCSISNIETVIEFQQKNNRLLEITREFSVNAGVTTPV | |
| | STYMLTNSELLSLINDMPITNDQKKLMSNNVQIVRQQSYSI | |
| | MCIIKEEVLAYVVQLPLYGVIDTPCWKLHTSPLCTTNTKEG | |
| | SNICLTRTDRGWYCDNAGSVSFFPQAETCKVQSNRVFCDT | |
| | MNSLTLPSEVNLCNVDIFNPKYDCKIMTSKTDVSSSVITSLG | |
| | AIVSCYGKTKCTASNKNRGIIKTFSNGCDYVSNKGVDTVS | |
| | VGNTLYYVNKQEGKSLYVKGEPIINFYDPLVFPSARFDASI | |
| | SQVNEAINQSLAFIRKSDELLGSGGSGSGSGGSEKAAKAEE | |
| | AARKMEELFKKHKIVAVLRANSVEEAIEKAVAVFAGGVH | |
| | LIEITFTVPDADTVIKALSVLKEKGAIIGAGTVTSVEQARKA | |
| | VESGAEFIVSPHLDEEISQFAKEKGVFYMPGVMTPTELVKA | |
| | MKLGHTILKLFPGEVVGPQFVKAMKGPFPNVKFVPTGGVN | |
| | LDNVAEWFKAGVLAVGVGSALVKGTPDEVREKAKAFVE | |
| | KIRGATELEHHHHHH | |
| RSV/A.013 | MELLILKANAITTILTAVTFCFASGQNITEEFYQSTCSAVSK | 77 |
| | GYLSALRTGWYTSVITIELSNIKENKCNGTDAKVKLIKQEL | |
| | DKYKNAVTELQLLMQSTPATNNRARRELPRFMNYTLNNA | |
| | KKTNVTLSKKRKRRFLGFLLGVGSAIASGVAVCKVLHLEGE | |
| | VNKIKSALLSTNKAVVSLSNGVSVLTFKVLDLKNYIDKQLL | |
| | PILNKQSCSISNIETVIEFQQKNNRLLEITREFSVNAGVTTP | |
| | VSTYMLTNSELLSLINDMPITNDQKKLMSNNVQIVRQQSYSI | |
| | MCIIKEEVLAYVVQLPLYGVIDTPCWKLHTSPLCTTNTKEG | |
| | SNICLTRTDRGWYCDNAGSVSFFPQAETCKVQSNRVFCDT | |
| | MNSLTLPSEVNLCNVDIFNPKYDCKIMTSKTDVSSSVITSLG | |
| | AIVSCYGKTKCTASNKNRGIIKTFSNGCDYVSNKGVDTVS | |
| | VGNTLYYVNKQEGKSLYVKGEPIINFYDPLVFPSDEFDASIS | |
| | QVNEKINQSREIIRAINIVRKIASEKGSGGSGSGSGGSEKAA | |
| | KAEEAARKMEELFKKHKIVAVLRANSVEEAIEKAVAVFAG | |
| | GVHLIEITFTVPDADTVIKALSVLKEKGAIIGAGTVTSVEQA | |
| | RKAVESGAEFIVSPHLDEEISQFAKEKGVFYMPGVMTPTEL | |
| | VKAMKLGHTILKLFPGEVVGPQFVKAMKGPFPNVKFVPTG | |
| | GVNLDNVAEWFKAGVLAVGVGSALVKGTPDEVREKAKA | |
| | FVEKIRGATELEHHHHHH | |
| RSV/A.015 | MELLILKANAITTILTAVTFCFASGQNITEEFYQSTCSAVSK | 78 |
| | GYLSALRTGWYTSVITIELSNIKENKCNGTDAKVKLIKQEL | |
| | DKYKNAVTELQLLMQSTPATNNRARRELPRFMNYTLNNA | |
| | KKTNVTLSKKRKRRFLGFLLGVGSAIASGVAVCKVLHLEGE | |
| | VNKIKSALLSTNKAVVSLSNGVSVLTFKVLDLKNYIDKQLL | |
| | PILNKQSCSISNIETVIEFQQKNNRLLEITREFSVNAGVTTP | |
| | VSTYMLTNSELLSLINDMPITNDQKKLMSNNVQIVRQQSYSI | |
| | MCIIKEEVLAYVVQLPLYGVIDTPCWKLHTSPLCTTNTKEG | |
| | SNICLTRTDRGWYCDNAGSVSFFPQAETCKVQSNRVFCDT | |
| | MNSLTLPSEVNLCNVDIFNPKYDCKIMTSKDDVSSSVITSL | |
| | GAIVSCYGKTKCTASNKNRGIIKTFSNGCDYVSNKGVDTV | |
| | SVGNTLYYVNKQEGKSLYVKGEPIINFYDPLVFPSARFAAS | |
| | ISQVNEAINQSLAFIRKSDELLGSGGSGSGSGGSEKAAKAEE | |
| | AARKMEELFKKHKIVAVLRANSVEEAIEKAVAVFAGGVH | |
| | LIEITFTVPDADTVIKALSVLKEKGAIIGAGTVTSVEQARKA | |
| | VESGAEFIVSPHLDEEISQFAKEKGVFYMPGVMTPTELVKA | |
| | MKLGHTILKLFPGEVVGPQFVKAMKGPFPNVKFVPTGGVN | |
| | LDNVAEWFKAGVLAVGVGSALVKGTPDEVREKAKAFVE | |
| | KIRGATELEHHHHHH | |
| RSV/A.016 | MELLILKANAITTILTAVTFCFASGQNITEEFYQSTCSAVSK | 79 |
| | GYLSALRTGWYTSVITIELSNIKENKCNGTDAKVKLIKQEL | |
| | DKYKNAVTELQLLMQSTPATNNRARRELPRFMNYTLNNA | |
| | KKTNVTLSKKRKRRFLGFLLGVGSAIASGVAVCKVLHLEGE | |
| | VNKIKSALLSTNKAVVSLSNGVSVLTFKVLDLKNYIDKQLL | |
| | PILNKQSCSISNIETVIEFQQKNNRLLEITREFSVNAGVTTPV | |
| | STYMLTNSELLSLINDMPITNDQKKLMSNNVQIVRQQSYSI | |
| | MCIIKEEVLAYVVQLPLYGVIDTPCWKLHTSPLCTTNTKEG | |
| | SNICLTRTDRGWYCDNAGSVSFFPQAETCKVQSNRVFCDT | |
| | MNSLTLPSEVNLCNVDIFNPKYDCKIMTSKDDVSSSVITSL | |
| | GAIVSCYGKTKCTASNKNRGIIKTFSNGCDYVSNKGVDTV | |
| | SVGNTLYYVNKQEGKSLYVKGEPIINFYDPLVFPSARWAA | |
| | SISQVNEAINQSLAFIRKSDELLGSGGSGSGSGGSEKAAKAE | |
| | EAARKMEELFKKHKIVAVLRANSVEEAIEKAVAVFAGGV | |
| | HLIEITFTVPDADTVIKALSVLKEKGAIIGAGTVTSVEQARK | |
| | AVESGAEFIVSPHLDEEISQFAKEKGVFYMPGVMTPTELVK | |
| | AMKLGHTILKLFPGEVVGPQFVKAMKGPFPNVKFVPTGGV | |
| | NLDNVAEWFKAGVLAVGVGSALVKGTPDEVREKAKAFV | |
| | EKIRGATELEHHHHHH | |
| RSV/A.017 | MELLILKANAITTILTAVTFCFASGQNITEEFYQSTCSAVSK | 80 |
| | GYLSALRTGWYTSVITIELSNIKEVKCNGTDAKVKLIKQEL | |
| | DKYKNAVTELQLLMQSTPATNNRARRELPRFMNYTLNNA | |
| | KKTNVTLSKKRKRRFLGFLLGVGSAIASGVAVCKVLHLEGE | |
| | VNKIKSALLSTNKAVVSLSNGVSVLTFKVLDLKNYIDKQLL | |
| | PILNKQSCSISNIETVIEFQQKNNRLLEITREFSVNAGVTTPV | |
| | STYMLTNSELLSLINDMPITNDQKKLMSNNVQIVRQQSYSI | |
| | MCIIKEEVLAYVVQLPLYGVIDTPCWKLHTSPLCTTNTKEG | |
| | SNICLTRTDRGWYCDNAGSVSFFPQAETCKVQSNRVFCDT | |
| | MNSLTLPSEVNLCNVDIFNPKYDCKIMTSKTDVSSSVITSLG | |
| | AIVSCYGKTKCTASNKNRGIIKTFSNGCDYVSNKGVDTVS | |
| | VGNTLYYVNKQEGKSLYVKGEPIINFYDPLVFPSDRFDASI | |
| | SQVNEAINQSLAFIRKSDELLGSGGSGSGSGGSEKAAKAEE | |
| | AARKMEELFKKHKIVAVLRANSVEEAIEKAVAVFAGGVH | |
| | LIEITFTVPDADTVIKALSVLKEKGAIIGAGTVTSVEQARKA | |
| | VESGAEFIVSPHLDEEISQFAKEKGVFYMPGVMTPTELVKA | |
| | MKLGHTILKLFPGEVVGPQFVKAMKGPFPNVKFVPTGGVN | |
| | LDNVAEWFKAGVLAVGVGSALVKGTPDEVREKAKAFVE | |
| | KIRGATELEHHHHHH | |
| RSV/A.018 | MELLILKANAITTILTAVTFCFASGQNITEEFYQSTCSAVSK | 81 |
| | GYLSALRTGWYTSVITIELSNIKEVKCNGTDAKVKLIKQEL | |
| | DKYKNAVTELQLLMQSTPATNNRARRELPRFMNYTLNNA | |
| | KKTNVTLSKKRKRRFLGFLLGVGSAIASGVAVCKVLHLEGE | |
| | VNKIKSALLSTNKAVVSLSNGVSVLTFKVLDLKNYIDKQLL | |
| | PILNKQSCSISNIETVIEFQQKNNRLLEITREFSVNAGVTTPV | |
| | SPYMLTNSELLSLINDMPITNDQKKLMSNNVQIVRQQSYSI | |
| | MCIIKEEVLAYVVQLPLYGVIDTPCWKLHTSPLCTTNTKEG | |
| | SNICLTRTDRGWYCDNAGSVSFFPQAETCKVQSNRVFCDT | |
| | MNSLTLPSEVNLCNVDIFNPKYDCKIMTSKTDVSSSVITSLG | |
| | AIVSCYGKTKCTASNKNRGIIKTFSNGCDYVSNKGVDTVS | |
| | VGNTLYYVNKQEGKSLYVKGEPIINFYDPLVFPSDRFDASI | |
| | SQVNEAINQSLAFIRKSDELLGSGGSGSGSGGSEKAAKAEE | |
| | AARKMEELFKKHKIVAVLRANSVEEAIEKAVAVFAGGVH | |
| | LIEITFTVPDADTVIKALSVLKEKGAIIGAGTVTSVEQARKA | |
| | VESGAEFIVSPHLDEEISQFAKEKGVFYMPGVMTPTELVKA | |
| | MKLGHTILKLFPGEVVGPQFVKAMKGPFPNVKFVPTGGVN | |
| | LDNVAEWFKAGVLAVGVGSALVKGTPDEVREKAKAFVE | |
| | KIRGATELEHHHHHH | |
| RSV/A.019 | MELLILKANAITTILTAVTFCFASGQNITEEFYQSTCSAVSK | 82 |
| | GYLSALRTGWYTSVITIELSNIKEVKCNGTDAKVKLIKQEL | |
| | DKYKNAVTELQLLMQSTPATNNRARRELPRFMNYTLNNA | |
| | KKTNVTLSKKRKRRFLGFLLGVGSAIASGVAVCKVLHLEGE | |
| | VNKIKSALLSTNKAVVSLSNGVSVLTFKVLDLKNYIDKQLL | |
| | PILNKQSCSISNIETVIEFQQKNNRLLEITREFSVNAGVTTPV | |
| | STYMLTNSELLSLINDMPITNDQKKLMSNNVQIVRQQSYSI | |
| | MCIIKEEVLAYVVQLPLYGVIDTPCWKLHTSPLCTTNTKEG | |
| | SNICLTRTDRGWYCDNAGSVSFFPQAETCKVQSNRVFCDT | |
| | MNSLTLPSEVNLCNVDIFNPKYDCKIMTSKDDVSSSVITSL | |
| | GAIVSCYGKTKCTASNKNRGIIKTFSNGCDYVSNKGVDTV | |
| | SVGNTLYYVNKQEGKSLYVKGEPIINFYDPLVFPSDRFAAS | |
| | ISQVNEAINQSLAFIRKSDELLGSGGSGSGSGGSEKAAKAEE | |
| | AARKMEELFKKHKIVAVLRANSVEEAIEKAVAVFAGGVH | |
| | LIEITFTVPDADTVIKALSVLKEKGAIIGAGTVTSVEQARKA | |
| | VESGAEFIVSPHLDEEISQFAKEKGVFYMPGVMTPTELVKA | |
| | MKLGHTILKLFPGEVVGPQFVKAMKGPFPNVKFVPTGGVN | |
| | LDNVAEWFKAGVLAVGVGSALVKGTPDEVREKAKAFVE | |
| | KIRGATELEHHHHHH | |
| RSV/A.020 | MELLILKANAITTILTAVTFCFASGQNITEEFYQSTCSAVSK | 83 |
| | GYLSALRTGWYTSVITIELSNIKENKCNGTDAKVKLIKQEL | |
| | DKYKNAVTELQLLMQSTPATNNRARRELPRFMNYTLNNA | |
| | KKTNVTLSKKRKRRFLGFLLGVGSAIASGVAVCKVLHLEGE | |
| | VNKIKSALLSTNKAVVSLSNGVSVLTFKVLDLKNYIDKQLL | |
| | PILNKQSCSISNIETVIEFQQKNNRLLEITREFSVNAGVTTPV | |
| | STYMLTNSELLSLINDMPITNDQKKLMSNNVQIVRQQSYSI | |
| | MCIIKEEVLAYVVQLPLYGVIDTPCWKLHTSPLCTTNTKEG | |
| | SNICLTRTDRGWYCDNAGSVSFFPQAETCKVQSNRVFCDT | |
| | MNSLTLPSEVNLCNVDIFNPKYDCKIMTSKTDVSSSVITSLG | |
| | AIVSCYGKTKCTASNKNRGIIKTFSNGCDYVSNKGVDTVS | |
| | VGNTLYYVNKQEGKSLYVKGEPIINFYDPLVFPSDRFDASI | |
| | SQVNEAINQSREIIRAINIVRKIASEKGSGGSGSGSGGSEKA | |
| | AKAEEAARKMEELFKKHKIVAVLRANSVEEAIEKAVAVFA | |
| | GGVHLIEITFTVPDADTVIKALSVLKEKGAIIGAGTVTSVEQ | |
| | ARKAVESGAEFIVSPHLDEEISQFAKEKGVFYMPGVMTPTE | |
| | LVKAMKLGHTILKLFPGEVVGPQFVKAMKGPFPNVKFVPT | |
| | GGVNLDNVAEWFKAGVLAVGVGSALVKGTPDEVREKAK | |
| | AFVEKIRGATELEHHHHHH | |
| RSV/A.021 | MELLILKANAITTILTAVTFCFASGQNITEEFYQSTCSAVSK | 84 |
| | GYLSALRTGWYTSVITIELSNIKENKCNGTDAKVKLIKQEL | |
| | DKYKNAVTELQLLMQSTPATNNRARRELPRFMNYTLNNA | |
| | KKTNVTLSKKRKRRFLGFLLGVGSAIASGVAVCKVLHLEGE | |
| | VNKIKSALLSTNKAVVSLSNGVSVLTFKVLDLKNYIDKQLL | |
| | PILNKQSCSISNIETVIEFQQKNNRLLEITREFSVNAGVTTPV | |
| | SPYMLTNSELLSLINDMPITNDQKKLMSNNVQIVRQQSYSI | |
| | MCIIKEEVLAYVVQLPLYGVIDTPCWKLHTSPLCTTNTKEG | |
| | SNICLTRTDRGWYCDNAGSVSFFPQAETCKVQSNRVFCDT | |
| | MNSLTLPSEVNLCNVDIFNPKYDCKIMTSKTDVSSSVITSLG | |
| | AIVSCYGKTKCTASNKNRGIIKTFSNGCDYVSNKGVDTVS | |
| | VGNTLYYVNKQEGKSLYVKGEPIINFYDPLVFPSDRFDASI | |
| | SQVNEAINQSREIIRAINIVRKIASEKGSGGSGSGSGGSEKA | |
| | AKAEEAARKMEELFKKHKIVAVLRANSVEEAIEKAVAVFA | |
| | GGVHLIEITFTVPDADTVIKALSVLKEKGAIIGAGTVTSVEQ | |
| | ARKAVESGAEFIVSPHLDEEISQFAKEKGVFYMPGVMTPTE | |
| | LVKAMKLGHTILKLFPGEVVGPQFVKAMKGPFPNVKFVPT | |
| | GGVNLDNVAEWFKAGVLAVGVGSALVKGTPDEVREKAK | |
| | AFVEKIRGATELEHHHHHH | |
| RSV/A.022 | MELLILKANAITTILTAVTFCFASGQNITEEFYQSTCSAVSK | 85 |
| | GYLSALRTGWYTSVITIELSNIKENKCNGTDAKVKLIKQEL | |
| | DKYKNAVTELQLLMQSTPATNNRARRELPRFMNYTLNNA | |
| | KKTNVTLSKKRKRRFLGFLLGVGSAIASGVAVCKVLHLEGE | |
| | VNKIKSALLSTNKAVVSLSNGVSVLTFKVLDLKNYIDKQLL | |
| | PILNKQSCSISNIETVIEFQQKNNRLLEITREFSVNAGVTTPV | |
| | SPYMLTNSELLSLINDMPITNDQKKLMSNNVQIVRQQSYSI | |
| | MCIIKEEVLAYVVQLPLYGVIDTPCWKLHTSPLCTTNTKEG | |
| | SNICLTRTDRGWYCDNAGSVSFFPQAETCKVQSNRVFCDT | |
| | MNSLTLPSEVNLCNVDIFNPKYDCKIMTSKDDVSSSVITSL | |
| | GAIVSCYGKTKCTASNKNRGIIKTFSNGCDYVSNKGVDTV | |
| | SVGNTLYYVNKQEGKSLYVKGEPIINFYDPLVFPSDRWAA | |
| | SISQVNEAINQSREIIRAINIVRKIASEKGSGGSGSGSGGSEK | |
| | AAKAEEAARKMEELFKKHKIVAVLRANSVEEAIEKAVAVF | |
| | AGGVHLIEITFTVPDADTVIKALSVLKEKGAIIGAGTVTSVE | |
| | QARKAVESGAEFIVSPHLDEEISQFAKEKGVFYMPGVMTPT | |
| | ELVKAMKLGHTILKLFPGEVVGPQFVKAMKGPFPNVKFVP | |
| | TGGVNLDNVAEWFKAGVLAVGVGSALVKGTPDEVREKA | |
| | KAFVEKIRGATELEHHHHHH | |
| RSV/A.023 | MELLILKANAITTILTAVTFCFASGQNITEEFYQSTCSAVSK | 86 |
| | GYLSALRTGWYTSVITIELSNIKENKCNGTDAKVKLIKQEL | |
| | DKYKNAVTELQLLMQSTPATNNRARRELPRFMNYTLNNA | |
| | KKTNVTLSKKRKRRFLGFLLGVGSAIASGVAVCKVLHLEGE | |
| | VNKIKSALLSTNKAVVSLSNGVSVLTFKVLDLKNYIDKQLL | |
| | PILNKQSCSISNIETVIEFQQKNNRLLEITREFSVNAGVTTPV | |
| | STYMLTNSELLSLINDMPITNDQKKLMSNNVQIVRQQSYSI | |
| | MCIIKEEVLAYVVQLPLYGVIDTPCWKLHTSPLCTTNTKEG | |
| | SNICLTRTDRGWYCDNAGSVSFFPQAETCKVQSNRVFCDT | |
| | MNSLTLPSEVNLCNVDIFNPKYDCKIMTSKDDVSSSVITSL | |
| | GAIVSCYGKTKCTASNKNRGIIKTFSNGCDYVSNKGVDTV | |
| | SVGNTLYYVNKQEGKSLYVKGEPIINFYDPLVFPSDRFAAS | |
| | ISQVNEAINQSREIIRAINIVRKIASEKGSGGSGSGSGGSEKA | |
| | AKAEEAARKMEELFKKHKIVAVLRANSVEEAIEKAVAVFA | |
| | GGVHLIEITFTVPDADTVIKALSVLKEKGAIIGAGTVTSVEQ | |
| | ARKAVESGAEFIVSPHLDEEISQFAKEKGVFYMPGVMTPTE | |
| | LVKAMKLGHTILKLFPGEVVGPQFVKAMKGPFPNVKFVPT | |
| | GGVNLDNVAEWFKAGVLAVGVGSALVKGTPDEVREKAK | |
| | AFVEKIRGATELEHHHHHH | |
| RSV/A.024 | MELLILKANAITTILTAVTFCFASGQNITEEFYQSTCSAVSK | 87 |
| | GYLSALRTGWYTSVITIELSNIKENKCNGTDAKVKLIKQEL | |
| | DKYKNAVTELQLLMQSTPATNNRARRELPRFMNYTLNNA | |
| | KKTNVTLSKKRKRRFLGFLLGVGSAIASGVAVCKVLHLEGE | |
| | VNKIKSALLSTNKAVVSLSNGVSVLTFKVLDLKNYIDKQLL | |
| | PILNKQSCSISNIETVIEFQQKNNRLLEITREFSVNAGVTTPV | |
| | SPYMLTNSELLSLINDMPITNDQKKLMSNNVQIVRQQSYSI | |
| | MCIIKEEVLAYVVQLPLYGVIDTPCWKLHTSPLCTTNTKEG | |
| | SNICLTRTDRGWYCDNAGSVSFFPQAETCKVQSNRVFCDT | |
| | MNSLTLPSEVNLCNVDIFNPKYDCKIMTSKDDVSSSVITSL | |
| | GAIVSCYGKTKCTASNKNRGIIKTFSNGCDYVSNKGVDTV | |
| | SVGNTLYYVNKQEGKSLYVKGEPIINFYDPLVFPSDRFAAS | |
| | ISQVNEAINQSREIIRAINIVRKIASEKGSGGSGSGSGGSEKA | |
| | AKAEEAARKMEELFKKHKIVAVLRANSVEEAIEKAVAVFA | |
| | GGVHLIEITFTVPDADTVIKALSVLKEKGAIIGAGTVTSVEQ | |
| | ARKAVESGAEFIVSPHLDEEISQFAKEKGVFYMPGVMTPTE | |
| | LVKAMKLGHTILKLFPGEVVGPQFVKAMKGPFPNVKFVPT | |
| | GGVNLDNVAEWFKAGVLAVGVGSALVKGTPDEVREKAK | |
| | AFVEKIRGATELEHHHHHH | |
| RSV/A.025 | MELLILKANAITTILTAVTFCFASGQNITEEFYQSTCSAVSK | 88 |
| | GYLSALRTGWYTSVITIELSNIKENKCNGTDAKVKLIKQEL | |
| | DKYKNAVTELQLLMQSTPATNNRARRELPRFMNYTLNNA | |
| | KKTNVTLSKKRKRRFLGFLLGVGSAIASGVAVCKVLHLEGE | |
| | VNKIKSALLSTNKAVVSLSNGVSVLTFKVLDLKNYIDKQLL | |
| | PILNKQSCSISNIETVIEFQQKNNRLLEITREFSVNAGVTTPV | |
| | SPYMLTNSELLSLINDMPITNDQKKLMSNNVQIVRQQSYSI | |
| | MCIIKEEVLAYVVQLPLYGVIDTPCWKLHTSPLCTTNTKEG | |
| | SNICLTRTDRGWYCDNAGSVSFFPQAETCKVQSNRVFCDT | |
| | MNSLTLPSEVNLCNVDIFNPKYDCKIMTSKDDVSSSVITSL | |
| | GAIVSCYGKTKCTASNKNRGIIKTFSNGCDYVSNKGVDTV | |
| | SVGNTLYYVNKQEGKSLYVKGEPIINFYDPLVFPSARFAAS | |
| | ISQVNEAINQSREIIRAINIVRKIASEKGSGGSGSGSGGSEKA | |
| | AKAEEAARKMEELFKKHKIVAVLRANSVEEAIEKAVAVFA | |
| | GGVHLIEITFTVPDADTVIKALSVLKEKGAIIGAGTVTSVEQ | |
| | ARKAVESGAEFIVSPHLDEEISQFAKEKGVFYMPGVMTPTE | |
| | LVKAMKLGHTILKLFPGEVVGPQFVKAMKGPFPNVKFVPT | |
| | GGVNLDNVAEWFKAGVLAVGVGSALVKGTPDEVREKAK | |
| | AFVEKIRGATELEHHHHHH | |
| RSV/A.026 | MELLILKANAITTILTAVTFCFASGQNITEEFYQSTCSAVSK | 89 |
| | GYLSALRTGWYTSVITIELSNIKENKCNGTDAKVKLIKQEL | |
| | DKYKNAVTELQLLMQSTPATNNRARRELPRFMNYTLNNA | |
| | KKTNVTLSKKRKRRFLGFLLGVGSAIASGVAVCKVLHLEGE | |
| | VNKIKSALLSTNKAVVSLSNGVSVLTFKVLDLKNYIDKQLL | |
| | PILNKQSCSISNIETVIEFQQKNNRLLEITREFSVNAGVTTPV | |
| | STYMLTNSELLSLINDMPITNDQKKLMSNNVQIVRQQSYSI | |
| | MCIIKEEVLAYVVQLPLYGVIDTPCWKLHTSPLCTTNTKEG | |
| | SNICLTRTDRGWYCDNAGSVSFFPQAETCKVQSNRVFCDT | |
| | MNSLTLPSEVNLCNVDIFNPKYDCKIMTSKDDVSSSVITSL | |
| | GAIVSCYGKTKCTASNKNRGIIKTFSNGCDYVSNKGVDTV | |
| | SVGNTLYYVNKQEGKSLYVKGEPIINFYDPLVFPSARWAA | |
| | SISQVNEAINQSREIIRAINIVRKIASEKGSGGSGSGSGGSEK | |
| | AAKAEEAARKMEELFKKHKIVAVLRANSVEEAIEKAVAVF | |
| | AGGVHLIEITFTVPDADTVIKALSVLKEKGAIIGAGTVTSVE | |
| | QARKAVESGAEFIVSPHLDEEISQFAKEKGVFYMPGVMTPT | |
| | ELVKAMKLGHTILKLFPGEVVGPQFVKAMKGPFPNVKFVP | |
| | TGGVNLDNVAEWFKAGVLAVGVGSALVKGTPDEVREKA | |
| | KAFVEKIRGATELEHHHHHH | |
| RSV/B.002 | MELLIHRSSAIFLTLAINALYLTSSQNITEEFYQSTCSAVSRG | 90 |
| | YLSALRTGWYTSVITIELSNIKETKCNGTDTKVKLIKQELD | |
| | KYKNAVTELQLLMQNTPAVNNRARREAPQYMNYTINTTK | |
| | NLNVSISKKRKRRFLGFLLGVGSAIASGIAVCKVLHLEGEV | |
| | NKIKNALQLTNKAVVSLSNGVSVLTFRVLDLKNYINNQLL | |
| | PMLNRQSCRISNIETVIEFQQKNSRLLEITREFSVNAGVTTPL | |
| | STYMLTNSELLSLINDMPITNDQKKLMSSNVQIVRQQSYSI | |
| | MCIIKEEVLAYVVQLPIYGVIDTPCWKLHTSPLCTTNIKEGS | |
| | NICLTRTDRGWYCDNAGSVSFFPQADTCKVQSNRVFCDT | |
| | MNSLTLPSEVSLCNTDIFNSKYDCKIMTSKTDISSSVITSLG | |
| | AIVSCYGKTKCTASNKNRGIIKTFSNGCDYVSNKGVDTVS | |
| | VGNTLYYVNKLEGKNLYVKGEPIINYYDPLVFPSDEFDASI | |
| | SQVNEKINQSLAFIRRSDELLGSGGSGSGSGGSEKAAKAEE | |
| | AARKMEELFKKHKIVAVLRANSVEEAIEKAVAVFAGGVH | |
| | LIEITFTVPDADTVIKALSVLKEKGAIIGAGTVTSVEQARKA | |
| | VESGAEFIVSPHLDEEISQFAKEKGVFYMPGVMTPTELVKA | |
| | MKLGHTILKLFPGEVVGPQFVKAMKGPFPNVKFVPTGGVN | |
| | LDNVAEWFKAGVLAVGVGSALVKGTPDEVREKAKAFVE | |
| | KIRGATELEHHHHHH | |
| RSV/B.081 | MELLIHRSSAIFLTLAINALYLTSSQNITEEFYQSTCSAVSRG | 91 |
| | YLSALRTGWYTSVITIELSNIKETKCNGTDTKVKLIKQELD | |
| | KYKNAVTELQLLMQNTPAVNNRARREAPQYMNYTINTTK | |
| | NLNVSISKKRKRRFLGFLLGVGSAIASGIAVCKVLHLEGEV | |
| | NKIKNALQLTNKAVVSLSNGVSVLTFRVLDLKNYINNQLL | |
| | PMLNRQSCRISNIETVIEFQQKNSRLLEITREFSVNAGVTTPL | |
| | STYMLTNSELLSLINDMPITNDQKKLMSSNVQIVRQQSYSI | |
| | MCIIKEEVLAYVVQLPIYGVIDTPCWKLHTSPLCTTNIKEGS | |
| | NICLTRTDRGWYCDNAGSVSFFPQADTCKVQSNRVFCDT | |
| | MNSLTLPSEVSLCNTDIFNSKYDCKIMTSKDDISSSVITSLG | |
| | AIVSCYGKTKCTASNKNRGIIKTFSNGCDYVSNKGVDTVS | |
| | VGNTLYYVNKLEGKNLYVKGEPIINYYDPLVFPSARFAASI | |
| | SQVNEAINQSLAFIRRSDELLGSGGSGSGSGGSEKAAKAEE | |
| | AARKMEELFKKHKIVAVLRANSVEEAIEKAVAVFAGGVH | |
| | LIEITFTVPDADTVIKALSVLKEKGAIIGAGTVTSVEQARKA | |
| | VESGAEFIVSPHLDEEISQFAKEKGVFYMPGVMTPTELVKA | |
| | MKLGHTILKLFPGEVVGPQFVKAMKGPFPNVKFVPTGGVN | |
| | LDNVAEWFKAGVLAVGVGSALVKGTPDEVREKAKAFVE | |
| | KIRGATELEHHHHHH | |
| RSV/B.093 | MELLIHRSSAIFLTLAINALYLTSSQNITEEFYQSTCSAVSRG | 92 |
| | YLSALRTGWYTSVITIELSNIKETKCNGTDTKVKLIKQELD | |
| | KYKNAVTELQLLMQNTPAVNNRARREAPQYMNYTINTTK | |
| | NLNVSISKKRKRRFLGFLLGVGSAIASGIAVCKVLHLEGEV | |
| | NKIKNALQLTNKAVVSLSNGVSVLTFRVLDLKNYINNQLL | |
| | PMLNRQSCRISNIETVIEFQQKNSRLLEITREFSVNAGVTTPL | |
| | STYMLTNSELLSLINDMPITNDQKKLMSSNVQIVRQQSYSI | |
| | MCIIKEEVLAYVVOLPIYGVIDTPCWKLHTSPLCTTNIKEGS | |
| | NICLTRTDRGWYCDNAGSVSFFPQADTCKVQSNRVFCDT | |
| | MNSLTLPSEVSLCNTDIFNSKYDCKIMTSKDDISSSVITSLG | |
| | AIVSCYGKTKCTASNKNRGIIKTFSNGCDYVSNKGVDTVS | |
| | VGNTLYYVNKLEGKNLYVKGEPIINYYDPLVFPSARWAAS | |
| | ISQVNEAINQSLAFIRRSDELLGSGGSGSGSGGSEKAAKAEE | |
| | AARKMEELFKKHKIVAVLRANSVEEAIEKAVAVFAGGVH | |
| | LIEITFTVPDADTVIKALSVLKEKGAIIGAGTVTSVEQARKA | |
| | VESGAEFIVSPHLDEEISQFAKEKGVFYMPGVMTPTELVKA | |
| | MKLGHTILKLFPGEVVGPQFVKAMKGPFPNVKFVPTGGVN | |
| | LDNVAEWFKAGVLAVGVGSALVKGTPDEVREKAKAFVE | |
| | KIRGATELEHHHHHH | |
| RSV/B.099 | MELLIHRSSAIFLTLAINALYLTSSQNITEEFYQSTCSAVSRG | 93 |
| | YLSALRTGWYTSVITIELSNIKEVKCNGTDTKVKLIKQELD | |
| | KYKNAVTELQLLMQNTPAVNNRARREAPQYMNYTINTTK | |
| | NLNVSISKKRKRRFLGFLLGVGSAIASGIAVCKVLHLEGEV | |
| | NKIKNALQLTNKAVVSLSNGVSVLTFRVLDLKNYINNQLL | |
| | PMLNRQSCRISNIETVIEFQQKNSRLLEITREFSVNAGVTTPL | |
| | STYMLTNSELLSLINDMPITNDQKKLMSSNVQIVRQQSYSI | |
| | MCIIKEEVLAYVVQLPIYGVIDTPCWKLHTSPLCTTNIKEGS | |
| | NICLTRTDRGWYCDNAGSVSFFPQADTCKVQSNRVFCDT | |
| | MNSLTLPSEVSLCNTDIFNSKYDCKIMTSKTDISSSVITSLG | |
| | AIVSCYGKTKCTASNKNRGIIKTFSNGCDYVSNKGVDTVS | |
| | VGNTLYYVNKLEGKNLYVKGEPIINYYDPLVFPSDRFDASI | |
| | SQVNEAINQSLAFIRRSDELLGSGGSGSGSGGSEKAAKAEE | |
| | AARKMEELFKKHKIVAVLRANSVEEAIEKAVAVFAGGVH | |
| | LIEITFTVPDADTVIKALSVLKEKGAIIGAGTVTSVEQARKA | |
| | VESGAEFIVSPHLDEEISQFAKEKGVFYMPGVMTPTELVKA | |
| | MKLGHTILKLFPGEVVGPQFVKAMKGPFPNVKFVPTGGVN | |
| | LDNVAEWFKAGVLAVGVGSALVKGTPDEVREKAKAFVE | |
| | KIRGATELEHHHHHH | |
| RSV/B.100 | MELLIHRSSAIFLTLAINALYLTSSQNITEEFYQSTCSAVSRG | 94 |
| | YLSALRTGWYTSVITIELSNIKEVKCNGTDTKVKLIKQELD | |
| | KYKNAVTELQLLMQNTPAVNNRARREAPQYMNYTINTTK | |
| | NLNVSISKKRKRRFLGFLLGVGSAIASGIAVCKVLHLEGEV | |
| | NKIKNALQLTNKAVVSLSNGVSVLTFRVLDLKNYINNQLL | |
| | PMLNRQSCRISNIETVIEFQQKNSRLLEITREFSVNAGVTTPL | |
| | SPYMLTNSELLSLINDMPITNDQKKLMSSNVQIVRQQSYSI | |
| | MCIIKEEVLAYVVQLPIYGVIDTPCWKLHTSPLCTTNIKEGS | |
| | NICLTRTDRGWYCDNAGSVSFFPQADTCKVQSNRVFCDT | |
| | MNSLTLPSEVSLCNTDIFNSKYDCKIMTSKTDISSSVITSLG | |
| | AIVSCYGKTKCTASNKNRGIIKTFSNGCDYVSNKGVDTVS | |
| | VGNTLYYVNKLEGKNLYVKGEPIINYYDPLVFPSDRFDASI | |
| | SQVNEAINQSLAFIRRSDELLGSGGSGSGSGGSEKAAKAEE | |
| | AARKMEELFKKHKIVAVLRANSVEEAIEKAVAVFAGGVH | |
| | LIEITFTVPDADTVIKALSVLKEKGAIIGAGTVTSVEQARKA | |
| | VESGAEFIVSPHLDEEISQFAKEKGVFYMPGVMTPTELVKA | |
| | MKLGHTILKLFPGEVVGPQFVKAMKGPFPNVKFVPTGGVN | |
| | LDNVAEWFKAGVLAVGVGSALVKGTPDEVREKAKAFVE | |
| | KIRGATELEHHHHHH | |
| RSV/B.123 | MELLIHRSSAIFLTLAINALYLTSSQNITEEFYQSTCSAVSRG | 95 |
| | YLSALRTGWYTSVITIELSNIKEVKCNGTDTKVKLIKQELD | |
| | KYKNAVTELQLLMQNTPAVNNRARREAPQYMNYTINTTK | |
| | NLNVSISKKRKRRFLGFLLGVGSAIASGIAVCKVLHLEGEV | |
| | NKIKNALQLTNKAVVSLSNGVSVLTFRVLDLKNYINNQLL | |
| | PMLNRQSCRISNIETVIEFQQKNSRLLEITREFSVNAGVTTPL | |
| | STYMLTNSELLSLINDMPITNDQKKLMSSNVQIVRQQSYSI | |
| | MCIIKEEVLAYVVOLPIYGVIDTPCWKLHTSPLCTTNIKEGS | |
| | NICLTRTDRGWYCDNAGSVSFFPQADTCKVQSNRVFCDT | |
| | MNSLTLPSEVSLCNTDIFNSKYDCKIMTSKDDISSSVITSLG | |
| | AIVSCYGKTKCTASNKNRGIIKTFSNGCDYVSNKGVDTVS | |
| | VGNTLYYVNKLEGKNLYVKGEPIINYYDPLVFPSDRFAASI | |
| | SQVNEAINQSLAFIRRSDELLGSGGSGSGSGGSEKAAKAEE | |
| | AARKMEELFKKHKIVAVLRANSVEEAIEKAVAVFAGGVH | |
| | LIEITFTVPDADTVIKALSVLKEKGAIIGAGTVTSVEQARKA | |
| | VESGAEFIVSPHLDEEISQFAKEKGVFYMPGVMTPTELVKA | |
| | MKLGHTILKLFPGEVVGPQFVKAMKGPFPNVKFVPTGGVN | |
| | LDNVAEWFKAGVLAVGVGSALVKGTPDEVREKAKAFVE | |
| | KIRGATELEHHHHHH | |
| RSV/B.147 | MELLIHRSSAIFLTLAINALYLTSSQNITEEFYQSTCSAVSRG | 96 |
| | YLSALRTGWYTSVITIELSNIKETKCNGTDTKVKLIKQELD | |
| | KYKNAVTELQLLMQNTPAVNNRARREAPQYMNYTINTTK | |
| | NLNVSISKKRKRRFLGFLLGVGSAIASGIAVCKVLHLEGEV | |
| | NKIKNALQLTNKAVVSLSNGVSVLTFRVLDLKNYINNQLL | |
| | PMLNRQSCRISNIETVIEFQQKNSRLLEITREFSVNAGVTTPL | |
| | STYMLTNSELLSLINDMPITNDQKKLMSSNVQIVRQQSYSI | |
| | MCIIKEEVLAYVVQLPIYGVIDTPCWKLHTSPLCTTNIKEGS | |
| | NICLTRTDRGWYCDNAGSVSFFPQADTCKVQSNRVFCDT | |
| | MNSLTLPSEVSLCNTDIFNSKYDCKIMTSKTDISSSVITSLG | |
| | AIVSCYGKTKCTASNKNRGIIKTFSNGCDYVSNKGVDTVS | |
| | VGNTLYYVNKLEGKNLYVKGEPIINYYDPLVFPSDRFDASI | |
| | SQVNEAINQSREIIRAINIVRKIASEKGSGGSGSGSGGSEKA | |
| | AKAEEAARKMEELFKKHKIVAVLRANSVEEAIEKAVAVFA | |
| | GGVHLIEITFTVPDADTVIKALSVLKEKGAIIGAGTVTSVEQ | |
| | ARKAVESGAEFIVSPHLDEEISQFAKEKGVFYMPGVMTPTE | |
| | LVKAMKLGHTILKLFPGEVVGPQFVKAMKGPFPNVKFVPT | |
| | GGVNLDNVAEWFKAGVLAVGVGSALVKGTPDEVREKAK | |
| | AFVEKIRGATELEHHHHHH | |
| RSV/B.148 | MELLIHRSSAIFLTLAINALYLTSSQNITEEFYQSTCSAVSRG | 97 |
| | YLSALRTGWYTSVITIELSNIKETKCNGTDTKVKLIKQELD | |
| | KYKNAVTELQLLMQNTPAVNNRARREAPQYMNYTINTTK | |
| | NLNVSISKKRKRRFLGFLLGVGSAIASGIAVCKVLHLEGEV | |
| | NKIKNALQLTNKAVVSLSNGVSVLTFRVLDLKNYINNQLL | |
| | PMLNRQSCRISNIETVIEFQQKNSRLLEITREFSVNAGVTTPL | |
| | SPYMLTNSELLSLINDMPITNDQKKLMSSNVQIVRQQSYSI | |
| | MCIIKEEVLAYVVQLPIYGVIDTPCWKLHTSPLCTTNIKEGS | |
| | NICLTRTDRGWYCDNAGSVSFFPQADTCKVQSNRVFCDT | |
| | MNSLTLPSEVSLCNTDIFNSKYDCKIMTSKTDISSSVITSLG | |
| | AIVSCYGKTKCTASNKNRGIIKTFSNGCDYVSNKGVDTVS | |
| | VGNTLYYVNKLEGKNLYVKGEPIINYYDPLVFPSDRFDASI | |
| | SQVNEAINQSREIIRAINIVRKIASEKGSGGSGSGSGGSEKA | |
| | AKAEEAARKMEELFKKHKIVAVLRANSVEEAIEKAVAVFA | |
| | GGVHLIEITFTVPDADTVIKALSVLKEKGAIIGAGTVTSVEQ | |
| | ARKAVESGAEFIVSPHLDEEISQFAKEKGVFYMPGVMTPTE | |
| | LVKAMKLGHTILKLFPGEVVGPQFVKAMKGPFPNVKFVPT | |
| | GGVNLDNVAEWFKAGVLAVGVGSALVKGTPDEVREKAK | |
| | AFVEKIRGATELLEHHHHHH | |
| RSV/B.160 | MELLIHRSSAIFLTLAINALYLTSSQNITEEFYQSTCSAVSRG | 98 |
| | YLSALRTGWYTSVITIELSNIKETKCNGTDTKVKLIKQELD | |
| | KYKNAVTELQLLMQNTPAVNNRARREAPQYMNYTINTTK | |
| | NLNVSISKKRKRRFLGFLLGVGSAIASGIAVCKVLHLEGEV | |
| | NKIKNALQLTNKAVVSLSNGVSVLTFRVLDLKNYINNQLL | |
| | PMLNRQSCRISNIETVIEFQQKNSRLLEITREFSVNAGVTTPL | |
| | SPYMLTNSELLSLINDMPITNDQKKLMSSNVQIVRQQSYSI | |
| | MCIIKEEVLAYVVQLPIYGVIDTPCWKLHTSPLCTTNIKEGS | |
| | NICLTRTDRGWYCDNAGSVSFFPQADTCKVQSNRVFCDT | |
| | MNSLTLPSEVSLCNTDIFNSKYDCKIMTSKDDISSSVITSLG | |
| | AIVSCYGKTKCTASNKNRGIIKTFSNGCDYVSNKGVDTVS | |
| | VGNTLYYVNKLEGKNLYVKGEPIINYYDPLVFPSDRWAAS | |
| | ISQVNEAINQSREIIRAINIVRKIASEKGSGGSGSGSGGSEKA | |
| | AKAEEAARKMEELFKKHKIVAVLRANSVEEAIEKAVAVFA | |
| | GGVHLIEITFTVPDADTVIKALSVLKEKGAIIGAGTVTSVEQ | |
| | ARKAVESGAEFIVSPHLDEEISQFAKEKGVFYMPGVMTPTE | |
| | LVKAMKLGHTILKLFPGEVVGPQFVKAMKGPFPNVKFVPT | |
| | GGVNLDNVAEWFKAGVLAVGVGSALVKGTPDEVREKAK | |
| | AFVEKIRGATELEHHHHHH | |
| RSV/B.171 | MELLIHRSSAIFLTLAINALYLTSSQNITEEFYQSTCSAVSRG | 99 |
| | YLSALRTGWYTSVITIELSNIKETKCNGTDTKVKLIKQELD | |
| | KYKNAVTELQLLMQNTPAVNNRARREAPQYMNYTINTTK | |
| | NLNVSISKKRKRRFLGFLLGVGSAIASGIAVCKVLHLEGEV | |
| | NKIKNALQLTNKAVVSLSNGVSVLTFRVLDLKNYINNQLL | |
| | PMLNRQSCRISNIETVIEFQQKNSRLLEITREFSVNAGVTTPL | |
| | STYMLTNSELLSLINDMPITNDQKKLMSSNVQIVRQQSYSI | |
| | MCIIKEEVLAYVVQLPIYGVIDTPCWKLHTSPLCTTNIKEGS | |
| | NICLTRTDRGWYCDNAGSVSFFPQADTCKVQSNRVFCDT | |
| | MNSLTLPSEVSLCNTDIFNSKYDCKIMTSKDDISSSVITSLG | |
| | AIVSCYGKTKCTASNKNRGIIKTFSNGCDYVSNKGVDTVS | |
| | VGNTLYYVNKLEGKNLYVKGEPIINYYDPLVFPSDRFAASI | |
| | SQVNEAINQSREIIRAINIVRKIASEKGSGGSGSGSGGSEKA | |
| | AKAEEAARKMEELFKKHKIVAVLRANSVEEAIEKAVAVFA | |
| | GGVHLIEITFTVPDADTVIKALSVLKEKGAIIGAGTVTSVEQ | |
| | ARKAVESGAEFIVSPHLDEEISQFAKEKGVFYMPGVMTPTE | |
| | LVKAMKLGHTILKLFPGEVVGPQFVKAMKGPFPNVKFVPT | |
| | GGVNLDNVAEWFKAGVLAVGVGSALVKGTPDEVREKAK | |
| | AFVEKIRGATELEHHHHHH | |
| RSV/B.172 | MELLIHRSSAIFLTLAINALYLTSSQNITEEFYQSTCSAVSRG | 100 |
| | YLSALRTGWYTSVITIELSNIKETKCNGTDTKVKLIKQELD | |
| | KYKNAVTELQLLMQNTPAVNNRARREAPQYMNYTINTTK | |
| | NLNVSISKKRKRRFLGFLLGVGSAIASGIAVCKVLHLEGEV | |
| | NKIKNALQLTNKAVVSLSNGVSVLTFRVLDLKNYINNQLL | |
| | PMLNRQSCRISNIETVIEFQQKNSRLLEITREFSVNAGVTTPL | |
| | SPYMLTNSELLSLINDMPITNDQKKLMSSNVQIVRQQSYSI | |
| | MCIIKEEVLAYVVOLPIYGVIDTPCWKLHTSPLCTTNIKEGS | |
| | NICLTRTDRGWYCDNAGSVSFFPQADTCKVQSNRVFCDT | |
| | MNSLTLPSEVSLCNTDIFNSKYDCKIMTSKDDISSSVITSLG | |
| | AIVSCYGKTKCTASNKNRGIIKTFSNGCDYVSNKGVDTVS | |
| | VGNTLYYVNKLEGKNLYVKGEPIINYYDPLVFPSDRFAASI | |
| | SQVNEAINQSREIIRAINIVRKIASEKGSGGSGSGSGGSEKA | |
| | AKAEEAARKMEELFKKHKIVAVLRANSVEEAIEKAVAVFA | |
| | GGVHLIEITFTVPDADTVIKALSVLKEKGAIIGAGTVTSVEQ | |
| | ARKAVESGAEFIVSPHLDEEISQFAKEKGVFYMPGVMTPTE | |
| | LVKAMKLGHTILKLFPGEVVGPQFVKAMKGPFPNVKFVPT | |
| | GGVNLDNVAEWFKAGVLAVGVGSALVKGTPDEVREKAK | |
| | AFVEKIRGATELEHHHHHH | |
| RSV/B.178 | MELLIHRSSAIFLTLAINALYLTSSQNITEEFYQSTCSAVSRG | 101 |
| | YLSALRTGWYTSVITIELSNIKETKCNGTDTKVKLIKQELD | |
| | KYKNAVTELQLLMQNTPAVNNRARREAPQYMNYTINTTK | |
| | NLNVSISKKRKRRFLGFLLGVGSAIASGIAVCKVLHLEGEV | |
| | NKIKNALQLTNKAVVSLSNGVSVLTFRVLDLKNYINNQLL | |
| | PMLNRQSCRISNIETVIEFQQKNSRLLEITREFSVNAGVTTPL | |
| | SPYMLTNSELLSLINDMPITNDQKKLMSSNVQIVRQQSYSI | |
| | MCIIKEEVLAYVVQLPIYGVIDTPCWKLHTSPLCTTNIKEGS | |
| | NICLTRTDRGWYCDNAGSVSFFPQADTCKVQSNRVFCDT | |
| | MNSLTLPSEVSLCNTDIFNSKYDCKIMTSKDDISSSVITSLG | |
| | AIVSCYGKTKCTASNKNRGIIKTFSNGCDYVSNKGVDTVS | |
| | VGNTLYYVNKLEGKNLYVKGEPIINYYDPLVFPSARFAASI | |
| | SQVNEAINQSREIIRAINIVRKIASEKGSGGSGSGSGGSEKA | |
| | AKAEEAARKMEELFKKHKIVAVLRANSVEEAIEKAVAVFA | |
| | GGVHLIEITFTVPDADTVIKALSVLKEKGAIIGAGTVTSVEQ | |
| | ARKAVESGAEFIVSPHLDEEISQFAKEKGVFYMPGVMTPTE | |
| | LVKAMKLGHTILKLFPGEVVGPQFVKAMKGPFPNVKFVPT | |
| | GGVNLDNVAEWFKAGVLAVGVGSALVKGTPDEVREKAK | |
| | AFVEKIRGATELEHHHHHH | |
| RSV/B.189 | MELLIHRSSAIFLTLAINALYLTSSQNITEEFYQSTCSAVSRG | 102 |
| | YLSALRTGWYTSVITIELSNIKETKCNGTDTKVKLIKQELD | |
| | KYKNAVTELQLLMQNTPAVNNRARREAPQYMNYTINTTK | |
| | NLNVSISKKRKRRFLGFLLGVGSAIASGIAVCKVLHLEGEV | |
| | NKIKNALQLTNKAVVSLSNGVSVLTFRVLDLKNYINNQLL | |
| | PMLNRQSCRISNIETVIEFQQKNSRLLEITREFSVNAGVTTPL | |
| | STYMLTNSELLSLINDMPITNDQKKLMSSNVQIVRQQSYSI | |
| | MCIIKEEVLAYVVQLPIYGVIDTPCWKLHTSPLCTTNIKEGS | |
| | NICLTRTDRGWYCDNAGSVSFFPQADTCKVQSNRVFCDT | |
| | MNSLTLPSEVSLCNTDIFNSKYDCKIMTSKDDISSSVITSLG | |
| | AIVSCYGKTKCTASNKNRGIIKTFSNGCDYVSNKGVDTVS | |
| | VGNTLYYVNKLEGKNLYVKGEPIINYYDPLVFPSARWAAS | |
| | ISQVNEAINQSREIIRAINIVRKIASEKGSGGSGSGSGGSEKA | |
| | AKAEEAARKMEELFKKHKIVAVLRANSVEEAIEKAVAVFA | |
| | GGVHLIEITFTVPDADTVIKALSVLKEKGAIIGAGTVTSVEQ | |
| | ARKAVESGAEFIVSPHLDEEISQFAKEKGVFYMPGVMTPTE | |
| | LVKAMKLGHTILKLFPGEVVGPQFVKAMKGPFPNVKFVPT | |
| | GGVNLDNVAEWFKAGVLAVGVGSALVKGTPDEVREKAK | |
| | AFVEKIRGATELEHHHHHH | |
| RSV/B.195 | MELLIHRSSAIFLTLAINALYLTSSQNITEEFYQSTCSAVSRG | 103 |
| | YLSALRTGWYTSVITIELSNIKETKCNGTDTKVKLIKQELD | |
| | KYKNAVTELQLLMQNTPAVNNRARREAPQYMNYTINTTK | |
| | NLNVSISKKRKRRFLGFLLGVGSAIASGIAVCKVLHLEGEV | |
| | NKIKNALQLTNKAVVSLSNGVSVLTFRVLDLKNYINNQLL | |
| | PMLNRQSCRISNIETVIEFQQKNSRLLEITREFSVNAGVTTPL | |
| | STYMLTNSELLSLINDMPITNDQKKLMSSNVQIVRQQSYSI | |
| | MCIIKEEVLAYVVQLPIYGVIDTPCWKLHTSPLCTTNIKEGS | |
| | NICLTRTDRGWYCDNAGSVSFFPQADTCKVQSNRVFCDT | |
| | MNSLTLPSEVSLCNTDIFNSKYDCKIMTSKTDISSSVITSLG | |
| | AIVSCYGKTKCTASNKNRGIIKTFSNGCDYVSNKGVDTVS | |
| | VGNTLYYVNKLEGKNLYVKGEPIINYYDPLVFPSDEFDASI | |
| | SQVNEKINQSREIIRAINIVRKIASEKGSGGSGSGSGGSEKA | |
| | AKAEEAARKMEELFKKHKIVAVLRANSVEEAIEKAVAVFA | |
| | GGVHLIEITFTVPDADTVIKALSVLKEKGAIIGAGTVTSVEQ | |
| | ARKAVESGAEFIVSPHLDEEISQFAKEKGVFYMPGVMTPTE | |
| | LVKAMKLGHTILKLFPGEVVGPQFVKAMKGPFPNVKFVPT | |
| | GGVNLDNVAEWFKAGVLAVGVGSALVKGTPDEVREKAK | |
| | AFVEKIRGATELEHHHHHH | |

Relative expression and antibody binding of each design are shown in Table 15.

TABLE 15
Relative expression and antibody binding by BLI

| Construct # | Expression | D25 | AM14 | 4D7 | Palivizumab |
|---|---|---|---|---|---|
| RSV/A.03 | +++ | +++ | ++ | ++ | +++ |
| RSV/B.001 | +++ | +++ | ++ | ++ | |
| RSV/B.002 | +++ | +++ | ++ | ++ | +++ |
| RSV/B.008 | + | +++ | ++++ | ++ | |
| RSV/B.030 | ++ | +++ | ++ | ++ | |
| RSV/B.032 | ++ | +++ | ++ | ++ | |
| RSV/B.040 | ++ | +++ | +++ | + | |
| RSV/B.051 | +++ | +++ | +++ | ++ | +++ |
| RSV/B.052 | +++ | +++ | +++ | ++ | +++ |
| RSV/B.053 | ++ | +++ | +++ | ++ | ++ |
| RSV/B.054 | ++ | +++ | ++ | ++ | ++ |
| RSV/B.055 | ++ | +++ | ++ | ++ | +++ |
| RSV/B.056 | + | +++ | ++ | ++ | ++ |
| RSV/B.057 | +++ | +++ | ++++ | ++ | ++ |
| RSV/B.058 | +++ | +++ | ++++ | +++ | ++ |
| RSV/B.059 | + | +++ | +++ | ++ | ++ |
| RSV/B.060 | ++ | +++ | +++ | ++ | ++ |
| RSV/B.061 | ++ | +++ | +++ | + | ++ |
| RSV/B.062 | + | +++ | +++ | +++ | +++ |
| RSV/B.063 | +++ | +++ | +++ | + | +++ |
| RSV/B.064 | +++ | +++ | +++ | ++ | ++++ |
| RSV/B.065 | ++ | +++ | +++ | ++ | ++ |
| RSV/B.066 | +++ | +++ | ++ | ++ | +++ |
| RSV/B.067 | +++ | +++ | ++ | ++ | +++ |
| RSV/B.068 | + | +++ | +++ | ++ | +++ |
| RSV/B.069 | +++ | +++ | +++ | ++ | +++ |
| RSV/B.070 | ++ | +++ | +++ | ++ | ++ |
| RSV/B.071 | + | +++ | +++ | +++ | |
| RSV/B.072 | + | +++ | ++ | +++ | |
| RSV/B.073 | + | +++ | ++ | +++ | |
| RSV/B.074 | + | +++ | +++ | ++++ | |
| RSV/B.075 | +++ | +++ | +++ | + | |
| RSV/B.076 | | +++ | | | |
| RSV/B.077 | ++ | +++ | +++ | + | ++ |
| RSV/B.078 | +++ | +++ | ++ | ++ | |
| RSV/B.079 | +++ | +++ | ++ | ++ | |
| RSV/B.080 | + | +++ | ++ | +++ | |
| RSV/B.081 | ++++ | +++ | ++++ | ++ | |
| RSV/B.082 | +++ | +++ | ++++ | ++ | |
| RSV/B.083 | + | +++ | +++ | ++ | ++ |
| RSV/B.084 | ++ | +++ | +++ | + | |
| RSV/B.085 | ++ | +++ | +++ | + | |
| RSV/B.086 | + | +++ | +++ | +++ | |
| RSV/B.087 | ++++ | +++ | ++++ | ++ | |
| RSV/B.088 | ++++ | +++ | ++++ | ++ | |
| RSV/B.089 | +++ | +++ | +++ | ++ | |
| RSV/B.090 | +++ | +++ | +++ | ++ | |
| RSV/B.091 | +++ | +++ | ++ | + | |
| RSV/B.092 | + | +++ | ++ | ++ | |
| RSV/B.093 | +++ | +++ | ++++ | + | |
| RSV/B.094 | +++ | +++ | ++++ | ++ | |
| RSV/B.095 | ++ | +++ | +++ | ++ | |
| RSV/B.096 | +++ | +++ | ++++ | ++ | |
| RSV/B.097 | +++ | +++ | +++ | ++ | |
| RSV/B.098 | ++ | +++ | +++ | +++ | |
| RSV/B.099 | +++ | +++ | +++ | + | ++ |
| RSV/B.100 | +++ | +++ | +++ | + | ++ |
| RSV/B.101 | ++ | +++ | +++ | + | ++ |
| RSV/B.102 | ++ | +++ | ++ | + | ++ |
| RSV/B.103 | ++ | +++ | ++ | + | ++ |
| RSV/B.104 | + | +++ | +++ | +++ | +++ |
| RSV/B.105 | + | +++ | +++ | +++ | +++ |
| RSV/B.106 | + | +++ | +++ | +++ | +++ |
| RSV/B.107 | + | +++ | +++ | − | + |
| RSV/B.108 | ++ | +++ | ++++ | +++ | ++ |
| RSV/B.109 | ++ | +++ | +++ | + | ++ |
| RSV/B.110 | + | +++ | +++ | +++ | ++ |
| RSV/B.111 | +++ | +++ | +++ | ++ | |
| RSV/B.112 | ++ | +++ | ++ | ++ | +++ |
| RSV/B.113 | + | +++ | ++ | ++++ | +++ |
| RSV/B.114 | + | +++ | ++ | +++ | +++ |
| RSV/B.115 | ++ | +++ | ++ | − | +++ |
| RSV/B.116 | + | +++ | ++ | + | ++ |
| RSV/B.117 | +++ | +++ | +++ | + | ++ |
| RSV/B.118 | ++ | +++ | ++++ | ++ | +++ |
| RSV/B.119 | + | +++ | +++ | ++++ | ++++ |
| RSV/B.120 | + | +++ | ++ | ++++ | +++ |
| RSV/B.121 | + | +++ | ++ | ++++ | +++ |
| RSV/B.122 | + | +++ | ++ | ++++ | +++ |
| RSV/B.123 | ++++ | +++ | +++ | + | +++ |
| RSV/B.124 | ++++ | +++ | | + | +++ |
| RSV/B.125 | ++ | +++ | +++ | ++ | ++ |
| RSV/B.126 | + | +++ | ++ | +++ | +++ |
| RSV/B.127 | + | +++ | ++ | +++ | +++ |
| RSV/B.128 | + | +++ | +++ | ++++ | +++ |
| RSV/B.129 | + | +++ | +++ | +++ | +++ |
| RSV/B.130 | + | +++ | +++ | +++ | +++ |
| RSV/B.131 | + | +++ | +++ | ++ | +++ |
| RSV/B.132 | + | +++ | +++ | +++ | +++ |
| RSV/B.133 | + | +++ | +++ | +++ | +++ |
| RSV/B.134 | + | +++ | ++ | ++++ | +++ |
| RSV/B.135 | + | +++ | +++ | +++ | +++ |
| RSV/B.136 | + | +++ | ++ | ++ | +++ |
| RSV/B.137 | + | +++ | ++ | ++++ | +++ |
| RSV/B.138 | + | +++ | ++ | ++++ | +++ |
| RSV/B.139 | ++ | +++ | ++ | ++ | +++ |
| RSV/B.140 | + | +++ | ++ | +++ | +++ |
| RSV/B.141 | ++ | +++ | +++ | ++ | +++ |
| RSV/B.142 | ++ | +++ | ++ | ++ | +++ |
| RSV/B.143 | + | +++ | ++ | +++ | +++ |
| RSV/B.144 | + | +++ | ++ | +++ | +++ |
| RSV/B.145 | + | +++ | ++ | +++ | +++ |
| RSV/B.146 | + | +++ | ++ | ++++ | ++++ |
| RSV/B.147 | ++++ | +++ | +++ | + | N/A |
| RSV/B.148 | ++++ | +++ | +++ | + | N/A |
| RSV/B.149 | + | +++ | ++ | ++ | N/A |
| RSV/B.150 | ++ | +++ | +++ | − | N/A |
| RSV/B.151 | ++ | +++ | ++++ | − | N/A |
| RSV/B.152 | + | ++++ | +++ | − | N/A |
| RSV/B.153 | +++ | +++ | +++ | + | N/A |
| RSV/B.154 | +++ | +++ | +++ | + | N/A |
| RSV/B.155 | + | +++ | ++ | + | N/A |
| RSV/B.156 | ++ | +++ | +++ | + | N/A |
| RSV/B.157 | + | +++ | +++ | + | N/A |
| RSV/B.158 | + | +++ | ++ | +++ | N/A |
| RSV/B.159 | +++ | +++ | +++ | ++ | N/A |
| RSV/B.160 | ++++ | +++ | +++ | + | N/A |
| RSV/B.161 | ++ | +++ | ++ | − | N/A |
| RSV/B.162 | ++ | ++++ | ++++ | − | N/A |
| RSV/B.163 | +++ | +++ | ++ | + | N/A |
| RSV/B.164 | ++ | +++ | ++ | + | N/A |
| RSV/B.165 | +++ | +++ | +++ | + | N/A |
| RSV/B.166 | ++ | +++ | ++ | +++ | N/A |
| RSV/B.167 | + | +++ | ++ | − | N/A |
| RSV/B.168 | + | +++ | ++ | − | N/A |
| RSV/B.169 | + | + | + | − | N/A |
| RSV/B.170 | + | +++ | + | − | N/A |
| RSV/B.171 | +++ | +++ | +++ | + | N/A |
| RSV/B.172 | ++++ | +++ | +++ | + | N/A |
| RSV/B.173 | ++ | +++ | +++ | +++ | N/A |
| RSV/B.174 | +++ | +++ | +++ | ++ | N/A |
| RSV/B.175 | ++ | +++ | ++ | +++ | N/A |
| RSV/B.176 | + | +++ | ++ | +++ | N/A |
| RSV/B.177 | ++ | +++ | +++ | +++ | N/A |
| RSV/B.178 | +++ | +++ | +++ | + | N/A |
| RSV/B.179 | + | +++ | ++ | ++ | N/A |
| RSV/B.180 | +++ | +++ | +++ | + | N/A |
| RSV/B.181 | ++ | +++ | ++ | + | N/A |
| RSV/B.182 | ++ | ++ | ++ | + | N/A |
| RSV/B.183 | +++ | +++ | ++ | ++ | N/A |
| RSV/B.184 | ++++ | +++ | +++ | + | N/A |
| RSV/B.185 | ++ | +++ | ++ | ++ | N/A |
| RSV/B.186 | ++ | +++ | ++ | + | N/A |
| RSV/B.187 | ++ | +++ | ++ | + | N/A |
| RSV/B.188 | ++ | +++ | ++ | +++ | N/A |
| RSV/B.189 | ++++ | +++ | +++ | − | N/A |
| RSV/B.190 | ++++ | +++ | +++ | + | N/A |
| RSV/B.191 | ++ | +++ | ++ | ++ | N/A |
| RSV/B.192 | ++ | +++ | +++ | + | N/A |
| RSV/B.193 | + | + | + | − | N/A |
| RSV/B.194 | + | ++ | + | + | N/A |

Mutations of designed constructs used in the experiments are shown in Table 16. All sequences featured the ectodomain of RSV F (with DS-Cav1 mutations) genetically fused to I53-50ΔΔcys (SEQ ID NO: 64) with a flexible glycine- and serine-based linker. Designs that contain a C-terminal alpha-helical segment place this segment at the C-terminus of the ectodomain as described earlier, and prior to the flexible linker. SEQ ID NO: 1 is used for the reference sequence. In each case, the signal peptide at the N terminus or the tag at the C terminus may be replaced with known alternatives or deleted. “o” indicated that an amino acid substitution was used.

TABLE 16
Mutations of constructs used in the experiments

| | | T58M | | | | |
|---|---|---|---|---|---|---|
| | | V1541 | | | | Alpha- |
| | | V296A | | R235Y | | helical |
| Construct # | Space 1 | A298L | T249P | E232A | T67V | segment¹ |
| RSV/A.03 | | | | | | |
| RSV/B.001 | | | | | | |
| RSV/B.002 | | | | | | |
| RSV/B.008 | D486A + E487R + K498A | | | | | |
| RSV/B.030 | | | o | | | |
| RSV/B.032 | | | o | o | | |
| RSV/B.040 | | | | | | o |
| RSV/B.051 | E487R + K498A | | | | | |
| RSV/B.052 | E487R + K498A | | o | | | |
| RSV/B.053 | E487R + K498A | | o | o | | |
| RSV/B.054 | E487R + K498A | o | | | | |
| RSV/B.055 | E487R + K498A | o | o | | | |
| RSV/B.056 | E487R + K498A | o | o | o | | |
| RSV/B.057 | D486A + E487R + K498A | | | | | |
| RSV/B.058 | D486A + E487R + K498A | | o | | | |
| RSV/B.059 | D486A + E487R + K498A | | o | o | | |
| RSV/B.060 | D486A + E487R + K498A | o | | | | |
| RSV/B.061 | D486A + E487R + K498A | o | o | | | |
| RSV/B.062 | D486A + E487R + K498A | o | o | o | | |
| RSV/B.063 | F488W + D489A + T400D + | | | | | |
| | E487R + K498A | | | | | |
| RSV/B.064 | F488W + D489A + T400D + | | o | | | |
| | E487R + K498A | | | | | |
| RSV/B.065 | F488W + D489A + T400D + | | o | o | | |
| | E487R + K498A | | | | | |
| RSV/B.066 | F488W + D489A + T400D + | o | | | | |
| | E487R + K498A | | | | | |
| RSV/B.067 | F488W + D489A + T400D + | o | o | | | |
| | E487R + K498A | | | | | |
| RSV/B.068 | F488W + D489A + T400D + | o | o | o | | |
| | E487R + K498A | | | | | |
| RSV/B.069 | Q494M, S4851, K399A, | | | | | |
| | D486A + 487M + 498A | | | | | |
| RSV/B.070 | Q494M, S4851, K399A, | | o | | | |
| | D486A + 487M + 498A | | | | | |
| RSV/B.071 | Q494M, S4851, K399A, | | o | o | | |
| | D486A + 487M + 498A | | | | | |
| RSV/B.072 | Q494M, S4851, K399A, | o | | | | |
| | D486A + 487M + 498A | | | | | |
| RSV/B.073 | Q494M, S4851, K399A, | o | o | | | |
| | D486A + 487M + 498A | | | | | |
| RSV/B.074 | Q494M, S4851, K399A, | o | o | o | | |
| | D486A + 487M + 498A | | | | | |
| RSV/B.075 | D489A + T400D + E487R + | | | | | |
| | K498A | | | | | |
| RSV/B.076 | D489A + T400D + E487R + | | o | | | |
| | K498A | | | | | |
| RSV/B.077 | D489A + T400D + E487R + | | o | o | | |
| | K498A | | | | | |
| RSV/B.078 | D489A + T400D + E487R + | o | | | | |
| | K498A | | | | | |
| RSV/B.079 | D489A + T400D + E487R + | o | o | | | |
| | K498A | | | | | |
| RSV/B.080 | D489A + T400D + E487R + | o | o | o | | |
| | K498A | | | | | |
| RSV/B.081 | D489A + T400D + E487R + | | | | | |
| | K498A + D486A | | | | | |
| RSV/B.082 | D489A + T400D + E487R + | | o | | | |
| | K498A + D486A | | | | | |
| RSV/B.083 | D489A + T400D + E487R + | | o | o | | |
| | K498A + D486A | | | | | |
| RSV/B.084 | D489A + T400D + E487R + | o | | | | |
| | K498A + D486A | | | | | |
| RSV/B.085 | D489A + T400D + E487R + | o | o | | | |
| | K498A + D486A | | | | | |
| RSV/B.086 | D489A + T400D + E487R + | o | o | o | | |
| | K498A + D486A | | | | | |
| RSV/B.087 | F140W + D489A + T400D + | | | | | |
| | E487R + K498A + D486A | | | | | |
| RSV/B.088 | F140W + D489A + T400D + | | o | | | |
| | E487R + K498A + D486A | | | | | |
| RSV/B.089 | F140W + D489A + T400D + | | o | o | | |
| | E487R + K498A + D486A | | | | | |
| RSV/B.090 | F140W + D489A + T400D + | o | | | | |
| | E487R + K498A + D486A | | | | | |
| RSV/B.091 | F140W + D489A + T400D + | o | o | | | |
| | E487R + K498A + D486A | | | | | |
| RSV/B.092 | F140W + D489A + T400D + | o | o | o | | |
| | E487R + K498A + D486A | | | | | |
| RSV/B.093 | F488W + D489A + T400D + | | | | | |
| | E487R + K498A + D486A | | | | | |
| RSV/B.094 | F488W + D489A + T400D + | | o | | | |
| | E487R + K498A + D486A | | | | | |
| RSV/B.095 | F488W + D489A + T400D + | | o | o | | |
| | E487R + K498A + D486A | | | | | |
| RSV/B.096 | F488W + D489A + T400D + | o | | | | |
| | E487R + K498A + D486A | | | | | |
| RSV/B.097 | F488W + D489A + T400D + | o | o | | | |
| | E487R + K498A + D486A | | | | | |
| RSV/B.098 | F488W + D489A + T400D + | o | o | o | | |
| | E487R + K498A + D486A | | | | | |
| RSV/B.099 | E487R + K498A | | | | o | |
| RSV/B.100 | E487R + K498A | | o | | o | |
| RSV/B.101 | E487R + K498A | | o | o | o | |
| RSV/B.102 | E487R + K498A | o | | | o | |
| RSV/B.103 | E487R + K498A | o | o | | o | |
| RSV/B.104 | E487R + K498A | o | o | o | o | |
| RSV/B.105 | D486A + E487R + K498A | | | | o | |
| RSV/B.106 | D486A + E487R + K498A | | o | | o | |
| RSV/B.107 | D486A + E487R + K498A | | o | o | o | |
| RSV/B.108 | D486A + E487R + K498A | o | | | O | |
| RSV/B.109 | D486A + E487R + K498A | o | o | | o | |
| RSV/B.110 | D486A + E487R + K498A | o | o | o | o | |
| RSV/B.111 | F488W + D489A + T400D + | | | | o | |
| | E487R + K498A | | | | | |
| RSV/B.112 | F488W + D489A + T400D + | | o | | o | |
| | E487R + K498A | | | | | |
| RSV/B.113 | F488W + D489A + T400D + | | o | o | o | |
| | E487R + K498A | | | | | |
| RSV/B.114 | F488W + D489A + T400D + | o | | | o | |
| | E487R + K498A | | | | | |
| RSV/B.115 | F488W + D489A + T400D + | o | o | | o | |
| | E487R + K498A | | | | | |
| RSV/B.116 | F488W + D489A + T400D + | o | o | o | o | |
| | E487R + K498A | | | | | |
| RSV/B.117 | Q494M, S4851, K399A, | | | | o | |
| | D486A + 487M + 498A | | | | | |
| RSV/B.118 | Q494M, S4851, K399A, | | o | | o | |
| | D486A + 487M + 498A | | | | | |
| RSV/B.119 | Q494M, S4851, K399A, | | o | o | o | |
| | D486A + 487M + 498A | | | | | |
| RSV/B.120 | Q494M, S4851, K399A, | o | | | o | |
| | D486A + 487M + 498A | | | | | |
| RSV/B.121 | Q494M, S4851, K399A, | o | o | | o | |
| | D486A + 487M + 498A | | | | | |
| RSV/B.122 | Q494M, S4851, K399A, | o | o | o | o | |
| | D486A + 487M + 498A | | | | | |
| RSV/B.123 | D489A + T400D + E487R + | | | | o | |
| | K498A | | | | | |
| RSV/B.124 | D489A + T400D + E487R + | | o | | o | |
| | K498A | | | | | |
| RSV/B.125 | D489A + T400D + E487R + | | o | o | o | |
| | K498A | | | | | |
| RSV/B.126 | D489A + T400D + E487R + | o | | | o | |
| | K498A | | | | | |
| RSV/B.127 | D489A + T400D + E487R + | o | o | | o | |
| | K498A | | | | | |
| RSV/B.128 | D489A + T400D + E487R + | o | o | o | o | |
| | K498A | | | | | |
| RSV/B.129 | D489A + T400D + E487R + | | | | o | |
| | K498A + D486A | | | | | |
| RSV/B.130 | D489A + T400D + E487R + | | o | | o | |
| | K498A + D486A | | | | | |
| RSV/B.131 | D489A + T400D + E487R + | | o | o | o | |
| | K498A + D486A | | | | | |
| RSV/B.132 | D489A + T400D + E487R + | o | | | o | |
| | K498A + D486A | | | | | |
| RSV/B.133 | D489A + T400D + E487R + | o | o | | o | |
| | K498A + D486A | | | | | |
| RSV/B.134 | D489A + T400D + E487R + | o | o | o | o | |
| | K498A + D486A | | | | | |
| RSV/B.135 | F140W + D489A + T400D + | | | | o | |
| | E487R + K498A + D486A | | | | | |
| RSV/B.136 | F140W + D489A + T400D + | | o | | o | |
| | E487R + K498A + D486A | | | | | |
| RSV/B.137 | F140W + D489A + T400D + | | o | O | o | |
| | E487R + K498A + D486A | | | | | |
| RSV/B.138 | F140W + D489A + T400D + | o | | | o | |
| | E487R + K498A + D486A | | | | | |
| RSV/B.139 | F140W + D489A + T400D + | o | o | | o | |
| | E487R + K498A + D486A | | | | | |
| RSV/B.140 | F140W + D489A + T400D + | o | o | O | o | |
| | E487R + K498A + D486A | | | | | |
| RSV/B.141 | F488W + D489A + T400D + | | | | o | |
| | E487R + K498A + D486A | | | | | |
| RSV/B.142 | F488W + D489A + T400D + | | o | | o | |
| | E487R + K498A + D486A | | | | | |
| RSV/B.143 | F488W + D489A + T400D + | | o | o | o | |
| | E487R + K498A + D486A | | | | | |
| RSV/B.144 | F488W + D489A + T400D + | o | | | o | |
| | E487R + K498A + D486A | | | | | |
| RSV/B.145 | F488W + D489A + T400D + | o | o | | o | |
| | E487R + K498A + D486A | | | | | |
| RSV/B.146 | F488W + D489A + T400D + | o | o | o | o | |
| | E487R + K498A + D486A | | | | | |
| RSV/B.147 | E487R + K498A | | | | | o |
| RSV/B.148 | E487R + K498A | | o | | | o |
| RSV/B.149 | E487R + K498A | | o | o | | o |
| RSV/B.150 | E487R + K498A | o | | | | o |
| RSV/B.151 | E487R + K498A | o | o | | | o |
| RSV/B.152 | E487R + K498A | o | o | o | | o |
| RSV/B.153 | D486A + E487R + K498A | | | | | o |
| RSV/B.154 | D486A + E487R + K498A | | o | | | o |
| RSV/B.155 | D486A + E487R + K498A | | o | o | | o |
| RSV/B.156 | D486A + E487R + K498A | o | | | | o |
| RSV/B.157 | D486A + E487R + K498A | o | o | | | o |
| RSV/B.158 | D486A + E487R + K498A | o | o | o | | o |
| RSV/B.159 | F488W + D489A + T400D + | | | | | o |
| | E487R + K498A | | | | | |
| RSV/B.160 | F488W + D489A + T400D + | | o | | | o |
| | E487R + K498A | | | | | |
| RSV/B.161 | F488W + D489A + T400D + | | o | o | | o |
| | E487R + K498A | | | | | |
| RSV/B.162 | F488W + D489A + T400D + | o | | | | o |
| | E487R + K498A | | | | | |
| RSV/B.163 | F488W + D489A + T400D + | o | o | | | o |
| | E487R + K498A | | | | | |
| RSV/B.164 | F488W + D489A + T400D + | o | o | o | | o |
| | E487R + K498A | | | | | |
| RSV/B.165 | Q494M, S4851, K399A, | | | | | o |
| | D486A + 487M + 498A | | | | | |
| RSV/B.166 | Q494M, S4851, K399A, | | o | | | o |
| | D486A + 487M + 498A | | | | | |
| RSV/B.167 | Q494M, S4851, K399A, | | o | o | | o |
| | D486A + 487M + 498A | | | | | |
| RSV/B.168 | Q494M, S4851, K399A, | o | | | | o |
| | D486A + 487M + 498A | | | | | |
| RSV/B.169 | Q494M, S4851, K399A, | o | o | | | o |
| | D486A + 487M + 498A | | | | | |
| RSV/B.170 | Q494M, S4851, K399A, | o | o | o | | o |
| | D486A + 487M + 498A | | | | | |
| RSV/B.171 | D489A + T400D + E487R + | | | | | o |
| | K498A | | | | | |
| RSV/B.172 | D489A + T400D + E487R + | | o | | | o |
| | K498A | | | | | |
| RSV/B.173 | D489A + T400D + E487R + | | o | o | | o |
| | K498A | | | | | |
| RSV/B.174 | D489A + T400D + E487R + | o | | | | o |
| | K498A | | | | | |
| RSV/B.175 | D489A + T400D + E487R + | o | o | | | o |
| | K498A | | | | | |
| RSV/B.176 | D489A + T400D + E487R + | o | o | o | | o |
| | K498A | | | | | |
| RSV/B.177 | D489A + T400D + E487R + | | | | | o |
| | K498A + D486A | | | | | |
| RSV/B.178 | D489A + T400D + E487R + | | o | | | o |
| | K498A + D486A | | | | | |
| RSV/B.179 | D489A + T400D + E487R + | | o | o | | o |
| | K498A + D486A | | | | | |
| RSV/B.180 | D489A + T400D + E487R + | o | | | | o |
| | K498A + D486A | | | | | |
| RSV/B.181 | D489A + T400D + E487R + | o | o | | | o |
| | K498A + D486A | | | | | |
| RSV/B.182 | D489A + T400D + E487R + | o | o | o | | o |
| | K498A + D486A | | | | | |
| RSV/B.183 | F140W + D489A + T400D + | | | | | o |
| | E487R + K498A + D486A | | | | | |
| RSV/B.184 | F140W + D489A + T400D + | | o | | | o |
| | E487R + K498A + D486A | | | | | |
| RSV/B.185 | F140W + D489A + T400D + | | o | o | | o |
| | E487R + K498A + D486A | | | | | |
| RSV/B.186 | F140W + D489A + T400D + | o | | | | o |
| | E487R + K498A + D486A | | | | | |
| RSV/B.187 | F140W + D489A + T400D + | o | o | | | o |
| | E487R + K498A + D486A | | | | | |
| RSV/B.188 | F140W + D489A + T400D + | o | o | o | | o |
| | E487R + K498A + D486A | | | | | |
| RSV/B.189 | F488W + D489A + T400D + | | | | | o |
| | E487R + K498A + D486A | | | | | |
| RSV/B.190 | F488W + D489A + T400D + | | o | | | o |
| | E487R + K498A + D486A | | | | | |
| RSV/B.191 | F488W + D489A + T400D + | | o | o | | o |
| | E487R + K498A + D486A | | | | | |
| RSV/B.192 | F488W + D489A + T400D + | o | | | | o |
| | E487R + K498A + D486A | | | | | |
| RSV/B.193 | F488W + D489A + T400D + | o | o | | | o |
| | E487R + K498A + D486A | | | | | |
| RSV/B.194 | F488W + D489A + T400D + | o | o | o | | o |
| | E487R + K498A + D486A | | | | | |
| RSV/B.195 | | | | | | o |
| RSV/A.013 | | | | | | o |
| RSV/A.023 | D489A + T400D + E487R + | | | | | o |
| | K498A | | | | | |

¹500-NQSREIIRAINIVRKIASEK-519

To test whether these stabilizing modifications are generalizable outside of RSV/B-based antigens, two novel designs were also evaluated in the context of an RSV/A antigen sequence (RSV/A.013 and RSV/A.023). Both designs contained DS-Cav1 mutations and were genetically fused to I53-50ΔΔcys, with RSV/A.013 adding a C-terminal alpha-helical segment (equivalent to the RSV/B.195 design) and RSV/A.023 adding both a C-terminal alpha-helical segment and D489A, T400D, E487R and K498A mutations (equivalent to the RSV/B.171 design). Sequences and mutations for these designs are further detailed in Table 14 and Table 16 respectively. Both thermal stability and storage stability at 40° C. were strongly increased relative to the RSV/A.03 design, which did not include a C-terminal alpha-helical segment or D489A, T400D, E487R and K498A mutations (Table 12). RSV/A.013 and RSV/A.023 showed increases in melting temperature of 4.5° C. and 19.0° C. relative to RSV/A.03, which demonstrates that the C-terminal alpha-helical segment can be alone used to improve the thermal stability of both RSV/A and RSV/B antigens, and that the combination of the C-terminal alpha-helical segment with further stabilizing mutations can more rigorously improve the thermal stability of both RSV/A and RSV/B antigens. Further, both RSV/A.013 and RSV/A.023 were capable of in vitro assembly into nanostructures with addition of I53-50B as evaluated by DLS (Table 13).

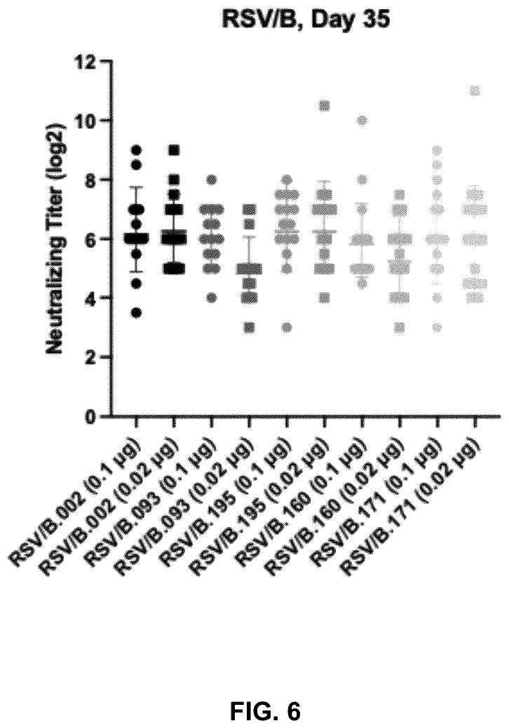

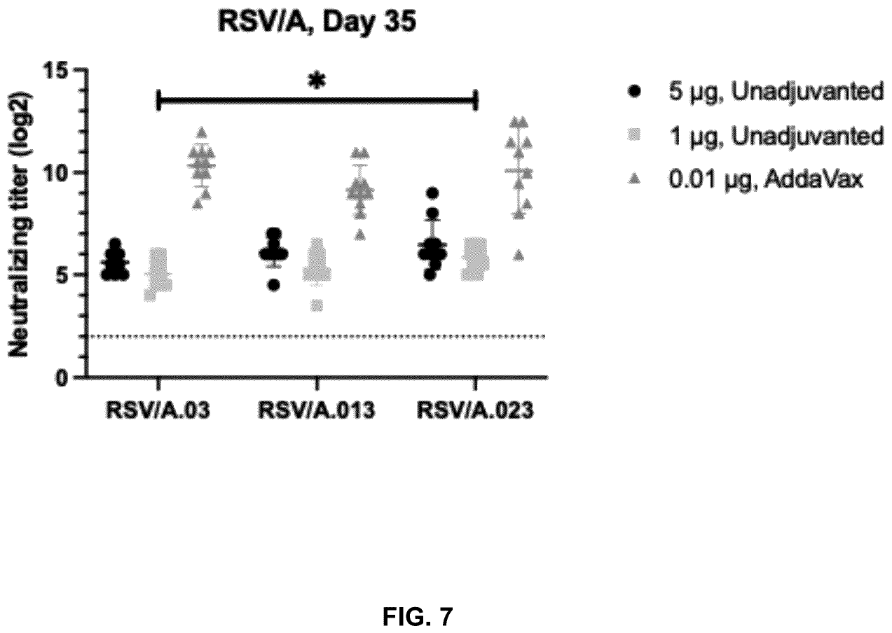

In order to evaluate the immunogenicity of different designs based on either RSV/B or RSV/A, two in vivo studies were performed in BALB/c mice. In one study, RSV/B neutralizing titers elicited by immunization with either a 0.02 μg or 0.1 μg dose of assembled nanostructures based on RSV/B.002, RSV/B.093, RSV/B.195, RSV/B.160, or RSV/B.171 were evaluated, all of which were adjuvanted with AddaVax™ (FIG. 6). No statistically significant differences between any of the designs were observed at either dose. Similarly, no statistically significant differences were observed between mice immunized with either a 5 μg unadjuvanted or 0.01 μg AddaVax-adjuvanted dose of assembled nanostructures based on RSV/A.03, RSV/A.013, or RSV/A.023 (FIG. 7). However, mice immunized with 1 μg of unadjuvanted RSV/A.023 nanostructure did have significantly higher RSV/A neutralizing titers than mice immunized with the same dose of unadjuvanted RSV/A.03.

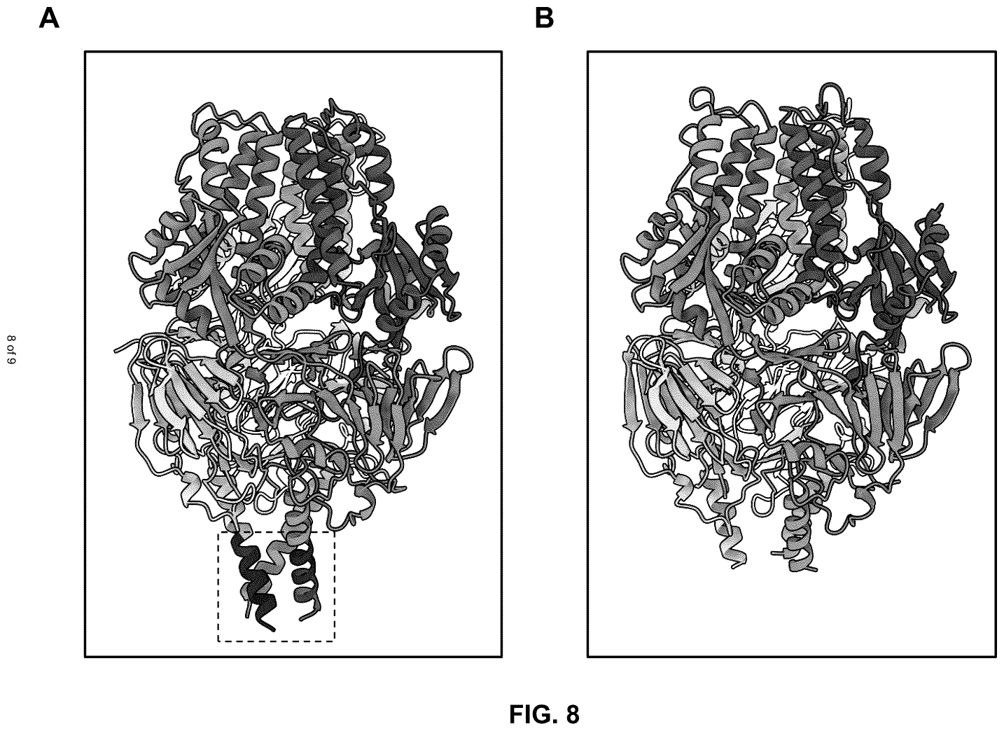

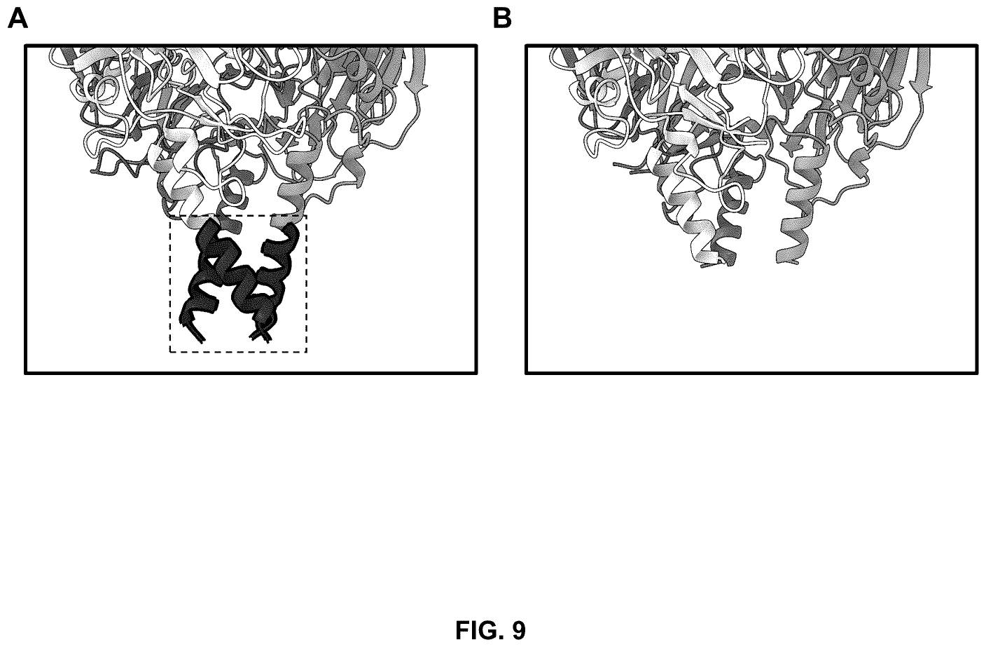

A cryo-EM structure was solved for the F ectodomain of RSV/A.023 at 3.35 Å. Both the F ectodomain and the C-terminal helix-forming segment were resolved, while the I53-50ΔΔcys domains fused to the antigen were not resolved due the flexibility of the linker between these domains and the antigen ectodomain. The overall structure of the F ectodomain (FIG. 8A) was similar to a published cryo-EM structure of a soluble DS-Cav1 construct (FIG. 8B, PDB 7LUE), suggesting that the F ectodomain of RSV/A.023 is properly formed for use in vaccines. The C-terminal helix-forming segment formed hydrophobic interactions between each subunit as intended by the design protocols (FIG. 9A), and confirmed the addition of this structural segment relative to public structures of published designs for stabilized RSV F ectodomains (FIG. 9B).

### Materials and Methods

Small-Scale Transfection: A variety of RSV/B designed were screened for expression, antigenicity and thermal stability via 96 deep well transfections. Expi293 cells in log phase growth were counted and seeded at 2.5×10⁶ cells/ml. Cells were incubated overnight at 36° C. with shaking (120 rpm). The next day the cells were counted and diluted to 3×106 cells/ml in 0.6 ml per well. Cells were transiently transfected as follows. A 5× master mix of 1000 μg plasmid DNA was diluted to 35 ml final volume with OptiMEM™ and gently mixed in a separate 96 deep well plate. A 5× master mix of Transporter 5 transfection reagent was diluted to a final volume of 35 ml with OptiMEM™ and gently mixed. The Diluted Transporter 5 was added to the diluted DNA, mixed, incubated at room temperature for 10 minutes and then 42 μl was added dropwise to each well while gently shaking plate. Cells were placed back in the incubator, shaking at 1050 rpm in for 4 days.

Biolayer Interferometry: Antibodies 16A8 (ATUM), AM14, 4D7, D25, and Palivizumab (Creative Biolabs) were normalized in concentration to 10 μg/mL in BLI assay buffer (PBS, 0.5% BSA, 0.05% Tween 20, pH 7.4) in a sufficient volume to load 80 μL per well of a black 384-well microplate (Thermo Scientific, 460518). Briefly, on an Octet rh16 instrument, Protein G biosensors (Sartorius, 18-5082) were dipped into assay buffer for 60 seconds to achieve a baseline. Next, the biosensors were dipped into each antibody for 60 s in order to immobilize the antibodies present but without reaching saturation, followed by an additional baseline step. The immobilized antibodies were allowed to associate with 80 μL of RSV/B supernatant for 120 seconds, and then the biosensors were dipped back into assay buffer for 120 seconds to observe any possible dissociation. 16A8 is a monoclonal antibody that recognizes I53-50A and was used to estimate relative expression levels. AM14, D25, 4D7, and Palivizumab are specific to RSV F protein.

Large-Scale Transfection: Based on the data from the 96 deep well screen, a subset of constructs were expressed transiently at the 1-liter scale. Expi293 cells in log phase growth were counted and seeded in 220 ml at 2.5×106 cells/ml in each of four 1L flasks (total volume 880 ml). Cells were incubated overnight at 36° C. with shaking (120 rpm). The next day the cells were counted and diluted to 3×10⁶ cells/ml in 232.5 ml per IL flask. Cells were transiently transfected as follows. 1000 μg plasmid DNA was diluted to 35 ml final volume with OptiMEM™ and gently mixed. 2.5 ml of Transporter 5 transfection reagent was diluted to a final volume of 35 ml with OptiMEM™ and gently mixed. The Diluted Transporter 5 was added to the diluted DNA, mixed, incubated at room temperature for 10 minutes and then 17.5 ml added dropwise to each 1 L flask while gently swirling the flask. Cells were placed back in the incubator, shaking for 4 days. A temperature shift to 33° C. was incorporated the day after transfection to increase protein yields.

Immobilized Metal Affinity Chromatography: Four mL of Ni²⁺ IMAC resin (Indigo, Cube Biotech cat #75103) per one liter of cell supernatant was equilibrated into IMAC wash buffer (20 mM Tris pH 8.0, 300 mM NaCl, 30 mM imidazole). Tris pH 8.0 was added at 50 mM per liter and NaCl was added to 300 mM per liter. Cell supernatants were batch bound overnight at 4° C. with stir bar agitation. After overnight incubation, cell supernatants were transferred to gravity columns and flow through was collected. Resin was then washed with 40 mL of IMAC wash buffer and flow through buffer was collected. Columns were sealed and eight mL of IMAC elution buffer (20 mM Tris pH 8.0, 300 mM NaCl, 500 mM imidazole) was added to each column and allowed to incubate for ten minutes. Column was unstopped and elution flow through was collected. Elution incubation was repeated twice. SDS-PAGE gel was done to confirm protein of interest was captured in elution fractions.

Differential Scanning Fluorimetery: Nano-DSF thermal ramp was used to estimate the Tonset and melting temperature (Tm) of antigen samples using SYPRO Orange Protein Gel Stain (Invitrogen) on an UNcle Nano-DSF (UNchained Laboratories). Antigen samples were normalized to a concentration of ˜1 mg/mL (or 0.3-0.45 mg/mL for low expressing constructs) by adding antigen samples to PCR tubes then adding buffer (20 mM Tris pH 7.4, 250 mM NaCl, 4% sucrose) to a final volume of 31.5 μL. SYPRO was diluted from 5000× to a 200× working stock solution by adding 4 μL of SYPRO to 96 μL of buffer. Then, 3.5 μL of the 200× stock solution was added to each PCR tube to bring SYPRO to 20×. Antigen sample dilutions with SYPRO were applied to quartz capillary cassettes (UNi, UNchained Laboratories) in triplicate and placed in the UNcle. Data were collected using a temperature ramp from 15° C. to 95° C. (holding samples at 15° C. for 300 seconds prior to data collection), collecting data at 1° C. increments. Improved Tonset and Tm were observed for all constructs compared to RSV/A.03 and RSV/B.002.

Accelerated Storage: Binding of RSV F specific antibodies were assessed on trimeric antigen-I53-50ΔΔcys fusion proteins following incubation of the tantigen samples at 4° C. or 40° C. for 7 days. Antibodies were normalized in concentration to 10 μg/mL in BLI assay buffer (PBS, 0.5% BSA, 0.05% Tween 20, pH 7.4) in a sufficient volume to load 80 μL per well of a black 384-well microplate (Thermo Scientific, 460518). Briefly, on an Octet rh16 instrument, Protein G biosensors (Sartorius, 18-5082) were dipped into assay buffer for 60 seconds to achieve a baseline. Next, the biosensors were dipped into each antibody for 60 seconds in order to immobilize the antibodies present but without reaching saturation, followed by an additional baseline step. The immobilized antibodies were allowed to associate with 80 μL of purified RSV antigen (normalized in concentration to 10 μg/mL) that was incubated at either 4° C. and 40° C. for 7 days for 120 seconds, and then the biosensors were dipped back into assay buffer for 120 seconds to observe any possible dissociation. The new designs have higher AM14 binding and lower 4D7 binding than the controls (RSV/A.03 and RSV/B.002) indicating less postfusion character and a more compact trimer. Decreased D25 and AM14 binding and increased 4D7 binding was observed for RSV/A.03 and RSV/B.002 following 7 days at 40° C. while binding of all Abs was unaffected by 7 days at 40° C. for the other constructs tested.

Assembly: Molar concentrations for RSV/B- or RSV/A trimers fused to I53-50ΔΔcys and I53-50B (second component, using the sequence of I53-50B.4PosT1, SEQ ID NO:46) were determined using UV-Vis spectroscopy. Absorbance values at 280 nm were collected and divided by calculated molar extinction coefficients (ExPASy). The assembly reaction to produce RSV/B antigen-bearing nanostructures was performed in vitro with the addition of components as follows: RSV F trimers fused to I53-50ΔΔcys were added to PCR tubes in 1.5× molar excess of I53-50B, then assembly buffer (20 mM Tris pH 7.4, 250 mM NaCl, 4% sucrose) was added to the sample in PCR tubes, and finally I53-50B was added to the reaction to bring the assemblies to a final concentration of 7.5 μM and a final volume of 47.5 μL. The reaction was incubated at ambient temperature for ˜30 minutes with gentle rocking prior to subsequent measurement by dynamic light scattering (DLS). All constructs were assembly competent under the conditions tested. Prior to nsEM analysis or immunogenicity studies, assembled nanostructures were further purified by size exclusion chromatography over a Superose 6 Increase 10/300 GL column into 20 mM Tris pH 7.4, 250 mM NaCl, 4% sucrose.

Dynamic Light Scattering: Dynamic Light Scattering (DLS) was used to measure hydrodynamic diameter (Dh) and polydispersity (% Pd) of RSV/B nanostructure assemblies on an UNcle Nano-DSF (UNchained Laboratories). The set up included increased viscosity due to 4% sucrose in the buffer that was accounted for by the UNcle Client Software in Dh measurements. RSV/B nanostructure assemblies were applied to quartz capillary cassettes (UNi, UNchained Laboratories) in triplicates and measured using the laser autoattenuation with 10 acquisitions per sample and 5 seconds per acquisition. Data were collected at 22° C. and all tested constructs resulted in monodisperse nanostructures of the expected size.

Electron Microscopy: For negative stain electron microscopy (nsEM), RSV F protein-nanostructure pre- and post-freeze samples were diluted to 75 μg/mL in 20 mM Tris pH 8.0, 150 mM NaCl, 5% Glycerol and 3 μL of sample was applied to the carbon side of two glow-discharged (Pelco EasiGLOW) thick carbon copper 400 mesh grids (EMS, CF400-Cu-TH). Samples were incubated on the grids for ˜1 minute, then blotted away using grade 1 filter paper (Whatman). Immediately, 3 μL of 0.75% UF stain was applied to the carbon side of the girds and incubated for ˜1 minute. The stain was blotted away using filter paper and the application of stain and blotting was repeated 2 more times. The grids were allowed to air dry for 5 minutes prior to imaging on a Talos L120C electron microscope at 57K magnification, Gatan camera. Micrographs shows correct self-assembly of monodisperse nanostructures.

Immunogenicity studies: Two immunogenicity studies were undertaken in 6-8-week-old, female BALB/c mice to evaluate the neutralizing antibody response elicited by RSV/A and RSV/B designs. In order to evaluate nanostructures based on RSV/A designs RSV/A.03, RSV/A.013, and RSV/A.023, mice were immunized with either 0.01 μg, 1 μg, or 5 μg of nanostructure protein. The 0.01 μg dose was adjuvanted with oil-in-water emulsion, AddaVax™, while the 1 μg and 5 μg doses were unadjuvanted. Mice were immunized on days 0 and 21 before being sacrificed on Day 35. Serum collected on Day 35 was used to perform a neutralization assay with the RSV/A Tracy strain. Nanostructures displaying RSV/B designs RSV/B.002, RSV/B.093, RSV/B.195, RSV/B.160, and RSV/B.171 were similarly evaluated. Mice were immunized on days 0 and 21 with either a 0.02 μg or 0.1 μg dose of nanostructure sample adjuvanted with AddaVax™. Serum samples collected during the terminal bleed on Day 35 were used to perform a neutralization assay with RSV/B strain 18537. Both the RSV/A and RSV/B neutralization assays were performed in Hep-2 cells. Two-fold serial dilutions of serum samples were prepared in 96-well plates. An equal volume of virus was added to each dilution and incubated for 1.5 hours before the addition of Hep-2 cells. Plates were incubated for 6-8 days before being fixed and stained with 10% neutral formalin and 0.01% crystal violet. Neutralizing antibody titers were defined as the final dilution at which there was a 50% reduction in viral cytopathic effect. Statistically significant differences between groups immunized with different designs at the same dose were determined by one-way ANOVA.

Cryo-electron microscopy: IMAC-purified trimeric RSV/A.023 sample was further purified over a Superdex 200 Increase 10/300 GL column unto 20 mM Tris pH 7.4, 250 mM NaCl, and further concentrated to 0.88 mg/mL prior to grid preparation. The concentrated sample was next frozen using a Quantifoil R 1.2/1.3 AU 300 holey grid. Data collection was performed using a Glacios 200 keV microscope equipped with a Falcon IV detector (0.91 Å/pixel). A C3-symmetric model of RSVA023 was rebuilt from PDB 4MMU using COOT. The final atomic structure was refined in Phenix and validated using MolProbity and the half-map cross validation method. Structural analysis was performed using COOT, Chimera and PyMol.

### Example 4. Diffusion Methods to Generate a C Terminus

Relaxed structures used as input for Rosetta Remodel were also used as input for RFdiffusion, except that only the C-terminal helices and neighboring residues were used as input for diffusion. This significantly reduces computational time. Positions with WT sequence identities from Rosetta Remodel were likewise preserved for diffusion. Unlike with Rosetta Remodel all structural data of subsequent residues was ignored. This is a limitation of RFdiffusion, not a scientific constraint placed on the design problem. Intra-protomer and inter-protomer weights were both set to 1, with a quadratic guide decay and a guide scale of 2. The non-standard weights Base_epoch8_ckpt.pt were applied and C3 symmetry was enforced. C-terminal lengths varied between 12 and 31 residues depending on the source virus, and 15 remodeled helices were generated for each length. Sequences were generated using Protein MPNN, with a sampling temperature of 0.4 and a negative bias against C (−10), and F, G, P, W, and Y (−1). 100 sequences were generated for each remodeled domain. The top 2% based on MPNN sample score were selected for computational characterization.

### Abbreviations

RSV Respiratory Syncytial Virus
REU Rosetta Energy Unit
PDB Protein Data Bank
EDTA ethylenediaminetetraacetic acid
DLS Dynamic Light Scattering
nsEM negative-stain electron microscopy
UNcle UNchained Laboratories
UNi UNchained Laboratories

### INCORPORATION BY REFERENCE

The entire disclosure of each of the patent document or scientific document referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. The scope of the invention is thus indicated by the appended claims rather than by the foregoing description, and all changes that come within the meaning and range of equivalency of the claims are intended to be embraced therein.

1. A recombinant polypeptide, comprising an engineered ectodomain of a Respiratory Syncytial Virus (RSV) fusion (F) protein, wherein the ectodomain comprises:
(a) a C-terminal helix-forming segment, between about residue 500 and about residue 530 relative to SEQ ID NO: 1, comprising one or more amino acid substitutions selected such that the segment forms a stable alpha-helical homotrimer;
(b) one, two, three or more amino acid substitutions at positions 140, 399, 400, 485, 486, 487, 488, 489, 494, or 498 relative to SEQ ID NO: 1;
(c) one, two, three or more amino acid substitutions at positions 56, 58, 154, 187, 296, or 298 relative to SEQ ID NO: 1;
(d) one, two, three or more amino acid substitutions at positions 75, 216, 218, or 219 relative to SEQ ID NO: 1;
(e) one, two, three or more amino acid substitutions at positions 92, 232, 235, 238, 249, 250, or 254 relative to SEQ ID NO: 1;
(f) one, two, three or more amino acid substitutions at positions 67, 137, or 339 relative to SEQ ID NO: 1;
(g) a substitution of a non-cleavable linker in place of a furin cleavage site at about residue 100 to about residue 140 relative to SEQ ID NO: 1; or
(h) any combination of (a)-(g).

2. The polypeptide of claim 1, wherein the ectodomain comprises (a) the C-terminal helix-forming segment, between about residue 500 and about residue 530 relative to SEQ ID NO: 1, comprising one or more amino acid substitutions selected such that the segment forms a stable alpha-helical homotrimer.

3. The polypeptide of claim 2, wherein the C-terminal helix-forming segment comprises between about 10 and about 30 residues.

4. (canceled)

5. The polypeptide of claim 2, wherein the segment comprises:
(a) an amino acid substitution at position F505 relative to SEQ ID NO: 1, wherein F is substituted with A, I, L, M, V, G, T;
(b) an amino acid substitution at position I506 relative to SEQ ID NO: 1, wherein I is substituted with any amino acids except P, preferably D, E, K, N, Q, R, S, T, Y or A, I, L, V;
(c) an amino acid substitution at position R507 relative to SEQ ID NO: 1, wherein R is substituted with any amino acids except P, preferably D, E, K, N, Q, R, S, T, Y or A, I, L, V;
(d) an amino acid substitution at position K508 relative to SEQ ID NO: 1, wherein K is substituted with K, Q, R, preferably A, V, T, I;
(e) an amino acid substitution at position S509 relative to SEQ ID NO: 1, wherein S is substituted with A, I, L, M, V, F, W, Y, G, T, preferably A, I, L, M, V;
(f) an amino acid substitution at position D510 relative to SEQ ID NO: 1, wherein D is substituted with any amino acids, preferably D, E, K, N, Q, R, S, T, Y;
(g) an amino acid substitution at position E511 relative to SEQ ID NO: 1, wherein E is substituted with any amino acids;
(h) an amino acid substitution at position L512 relative to SEQ ID NO: 1, wherein L is substituted with D, E, K, N, Q, R, S, T, Y, preferably A, I, L, M, V, F, W, Y, G, T;
(i) an amino acid substitution at position L513 relative to SEQ ID NO: 1, wherein L is substituted with any amino acids, preferably A, I, L, M, V, F, W, Y, G, more preferably D, E, K, N, Q, R, S, T, Y;
(j) an amino acid substitution at position H514 relative to SEQ ID NO: 1, wherein H is substituted with any amino acids except P, preferably D, E, K, N, Q, R, S, T, Y;
(k) an amino acid substitution at position N515 relative to SEQ ID NO: 1, wherein N is substituted with any amino acids except P, preferably A, I, L, M, V, F, W, Y, G;
(l) an amino acid substitution at position V516 relative to SEQ ID NO: 1, wherein V is substituted with A, I, L, M, V, F, W, Y, G, or T, S, K;
(m) an amino acid substitution at position N517 relative to SEQ ID NO: 1, wherein N is substituted with any amino acid except P, preferably D, E, K, N, Q, R, S, T, Y;
(n) an amino acid substitution at position T518 relative to SEQ ID NO: 1, wherein T is substituted with Any except P, preferably D, E, K, N, Q, R, S, T, Y;
(o) an amino acid substitution at position G519 relative to SEQ ID NO: 1, wherein G is substituted with any amino acid except P, preferably D, E, K, N, Q, R, S, T, Y; and/or
(p) any combination of (a)-(o).

6. The polypeptide of claim 2, wherein the segment comprises:
(a) an amino acid substitution at position L503 relative to SEQ ID NO: 1, wherein L is substituted with Q, V, K, R, N, L;
(b) an amino acid substitution at position A504 relative to SEQ ID NO: 1, wherein A is substituted with any amino acids except P, preferably S, T, L, A, Q, K, E, Y;
(c) an amino acid substitution at position F505 relative to SEQ ID NO: 1, wherein F is substituted with I, V, N, T, L;
(d) an amino acid substitution at position I506 relative to SEQ ID NO: 1, wherein I is substituted with any amino acids except P, preferably Q, N, K, R, V, S;
(e) an amino acid substitution at position R507 relative to SEQ ID NO: 1, wherein R is substituted with any amino acids except P, preferably A, N, K, E, D, Q;
(f) an amino acid substitution at position K508 relative to SEQ ID NO: 1, wherein K is substituted with T, M, V, R;
(g) an amino acid substitution at position S509 relative to SEQ ID NO: 1, wherein S is substituted with T, I, K, Q, M, E, V, S;
(h) an amino acid substitution at position D510 relative to SEQ ID NO: 1, wherein D is substituted with S, K, N, D, E;
(i) an amino acid substitution at position E511 relative to SEQ ID NO: 1, wherein E is substituted with R, S, E, K, A, T, L;
(j) an amino acid substitution at position L512 relative to SEQ ID NO: 1, wherein L is substituted with V, N, T, L;
(k) an amino acid substitution at position L513 relative to SEQ ID NO: 1, wherein L is substituted with D, T, H, K, E, N, R;
(l) an amino acid substitution at position H514 relative to SEQ ID NO: 1, wherein H is substituted with A, N, E, S, V, K, T, D;
(m) an amino acid substitution at position N515 relative to SEQ ID NO: 1, wherein N is substituted with I, E, L, T, Q;
(n) an amino acid substitution at position V516 relative to SEQ ID NO: 1, wherein V is substituted with E, I, K, N, R, Q;
(o) an amino acid substitution at position N517 relative to SEQ ID NO: 1, wherein N is substituted with A, S, K, E, R;
(p) an amino acid substitution at position T518 relative to SEQ ID NO: 1, wherein T is substituted with K, S, Q, R, D, E;
(q) an amino acid substitution at position G519 relative to SEQ ID NO: 1, wherein G is substituted with V, L, I;
(r) an amino acid substitution at position 1520 relative to SEQ ID NO: 1, wherein I is substituted with K, Q, E, N, T;
(s) an amino acid substitution at position P521 relative to SEQ ID NO: 1, wherein P is substituted with H, D, E, K, R, N, Q;
(t) an amino acid substitution at position E522 relative to SEQ ID NO: 1, wherein E is substituted with L, R, I, V;
(u) an amino acid substitution at position A523 relative to SEQ ID NO: 1, wherein A is substituted with E, V, L, K, R I;
(v) an amino acid substitution at position P524 relative to SEQ ID NO: 1, wherein P is substituted with A, K, T, E, R;
(w) an amino acid substitution at position R525 relative to SEQ ID NO: 1, wherein R is substituted with H, R, S, L, N, E, D;
(x) an amino acid substitution at position D526 relative to SEQ ID NO: 1, wherein D is substituted with I, L, V, R;
(y) an amino acid substitution at position G527 relative to SEQ ID NO: 1, wherein G is substituted with E, K, Q, D;
(z) an amino acid substitution at position Q528 relative to SEQ ID NO: 1, wherein Q is substituted with D, K, S, R, A;
(aa) an amino acid substitution at position A529 relative to SEQ ID NO: 1, wherein A is substituted with T, L;
(ab) an amino acid substitution at position Y530 relative to SEQ ID NO: 1, wherein Y is substituted with L, E, T;
(ac) an amino acid substitution at position V531 relative to SEQ ID NO: 1, wherein V is substituted with A, R, K;
(ad) an amino acid substitution at position R532 relative to SEQ ID NO: 1, wherein R is substituted with V, A; and/or
(aa) an amino acid substitution at position A529 relative to SEQ ID NO: 1, wherein A is substituted with T, L;
(ab) an amino acid substitution at position Y530 relative to SEQ ID NO: 1, wherein Y is substituted with L, E, T;
(ac) an amino acid substitution at position V531 relative to SEQ ID NO: 1, wherein V is substituted with A, R, K;
(ad) an amino acid substitution at position R532 relative to SEQ ID NO: 1, wherein R is substituted with V, A; and/or
(ae) any combination of (a)-(ad).

7. (canceled)

8. The polypeptide of claim 2, wherein the segment comprises the polypeptide sequence NQSREIIRAINIVRKIASEK (SEQ ID NO: 10), NQSALWLEAAKYVKQAREKS (SEQ ID NO: 11), NQSAKNAEAAKIAEETKRKD (SEQ ID NO: 12), or NQSRETAKAVSAVK (SEQ ID NO: 75), or a polypeptide sequence having between 1 and 5 amino acid substitutions thereto.

9.-10. (canceled)

11. The polypeptide of claim 1, wherein the ectodomain comprises (b) one, two, three or more amino acid substitutions at positions 140, 399, 400, 485, 486, 487, 488, 489, 494, or 498 relative to SEQ ID NO: 1.

12. The polypeptide of claim 1, wherein the ectodomain comprises one or more of the following sets of amino acid substitutions relative to SEQ ID NO: 1:
E487R+K498A;
E487R+K498E;
E487K+K498E;
D486A+E487R+K498A;
D486Q+E487R+K498A;
D486E+E487A+D489A+T400D;
D486A+E487M+K498A;
E487Q;
D486S;
F488W+D489A+T400D+E487R+K498A;
F488W+D489A+T400D+E487R+K498A+D486A;
F488W+D489A+T400D+E487R+K498A+T249P;
F140W+D489A+T400D+E487R+K498A;
Q494I+S485I+K399A+487R+498A;
Q494M+S485I+K399A; D486A+487M+498A;
Q494L+S485A+K399V+D486A+487M+498A;
Q494M+S485A+K399V+D486A+487M+498A;
Q494A+S485F+K399V+D486A+487M+498Y;
D489A+T400D+E487R+K498A; or
D489A+T400D.

13.-17. (canceled)

18. The polypeptide of claim 1, wherein the polypeptide comprises, C-terminal to the ectodomain, a heterologous multimerization domain, wherein the multimerization domain comprises a polypeptide sequence at least 70%, at least 80%, at least 90%, at least 91%, at least 92%, at least 93%, at least 94%, at least 95%, at least 96%, at least 97%, at least 98%, at least 99%, or 100% identical to I53-50A (SEQ ID NO: 19) or I53-50A ΔCys (SEQ ID NO: 64).

19. The polypeptide of claim 1, wherein the ectodomain comprises the amino acid substitutions S155C, S290C, S190F, and V207L.

20. The polypeptide of claim 1, wherein the ectodomain comprises a polypeptide sequence at least 70%, at least 80%, at least 90%, at least 91%, at least 92%, at least 93%, at least 94%, at least 95%, at least 96%, at least 97%, at least 98%, at least 99%, or 100% identical to
(i) QNITEEFYQSTCSAVSRGYLSALRTGWYTSVITIELSNIKETKCNGTDTKVKLIKQELDKY KNAVTELQLLMQNTPAVNNRARREAPQYMNYTINTTKNLNVSISKKRKRRFLGFLLGVGSA IASGIAVCKVLHLEGEVNKIKNALQLTNKAVVSLSNGVSVLTFRVLDLKNYINNQLLPMLN RQSCRISNIETVIEFQQKNSRLLEITREFSVNAGVTTPLSTYMLTNSELLSLINDMPITND QKKLMSSNVQIVRQQSYSIMCIIKEEVLAYVVQLPIYGVIDTPCWKLHTSPLCTTNIKEGS NICLTRTDRGWYCDNAGSVSFFPQADTCKVQSNRVFCDTMNSLTLPSEVSLCNTDIFNSKY DCKIMTSKTDISSSVITSLGAIVSCYGKTKCTASNKNRGIIKTFSNGCDYVSNKGVDTVSV GNTLYYVNKLEGKNLYVKGEPIINYYDPLVFPSDEFDASISQVNEKINQSXXXXXXXXXXX XXXXXX (SEQ ID NO: 6), optionally lacking a p27 peptide shown in bold,
(ii) QNITEEFYQSTCSAVSKGYLSALRTGWYTSVITIELSNIKENKCNGTDAKVKLIKQELDK YKNAVTELQLLMQSTPATNNRARRELPRFMNYTLNNAKKTNVTLSKKRKRRFLGFLLGVGS AIASGVAVCKVLHLEGEVNKIKSALLSTNKAVVSLSNGVSVLTFKVLDLKNYIDKQLLPIL NKQSCSISNIETVIEFQQKNNRLLEITREFSVNAGVTTPVSTYMLTNSELLSLINDMPITN DQKKLMSNNVQIVRQQSYSIMCIIKEEVLAYVVQLPLYGVIDTPCWKLHTSPLCTTNTKEG SNICLTRTDRGWYCDNAGSVSFFPQAETCKVQSNRVFCDTMNSLTLPSEVNLCNVDIFNPK YDCKIMTSKTDVSSSVITSLGAIVSCYGKTKCTASNKNRGIIKTFSNGCDYVSNKGVDTVS VGNTLYYVNKQEGKSLYVKGEPIINFYDPLVFPSDEFDASISQVNEKINQSXXXXXXXXXX XXXXXXX (SEQ ID NO: 7), optionally lacking a p27 peptide shown in bold,
(iii) QNITEEFYQSTCSAVSRGYLSALRTGWYTSVITIELSNIKETKCNGTDTKVKLIKQELDK YKNAVTELQLLMQNTPAVNNRARREAPQYMNYTINTTKNLNVSISKKRKRRFLGFLLGVGS AIASGIAVCKVLHLEGEVNKIKNALQLTNKAVVSLSNGVSVLTFRVLDLKNYINNQLLPML NRQSCRISNIETVIEFQQKNSRLLEITREFSVNAGVTTPLSTYMLTNSELLSLINDMPITN DQKKLMSSNVQIVRQQSYSIMCIIKEEVLAYVVQLPIYGVIDTPCWKLHTSPLCTTNIKEG SNICLTRTDRGWYCDNAGSVSFFPQADTCKVQSNRVFCDTMNSLTLPSEVSLCNTDIFNSK YDCKIMTSKTDISSSVITSLGAIVSCYGKTKCTASNKNRGIIKTFSNGCDYVSNKGVDTVS VGNTLYYVNKLEGKNLYVKGEPIINYYDPLVFPSDEFDASISQVNEKINQSREIIRAINIV RKIASEK (SEQ ID NO: 8), optionally lacking a p27 peptide shown in bold, or
(iv) QNITEEFYQSTCSAVSKGYLSALRTGWYTSVITIELSNIKENKCNGTDAKVKLIKQELDK YKNAVTELQLLMQSTPATNNRARRELPRFMNYTLNNAKKTNVTLSKKRKRRFLGFLLGVGS AIASGVAVCKVLHLEGEVNKIKSALLSTNKAVVSLSNGVSVLTFKVLDLKNYIDKQLLPIL NKQSCSISNIETVIEFQQKNNRLLEITREFSVNAGVTTPVSTYMLTNSELLSLINDMPITN DQKKLMSNNVQIVRQQSYSIMCIIKEEVLAYVVQLPLYGVIDTPCWKLHTSPLCTTNTKEG SNICLTRTDRGWYCDNAGSVSFFPQAETCKVQSNRVFCDTMNSLTLPSEVNLCNVDIFNPK YDCKIMTSKTDVSSSVITSLGAIVSCYGKTKCTASNKNRGIIKTFSNGCDYVSNKGVDTVS VGNTLYYVNKQEGKSLYVKGEPIINFYDPLVFPSDEFDASISQVNEKINQSREIIRAINIV RKIASEK (SEQ ID NO: 9), optionally lacking a p27 peptide shown in bold.

21.-24. (canceled)

25. A trimeric protein complex comprising a polypeptide according to claim 1.

26.-29. (canceled)

29. A protein nanostructure comprising a trimeric component comprising a polypeptide according to claim 1.

30. The protein nanostructure of claim 29, wherein the protein nanostructure is a two-component nanostructure comprising the first, trimeric component and a second, pentameric component, wherein the first, trimeric component comprises an engineered ectodomain of a Respiratory Syncytial Virus (RSV) fusion (F) polypeptide and an I53-50A polypeptide.

31.-32. (canceled)

33. The protein nanostructure of claim 30, wherein the protein nanostructure is a two-component nanostructure comprising:
(i) the first, trimeric component, wherein the first trimeric component comprises an engineered ectodomain of a RSV F polypeptide comprising an amino acid substitutions at position S155C, S290C, S190F, and V207L relative to SEQ ID NO: 1 and a C-terminal helix-forming segment comprising the polypeptide sequence NQSREIIRAINIVRKIASEK (SEQ ID NO: 10); and
a multimerization domain comprising a polypeptide sequence at least 70%, at least 80%, at least 90%, at least 91%, at least 92%, at least 93%, at least 94%, at least 95%, at least 96%, at least 97%, at least 98%, at least 99%, or 100% identical to I53-50A (SEQ ID NO: 19) or I53-50A ΔCys (SEQ ID NO: 64); and/or
the second pentameric component, wherein the pentameric component comprises a polypeptide sequence at least 70%, at least 80%, at least 90%, at least 91%, at least 92%, at least 93%, at least 94%, at least 95%, at least 96%, at least 97%, at least 98%, at least 99%, or 100% identical to SEQ ID NO: 20 or 71;
(ii) the first, trimeric component, wherein the first trimeric component comprises an e engineered ectodomain of a RSV F polypeptide comprising an amino acid substitutions at position S155C, S290C, S190F, V207L, D489A, T400D, E487R, and K498A relative to SEQ ID NO: 1 and a C-terminal helix-forming segment comprising the polypeptide sequence NQSREIIRAINIVRKIASEK (SEQ ID NO: 10); and
a multimerization domain comprising a polypeptide sequence at least 70%, at least 80%, at least 90%, at least 91%, at least 92%, at least 93%, at least 94%, at least 95%, at least 96%, at least 97%, at least 98%, at least 99%, or 100% identical to I53-50A (SEQ ID NO: 19) or I53-50A ΔCys (SEQ ID NO: 64); and/or
the second pentameric component, wherein the pentameric component comprises a polypeptide sequence at least 70%, at least 80%, at least 90%, at least 91%, at least 92%, at least 93%, at least 94%, at least 95%, at least 96%, at least 97%, at least 98%, at least 99%, or 100% identical to SEQ ID NO: 20 or 71;
(iii) the first, trimeric component, wherein the first trimeric component comprises an engineered ectodomain of a RSV F polypeptide comprising an amino acid substitutions at position S155C, S290C, S190F, V207L, F488W, D489A, T400D, E487R, K498A, and T249P relative to SEQ ID NO: 1 and a C-terminal helix-forming segment comprising the polypeptide sequence NQSREIIRAINIVRKIASEK (SEQ ID NO: 10); and
a multimerization domain comprising a polypeptide sequence at least 70%, at least 80%, at least 90%, at least 91%, at least 92%, at least 93%, at least 94%, at least 95%, at least 96%, at least 97%, at least 98%, at least 99%, or 100% identical to I53-50A (SEQ ID NO: 19) or I53-50A ΔCys (SEQ ID NO: 64); and/or
the second pentameric component, wherein the pentameric component comprises a polypeptide sequence at least 70%, at least 80%, at least 90%, at least 91%, at least 92%, at least 93%, at least 94%, at least 95%, at least 96%, at least 97%, at least 98%, at least 99%, or 100% identical to SEQ ID NO: 20 or 71; or
(iv) the first, trimeric component, wherein the first trimeric component comprises an engineered ectodomain of a RSV F polypeptide comprising an amino acid substitutions at position S155C, S290C, S190F, V207L, F488W, D489A, T400D, E487R, K498A, and D486A relative to SEQ ID NO: 1 and a C-terminal helix-forming segment comprising the polypeptide sequence NQSREIIRAINIVRKIASEK (SEQ ID NO: 10); and
a multimerization domain comprising a polypeptide sequence at least 70%, at least 80%, at least 90%, at least 91%, at least 92%, at least 93%, at least 94%, at least 95%, at least 96%, at least 97%, at least 98%, at least 99%, or 100% identical to I53-50A (SEQ ID NO: 19) or I53-50A ΔCys (SEQ ID NO: 64); and/or
the second pentameric component, wherein the pentameric component comprises a polypeptide sequence at least 70%, at least 80%, at least 90%, at least 91%, at least 92%, at least 93%, at least 94%, at least 95%, at least 96%, at least 97%, at least 98%, at least 99%, or 100% identical to SEQ ID NO: 20 or 71.

34.-37. (canceled)

38. The protein nanostructure of claim 30, wherein the pentameric component comprises a polypeptide sequence at least 70%, at least 80%, at least 90%, at least 91%, at least 92%, at least 93%, at least 94%, at least 95%, at least 96%, at least 97%, at least 98%, at least 99%, or 100% identical to any one or more of SEQ ID NOs: 20, 44, 45, 52, 71, 73, 74.

39. A polynucleotide encoding the polypeptide of claim 1.

40. A delivery vehicle comprising a polynucleotide of claim 39, wherein the delivery vehicle is a lipid nanoparticle (LNP).

41. A pharmaceutical composition comprising a polypeptide according to claim 1.

42. (canceled)

43. A method of vaccinating a subject, generating an immune response in a subject, and/or treating or preventing RSV disease in a subject, the method comprising administering to the subject a pharmaceutical composition according to claim 41.

44.-50. (canceled)


SEQUENCE LISTING (320 sequences)

SEQ ID NO: 1 - protein, 574 aa, synthetic construct
MELLIHRSSAIFLTLAINALYLTSSQNITEEFYQSTCSAVSRGYLSALRTGWYTSVITIELSNIKETKCNGTDTKVKLIKQELDKYKNAVTELQLLMQNTPAVNNRARREAPQYMNYTINTTKNLNVSISKKRKRRFLGFLLGVGSAIASGIAVSKVLHLEGEVNKIKNALQLTNKAVVSLSNGVSVLTSRVLDLKNYINNQLLPMVNRQSCRISNIETVIEFQQKNSRLLEITREFSVNAGVTTPLSTYMLTNSELLSLINDMPITNDQKKLMSSNVQIVRQQSYSIMSIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSDEFDASISQVNEKINQSLAFIRRSDELLHNVNTGKSTTNIMITAITIVIIVVLLSLIAIGLLLYCKAKNTPVTLSKDQLSGINNIAFSK

SEQ ID NO: 2 - protein, 574 aa, synthetic construct
MELLIHRSSAIFLTLAINALYLTSSQNITEEFYQSTCSAVSRGYLSALRTGWYTSVITIELSNIKETKCNGTDTKVKLIKQELDKYKNAVTELQLLMQNTPAVNNRARREAPQYMNYTINTTKNLNVSISKKRKRRFLGFLLGVGSAIASGIAVCKVLHLEGEVNKIKNALQLTNKAVVSLSNGVSVLTCRVLDLKNYINNQLLPMLNRQSCRISNIETVIEFQQKNSRLLEITREFSVNAGVTTPLSTYMLTNSELLSLINDMPITNDQKKLMSSNVQIVRQQSYSIMCIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSDEFDASISQVNEKINQSLAFIRRSDELLHNVNTGKSTTNIMITAITIVIIVVLLSLIAIGLLLYCKAKNTPVTLSKDQLSGINNIAFSK

SEQ ID NO: 3 - protein, 549 aa, synthetic construct
QNITEEFYQSTCSAVSRGYLSALRTGWYTSVITIELSNIKETKCNGTDTKVKLIKQELDKYKNAVTELQLLMQNTPAVNNRARREAPQYMNYTINTTKNLNVSISKKRKRRFLGFLLGVGSAIASGIAVSKVLHLEGEVNKIKNALQLTNKAVVSLSNGVSVLTSRVLDLKNYINNQLLPMVNRQSCRISNIETVIEFQQKNSRLLEITREFSVNAGVTTPLSTYMLTNSELLSLINDMPITNDQKKLMSSNVQIVRQQSYSIMSIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSDEFDASISQVNEKINQSLAFIRRSDELLHNVNTGKSTTNIMITAITIVIIVVLLSLIAIGLLLYCKAKNTPVTLSKDQLSGINNIAFSK

SEQ ID NO: 4 - protein, 549 aa, synthetic construct
QNITEEFYQSTCSAVSRGYLSALRTGWYTSVITIELSNIKETKCNGTDTKVKLIKQELDKYKNAVTELQLLMQNTPAVNNRARREAPQYMNYTINTTKNLNVSISKKRKRRFLGFLLGVGSAIASGIAVCKVLHLEGEVNKIKNALQLTNKAVVSLSNGVSVLTCRVLDLKNYINNQLLPMLNRQSCRISNIETVIEFQQKNSRLLEITREFSVNAGVTTPLSTYMLTNSELLSLINDMPITNDQKKLMSSNVQIVRQQSYSIMCIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSDEFDASISQVNEKINQSLAFIRRSDELLHNVNTGKSTTNIMITAITIVIIVVLLSLIAIGLLLYCKAKNTPVTLSKDQLSGINNIAFSK

SEQ ID NO: 5 - protein, 488 aa, synthetic construct
QNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELL

SEQ ID NO: 6 - protein, 494 aa, synthetic construct
  VARIANT 85..111: note = Optionally lacking p27 peptide residues 85-111
QNITEEFYQSTCSAVSRGYLSALRTGWYTSVITIELSNIKETKCNGTDTKVKLIKQELDKYKNAVTELQLLMQNTPAVNNRARREAPQYMNYTINTTKNLNVSISKKRKRRFLGFLLGVGSAIASGIAVCKVLHLEGEVNKIKNALQLTNKAVVSLSNGVSVLTFRVLDLKNYINNQLLPMLNRQSCRISNIETVIEFQQKNSRLLEITREFSVNAGVTTPLSTYMLTNSELLSLINDMPITNDQKKLMSSNVQIVRQQSYSIMCIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSDEFDASISQVNEKINQSXXXXXXXXXXXXXXXXX

SEQ ID NO: 7 - protein, 494 aa, synthetic construct
  VARIANT 85..111: note = Optionally lacking p27 peptide residues 85-111
QNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSXXXXXXXXXXXXXXXXX

SEQ ID NO: 8 - protein, 494 aa, synthetic construct
  VARIANT 85..111: note = Optionally lacking p27 peptide residues 85-111
QNITEEFYQSTCSAVSRGYLSALRTGWYTSVITIELSNIKETKCNGTDTKVKLIKQELDKYKNAVTELQLLMQNTPAVNNRARREAPQYMNYTINTTKNLNVSISKKRKRRFLGFLLGVGSAIASGIAVCKVLHLEGEVNKIKNALQLTNKAVVSLSNGVSVLTFRVLDLKNYINNQLLPMLNRQSCRISNIETVIEFQQKNSRLLEITREFSVNAGVTTPLSTYMLTNSELLSLINDMPITNDQKKLMSSNVQIVRQQSYSIMCIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSDEFDASISQVNEKINQSREIIRAINIVRKIASEK

SEQ ID NO: 9 - protein, 494 aa, synthetic construct
  VARIANT 85..111: note = Optionally lacking p27 peptide residues 85-111
QNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSREIIRAINIVRKIASEK

SEQ ID NO: 10 - protein, 20 aa, synthetic construct
NQSREIIRAINIVRKIASEK

SEQ ID NO: 11 - protein, 20 aa, synthetic construct
NQSALWLEAAKYVKQAREKS

SEQ ID NO: 12 - protein, 20 aa, synthetic construct
NQSAKNAEAAKIAEETKRKD

SEQ ID NO: 13 - protein, 206 aa, synthetic construct
EGMDPLAVLAESRLLPLLTVRGGEDLAGLATVLELMGVGALEITLRTEKGLEALKALRKSGLLLGAGTVRSPKEAEAALEAGAAFLVSPGLLEEVAALAQARGVPYLPGVLTPTEVERALALGLSALKFFPAEPFQGVRVLRAYAEVFPEVRFLPTGGIKEEHLPHYAALPNLLAVGGSWLLQGDLAAVMKKVKAAKALLSPQAPG

SEQ ID NO: 14 - protein, 155 aa, synthetic construct
TKKVGIVDTTFARVDMAEAAIRTLKALSPNIKIIRKTVPGIKDLPVACKKLLEEEGCDIVMALGMPGKAEKDKVCAHEASLGLMLAQLMTNKHIIEVFVHEDEAKDDDELDILALVRAIEHAANVYYLLFKPEYLTRMAGKGLRQGREDAGPARE

SEQ ID NO: 15 - protein, 155 aa, synthetic construct
TKKVGIVDTTFARVDMASAAILTLKMESPNIKIIRKTVPGIKDLPVACKKLLEEEGCDIVMALGMPGKAEKDKVCAHEASLGLMLAQLMTNKHIIEVFVHEDEAKDDAELKILAARRAIEHALNVYYLLFKPEYLTRMAGKGLRQGFEDAGPARE

SEQ ID NO: 16 - protein, 208 aa, synthetic construct
STINNQLKALKVIPVIAIDNAEDIIPLGKVLAENGLPAAEITFRSSAAVKAIMLLRSAQPEMLIGAGTILNGVQALAAKEAGATFVVSPGFNPNTVRACQIIGIDIVPGVNNPSTVEAALEMGLTTLKFFPAEASGGISMVKSLVGPYGDIRLMPTGGITPSNIDNYLAIPQVLACGGTWMVDKKLVTNGEWDEIARLTREIVEQVNP

SEQ ID NO: 17 - protein, 113 aa, synthetic construct
PIFTLNTNIKATDVPSDFLSLTSRLVGLILSKPGSYVAVHINTDQQLSFGGSTNPAAFGTLMSIGGIEPSKNRDHSAVLFDHLNAMLGIPKNRMYIHFVNLNGDDVGWNGTTF

SEQ ID NO: 18 - protein, 156 aa, synthetic construct
NQHSHKDYETVRIAVVRARWHADIVDACVEAFEIAMAAIGGDRFAVDVFDVPGAYEIPLHARTLAETGRYGAVLGTAFVVNGGIYRHEFVASAVIDGMMNVQLSTGVPVLSAVLTPHRYRDSAEHHRFFAAHFAVKGVEAARACIEILAAREKIAA

SEQ ID NO: 19 - protein, 202 aa, synthetic construct
EELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPDEVREKAKAFVEKIRGCTE

SEQ ID NO: 20 - protein, 156 aa, synthetic construct
NQHSHKDYETVRIAVVRARWHAEIVDACVSAFEAAMADIGGDRFAVDVFDVPGAYEIPLHARTLAETGRYGAVLGTAFVVNGGIYRHEFVASAVIDGMMNVQLSTGVPVLSAVLTPHRYRDSDAHTLLFLALFAVKGMEAARACVEILAAREKIAA

SEQ ID NO: 21 - protein, 176 aa, synthetic construct
FTKSGDDGNTNVINKRVGKDSPLVNFLGDLDELNSFIGFAISKIPWEDMKKDLERVQVELFEIGEDLSTQSSKKKIDESYVLWLLAATAIYRIESGPVKLFVIPGGSEEASVLHVTRSVARRVERNAVKYTKELPEINRMIIVYLNRLSSLLFAMALVANKRRNQSEKIYEIGKSW

SEQ ID NO: 22 - protein, 156 aa, synthetic construct
NQHSHKDYETVRIAVVRARWHADIVDQCVRAFEEAMADAGGDRFAVDVFDVPGAYEIPLHARTLAETGRYGAVLGTAFVVNGGIYRHEFVASAVIDGMMNVQLSTGVPVLSAVLTPHRYRSSREHHEFFREHFMVKGVEAAAACITILAAREKIAA

SEQ ID NO: 23 - protein, 200 aa, synthetic construct
GHTKGPTPQQHDGSALRIGIVHARWNKTIIMPLLIGTIAKLLECGVKASNIVVQSVPGSWELPIAVQRLYSASQLQTPSSGPSLSAGDLLGSSTTDLTALPTTTASSTGPFDALIAIGVLIKGETMHFEYIADSVSHGLMRVQLDTGVPVIFGVLTVLTDDQAKARAGVIEGSHNHGEDWGLAAVEMGVRRRDWAAGKTE

SEQ ID NO: 24 - protein, 236 aa, synthetic construct
YEVDHADVYDLFYLGRGKDYAAEASDIADLVRSRTPEASSLLDVACGTGTHLEHFTKEFGDTAGLELSEDMLTHARKRLPDATLHQGDMRDFQLGRKFSAVVSMFSSVGYLKTVAELGAAVASFAEHLEPGGVVVVEPWWFPETFADGWVSADVVRRDGRTVARVSHSVREGNATRMEVHFTVADPGKGVRHFSDVHLITLFHQREYEAAFMAAGLRVEYLEGGPSGRGLFVGVPA

SEQ ID NO: 25 - protein, 137 aa, synthetic construct
GMKEKFVLIITHGDFGKGLLSGAEVIIGKQENVHTVGLNLGDNIEKVAKEVMRIIIAKLAEDKEIIIVVDLFGGSPFNIALEMMKTFDVKVITGINMPMLVELLTSINVYDTTELLENISKIGKDGIKVIEKSSLKM

SEQ ID NO: 26 - protein, 153 aa, synthetic construct
KYDGSKLRIGILHARWNLEIIAALVAGAIKRLQEFGVKAENIIIETVPGSFELPYGSKLFVEKQKRLGKPLDAIIPIGVLIKGSTMHFEYICDSTTHQLMKLNFELGIPVIFGVLTCLTDEQAEARAGLIEGKMHNHGEDWGAAAVEMATKFN

SEQ ID NO: 27 - protein, 163 aa, synthetic construct
AVKGLGEVDQKYDGSKLRIGILHARWNRKIILALVAGAVLRLLEFGVKAENIIIETVPGSFELPYGSKLFVEKQKRLGKPLDAIIPIGVLIKGSTMHFEYICDSTTHQLMKLNFELGIPVIFGVLTCLTDEQAEARAGLIEGKMHNHGEDWGAAAVEMATKFN

SEQ ID NO: 28 - protein, 174 aa, synthetic construct
GANWYLDNESSRLSFTSTKNADIAEVHRFLVLHGKVDPKGLAEVEVETESISTGIPLRDMLLRVLVFQVSKFPVAQINAQLDMRPINNLAPGAQLELRLPLTVSLRGKSHSYNAELLATRLDERRFQVVTLEPLVIHAQDFDMVRAFNALRLVAGLSAVSLSVPVGAVLIFTAR

SEQ ID NO: 29 - protein, 207 aa, synthetic construct
TDYIRDGSAIKALSFAIILAEADLRHIPQDLQRLAVRVIHACGMVDVANDLAFSEGAGKAGRNALLAGAPILCDARMVAEGITRSRLPADNRVIYTLSDPSVPELAKKIGNTRSAAALDLWLPHIEGSIVAIGNAPTALFRLFELLDAGAPKPALIIGMPVGFVGAAESKDELAANSRGVPYVIVRGRRGGSAMTAAAVNALASERE

SEQ ID NO: 30 - protein, 127 aa, synthetic construct
ITVFGLKSKLAPRREKLAEVIYSSLHLGLDIPKGKHAIRFLCLEKEDFYYPFDRSDDYTVIEINLMAGRSEETKMLLIFLLFIALERKLGIRAHDVEITIKEQPAHCWGFRGRTGDSARDLDYDIYV

SEQ ID NO: 31 - protein, 234 aa, synthetic construct
GSDLQKLQRFSTCDISDGLLNVYNIPTGGYFPNLTAISPPQNSSIVGTAYTVLFAPIDDPRPAVNYIDSVPPNSILVLALEPHLQSQFHPFIKITQAMYGGLMSTRAQYLKSNGTVVFGRIRDVDEHRTLNHPVFAYGVGSCAPKAVVKAVGTNVQLKILTSDGVTQTICPGDYIAGDNNGIVRIPVQETDISKLVTYIEKSIEVDRLVSEAIKNGLPAKAAQTARRMVLKDYI

SEQ ID NO: 32 - protein, 161 aa, synthetic construct
SGMRVYLGADHAGYELKQAIIAFLKMTGHEPIDCGALRYDADDDYPAFCIAAATRTVADPGSLGIVLGGSGNGEQIAANKVPGARCALAWSVQTAALAREHNNAQLIGIGGRMHTLEEALRIVKAFVTTPWSKAQRHQRRIDILAEYERTHEAPPVPGAPA

SEQ ID NO: 33 - protein, 156 aa, synthetic construct
GDDARIAAIGDVDELNSQIGVLLAEPLPDDVRAALSAIQHDLFDLGGELCIPGHAAITEDHLLRLALWLVHYNGQLPPLEEFILPGGARGAALAHVCRTVCRRAERSIKALGASEPLNIAPAAYVNLLSDLLFVLARVLNRAAGGADVLWDRTRAH

SEQ ID NO: 34 - protein, 156 aa, synthetic construct
ILSAEQSFTLRHPHGQAAALAFVREPAAALAGVQRLRGLDSDGEQVWGELLVRVPLLGEVDLPFRSEIVRTPQGAELRPLTLTGERAWVAVSGQATAAEGGEMAFAFQFQAHLATPEAEGEGGAAFEVMVQAAAGVTLLLVAMALPQGLAAGLPPA

SEQ ID NO: 35 - protein, 155 aa, synthetic construct
TKKVGIVDTTFARVDMASAAILTLKMESPNIKIIRKTVPGIKDLPVACKKLLEEEGCDIVMALGMPGKKEKDKVCAHEASLGLMLAQLMTNKHIIEVFVHEDEAKDDAELKILAARRAIEHALNVYYLLFKPEYLTRMAGKGLRQGFEDAGPARE

SEQ ID NO: 36 - protein, 208 aa, synthetic construct
DDINNQLKRLKVIPVIAIDNAEDIIPLGKVLAENGLPAAEITFRSSAAVKAIMLLRSAQPEMLIGAGTILNGVQALAAKEAGADFVVSPGFNPNTVRACQIIGIDIVPGVNNPSTVEQALEMGLTTLKFFPAEASGGISMVKSLVGPYGDIRLMPTGGITPDNIDNYLAIPQVLACGGTWMVDKKLVRNGEWDEIARLTREIVEQVNP

SEQ ID NO: 37 - protein, 113 aa, synthetic construct
PIFTLNTNIKADDVPSDFLSLTSRLVGLILSKPGSYVAVHINTDQQLSFGGSTNPAAFGTLMSIGGIEPDKNRDHSAVLFDHLNAMLGIPKNRMYIHFVNLNGDDVGWNGTTF

SEQ ID NO: 38 - protein, 113 aa, synthetic construct
PIFTLNTNIKADDVPSDFLSLTSRLVGLILSEPGSYVAVHINTDQQLSFGGSTNPAAFGTLMSIGGIEPDKNEDHSAVLFDHLNAMLGIPKNRMYIHFVDLDGDDVGWNGTTF

SEQ ID NO: 39 - protein, 156 aa, synthetic construct
NQHSHKDHETVRIAVVRARWHADIVDACVEAFEIAMAAIGGDRFAVDVFDVPGAYEIPLHARTLAETGRYGAVLGTAFVVNGGIYRHEFVASAVIDGMMNVQLDTGVPVLSAVLTPHRYRDSDEHHRFFAAHFAVKGVEAARACIEILNAREKIAA

SEQ ID NO: 40 - protein, 156 aa, synthetic construct
NQHSHKDHETVRIAVVRARWHADIVDACVEAFEIAMAAIGGDRFAVDVFDVPGAYEIPLHARTLAETGRYGAVLGTAFVVDGGIYDHEFVASAVIDGMMNVQLDTGVPVLSAVLTPHEYEDSDEDHEFFAAHFAVKGVEAARACIEILNAREKIAA

SEQ ID NO: 41 - protein, 202 aa, synthetic construct
EELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHDILKLFPGEVVGPQFVKAMKGPFPNVKFVPTGGVNLDNVCEWFKAGVLAVGVGDALVKGDPDEVREKAKKFVEKIRGCTE

SEQ ID NO: 42 - protein, 202 aa, synthetic construct
EELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHDILKLFPGEVVGPEFVEAMKGPFPNVKFVPTGGVDLDDVCEWFDAGVLAVGVGDALVEGDPDEVREDAKEFVEEIRGCTE

SEQ ID NO: 43 - protein, 202 aa, synthetic construct
EELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHDILKLFPGEVVGPQFVKAMKGPFPNVKFVPTGGVNLDNVCKWFKAGVLAVGVGKALVKGKPDEVREKAKKFVKKIRGCTE

SEQ ID NO: 44 - protein, 156 aa, synthetic construct
NQHSHKDHETVRIAVVRARWHAEIVDACVSAFEAAMRDIGGDRFAVDVFDVPGAYEIPLHARTLAETGRYGAVLGTAFVVNGGIYRHEFVASAVIDGMMNVQLDTGVPVLSAVLTPHRYRDSDAHTLLFLALFAVKGMEAARACVEILAAREKIAA

SEQ ID NO: 45 - protein, 156 aa, synthetic construct
NQHSHKDHETVRIAVVRARWHAEIVDACVSAFEAAMRDIGGDRFAVDVFDVPGAYEIPLHARTLAETGRYGAVLGTAFVVDGGIYDHEFVASAVIDGMMNVQLDTGVPVLSAVLTPHEYEDSDADTLLFLALFAVKGMEAARACVEILAAREKIAA

SEQ ID NO: 46 - protein, 156 aa, synthetic construct
NQHSHKDHETVRIAVVRARWHAEIVDACVSAFEAAMRDIGGDRFAVDVFDVPGAYEIPLHARTLAETGRYGAVLGTAFVVNGGIYRHEFVASAVINGMMNVQLNTGVPVLSAVLTPHNYDKSKAHTLLFLALFAVKGMEAARACVEILAAREKIAA

SEQ ID NO: 47 - protein, 156 aa, synthetic construct
TKKVGIVDTTFARVDMASAAILTLKMESPNIKIIRKTVPGIKDLPVACKKLLEEEGCDIVMALGMPGKAKEKDKVCAHEASLGLMLAQLMTNKHIIEVFVHEDEAKDDAELKILAARRAIEHALNVYYLLFKPEYLTRMAGKGLRQGFEDAGPARE

SEQ ID NO: 48 - protein, 215 aa, synthetic construct
SDTDINNQLKARLKVIPVIAIDNAEDIIPLGKVLAENGLPAAEITFRSSAAVKAIMLLRSAQPEMLIGAGTILNGVQALAAKEAGATDFVVSPGFNPNTVRACQIIGIDIVPGVNNPSTVEAQALEMGLTTLKFFPAEASGGISMVKSLVGPYGDIRLMPTGGITPSDNIDNYLAIPQVLACGGTWMVDKKLVTRNGEWDEIARLTREIVEQVNP

SEQ ID NO: 49 - protein, 119 aa, synthetic construct
PIFTLNTNIKATDDVPSDFLSLTSRLVGLILSKEPGSYVAVHINTDQQLSFGGSTNPAAFGTLMSIGGIEPSDKNREDHSAVLFDHLNAMLGIPKNRMYIHFVNDLNDGDDVGWNGTTF

SEQ ID NO: 50 - protein, 166 aa, synthetic construct
NQHSHKDYHETVRIAVVRARWHADIVDACVEAFEIAMAAIGGDRFAVDVFDVPGAYEIPLHARTLAETGRYGAVLGTAFVVNDGGIYRDHEFVASAVIDGMMNVQLSDTGVPVLSAVLTPHREYREDSADEHDHREFFAAHFAVKGVEAARACIEILANAREKIAA

SEQ ID NO: 51 - protein, 219 aa, synthetic construct
EELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHTDILKLFPGEVVGPQEFVKEAMKGPFPNVKFVPTGGVNDLDNDVCEKWFKDAGVLAVGVGSKDALVKEGTDKPDEVREKDAKAEKFVEKKEIRGCTE

SEQ ID NO: 52 - protein, 170 aa, synthetic construct
NQHSHKDYHETVRIAVVRARWHAEIVDACVSAFEAAMARDIGGDRFAVDVFDVPGAYEIPLHARTLAETGRYGAVLGTAFVVNDGGIYRDHEFVASAVIDNGMMNVQLSDNTGVPVLSAVLTPHRENYRDEDKSDKAHDTLLFLALFAVKGMEAARACVEILAAREKIAA

SEQ ID NO: 53 - protein, 158 aa, synthetic construct
GEVPIGDPKELNGMEIAAVYLQPIEMEPRGIDLAASLADIHLEADIHALKNNPNGFPEGFWMPYLTIAYALANADTGAIKTGTLMPMVADDGPHYGANIAMEKDKKGGFGVGTYALTFLISNPEKQGFGRHVDEETGVGKWFEPFVVTYFFKYTGTPK

SEQ ID NO: 54 - protein, 183 aa, synthetic construct
SQAIGILELTSIAKGMELGDAMLKSANVDLLVSKTISPGKFLLMLGGDIGAIQQAIETGTSQAGEMLVDSLVLANIHPSVLPAISGLNSVDKRQAVGIVETWSVAACISAADLAVKGSNVTLVRVHMAFGIGGKCYMVVAGDVLDVAAAVATASLAAGAKGLLVYASIIPRPHEAMWRQMVEG

SEQ ID NO: 55 - protein, 102 aa, synthetic construct
EEVVLITVPSALVAVKIAHALVEERLAACVNIVPGLTSIYRWQGSVVSDHELLLLVKTTTHAFPKLKERVKALHPYTVPEIVALPIAEGNREYLDWLRENTG

SEQ ID NO: 56 - protein, 121 aa, synthetic construct
VRGIRGAITVEEDTPAAILAATIELLLKMLEANGIQSYEELAAVIFTVTEDLTSAFPAEAARLIGMHRVPLLSAREVPVPGSLPRVIRVLALWNTDTPQDRVRHVYLNEAVRLRPDLESAQ

SEQ ID NO: 57 - protein, 176 aa, synthetic construct
SKAKIGIVTVSDRASAGITADISGKAIILALNLYLTSEWEPIYQVIPDEQDVIETTLIKMADEQDCCLIVTTGGTGPAKRDVTPEATEAVCDRMMPGFGELMRAESLKEVPTAILSRQTAGLRGDSLIVNLPGDPASISDCLLAVFPAIPYCIDLMEGPYLECNEAMIKPFRPKAK

SEQ ID NO: 58 - protein, 121 aa, synthetic construct
VRGIRGAITVNSDTPTSIIIATILLLEKMLEANGIQSYEELAAVIFTVTEDLTSAFPAEAARQIGMHRVPLLSAREVPVPGSLPRVIRVLALWNTDTPQDRVRHVYLSEAVRLRPDLESAQ

SEQ ID NO: 59 - protein, 171 aa, synthetic construct
RITTKVGDKGSTRLFGGEEVWKDSPIIEANGTLDELTSFIGEAKHYVDEEMKGILEEIQNDIYKIMGEIGSKGKIEGISEERIAWLLKLILRYMEMVNLKSFVLPGGTLESAKLDVCRTIARRALRKVLTVTREFGIGAEAAAYLLALSDLLFLLARVIEIEKNKLKEVRS

SEQ ID NO: 60 - protein, 122 aa, synthetic construct
PHLVIEATANLRLETSPGELLEQANKALFASGQFGEADIKSRFVTLEAYRQGTAAVERAYLHACLSILDGRDIATRTLLGASLCAVLAEAVAGGGEEGVQVSVEVREMERLSYAKRVVARQR

SEQ ID NO: 61 - protein, 157 aa, synthetic construct
ESVNTSFLSPSLVTIRDFDNGQFAVLRIGRTGFPADKGDIDLCLDKMIGVRAAQIFLGDDTEDGFKGPHIRIRCVDIDDKHTYNAMVYVDLIVGTGASEVERETAEEEAKLALRVALQVDIADEHSCVTQFEMKLREELLSSDSFHPDKDEYYKDFL

SEQ ID NO: 62 - protein, 112 aa, synthetic construct
PVIQTFVSTPLDHHKRLLLAIIYRIVTRVVLGKPEDLVMMTFHDSTPMHFFGSTDPVACVRVEALGGYGPSEPEKVTSIVTAAITAVCGIVADRIFVLYFSPLHCGWNGTNF

SEQ ID NO: 63 - protein, 102 aa, synthetic construct
EEVVLITVPSALVAVKIAHALVEERLAACVNIVPGLTSIYREEGSVVSDHELLLLVKTTTDAFPKLKERVKELHPYEVPEIVALPIAEGNREYLDWLRENTG

SEQ ID NO: 64 - protein, 202 aa, synthetic construct
EELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATE

SEQ ID NO: 65 - protein, 119 aa, synthetic construct
NLAEKMYKAGNAMYRKGQYTIAIIAYTLALLKDPNNAEAWYNLGNAAYKKGEYDEAIEAYQKALELDPNNAEAWYNLGNAYYKQGDYDEAIEYYKKALRLDPRNVDAIENLIEAEEKQG

SEQ ID NO: 66 - protein, 119 aa, synthetic construct
EEAELAYLLGELAYKLGEYRIAIRAYRIALKRDPNNAEAWYNLGNAYYKQGDYREAIRYYLRALKLDPENAEAWYNLGNALYKQGKYDLAIIAYQAALEEDPNNAEAKQNLGNAKQKQG

SEQ ID NO: 67 - protein, 119 aa, synthetic construct
NSAEAMYKMGNAAYKQGDYILAIIAYLLALEKDPNNAEAWYNLGNAAYKQGDYDEAIEYYQKALELDPNNAEAWYNLGNAYYKQGDYDEAIEYYEKALELDPNNAEALKNLLEAIAEQD

SEQ ID NO: 68 - protein, 187 aa, synthetic construct
TDPLAVILYIAILKAEKSIARAKAAEALGKIGDERAVEPLIKALKDEDALVRAAAADALGQIGDERAVEPLIKALKDEEGLVRASAAIALGQIGDERAVQPLIKALTDERDLVRVAAAVALGRIGDEKAVRPLIIVLKDEEGEVREAAAIALGSIGGERVRAAMEKLAERGTGFARKVAVNYLETHK

SEQ ID NO: 69 - protein, 119 aa, synthetic construct
EEAELAYLLGELAYKLGEYRIAIRAYRIALKRDPNNAEAWYNLGNAYYKQGDYDEAIEYYQKALELDPNNAEAWYNLGNAYYKQGDYDEAIEYYEKALELDPENLEALQNLLNAMDKQG

SEQ ID NO: 70 - protein, 279 aa, synthetic construct
IEEVVAEMIDILAESSKKSIEELARAADNKTTEKAVAEAIEEIARLATAAIQLIEALAKNLASEEFMARAISAIAELAKKAIEAIYRLADNHTTDTFMARAIAAIANLAVTAILAIAALASNHTTEEFMARAISAIAELAKKAIEAIYRLADNHTTDKFMAAAIEAIALLATLAILAIALLASNHTTEKFMARAIMAIAILAAKAIEAIYRLADNHTSPTYIEKAIEAIEKIARKAIKAIEMLAKNITTEEYKEKAKKIIDIIRKLAKMAIKKLEDNRT

SEQ ID NO: 71 - protein, 153 aa, synthetic construct
KYDGSKLRIGILHARWNAEIILALVLGALKRLQEFGVKRENIIIETVPGSFELPYGSKLFVEKQKRLGKPLDAIIPIGVLIKGSTMHFEYICDSTTHQLMKLNFELGIPVIFGVLTCLTDEQAEARAGLIEGKMHNHGEDWGAAAVEMATKFN

SEQ ID NO: 72 - protein, 119 aa, synthetic construct
EEAELAYLLGELAYKLGEYRIAIRAYRIALKRDPNNAEAWYNLGNAYYKQGRYREAIEYYQKALELDPNNAEAWYNLGNAYYERGEYEEAIEYYRKALRLDPNNADAMQNLLNAKMREE

SEQ ID NO: 73 - protein, 153 aa, synthetic construct
KYDGSKLRIGILHARGNAEIILALVLGALKRLQEFGVKRENIIIETVPGSFELPYGSKLFVEKQKRLGKPLDAIIPIGVLIRGSTPHFDYIADSTTHQLMKLNFELGIPVIFGVITADTDEQAEARAGLIEGKMHNHGEDWGAAAVEMATKFN

SEQ ID NO: 74 - protein, 150 aa, synthetic construct
KYDGSKLRIGILHARGNAEIILELVLGALKRLQEFGVKRENIIIETVPGSFELPYGSKLFVEKQKRLGKPLDAIIPIGVLIRGSTAHFDYIADSTTHQLMKLNFELGIPVIFGVLTTESDEQAEERAGTKAGNHGEDWGAAAVEMATKFN

SEQ ID NO: 75 - protein, 14 aa, synthetic construct
NQSRETAKAVSAVK

SEQ ID NO: 76 - protein, 748 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSARFDASISQVNEAINQSLAFIRKSDELLGSGGSGSGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELEHHHHHH

SEQ ID NO: 77 - protein, 754 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSREIIRAINIVRKIASEKGSGGSGSGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELEHHHHHH

SEQ ID NO: 78 - protein, 748 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKDDVSSSVITSLGAIVSCYGKTKCTASNKNRGIIKTFSNGCDYVSNKGVDTVSVGNTLYYVNKQEGKSLYVKGEPIINFYDPLVFPSARFAASISQVNEAINQSLAFIRKSDELLGSGGSGSGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELEHHHHHH

SEQ ID NO: 79 - protein, 748 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKDDVSSSVITSLGAIVSCYGKTKCTASNKNRGIIKTFSNGCDYVSNKGVDTVSVGNTLYYVNKQEGKSLYVKGEPIINFYDPLVFPSARWAASISQVNEAINQSLAFIRKSDELLGSGGSGSGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELEHHHHHH

SEQ ID NO: 80 - protein, 748 aa, synthetic construct
MELLILKANAITTILTAVTFCFASGQNITEEFYQSTCSAVSKGYLSALRTGWYTSVITIELSNIKEVKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDRFDASISQVNEAINQSLAFIRKSDELLGSGGSGSGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELEHHHHHH

SEQ ID NO: 81 - protein, 748 aa, synthetic construct
MELLILKANAITTILTAVTFCFASGQNITEEFYQSTCSAVSKGYLSALRTGWYTSVITIELSNIKEVKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSPYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDRFDASISQVNEAINQSLAFIRKSDELLGSGGSGSGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELEHHHHHH

SEQ ID NO: 82 - protein, 748 aa, synthetic construct
MELLILKANAITTILTAVTFCFASGQNITEEFYQSTCSAVSKGYLSALRTGWYTSVITIELSNIKEVKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKDDVSSSVITSLGAIVSCYGKTKCTASNKNRGIIKTFSNGCDYVSNKGVDTVSVGNTLYYVNKQEGKSLYVKGEPIINFYDPLVFPSDRFAASISQVNEAINQSLAFIRKSDELLGSGGSGSGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELEHHHHHH

SEQ ID NO: 83 - protein, 754 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDRFDASISQVNEAINQSREIIRAINIVRKIASEKGSGGSGSGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELEHHHHHH

SEQ ID NO: 84 - protein, 754 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSPYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDRFDASISQVNEAINQSREIIRAINIVRKIASEKGSGGSGSGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELEHHHHHH

SEQ ID NO: 85 - protein, 754 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSPYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKDDVSSSVITSLGAIVSCYGKTKCTASNKNRGIIKTFSNGCDYVSNKGVDTVSVGNTLYYVNKQEGKSLYVKGEPIINFYDPLVFPSDRWAASISQVNEAINQSREIIRAINIVRKIASEKGSGGSGSGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELEHHHHHH

SEQ ID NO: 86 - protein, 754 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKDDVSSSVITSLGAIVSCYGKTKCTASNKNRGIIKTFSNGCDYVSNKGVDTVSVGNTLYYVNKQEGKSLYVKGEPIINFYDPLVFPSDRFAASISQVNEAINQSREIIRAINIVRKIASEKGSGGSGSGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELEHHHHHH

SEQ ID NO: 87 - protein, 754 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSPYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKDDVSSSVITSLGAIVSCYGKTKCTASNKNRGIIKTFSNGCDYVSNKGVDTVSVGNTLYYVNKQEGKSLYVKGEPIINFYDPLVFPSDRFAASISQVNEAINQSREIIRAINIVRKIASEKGSGGSGSGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELEHHHHHH

SEQ ID NO: 88 - protein, 754 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSPYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKDDVSSSVITSLGAIVSCYGKTKCTASNKNRGIIKTFSNGCDYVSNKGVDTVSVGNTLYYVNKQEGKSLYVKGEPIINFYDPLVFPSARFAASISQVNEAINQSREIIRAINIVRKIASEKGSGGSGSGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELEHHHHHH

SEQ ID NO: 89 - protein, 754 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKDDVSSSVITSLGAIVSCYGKTKCTASNKNRGIIKTFSNGCDYVSNKGVDTVSVGNTLYYVNKQEGKSLYVKGEPIINFYDPLVFPSARWAASISQVNEAINQSREIIRAINIVRKIASEKGSGGSGSGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELEHHHHHH

SEQ ID NO: 90 - protein, 748 aa, synthetic construct
MELLIHRSSAIFLTLAINALYLTSSQNITEEFYQSTCSAVSRGYLSALRTGWYTSVITIELSNIKETKCNGTDTKVKLIKQELDKYKNAVTELQLLMQNTPAVNNRARREAPQYMNYTINTTKNLNVSISKKRKRRFLGFLLGVGSAIASGIAVCKVLHLEGEVNKIKNALQLTNKAVVSLSNGVSVLTFRVLDLKNYINNQLLPMLNRQSCRISNIETVIEFQQKNSRLLEITREFSVNAGVTTPLSTYMLTNSELLSLINDMPITNDQKKLMSSNVQIVRQQSYSIMCIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSDEFDASISQVNEKINQSLAFIRRSDELLGSGGSGSGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELEHHHHHH

SEQ ID NO: 91 - protein, 748 aa, synthetic construct
MELLIHRSSAIFLTLAINALYLTSSQNITEEFYQSTCSAVSRGYLSALRTGWYTSVITIELSNIKETKCNGTDTKVKLIKQELDKYKNAVTELQLLMQNTPAVNNRARREAPQYMNYTINTTKNLNVSISKKRKRRFLGFLLGVGSAIASGIAVCKVLHLEGEVNKIKNALQLTNKAVVSLSNGVSVLTFRVLDLKNYINNQLLPMLNRQSCRISNIETVIEFQQKNSRLLEITREFSVNAGVTTPLSTYMLTNSELLSLINDMPITNDQKKLMSSNVQIVRQQSYSIMCIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKDDISSSVITSLGAIVSCYGKTKCTASNKNRGIIKTFSNGCDYVSNKGVDTVSVGNTLYYVNKLEGKNLYVKGEPIINYYDPLVFPSARFAASISQVNEAINQSLAFIRRSDELLGSGGSGSGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELEHHHHHH

SEQ ID NO: 92 - protein, 748 aa, synthetic construct
MELLIHRSSAIFLTLAINALYLTSSQNITEEFYQSTCSAVSRGYLSALRTGWYTSVITIELSNIKETKCNGTDTKVKLIKQELDKYKNAVTELQLLMQNTPAVNNRARREAPQYMNYTINTTKNLNVSISKKRKRRFLGFLLGVGSAIASGIAVCKVLHLEGEVNKIKNALQLTNKAVVSLSNGVSVLTFRVLDLKNYINNQLLPMLNRQSCRISNIETVIEFQQKNSRLLEITREFSVNAGVTTPLSTYMLTNSELLSLINDMPITNDQKKLMSSNVQIVRQQSYSIMCIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKDDISSSVITSLGAIVSCYGKTKCTASNKNRGIIKTFSNGCDYVSNKGVDTVSVGNTLYYVNKLEGKNLYVKGEPIINYYDPLVFPSARWAASISQVNEAINQSLAFIRRSDELLGSGGSGSGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELEHHHHHH

SEQ ID NO: 93 - protein, 748 aa, synthetic construct
MELLIHRSSAIFLTLAINALYLTSSQNITEEFYQSTCSAVSRGYLSALRTGWYTSVITIELSNIKEVKCNGTDTKVKLIKQELDKYKNAVTELQLLMQNTPAVNNRARREAPQYMNYTINTTKNLNVSISKKRKRRFLGFLLGVGSAIASGIAVCKVLHLEGEVNKIKNALQLTNKAVVSLSNGVSVLTFRVLDLKNYINNQLLPMLNRQSCRISNIETVIEFQQKNSRLLEITREFSVNAGVTTPLSTYMLTNSELLSLINDMPITNDQKKLMSSNVQIVRQQSYSIMCIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSDRFDASISQVNEAINQSLAFIRRSDELLGSGGSGSGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELEHHHHHH

SEQ ID NO: 94 - protein, 748 aa, synthetic construct
MELLIHRSSAIFLTLAINALYLTSSQNITEEFYQSTCSAVSRGYLSALRTGWYTSVITIELSNIKEVKCNGTDTKVKLIKQELDKYKNAVTELQLLMQNTPAVNNRARREAPQYMNYTINTTKNLNVSISKKRKRRFLGFLLGVGSAIASGIAVCKVLHLEGEVNKIKNALQLTNKAVVSLSNGVSVLTFRVLDLKNYINNQLLPMLNRQSCRISNIETVIEFQQKNSRLLEITREFSVNAGVTTPLSPYMLTNSELLSLINDMPITNDQKKLMSSNVQIVRQQSYSIMCIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSDRFDASISQVNEAINQSLAFIRRSDELLGSGGSGSGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELEHHHHHH

SEQ ID NO: 95 - protein, 748 aa, synthetic construct
MELLIHRSSAIFLTLAINALYLTSSQNITEEFYQSTCSAVSRGYLSALRTGWYTSVITIELSNIKEVKCNGTDTKVKLIKQELDKYKNAVTELQLLMQNTPAVNNRARREAPQYMNYTINTTKNLNVSISKKRKRRFLGFLLGVGSAIASGIAVCKVLHLEGEVNKIKNALQLTNKAVVSLSNGVSVLTFRVLDLKNYINNQLLPMLNRQSCRISNIETVIEFQQKNSRLLEITREFSVNAGVTTPLSTYMLTNSELLSLINDMPITNDQKKLMSSNVQIVRQQSYSIMCIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKDDISSSVITSLGAIVSCYGKTKCTASNKNRGIIKTFSNGCDYVSNKGVDTVSVGNTLYYVNKLEGKNLYVKGEPIINYYDPLVFPSDRFAASISQVNEAINQSLAFIRRSDELLGSGGSGSGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELEHHHHHH

SEQ ID NO: 96 - protein, 754 aa, synthetic construct
MELLIHRSSAIFLTLAINALYLTSSQNITEEFYQSTCSAVSRGYLSALRTGWYTSVITIELSNIKETKCNGTDTKVKLIKQELDKYKNAVTELQLLMQNTPAVNNRARREAPQYMNYTINTTKNLNVSISKKRKRRFLGFLLGVGSAIASGIAVCKVLHLEGEVNKIKNALQLTNKAVVSLSNGVSVLTFRVLDLKNYINNQLLPMLNRQSCRISNIETVIEFQQKNSRLLEITREFSVNAGVTTPLSTYMLTNSELLSLINDMPITNDQKKLMSSNVQIVRQQSYSIMCIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSDRFDASISQVNEAINQSREIIRAINIVRKIASEKGSGGSGSGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELEHHHHHH

SEQ ID NO: 97 - protein, 755 aa, synthetic construct
MELLIHRSSAIFLTLAINALYLTSSQNITEEFYQSTCSAVSRGYLSALRTGWYTSVITIELSNIKETKCNGTDTKVKLIKQELDKYKNAVTELQLLMQNTPAVNNRARREAPQYMNYTINTTKNLNVSISKKRKRRFLGFLLGVGSAIASGIAVCKVLHLEGEVNKIKNALQLTNKAVVSLSNGVSVLTFRVLDLKNYINNQLLPMLNRQSCRISNIETVIEFQQKNSRLLEITREFSVNAGVTTPLSPYMLTNSELLSLINDMPITNDQKKLMSSNVQIVRQQSYSIMCIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSDRFDASISQVNEAINQSREIIRAINIVRKIASEKGSGGSGSGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELLEHHHHHH

SEQ ID NO: 98 - protein, 754 aa, synthetic construct
MELLIHRSSAIFLTLAINALYLTSSQNITEEFYQSTCSAVSRGYLSALRTGWYTSVITIELSNIKETKCNGTDTKVKLIKQELDKYKNAVTELQLLMQNTPAVNNRARREAPQYMNYTINTTKNLNVSISKKRKRRFLGFLLGVGSAIASGIAVCKVLHLEGEVNKIKNALQLTNKAVVSLSNGVSVLTFRVLDLKNYINNQLLPMLNRQSCRISNIETVIEFQQKNSRLLEITREFSVNAGVTTPLSPYMLTNSELLSLINDMPITNDQKKLMSSNVQIVRQQSYSIMCIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKDDISSSVITSLGAIVSCYGKTKCTASNKNRGIIKTFSNGCDYVSNKGVDTVSVGNTLYYVNKLEGKNLYVKGEPIINYYDPLVFPSDRWAASISQVNEAINQSREIIRAINIVRKIASEKGSGGSGSGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELEHHHHHH

SEQ ID NO: 99 - protein, 754 aa, synthetic construct
MELLIHRSSAIFLTLAINALYLTSSQNITEEFYQSTCSAVSRGYLSALRTGWYTSVITIELSNIKETKCNGTDTKVKLIKQELDKYKNAVTELQLLMQNTPAVNNRARREAPQYMNYTINTTKNLNVSISKKRKRRFLGFLLGVGSAIASGIAVCKVLHLEGEVNKIKNALQLTNKAVVSLSNGVSVLTFRVLDLKNYINNQLLPMLNRQSCRISNIETVIEFQQKNSRLLEITREFSVNAGVTTPLSTYMLTNSELLSLINDMPITNDQKKLMSSNVQIVRQQSYSIMCIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKDDISSSVITSLGAIVSCYGKTKCTASNKNRGIIKTFSNGCDYVSNKGVDTVSVGNTLYYVNKLEGKNLYVKGEPIINYYDPLVFPSDRFAASISQVNEAINQSREIIRAINIVRKIASEKGSGGSGSGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELEHHHHHH

SEQ ID NO: 100 - protein, 754 aa, synthetic construct
MELLIHRSSAIFLTLAINALYLTSSQNITEEFYQSTCSAVSRGYLSALRTGWYTSVITIELSNIKETKCNGTDTKVKLIKQELDKYKNAVTELQLLMQNTPAVNNRARREAPQYMNYTINTTKNLNVSISKKRKRRFLGFLLGVGSAIASGIAVCKVLHLEGEVNKIKNALQLTNKAVVSLSNGVSVLTFRVLDLKNYINNQLLPMLNRQSCRISNIETVIEFQQKNSRLLEITREFSVNAGVTTPLSPYMLTNSELLSLINDMPITNDQKKLMSSNVQIVRQQSYSIMCIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKDDISSSVITSLGAIVSCYGKTKCTASNKNRGIIKTFSNGCDYVSNKGVDTVSVGNTLYYVNKLEGKNLYVKGEPIINYYDPLVFPSDRFAASISQVNEAINQSREIIRAINIVRKIASEKGSGGSGSGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELEHHHHHH

SEQ ID NO: 101 - protein, 754 aa, synthetic construct
MELLIHRSSAIFLTLAINALYLTSSQNITEEFYQSTCSAVSRGYLSALRTGWYTSVITIELSNIKETKCNGTDTKVKLIKQELDKYKNAVTELQLLMQNTPAVNNRARREAPQYMNYTINTTKNLNVSISKKRKRRFLGFLLGVGSAIASGIAVCKVLHLEGEVNKIKNALQLTNKAVVSLSNGVSVLTFRVLDLKNYINNQLLPMLNRQSCRISNIETVIEFQQKNSRLLEITREFSVNAGVTTPLSPYMLTNSELLSLINDMPITNDQKKLMSSNVQIVRQQSYSIMCIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKDDISSSVITSLGAIVSCYGKTKCTASNKNRGIIKTFSNGCDYVSNKGVDTVSVGNTLYYVNKLEGKNLYVKGEPIINYYDPLVFPSARFAASISQVNEAINQSREIIRAINIVRKIASEKGSGGSGSGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELEHHHHHH

SEQ ID NO: 102 - protein, 754 aa, synthetic construct
MELLIHRSSAIFLTLAINALYLTSSQNITEEFYQSTCSAVSRGYLSALRTGWYTSVITIELSNIKETKCNGTDTKVKLIKQELDKYKNAVTELQLLMQNTPAVNNRARREAPQYMNYTINTTKNLNVSISKKRKRRFLGFLLGVGSAIASGIAVCKVLHLEGEVNKIKNALQLTNKAVVSLSNGVSVLTFRVLDLKNYINNQLLPMLNRQSCRISNIETVIEFQQKNSRLLEITREFSVNAGVTTPLSTYMLTNSELLSLINDMPITNDQKKLMSSNVQIVRQQSYSIMCIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKDDISSSVITSLGAIVSCYGKTKCTASNKNRGIIKTFSNGCDYVSNKGVDTVSVGNTLYYVNKLEGKNLYVKGEPIINYYDPLVFPSARWAASISQVNEAINQSREIIRAINIVRKIASEKGSGGSGSGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELEHHHHHH

SEQ ID NO: 103 - protein, 754 aa, synthetic construct
MELLIHRSSAIFLTLAINALYLTSSQNITEEFYQSTCSAVSRGYLSALRTGWYTSVITIELSNIKETKCNGTDTKVKLIKQELDKYKNAVTELQLLMQNTPAVNNRARREAPQYMNYTINTTKNLNVSISKKRKRRFLGFLLGVGSAIASGIAVCKVLHLEGEVNKIKNALQLTNKAVVSLSNGVSVLTFRVLDLKNYINNQLLPMLNRQSCRISNIETVIEFQQKNSRLLEITREFSVNAGVTTPLSTYMLTNSELLSLINDMPITNDQKKLMSSNVQIVRQQSYSIMCIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSDEFDASISQVNEKINQSREIIRAINIVRKIASEKGSGGSGSGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELEHHHHHH

SEQ ID NO: 104 - skipped

SEQ ID NO: 105 - protein, 205 aa, synthetic construct
MKMEELFKKHKIVAVLRANSVEEAKKKALAVFLGGVHLIEITFTVPDADTVIKELSFLKEMGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPVEVAEKAKAFVEKIRGCTE

SEQ ID NO: 106 - protein, 205 aa, synthetic construct
MKIEELFKKHKIVAVLRANSVEEAKKKALAVFLGGVHLIEITFTVPDADTVIKELSFLKEMGAIIGAGTVTSVEQCRKAVESGAEFIVSPHLDEEISQFCKEKGVEYMPGVMTPTELVKAMKLGHTILKLFPGEVVGPQFVKAMKGPFPNVKFVPTGGVNLDNVCEWFKAGVLAVGVGSALVKGTPVEVAEKAKAFVEKIRGCTE

SEQ ID NO: 107 - protein, 205 aa, synthetic construct
MKMEELFKKHKIVAVLRANSVEEAKEKALAVFEGGVHLIEITFTVPDADTVIKELSFLKEKGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPDEVREKAKAFVEKIRGCTE

SEQ ID NO: 108 - protein, 205 aa, synthetic construct; identical to SEQ ID NO: 105

SEQ ID NO: 109 - protein, 205 aa, synthetic construct
MKIEELFKKHKIVAVLRANSVEEAKKKALAVFLGGVHLIEITFTVPDADTVIKELSFLKEMGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPVEVAEKAKAFVEKIRGCTE

SEQ ID NO: 110 - protein, 205 aa, synthetic construct
MKMEELFKKHKIVAVLRANSVEEAKKKALAVFLGGVHLIEITFTVPDADTVIKELSFLKEMGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVCEWFKAGVLAVGVGSALVKGTPVEVAEKAKAFVEKIRGCTE

SEQ ID NO: 111 - protein, 205 aa, synthetic construct
MKMEELFKKHKIVAVLRANSVEEAKMKALAVFVGGVHLIEITFTVPDADTVIKELSFLKELGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTIAEVAAKAAAFVEKIRGCTE

SEQ ID NO: 112 - protein, 205 aa, synthetic construct
MKMEELFKKHKIVAVLRANSVEEAKKKALAVFLGGVDLIEITFTVPDADTVIKELSFLKEMGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVQAVGVGEALNKGTPVEVAEKAKAFVEKIRGCTE

SEQ ID NO: 113 - protein, 205 aa, synthetic construct
MKMEELFKKHKIVAVLRANSVEEAKKKALAVFMGGVDLIEITFTVPDADTVIKELSFLKELGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVQAVGVGEALNKGTPAEVAEKAKAFVEKIRGCTE

SEQ ID NO: 114 - protein, 167 aa, synthetic construct
MLSKDIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKVELIGNENHGLYLADQYVKGIAKSRKS

SEQ ID NO: 115 - protein, 165 aa, synthetic construct
MLKPEMIEKLNEQMNLELYSSLLYQQMSAWCSYHTFEGAAAFLRRHAQEEMTHMQRLFDYLTDTGNLPRINTVESPFAEYSSLDELFQETYKHEQLITQKINELAHAAMTNQDYPTFNFLQWYVSEQHEEEKLFKSIIDKLSLAGKSGEGLYFIDKELSTLDAQN

SEQ ID NO: 116 - protein, 166 aa, synthetic construct
NFHQDCEAGLNRTVNLKFHSSYVYLSMASYFNRDDVALSNFAKFFRERSEEEKEHAEKLIEYQNQRGGRVFLQSVEKPERDDWANGLEALQTALKLQKSVNQALLDLHAVAADKSDPHMTDFLESPYLSESVETIKKLGDHITSLKKLWSSHPGMAEYLFNKHTLG

SEQ ID NO: 117 - protein, 169 aa, synthetic construct
QFSKDIEKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSGS

SEQ ID NO: 118 - protein, 172 aa, synthetic construct
SGESQVRQNFKPEMEEKLNEQMNLELYSSLLYQQMSAWCSYHTFEGAAAFLRRHAQEEMTHMQRLFDYLTDTGNLPRINTVESPFAEYSSLDELFQETYKHEQLITQKINELAHAAMTNQDYPTFNFLQWYVSEQHEEEKLFKSIIDKLSLAGKSGEGLYFIDKELSTLDGS

SEQ ID NO: 119 - protein, 20 aa, synthetic construct
NQSALLLEAAKYVKKAREKS

SEQ ID NO: 120 - protein, 20 aa, synthetic construct
NQSRKLLEAAEEMEKMLKTS

SEQ ID NO: 121 - protein, 20 aa, synthetic construct
NQSRKMLEAVEHAKKLKKES

SEQ ID NO: 122 - protein, 20 aa, synthetic construct
NQSRKMLEAVEKAKKLDKES

SEQ ID NO: 123 - protein, 20 aa, synthetic construct
NQSAKTEEAYQRTIKTQQKL

SEQ ID NO: 124 - protein, 21 aa, synthetic construct
NQSRDLDTAAKQVKEMLKEKS

SEQ ID NO: 125 - protein, 21 aa, synthetic construct
NQSRETEKTIRQVQEILKKWS

SEQ ID NO: 126 - protein, 21 aa, synthetic construct
NQSREVKEAIKIIKKILKKQS

SEQ ID NO: 127 - protein, 21 aa, synthetic construct
NQSREIKDAIKKAKEFIKTIK

SEQ ID NO: 128 - protein, 21 aa, synthetic construct
NQSREIETAIKKAKEFIKTIK

SEQ ID NO: 129 - protein, 19 aa, synthetic construct
NQSRKATETIKKFEESEKS

SEQ ID NO: 130 - protein, 21 aa, synthetic construct
NQSRDTIKVAIIVKELYKKIS

SEQ ID NO: 131 - protein, 22 aa, synthetic construct
NQSRKTLETIEWVKKVIKKQRS

SEQ ID NO: 132 - protein, 22 aa, synthetic construct
NQSRKTLETIEWVEKVIKKQRS

SEQ ID NO: 133 - protein, 18 aa, synthetic construct
NQSRKWNESSKKVQEQDS

SEQ ID NO: 134 - protein, 20 aa, synthetic construct
NQSRKTEKAIRLVLKWLKES

SEQ ID NO: 135 - protein, 23 aa, synthetic construct
NQSRDTLKAIEQTKRYLEELKKS

SEQ ID NO: 136 - protein, 21 aa, synthetic construct
NQSRSWDIAAKFVKTVLSNQS

SEQ ID NO: 137 - protein, 21 aa, synthetic construct
NQSRKTLEATEIAKKLAEDRS

SEQ ID NO: 138 - protein, 23 aa, synthetic construct
NQSLEILKAAKEAKKLIEDLRRS

SEQ ID NO: 139 - protein, 23 aa, synthetic construct
NQSKELLDAAKAVKKMLEKEKSS

SEQ ID NO: 140 - protein, 23 aa, synthetic construct
NQSKKLLDAADAVKKMLEKEKSS

SEQ ID NO: 141 - protein, 23 aa, synthetic construct
NQSKKVLETIRWIETVISRQRSS

SEQ ID NO: 142 - protein, 24 aa, synthetic construct
NQSADLKKVAELVKKLMEEAKKKS

SEQ ID NO: 143 - protein, 21 aa, synthetic construct
NQSTDTMKAARIMKEELKEKS

SEQ ID NO: 144 - protein, 24 aa, synthetic construct
NQSRKTEEALRRADTIIKQLASKS

SEQ ID NO: 145 - protein, 20 aa, synthetic construct
NQSKKLKSAADDVKKAKEKS

SEQ ID NO: 146 - protein, 20 aa, synthetic construct
NQSKELKSAAEDVKKAKEKS

SEQ ID NO: 147 - protein, 22 aa, synthetic construct
NQSRETKKATENVKTMLTKSKS

SEQ ID NO: 148 - protein, 21 aa, synthetic construct
NQSLELKKAAKAANTDLTKKS

SEQ ID NO: 149 - protein, 21 aa, synthetic construct
NQSLELKEAAKAANTDLTKKS

SEQ ID NO: 150 - protein, 24 aa, synthetic construct
NQSRKLEEIARIVEQKKRTEEKRS

SEQ ID NO: 151 - protein, 16 aa, synthetic construct
NQSAETKKAIERAREL

SEQ ID NO: 152 - protein, 16 aa, synthetic construct
NQSRDLKKAAEIAKKS

SEQ ID NO: 153 - protein, 16 aa, synthetic construct
NQSRTLLETAEIVTRS

SEQ ID NO: 154 - protein, 16 aa, synthetic construct
NQSRTLLETAEIVKRS

SEQ ID NO: 155 - protein, 16 aa, synthetic construct
NQSRKLDKAAEYVEKS

SEQ ID NO: 156 - protein, 17 aa, synthetic construct
NQSKEAKKAIETAKKLS

SEQ ID NO: 157 - protein, 17 aa, synthetic construct
NQSRKLETAAEKLKQTE

SEQ ID NO: 158 - protein, 17 aa, synthetic construct
NQSRLMLEAVKIAQSQS

SEQ ID NO: 159 - protein, 18 aa, synthetic construct
NQSRETKEAAESVKQMES

SEQ ID NO: 160 - protein, 18 aa, synthetic construct
NQSRRTLKAIEITLKLLS

SEQ ID NO: 161 - protein, 18 aa, synthetic construct
NQSRRTLTAITRVERKDS

SEQ ID NO: 162 - protein, 19 aa, synthetic construct
NQSKKLADAADWVETVKSS

SEQ ID NO: 163 - protein, 19 aa, synthetic construct
NQSKKTHSAIEWVERLVSS

SEQ ID NO: 164 - protein, 19 aa, synthetic construct
NQSADTKKAAEIAKKLAKS

SEQ ID NO: 165 - protein, 25 aa, synthetic construct
NQSQSIQATTSRVDAIEAKVKHLEA

SEQ ID NO: 166 - protein, 33 aa, synthetic construct
NQSVTINNMISSNTNEISSLQDRVKHIEDTLAL

SEQ ID NO: 167 - protein, 25 aa, synthetic construct
NQSKLVKKVIKETHEIKKKLEDLLK

SEQ ID NO: 168 - protein, 33 aa, synthetic construct
NQSRSNKKTKNKVKSIEKQVKEIEKRLEKLERA

SEQ ID NO: 169 - protein, 27 aa, synthetic construct
NQSQAIRETQDEVKNLNKRINKIVTSI

SEQ ID NO: 170 - protein, 29 aa, synthetic construct
NQSRAIKETQKRTTVLEEDLKRVKELLKS

SEQ ID NO: 171 - protein, 27 aa, synthetic construct
NQSRQIVEVMKEVEELRKRVENIEKNL

SEQ ID NO: 172 - protein, 31 aa, synthetic construct
NQSQKTRATEEALKKTQKEVTKLKKEIQKLT

SEQ ID NO: 173 - protein, 33 aa, synthetic construct
NQSRSNKKTKNKVKSIEKQVKEIEKRLEKLEKA

SEQ ID NO: 174 - protein, 31 aa, synthetic construct
NQSNTVRKTIETVNSLEKELKELRTEVDRLL

SEQ ID NO: 175 - protein, 27 aa, synthetic construct
NQSKEIRNTVKKVRTIEKRLNKLETSL

SEQ ID NO: 176 - protein, 27 aa, synthetic construct
NQSRTLKDTTELTKNLNKKLKKLEEEL

SEQ ID NO: 177 - protein, 29 aa, synthetic construct
NQSKYISNRIKENTDQIKKLEERVTELEA

SEQ ID NO: 178 - protein, 27 aa, synthetic construct
NQSLEIRQTSKRVESLERRVTQVERDR

SEQ ID NO: 179 - protein, 27 aa, synthetic construct
GYIPEAPRDGQAYVRKDGEWVLLSTFL

SEQ ID NO: 180 - protein, 488 aa, synthetic construct; identical to SEQ ID NO: 5

SEQ ID NO: 181 - protein, 488 aa, synthetic construct
QNITEEFYQSTCSAVSRGYFSALRTGWYTSVITIELSNITETKCNGTDTKVKLIKQELDKYKNAVTELQLLMQNTPAANNRARREAPQHMNYTINTTKNLNVSISKKRKRRFLGFLLGVGSAIASGIAVSKVLHLEGEVNKIKNALLSTNKAVVSLSNGVSVLTSKVLDLKNYINNQLLPIVNQQSCRIFNIETVIEFQQKNSRLLEITREFSVNAGVTTPLSTYMLTNSELLSLINDMPITNDQKKLMSSNVQIVRQQSYSIMSIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSDEFDASISQVNEKINQSLAFIRKSDELL

SEQ ID NO: 182 - protein, 488 aa, synthetic construct
QNITEEFYQSTCSAVSRGYFSALRTGWYTSVITIELSNITETKCNGTDTKVKLIKQELDKYKNAVTELQLLMQNTPAANNRARREAPQHMNYTINTTKNLNVSISKKRKRRFLGFLLGVGSAIASGIAVCKVLHLEGEVNKIKNALLSTNKAVVSLSNGVSVLTFKVLDLKNYINNQLLPILNQQSCRIFNIETVIEFQQKNSRLLEITREFSVNAGVTTPLSTYMLTNSELLSLINDMPITNDQKKLMSSNVQIVRQQSYSIMCIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSDEFDASISQVNEKINQSLAFIRKSDELL

SEQ ID NO: 183 - protein, 574 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPPTNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 184 - protein, 574 aa, synthetic construct
MELLIHRSSAIFLTLAVNALYLTSSQNITEEFYQSTCSAVSRGYFSALRTGWYTSVITIELSNIKETKCNGTDTKVKLIKQELDKYKNAVTELQLLMQNTPAANNRARREAPQYMNYTINTTKNLNVSISKKRKRRFLGFLLGVGSAIASGIAVSKVLHLEGEVNKIKNALLSTNKAVVSLSNGVSVLTSKVLDLKNYINNRLLPIVNQQSCRISNIETVIEFQQMNSRLLEITREFSVNAGVTTPLSTYMLTNSELLSLINDMPITNDQKKLMSSNVQIVRQQSYSIMSIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSDEFDASISQVNEKINQSLAFIRRSDELLHNVNTGKSTTNIMITTIIIVIIVVLLSLIAIGLLLYCKAKNTPVTLSKDQLSGINNIAFSK

SEQ ID NO: 185 - protein, 544 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLSAIGGYIPEAPRDGQAYVRKDGEWVLLSTFL

SEQ ID NO: 186 - protein, 6 aa, synthetic construct
QVKRNL

SEQ ID NO: 187 - protein, 8 aa, synthetic construct
STLAQKEY

SEQ ID NO: 188 - protein, 5 aa, synthetic construct
IVNTL

SEQ ID NO: 189 - protein, 6 aa, synthetic construct
QNKRVS

SEQ ID NO: 190 - protein, 6 aa, synthetic construct
ANKEDQ

SEQ ID NO: 191 - protein, 4 aa, synthetic construct
TMVR

SEQ ID NO: 192 - protein, 8 aa, synthetic construct
TIKQMEVS

SEQ ID NO: 193 - protein, 5 aa, synthetic construct
SKNDE

SEQ ID NO: 194 - protein, 7 aa, synthetic construct
RSEKATL

SEQ ID NO: 195 - protein, 4 aa, synthetic construct
VNTL

SEQ ID NO: 196 - protein, 7 aa, synthetic construct
DTHKENR

SEQ ID NO: 197 - protein, 8 aa, synthetic construct
ANESVKTD

SEQ ID NO: 198 - protein, 5 aa, synthetic construct
IELTQ

SEQ ID NO: 199 - protein, 6 aa, synthetic construct
EIKNRQ

SEQ ID NO: 200 - protein, 5 aa, synthetic construct
ASKER

SEQ ID NO: 201 - protein, 6 aa, synthetic construct
KSQRDE

SEQ ID NO: 202 - skipped

SEQ ID NO: 203 - protein, 5 aa, synthetic construct
KQENT

SEQ ID NO: 204 - protein, 7 aa, synthetic construct
HDEKRNQ

SEQ ID NO: 205 - protein, 4 aa, synthetic construct
LRIV

SEQ ID NO: 206 - protein, 5 aa, synthetic construct
EVLKR

SEQ ID NO: 207 - protein, 5 aa, synthetic construct
AKTER

SEQ ID NO: 208 - protein, 7 aa, synthetic construct
HRSLNED

SEQ ID NO: 209 - protein, 4 aa, synthetic construct
ILVR

SEQ ID NO: 210 - protein, 4 aa, synthetic construct
EKQD

SEQ ID NO: 211 - protein, 5 aa, synthetic construct
DKSRA

SEQ ID NO: 212-215 - skipped (4 entries)

SEQ ID NO: 216 - protein, 519 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPACNNRARRELPRFMNYTLNNAKKTNVTLSKKRKRRFLGFLLGVGSACASGVAVSKVLHLEGEVNKIKSALLSTNKAVVSLSNGVSVLTIKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSSEFDASISQVNEKINQSREIIRAINIVRKIASEK

SEQ ID NO: 217 - protein, 518 aa, synthetic construct
MELLILKANAITTILTAVTFCFASGQNITEEFYQSTCSAVSKGYLSALRTGWYHSVITIELSNIKENKCNGTDAKVKLIKQELDKYKNAVTELQLLMQSTPACNNRARRELPRFMNYTLNNAKKTNVTLSKKRKRRFLGFLLGVGSACASGVAVSKVLHLEGEVNKIKSALLSTNKAWSLSNGVSVLTIKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEILAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSSEFDASISQVNEKINQSREIIRAINIVRKIASEK

SEQ ID NO: 218 - protein, 519 aa, synthetic construct
MELLILKANAITTILTAVTFCFASGQNITEEFYQSTCSAVSKGYLSALRTGWYHCVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVCTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSSEFDASISQVNEKINQSREIIRAINIVRKIASEK

SEQ ID NO: 219 - protein, 519 aa, synthetic construct
MELLILKANAITTILTAVTFCFASGQNITEEFYQSTCSAVSKGYLSALRTGWYHCVITIELSNIKENKCNGTDAKVKLIKQELDKYKNAVTELQLLMQSTPATNNRARRELPRFMNYTLNNAKKTNVTLSKKRKRRFLGFLCGVGSAIASGVAVSKVLHLEGEVNKIKSALLSTNKAVVSLSNGVSVCTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEILAYVVQLPLYGVIDTPCWKLHTSPLCTTNTKEGSNICLTRTDRGWYCDNAGSVSFFPQAETCKVQSNRVFCDTMCSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSREIIRAINIVRKIASEK

SEQ ID NO: 220 - protein, 518 aa, synthetic construct
MELLILKANAITTILTAVTFCFASGQNITEEFYQSTCSAVSKGYLSALRTGWYTCVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVCTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYWQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSSEFDASISQVNEKINQSREIIRAINIVRKIASEK

SEQ ID NO: 221 - protein, 519 aa, synthetic construct
MELLILKANAITTILTAVTFCFASGQNITEEFYQSTCSAVSKGYLSALRTGWYHCVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVCTIKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSREIIRAINIVRKIASEK

SEQ ID NO: 222 - protein, 519 aa, synthetic construct
MELLILKANAITTILTAVTFCFASGQNITEEFYQSTCSAVSKGYLSALRTGWYTCVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVCTIKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSSEFDASISQVNEKINQSREIIRAINIVRKIASEK

SEQ ID NO: 223 - protein, 519 aa, synthetic construct
MELLILKANAITTILTAVTFCFASGQNITEEFYQSTCSAVSKGYLSALRTGWYHCVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVCTIKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSSEFDASISQVNEKINQSREIIRAINIVRKIASEK

SEQ ID NO: 224 - protein, 519 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTIKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSSEFDASISQVNEKINQSREIIRAINIVRKIASEK

SEQ ID NO: 225 - protein, 519 aa, synthetic construct
MELLILKANAITTILTAVTFCFASGQNITEEFYQSTCSAVSKGYLSALRTGWYHCVITIELSNIKENKCNGTDAKVKLIKQELDKYKNAVTELQLLMQSTPATNNRARRELPRFMNYTLNNAKKTNVTLSKKRKRRFLGFLCGVGSAIASGVAVSKVLHLEGEVNKIKSALLSTNKAVVSLSNGVSVCTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEILAYVVQLPLYGVIDTPCWKLHTSPLCTTNTKEGSNICLTRTDRGWYCDNAGSVSFFPQAETCKVQSNRVFCDTMCSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSSQFSASISQVNEKINQSREIIRAINIVRKIASEK

SEQ ID NO: 226 - protein, 518 aa, synthetic construct
MELLILKANAITTILTAVTFCFASGQNITEEFYQSTCSAVSKGYLSALRTGWYHSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTIKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMCIIKEEILAYWQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSREIIRAINIVRKIASEK

SEQ ID NO: 227 - protein, 518 aa, synthetic construct
MELLILKANAITTILTAVTFCFASGQNITEEFYQSTCSAVSKGYLSALRTGWYHCVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVCTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYWQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSSEFDASISQVNEKINQSREIIRAINIVRKIASEK

SEQ ID NO: 228 - protein, 509 aa, synthetic construct
MELLILKAAITTILTAVTFCFASGQNITEEFYQSTCSAVSKGYLSALRTGWYTSCITIELSNIKENKCNGTDAVKLIKQELDKYKNAVTELQLLMQSTPATNNRARRELPRFMYTLNNAKKTVTLSKKRKRRFLGFLLGVGSAIASGVAVSKVLHLEGECNKIKSALLSTNKAVVSLSNGVSVLTSKVLDLKNYIDKQLLPIVKQSCSISNIETVIEFQQKNNRLLEITREFSVAGVTTPVSTYMLTNSELLSLINDMPITNDQKKLMSNNVQIVRQQSYSIMSIIKEEVLAYWQLPLYGVIDTPCWKLHTSPLCTTNTKEGSNICLTRTDRGWYCDNAGSVSFFPQAETCKVQSNRVFCDTMSLTLPSEVNLCNVDIFNPKYDCKIMTSKTDVSSSVITSLGAIVSCYGKTKCTASNKNRGIIKTFSNGCDYVSNKGVDTVSVGNTLYYVKQEGKSLYVKGEPIINFYDPLVFPSDEFDASISQVEKINQSREIIRAINIVRKIASEK

SEQ ID NO: 229 - protein, 507 aa, synthetic construct
MELLILKAAITTILTAVTFCFASGQNITEEFYQSTCSAVSKGYLSALRTGWYTSVCTIELSNIKENKCNGTDAVKLIKQELDKYKNAVTELQLLMQSTPATNNRARRELPRFMYTLNNAKKTVTLSKKRKRRFLGFLLGVGSAIASGVAVSBVLHLEGEVKIKSALLSTNKAWSLSNGVSVLTCBVLDLKNYIDKQLLPIVKQSCSISNIETVIEFQQKNNRLLEITREFSVAGVTTPVSTYMLTNSELLSLINDMPITNDQKKLMSNNVQIVRQQSYSIMSIIKEEVLAYWQLPLYGVIDTPCWKLHTSPLCTTNTKEGSNICLTRTDRGWYCDNAGSVSFFPQAETCKVQSNRVFCDTMSLTLPSEVNLCNVDIFNPKYDCKIMTSKTDVSSSVITSLGAIVSCYGKTKCTASNKNRGIIKTFSNGCDYVSNKGVDTVSVGNTLYYVKQEGKSLYVKGEPIINFYDPLVFPSDEFDASISQVEKINQSREIIRAINIVRKIASEK

SEQ ID NO: 230 - protein, 509 aa, synthetic construct
MELLILKAAITTILTAVTFCFASGQNITEEFYQSTCSAVSKGYLSALRTGWYTSVICIELSNIKENKCNGTDAVKLIKQELDKYKNAVTELQLLMQSTPATNNRARRELPRFMYTLNNAKKTVTLSKKRKRRFLGFLLGVGSAIASGVAVSKVLHLEGECNKIKSALLSTNKAVVSLSNGVSVLTSKVLDLKNYIDKQLLPIVKQSCSISNIETVIEFQQKNNRLLEITREFSVAGVTTPVSTYMLTNSELLSLINDMPITNDQKKLMSNNVQIVRQQSYSIMSIIKEEVLAYWQLPLYGVIDTPCWKLHTSPLCTTNTKEGSNICLTRTDRGWYCDNAGSVSFFPQAETCKVQSNRVFCDTMSLTLPSEVNLCNVDIFNPKYDCKIMTSKTDVSSSVITSLGAIVSCYGKTKCTASNKNRGIIKTFSNGCDYVSNKGVDTVSVGNTLYYVKQEGKSLYVKGEPIINFYDPLVFPSDEFDASISQVEKINQSREIIRAINIVRKIASEK

SEQ ID NO: 231 - protein, 508 aa, synthetic construct
MELLILKAAITTILTAVTFCFASGQNITEEFYQSTCSAVSKGYLSALRTGWYTSVITIELSNIKENKCNGTDAVKLIKQELDKYKNAVTELQLLMQSTPATNNRARRELPRFMYTLNNAKKTVTLSKKRKRRFLGFLLGVGSAIASGVAVSBVLHLEGEVCKIKSALLSTNKAWSLSNGVSVLTSBVLDLKNYIDKQLLPIVKQSCSISNIETVIEFQQKNNRLLEITREFSVAGVTTPVSTYMLTNSELLSLINDMPITNDQKKLMSNNVQIVRQQSYSIMSIIKEECLAYWQLPLYGVIDTPCWKLHTSPLCTTNTKEGSNICLTRTDRGWYCDNAGSVSFFPQAETCKVQSNRVFCDTMSLTLPSEVNLCNVDIFNPKYDCKIMTSKTDVSSSVITSLGAIVSCYGKTKCTASNKNRGIIKTFSNGCDYVSNKGVDTVSVGNTLYYVKQEGKSLYVKGEPIINFYDPLVFPSDEFDASISQVEKINQSREIIRAINIVRKIASEK

SEQ ID NO: 232 - protein, 506 aa, synthetic construct
MELLILKAAITTILTAVTFCFASGQNITEEFYQSTCSAVSKGYLSALRTGWYTSVITIELSNIKENKCNGTDAKVKLIKQELDKYKNAVTELQLLMQSTPATIWRARRELPRFMYTLAKKTVTLSKKRKRRFLGFLLGVGSAIASGVAVSBVLHLEGEVKICSALLSTNKAWSLSNGVSVLTSBVLDLKNYIDKQLLPIVKQSCSISNIETVIEFQQKNNRLLEITREFSVAGVTTPVSTYMLTNSELLSLINDMPITNDQKKLMSNNVQIVRQQSYSIMSIIKEECLAYWQLPLYGVIDTPCWKLHTSPLCTTNTKEGSNICLTRTDRGWYCDNAGSVSFFPQAETCKVQSNRVFCDTMSLTLPSEVNLCNVDIFNPKYDCKIMTSKTDVSSSVITSLGAIVSCYGKTKCTASNKNRGIIKTFSNGCDYVSNKGVDTVSVGNTLYYVKQEGKSLYVKGEPIINFYDPLVFPSDEFDASISQVEKINQSREIIRAINIVRKIASEK

SEQ ID NO: 233 - protein, 508 aa, synthetic construct
MELLILKAAITTILTAVTFCFASGQNITEEFYQSTCSAVSKGYLSALRTGWYTSVITIELSNIKENKCNGTDAVKLIKQELDKYKNAVTELQLLMQSTPATNNRARRELPRFMYTLNNAKKTVTLSKKRKRRFLGFLLGVGSAIASGVAVSKVLHLEGEVKIKSALLSTNKAVVSLSNGVSVLTSKVLDLKNYIDKQLLPIVKQSCSISNIETVIEFQQKNNRLLEITREFSVAGVTTPVSTYMLTNSELLSLINDMPITNDQKKLMSNNVQIVRQQSYSIMSIIKEEVLAYWQLPLYGVIDTPCWKLHTSPLCTTNTKEGSNICLTRTDRGWYCDNAGSVSFFPQAETCKVQSNRVFCDTMSLTLPSEVNLCNVDIFNPKYDCKICTSKTDVSSSVITSLGAIVSCYGKTKCTASNKNRGIIKTFSNGCDYVSNKGVDTVSVGNTLYYVKQEGKSLYVKGEPIINFYDPLVCPSDEFDASISQVEKINQSREIIRAINIVRKIASEK

SEQ ID NO: 234 - protein, 519 aa, synthetic construct
MELLILKANAITTILTAVTFCFASGQNITEEFYQSTCSAVSKGYLSALRTGWYTSVITIELSNIKKNKCNGTDAKVKLIKQELDKYKNAVTELQLLMQSTQATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSREIIRAINIVRKIASEK

SEQ ID NO: 235 - protein, 519 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSREIIRAINIVRKIASEK

SEQ ID NO: 236 - protein, 519 aa, synthetic construct; identical to SEQ ID NO: 234

SEQ ID NO: 237 - protein, 519 aa, synthetic construct; identical to SEQ ID NO: 235

SEQ ID NO: 238 - protein, 496 aa, synthetic construct
MELLILKANAITTILTAVTFCFASGQNITEEFYQSTCSAVSKGYLSALRTGWYTSVITIELSNIKKNKCNGTDAKVKLIKQELDKYKNAVTELQLLMQSTQATNNRARQQQQRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSREIIRAINIVRKIASEK

SEQ ID NO: 239 - protein, 489 aa, synthetic construct
MELLILKTNAITAILAAVTLCFASSQNITEEFYQSTCSAVSKGYLSALRTGWYTSVITIELSNIKENKCNGTDAKVKLIKQELDKYKSAVTELQLLMQSTPATNNKFLGFLLGVGSAIASGIAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPLAETCKVQSNRVFCDTMNSLTLPSEVNLCNIDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSREIIRAINIVRKIASEK

SEQ ID NO: 240 - protein, 509 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKQKQQAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSREIIRAINIVRKIASEK

SEQ ID NO: 241 - protein, 495 aa, synthetic construct
METPAQLLFLLLLWLPDTTGFASGQNITEEFYQSTCSAVSKGYLSALRTGWYTSVITIELSNIKKNKCNGTDAKVKLIKQELDKYKNAVTELQLLMQSTQATNNRARQQQQRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSREIIRAINIVRKIASEK

SEQ ID NO: 242 - protein, 495 aa, synthetic construct; identical to SEQ ID NO: 241

SEQ ID NO: 243 - protein, 519 aa, synthetic construct; identical to SEQ ID NO: 235

SEQ ID NO: 244 - protein, 518 aa, synthetic construct
MELLILKANAITTILTAVTFCFASQNITEEFYQSTCSAVSKGYLSALRTGWYTSVITIELSNIKENKCNGTDAKVKLIKQELDKYKNAVTELQLLMQSTPATNNRARRELPRFMNYTLNNAKKINVILSKKRKRRFLGFLLGVGSAIASGVAVCKVLHLEGEVNKIKSALLSINKAVVSLSNGVSVLIFKVLDLKNYIDKQLLPILNKQSCSISNIETVIEFQQKNNRLLEITREFSVNAGVITPVSTYMLINSELLSLINDMPITNDQKKLMSNNVQIVRQQSYSIMCIIKEEVLAYVVQLPLYGVIDTPCWKLHISPLCTINTKEGSNICLTRIDRGWYCDNAGSVSFFPQAETCKVQSNRVFCDTMNSLTLPSEVNLCNVDIFNPKYDCKIMISKTDVSSSVITSLGAIVSCYGKTKCIASNKNRGIIKTFSNGCDYVSNKGVDTVSVGNTLYYVNKQEGKSLYVKGEPIINFYDPLVFPSDEFDASISQVNEKINQSREIIRAINIVRKIASEK

SEQ ID NO: 245 - protein, 15 aa, synthetic construct
NNQARGSGSGRSLGF

SEQ ID NO: 246 - protein, 15 aa, synthetic construct
NNQARGGSGGRSLGF

SEQ ID NO: 247 - protein, 15 aa, synthetic construct
NNGARGGSGGRSLGF

SEQ ID NO: 248 - protein, 15 aa, synthetic construct
NNQARGGSGGDSLGF

SEQ ID NO: 249 - protein, 17 aa, synthetic construct
NNQARGGSGSGGDSLGF

SEQ ID NO: 250 - protein, 14 aa, synthetic construct
NNQARGGSGGGDLG

SEQ ID NO: 251 - protein, 16 aa, synthetic construct
NNQARGGSGSGGDLGF

SEQ ID NO: 252 - protein, 4 aa, synthetic construct
GSGS

SEQ ID NO: 253 - protein, 7 aa, synthetic construct
GGSGEKP

SEQ ID NO: 254 - protein, 7 aa, synthetic construct
GGSGQKP

SEQ ID NO: 255 - protein, 8 aa, synthetic construct
GGSGGSGS

SEQ ID NO: 256 - protein, 10 aa, synthetic construct
GGSGGSGEKP

SEQ ID NO: 257 - protein, 10 aa, synthetic construct
GGSGGSGQKP

SEQ ID NO: 258 - protein, 12 aa, synthetic construct
GGSGGSGGSGGS

SEQ ID NO: 259 - protein, 12 aa, synthetic construct
GSGGSGSGSGGS

SEQ ID NO: 260 - protein, 15 aa, synthetic construct
GGGGGSGGGSGGGGS

SEQ ID NO: 261 - protein, 15 aa, synthetic construct
GGGGSGGGGSGGGGS

SEQ ID NO: 262 - protein, 16 aa, synthetic construct
GGSGGSGSGGSGGSGS

SEQ ID NO: 263 - protein, 17 aa, synthetic construct
GGGGSGGGGSGGGGSGG

SEQ ID NO: 264 - protein, 18 aa, synthetic construct
SGGGSGGSGSGGSGGSGS

SEQ ID NO: 265 - protein, 19 aa, synthetic construct
EPEGGSGGSGSGGSGGSGS

SEQ ID NO: 266 - protein, 20 aa, synthetic construct
YGGSGGSGGSGSGGSGGSGS

SEQ ID NO: 267 - protein, 24 aa, synthetic construct
GGSGGSGSGGSGGSGSGGSGSGGS

SEQ ID NO: 268 - protein, 24 aa, synthetic construct
GSGGSGGSGGSGGSGSGGSGGSGS

SEQ ID NO: 269 - protein, 30 aa, synthetic construct
KSDELLGSGGSGSGSGGSEKAAKAEEAARK

SEQ ID NO: 270 - protein, 26 aa, synthetic construct
DKIEEILSKIYHIENEIARIKKLIGE

SEQ ID NO: 271 - protein, 22 aa, synthetic construct
EKFHQIEKEFSEVEGRIQDLEK

SEQ ID NO: 272 - protein, 28 aa, synthetic construct
EDKIEEILSKIYHIENEIARIKKLIGEA

SEQ ID NO: 273 - protein, 29 aa, synthetic construct
GSGYIPEAPRDGQAYVRKDGEWVLLSTFL

SEQ ID NO: 274 - protein, 33 aa, synthetic construct
RMKQIEDKIEEILSKIYHIENEIARIKKLIGEA

SEQ ID NO: 275 - protein, 36 aa, synthetic construct
RMKQIEDKIEEILSKIYHIENEIARIKKLIGERGGR

SEQ ID NO: 276 - protein, 37 aa, synthetic construct
RMKQIEDKIEEILSKIYHIENEIARIKKLIGNRTGGR

SEQ ID NO: 277 - protein, 37 aa, synthetic construct
RMKQIEDKIENITSKIYHIENEIARIKKLIGNRTGGR

SEQ ID NO: 278 - protein, 37 aa, synthetic construct
RMKQIEDKIEEILSKIYNITNEIARIKKLIGNRTGGR

SEQ ID NO: 279 - protein, 37 aa, synthetic construct
RMKQIEDKIENITSKIYNITNEIARIKKLIGNRTGGR

SEQ ID NO: 280 - protein, 550 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPACNNRARRELPRFMNYTLNNAKKTNVTLSKKRKRRFLGFLLGVGSACASGVAVSKVLHLEGEVNKIKSALLSTNKAVVSLSNGVSVLTIKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSSEFDASISQVNEKINQSREIIRAINIVRKIASEKSAIGGYIPEAPRDGQAYVRKDGEWVLLSTFL

SEQ ID NO: 281 - protein, 549 aa, synthetic construct
MELLILKANAITTILTAVTFCFASGQNITEEFYQSTCSAVSKGYLSALRTGWYHSVITIELSNIKENKCNGTDAKVKLIKQELDKYKNAVTELQLLMQSTPACNNRARRELPRFMNYTLNNAKKTNVTLSKKRKRRFLGFLLGVGSACASGVAVSKVLHLEGEVNKIKSALLSTNKAWSLSNGVSVLTIKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEILAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSSEFDASISQVNEKINQSREIIRAINIVRKIASEKSAIGGYIPEAPRDGQAYVRKDGEWVLLSTFL

SEQ ID NO: 282 - protein, 550 aa, synthetic construct
MELLILKANAITTILTAVTFCFASGQNITEEFYQSTCSAVSKGYLSALRTGWYHCVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVCTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSSEFDASISQVNEKINQSREIIRAINIVRKIASEKSAIGGYIPEAPRDGQAYVRKDGEWVLLSTFL

SEQ ID NO: 283 - protein, 550 aa, synthetic construct
MELLILKANAITTILTAVTFCFASGQNITEEFYQSTCSAVSKGYLSALRTGWYHCVITIELSNIKENKCNGTDAKVKLIKQELDKYKNAVTELQLLMQSTPATNNRARRELPRFMNYTLNNAKKTNVTLSKKRKRRFLGFLCGVGSAIASGVAVSKVLHLEGEVNKIKSALLSTNKAVVSLSNGVSVCTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEILAYVVQLPLYGVIDTPCWKLHTSPLCTTNTKEGSNICLTRTDRGWYCDNAGSVSFFPQAETCKVQSNRVFCDTMCSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSREIIRAINIVRKIASEKSAIGGYIPEAPRDGQAYVRKDGEWVLLSTFL

SEQ ID NO: 284 - protein, 549 aa, synthetic construct
MELLILKANAITTILTAVTFCFASGQNITEEFYQSTCSAVSKGYLSALRTGWYTCVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVCTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYWQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSSEFDASISQVNEKINQSREIIRAINIVRKIASEKSAIGGYIPEAPRDGQAYVRKDGEWVLLSTFL

SEQ ID NO: 285 - protein, 550 aa, synthetic construct
MELLILKANAITTILTAVTFCFASGQNITEEFYQSTCSAVSKGYLSALRTGWYHCVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVCTIKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSREIIRAINIVRKIASEKSAIGGYIPEAPRDGQAYVRKDGEWVLLSTFL

SEQ ID NO: 286 - protein, 550 aa, synthetic construct
MELLILKANAITTILTAVTFCFASGQNITEEFYQSTCSAVSKGYLSALRTGWYTCVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVCTIKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSSEFDASISQVNEKINQSREIIRAINIVRKIASEKSAIGGYIPEAPRDGQAYVRKDGEWVLLSTFL

SEQ ID NO: 287 - protein, 550 aa, synthetic construct
MELLILKANAITTILTAVTFCFASGQNITEEFYQSTCSAVSKGYLSALRTGWYHCVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVCTIKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSSEFDASISQVNEKINQSREIIRAINIVRKIASEKSAIGGYIPEAPRDGQAYVRKDGEWVLLSTFL

SEQ ID NO: 288 - protein, 550 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTIKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSSEFDASISQVNEKINQSREIIRAINIVRKIASEKSAIGGYIPEAPRDGQAYVRKDGEWVLLSTFL

SEQ ID NO: 289 - protein, 550 aa, synthetic construct
MELLILKANAITTILTAVTFCFASGQNITEEFYQSTCSAVSKGYLSALRTGWYHCVITIELSNIKENKCNGTDAKVKLIKQELDKYKNAVTELQLLMQSTPATNNRARRELPRFMNYTLNNAKKTNVTLSKKRKRRFLGFLCGVGSAIASGVAVSKVLHLEGEVNKIKSALLSTNKAVVSLSNGVSVCTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEILAYVVQLPLYGVIDTPCWKLHTSPLCTTNTKEGSNICLTRTDRGWYCDNAGSVSFFPQAETCKVQSNRVFCDTMCSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSSQFSASISQVNEKINQSREIIRAINIVRKIASEKSAIGGYIPEAPRDGQAYVRKDGEWVLLSTFL

SEQ ID NO: 290 - protein, 549 aa, synthetic construct
MELLILKANAITTILTAVTFCFASGQNITEEFYQSTCSAVSKGYLSALRTGWYHSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTIKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMCIIKEEILAYWQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSREIIRAINIVRKIASEKSAIGGYIPEAPRDGQAYVRKDGEWVLLSTFL

SEQ ID NO: 291 - protein, 549 aa, synthetic construct
MELLILKANAITTILTAVTFCFASGQNITEEFYQSTCSAVSKGYLSALRTGWYHCVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVCTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYWQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSSEFDASISQVNEKINQSREIIRAINIVRKIASEKSAIGGYIPEAPRDGQAYVRKDGEWVLLSTFL

SEQ ID NO: 292 - protein, 540 aa, synthetic construct
MELLILKAAITTILTAVTFCFASGQNITEEFYQSTCSAVSKGYLSALRTGWYTSCITIELSNIKENKCNGTDAVKLIKQELDKYKNAVTELQLLMQSTPATNNRARRELPRFMYTLNNAKKTVTLSKKRKRRFLGFLLGVGSAIASGVAVSKVLHLEGECNKIKSALLSTNKAVVSLSNGVSVLTSKVLDLKNYIDKQLLPIVKQSCSISNIETVIEFQQKNNRLLEITREFSVAGVTTPVSTYMLTNSELLSLINDMPITNDQKKLMSNNVQIVRQQSYSIMSIIKEEVLAYWQLPLYGVIDTPCWKLHTSPLCTTNTKEGSNICLTRTDRGWYCDNAGSVSFFPQAETCKVQSNRVFCDTMSLTLPSEVNLCNVDIFNPKYDCKIMTSKTDVSSSVITSLGAIVSCYGKTKCTASNKNRGIIKTFSNGCDYVSNKGVDTVSVGNTLYYVKQEGKSLYVKGEPIINFYDPLVFPSDEFDASISQVEKINQSREIIRAINIVRKIASEKSAIGGYIPEAPRDGQAYVRKDGEWVLLSTFL

SEQ ID NO: 293 - protein, 538 aa, synthetic construct
MELLILKAAITTILTAVTFCFASGQNITEEFYQSTCSAVSKGYLSALRTGWYTSVCTIELSNIKENKCNGTDAVKLIKQELDKYKNAVTELQLLMQSTPATNNRARRELPRFMYTLNNAKKTVTLSKKRKRRFLGFLLGVGSAIASGVAVSBVLHLEGEVKIKSALLSTNKAWSLSNGVSVLTCBVLDLKNYIDKQLLPIVKQSCSISNIETVIEFQQKNNRLLEITREFSVAGVTTPVSTYMLTNSELLSLINDMPITNDQKKLMSNNVQIVRQQSYSIMSIIKEEVLAYWQLPLYGVIDTPCWKLHTSPLCTTNTKEGSNICLTRTDRGWYCDNAGSVSFFPQAETCKVQSNRVFCDTMSLTLPSEVNLCNVDIFNPKYDCKIMTSKTDVSSSVITSLGAIVSCYGKTKCTASNKNRGIIKTFSNGCDYVSNKGVDTVSVGNTLYYVKQEGKSLYVKGEPIINFYDPLVFPSDEFDASISQVEKINQSREIIRAINIVRKIASEKSAIGGYIPEAPRDGQAYVRKDGEWVLLSTFL

SEQ ID NO: 294 - protein, 540 aa, synthetic construct
MELLILKAAITTILTAVTFCFASGQNITEEFYQSTCSAVSKGYLSALRTGWYTSVICIELSNIKENKCNGTDAVKLIKQELDKYKNAVTELQLLMQSTPATNNRARRELPRFMYTLNNAKKTVTLSKKRKRRFLGFLLGVGSAIASGVAVSKVLHLEGECNKIKSALLSTNKAVVSLSNGVSVLTSKVLDLKNYIDKQLLPIVKQSCSISNIETVIEFQQKNNRLLEITREFSVAGVTTPVSTYMLTNSELLSLINDMPITNDQKKLMSNNVQIVRQQSYSIMSIIKEEVLAYWQLPLYGVIDTPCWKLHTSPLCTTNTKEGSNICLTRTDRGWYCDNAGSVSFFPQAETCKVQSNRVFCDTMSLTLPSEVNLCNVDIFNPKYDCKIMTSKTDVSSSVITSLGAIVSCYGKTKCTASNKNRGIIKTFSNGCDYVSNKGVDTVSVGNTLYYVKQEGKSLYVKGEPIINFYDPLVFPSDEFDASISQVEKINQSREIIRAINIVRKIASEKSAIGGYIPEAPRDGQAYVRKDGEWVLLSTFL

SEQ ID NO: 295 - protein, 539 aa, synthetic construct
MELLILKAAITTILTAVTFCFASGQNITEEFYQSTCSAVSKGYLSALRTGWYTSVITIELSNIKENKCNGTDAVKLIKQELDKYKNAVTELQLLMQSTPATNNRARRELPRFMYTLNNAKKTVTLSKKRKRRFLGFLLGVGSAIASGVAVSBVLHLEGEVCKIKSALLSTNKAWSLSNGVSVLTSBVLDLKNYIDKQLLPIVKQSCSISNIETVIEFQQKNNRLLEITREFSVAGVTTPVSTYMLTNSELLSLINDMPITNDQKKLMSNNVQIVRQQSYSIMSIIKEECLAYWQLPLYGVIDTPCWKLHTSPLCTTNTKEGSNICLTRTDRGWYCDNAGSVSFFPQAETCKVQSNRVFCDTMSLTLPSEVNLCNVDIFNPKYDCKIMTSKTDVSSSVITSLGAIVSCYGKTKCTASNKNRGIIKTFSNGCDYVSNKGVDTVSVGNTLYYVKQEGKSLYVKGEPIINFYDPLVFPSDEFDASISQVEKINQSREIIRAINIVRKIASEKSAIGGYIPEAPRDGQAYVRKDGEWVLLSTFL

SEQ ID NO: 296 - protein, 537 aa, synthetic construct
MELLILKAAITTILTAVTFCFASGQNITEEFYQSTCSAVSKGYLSALRTGWYTSVITIELSNIKENKCNGTDAKVKLIKQELDKYKNAVTELQLLMQSTPATIWRARRELPRFMYTLAKKTVTLSKKRKRRFLGFLLGVGSAIASGVAVSBVLHLEGEVKICSALLSTNKAWSLSNGVSVLTSBVLDLKNYIDKQLLPIVKQSCSISNIETVIEFQQKNNRLLEITREFSVAGVTTPVSTYMLTNSELLSLINDMPITNDQKKLMSNNVQIVRQQSYSIMSIIKEECLAYWQLPLYGVIDTPCWKLHTSPLCTTNTKEGSNICLTRTDRGWYCDNAGSVSFFPQAETCKVQSNRVFCDTMSLTLPSEVNLCNVDIFNPKYDCKIMTSKTDVSSSVITSLGAIVSCYGKTKCTASNKNRGIIKTFSNGCDYVSNKGVDTVSVGNTLYYVKQEGKSLYVKGEPIINFYDPLVFPSDEFDASISQVEKINQSREIIRAINIVRKIASEKSAIGGYIPEAPRDGQAYVRKDGEWVLLSTFL

SEQ ID NO: 297 - protein, 539 aa, synthetic construct
MELLILKAAITTILTAVTFCFASGQNITEEFYQSTCSAVSKGYLSALRTGWYTSVITIELSNIKENKCNGTDAVKLIKQELDKYKNAVTELQLLMQSTPATNNRARRELPRFMYTLNNAKKTVTLSKKRKRRFLGFLLGVGSAIASGVAVSKVLHLEGEVKIKSALLSTNKAVVSLSNGVSVLTSKVLDLKNYIDKQLLPIVKQSCSISNIETVIEFQQKNNRLLEITREFSVAGVTTPVSTYMLTNSELLSLINDMPITNDQKKLMSNNVQIVRQQSYSIMSIIKEEVLAYWQLPLYGVIDTPCWKLHTSPLCTTNTKEGSNICLTRTDRGWYCDNAGSVSFFPQAETCKVQSNRVFCDTMSLTLPSEVNLCNVDIFNPKYDCKICTSKTDVSSSVITSLGAIVSCYGKTKCTASNKNRGIIKTFSNGCDYVSNKGVDTVSVGNTLYYVKQEGKSLYVKGEPIINFYDPLVCPSDEFDASISQVEKINQSREIIRAINIVRKIASEKSAIGGYIPEAPRDGQAYVRKDGEWVLLSTFL

SEQ ID NO: 298 - protein, 549 aa, synthetic construct
METPAQLLFLLLLWLPDTTGFASGQNITEEFYQSTCSAVSKGYLSALRTGWYTSVITIELSNIKKNKCNGTDAKVKLIKQELDKYKNAVTELQLLMQSTQATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSREIIRAINIVRKIASEKSAIGGYIPEAPRDGQAYVRKDGEWVLLSTFL

SEQ ID NO: 299 - protein, 549 aa, synthetic construct
METPAQLLFLLLLWLPDTTGFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSREIIRAINIVRKIASEKSAIGGYIPEAPRDGQAYVRKDGEWVLLSTFL

SEQ ID NO: 300 - protein, 550 aa, synthetic construct
MELLILKANAITTILTAVTFCFASGQNITEEFYQSTCSAVSKGYLSALRTGWYTSVITIELSNIKKNKCNGTDAKVKLIKQELDKYKNAVTELQLLMQSTQATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSREIIRAINIVRKIASEKSAIGGYIPEAPRDGQAYVRKDGEWVLLSTFL

SEQ ID NO: 301 - protein, 550 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSREIIRAINIVRKIASEKSAIGGYIPEAPRDGQAYVRKDGEWVLLSTFL

SEQ ID NO: 302 - protein, 527 aa, synthetic construct
MELLILKANAITTILTAVTFCFASGQNITEEFYQSTCSAVSKGYLSALRTGWYTSVITIELSNIKKNKCNGTDAKVKLIKQELDKYKNAVTELQLLMQSTQATNNRARQQQQRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSREIIRAINIVRKIASEKSAIGGYIPEAPRDGQAYVRKDGEWVLLSTFL

SEQ ID NO: 303 - protein, 520 aa, synthetic construct
MELLILKTNAITAILAAVTLCFASSQNITEEFYQSTCSAVSKGYLSALRTGWYTSVITIELSNIKENKCNGTDAKVKLIKQELDKYKSAVTELQLLMQSTPATNNKFLGFLLGVGSAIASGIAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPLAETCKVQSNRVFCDTMNSLTLPSEVNLCNIDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSREIIRAINIVRKIASEKSAIGGYIPEAPRDGQAYVRKDGEWVLLSTFL

SEQ ID NO: 304 - protein, 550 aa, synthetic construct; identical to SEQ ID NO: 301

SEQ ID NO: 305 - protein, 549 aa, synthetic construct
MELLILKANAITTILTAVTFCFASQNITEEFYQSTCSAVSKGYLSALRTGWYTSVITIELSNIKENKCNGTDAKVKLIKQELDKYKNAVTELQLLMQSTPATNNRARRELPRFMNYTLNNAKKINVILSKKRKRRFLGFLLGVGSAIASGVAVCKVLHLEGEVNKIKSALLSINKAVVSLSNGVSVLIFKVLDLKNYIDKQLLPILNKQSCSISNIETVIEFQQKNNRLLEITREFSVNAGVITPVSTYMLINSELLSLINDMPITNDQKKLMSNNVQIVRQQSYSIMCIIKEEVLAYVVQLPLYGVIDTPCWKLHISPLCTINTKEGSNICLTRIDRGWYCDNAGSVSFFPQAETCKVQSNRVFCDTMNSLTLPSEVNLCNVDIFNPKYDCKIMISKTDVSSSVITSLGAIVSCYGKTKCIASNKNRGIIKTFSNGCDYVSNKGVDTVSVGNTLYYVNKQEGKSLYVKGEPIINFYDPLVFPSDEFDASISQVNEKINQSREIIRAINIVRKIASEKSAIGGYIPEAPRDGQAYVRKDGEWVLLSTFL

SEQ ID NO: 306 - protein, 244 aa, synthetic construct
METDTLLLWVLLLWVPGSTGDYKDEKMEELFKKHKIVAVLRANSVEEAKKKALAVFLGGVDLIEITFTVPDADTVIKELSFLKEMGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPVEVAEKAKAFVEKIRGCTEQKLISEEDLHHHHHH

SEQ ID NO: 307 - protein, 244 aa, synthetic construct
METDTLLLWVLLLWVPGSTGDYKDEKMEELFKKHKIVAVLRANSVEEAKKDALAVFLGGVHLIEITFTVPDADTVIKELSFLKEMGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPVEVAEKAKAFVEKIRGCTEQKLISEEDLHHHHHH

SEQ ID NO: 308 - protein, 244 aa, synthetic construct
METDTLLLWVLLLWVPGSTGDYKDEKMEELFKKHKIVAVLRANSVEEAKKNALAVFLGGVHLIEITFTVPDADTVIKELSFLKEMGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPVEVAEKAKAFVEKIRGCTEQKLISEEDLHHHHHH

SEQ ID NO: 309 - protein, 244 aa, synthetic construct
METDTLLLWVLLLWVPGSTGDYKDEKMEELFKKHKIVAVLRANSVEEAKKKALAVFLGGVHLIEITFTVPDADTVIKELSFLKEMGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVQAVGVGSALVKGTPVEVAEKAKAFVEKIRGCTEQKLISEEDLHHHHHH

SEQ ID NO: 310 - protein, 244 aa, synthetic construct
METDTLLLWVLLLWVPGSTGDYKDEKMEELFKKHKIVAVLRANSVEEAKKKALAVFLGGVHLIEITFTVPDADTVIKELSFLKEMGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVQAVGVGEALNKGTPVEVAEKAKAFVEKIRGCTEQKLISEEDLHHHHHH

SEQ ID NO: 311 - protein, 244 aa, synthetic construct
METDTLLLWVLLLWVPGSTGDYKDEKMEELFKKHKIVAVLRANSVEEAKKKALAVFLGGVDLIEITFTVPDADTVIKELSFLKEMGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVQAVGVGEALNKGTPVEVAEKAKAFVEKIRGCTEQKLISEEDLHHHHHH

SEQ ID NO: 312 - protein, 239 aa, synthetic construct
METDTLLLWVLLLWVPGSTGDMEELFKEHKIVAVLRANSVEEAKKKALAVFLGGVHLIEITFTVPDADTVIKELSFLKEMGAIIGAGTVTSVEQAREAVESGAEFIVSPHLDEEISQFAKEEGVFYMPGVMTPTELVKAMKLGHTILKLFPGEVVGPQFVEAMKGPFPNVKFVPTGGVNLCNVAEWFEAGVLAVGVGSALVEGTPVEVAEKAKAFVEKIEGATEQKLISEEDLHHHHHH

SEQ ID NO: 313 - protein, 239 aa, synthetic construct
METDTLLLWVLLLWVPGSTGDMEELFKEHKIVAVLRANSVEEAKKKALAVFLGGVDLIEITFTVPDADTVIKELSFLKEMGAIIGAGTVTSVEQAREAVESGAEFIVSPHLDEEISQFAKEEGVFYMPGVMTPTELVKAMKLGHTILKLFPGEVVGPQFVEAMKGPFPNVKFVPTGGVNLDNVAEWFEAGVQAVGVGEALNEGTPVEVAEKAKAFVEKIEGATEQKLISEEDLHHHHHH

SEQ ID NO: 314 - protein, 39 aa, synthetic construct
LVFPSDEFDASISQVNEKINQSLAFIRRSDELLHNVNTG

SEQ ID NO: 315 - protein, 39 aa, synthetic construct
LVFPSDEFDASISQVNEKINQSLAFIRRSXXXXXXXXXX

SEQ ID NO: 316 - protein, 19 aa, synthetic construct
HHHHHHHHHHHHHHHHHHH

SEQ ID NO: 317 - protein, 20 aa, synthetic construct
ISQVNEKINQSLAFIRRSDE

SEQ ID NO: 318 - protein, 26 aa, synthetic construct
ISQVNEKINQSLAFIRRSDELLHNVN

SEQ ID NO: 319 - protein, 28 aa, synthetic construct
ISQVNEKINQSREIIRAINIVRKIASEK

SEQ ID NO: 320 - protein, 12 aa, synthetic construct
EKAAKAEEAARK